(19) United States
(12) Patent Application Publication
(10) Pub. No.: US 2014/0370011 A1
(43) Pub. Date: Dec. 18, 2014

(54) METHODS OF TREATING OR PREVENTING COGNITIVE IMPAIRMENT USING INDANE ACETIC ACID DERIVATIVES

(71) Applicant: DARA BioSciences, Inc., Raleigh, NC (US)

(72) Inventor: Mary Katherine Delmedico, Raleigh, NC (US)

(73) Assignee: DARA BIOSCIENCES, INC., Raleigh, NC (US)

(21) Appl. No.: 14/477,114

(22) Filed: Sep. 4, 2014

Related U.S. Application Data
(63) Continuation-in-part of application No. 14/013,801, filed on Aug. 29, 2013.
Application No. 14/013,801 is a continuation-in-part of application No. 13/375,878, filed on Feb. 1, 2012, now abandoned.
(60) Provisional application No. 61/184,157, filed on Jun. 4, 2009.

(51) Int. Cl. C07D 263/32; A61K 31/421; A61K 31/192; A61K 45/06

(52) U.S. Cl. CPC C07D 263/32; A61K 45/06; A61K 31/421; A61K 31/192
USPC 424/133.1; 514/374; 514/17.8; 514/224.8; 514/165

(57) ABSTRACT

The present invention provides indane acetic acid and their derivatives and methods for the treating and/or preventing of cognitive disorders.

This application is a Continuation-In-Part of U.S. application Ser. No. 14/013,801 filed on Aug. 29, 2013 which is a Continuation-In-Part of application Ser. No. 13/375,878 filed on Feb. 1, 2012, which is a 371 of PCT application number PCT/US2010/037227 filed on Jun. 3, 2010, which claims priority of U.S. provisional application No. 61/184,157 filed on Jun. 4, 2009. All applications are incorporated herein in their entirety by reference.

### COPYRIGHT NOTICE

A portion of the disclosure of this patent contains material that is subject to copyright protection. The copyright owner has no objection to the reproduction by anyone of the patent document or the patent disclosure as it appears in the Patent and Trademark Office patent files or records, but otherwise reserves all copyrights whatsoever.

### FIELD OF THE INVENTION

The present invention generally relates to the use of indane acetic acids and their derivatives to treat a cognitive disorder.

### BACKGROUND OF THE INVENTION

There are a wide variety of cognitive disorders that plague the general population and cause an impairment of cognitive ability. The cause of a cognitive disorder may be unknown or uncertain. In other cases, the cognitive disorder may be associated with (that is, be caused by or occur in the presence of) other conditions characterized by damage to or loss of neurons or other structures involved in the transmission of signals between neurons. Cognitive impairment or reduction of cognitive functions commonly occurs in association with central nervous system (CNS) disorders or conditions. Cognition generally refers to the process by which knowledge is acquired, retained and used by subjects and includes attention, memory, producing and understanding language, reasoning, problem solving and decision making. Cognitive disorders are associated with temporary or permanent brain dysfunction. Their main symptoms include problems with memory, orientation, language, information processing, and the ability to focus and sustain attention on a task. Examples of CNS disorders or conditions that fall within the scope of the present invention include, but are not limited to, age-associated memory impairment (AAMI); mild cognitive impairment (MCI), delirium (aka acute confusional state); dementia (sometimes further classified as Alzheimer's or non-Alzheimer's type dementia); Alzheimer's disease; Parkinson's disease; Huntington's disease (aka chorea); mental retardation; (e.g., Rubenstein-Taybi and Downs Syndrome); cerebrovascular disease (e.g., vascular dementia, post-cardiac surgery); corticobasal degeneration; Creutzfeldt-Jacob disease; frontotemporal lobar degeneration; Multiple Sclerosis; affective disorders; psychotic disorders; autism (aka Kanner's Syndrome); neurotic disorders; attention deficit disorder (ADD); subdural hematoma; normal-pressure hydrocephalus; organic chronic brain syndrome; Pick disease; progressive supranuclear palsy; brain tumor; head trauma (postconcussional disorder) and brain trauma (see DSM-IV, APA 1994). Amnestic and cognitive disorders with or without an established cause are described in DSM-TV. Other cognitive disorders specified in DSM-IV (Diagnostic and Statistical Manual of Mental Disorders. 4th Edition) include learning, motor skills and communication skills disorders (DSM-IV 315.00-315.39).

Hence, cognitive disorders may be associated with neurodegenerative diseases, trauma to the brain, or with other injury to the brain, such as that caused by infection (e.g., encephalitis, meningitis, septicemia) or drug intoxication or abuse. Cognitive disorders may also be associated with other conditions which impair normal functioning of the central nervous system, including psychiatric disorders such as anxiety disorders, dissociative disorders, mood disorders, schizophrenia, and somatoform and factitious disorders; it may also be associated with conditions of the peripheral nervous system, such as chronic pain.

For the purposes of the present invention the terms cognitive impairment and cognitive disorder are deemed to cover the same therapeutic indications. Accordingly, the terms cognitive impairment and cognitive disorder are used interchangeably throughout this application.

Accordingly, there is an ongoing need for an effective treatment for the impairment of cognitive ability caused by these disorders.

### SUMMARY OF THE INVENTION

The present invention provides methods of treating cognitive disorders. The methods include administering to a subject in need thereof an effective amount of a compound of Formula I:

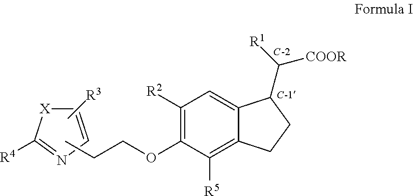

wherein in Formula I

R is H or C₁-C₆ alkyl;

R¹ is H, COOR, C₃-C₈ cycloalkyl, or
C₁-C₆ alkyl, C₂-C₆ alkenyl, or C₁-C₆ alkoxy, each of which may be unsubstituted or substituted with fluoro, methylenedioxyphenyl, or phenyl which may be unsubstituted or substituted with R⁶;

R² is H, halo, or C₁-C₆ alkyl which may be unsubstituted or substituted with C₁-C₆ alkoxy, oxo, fluoro, or

R² is phenyl, furyl, thienyl, pyrrolyl, oxazolyl, thiazolyl, imidazolyl, pyrazolyl, isoxazolyl, isothiazolyl, triazolyl, oxadiazolyl, thiadiazolyl, tetrazolyl, pyridyl, pyrrolidinyl, piperidinyl, tetrahydropyranyl, tetrahydrothiopyranyl, piperazinyl, or morpholinyl, each of which may be unsubstituted or substituted with R⁶;

R³ is H, CᵣC₆ alkyl, or phenyl, which may be unsubstituted or substituted with R⁶;

X is O or S;

R⁴ is C₁-C₆ alkyl or C₃-C₈ cycloalkyl, either of which may be unsubstituted or substituted with fluoro, oxo, or C₁-C₆ alkoxy which may be unsubstituted or substituted with C₁-C₆ alkoxy, or phenyl optionally substituted with R⁶, or
each of which may be substituted with phenyl, naphthyl, furyl, thienyl, pyrrolyl, tetrahydrofuryl, pyrrolidinyl, pyrrolinyl, tetrahydrothienyl, oxazolyl, thiazolyl, imidazolyl, pyrazolyl, isoxazolyl, isothiazolyl, triazolyl, oxadiazolyl, thiadiazolyl, tetrazolyl, pyridyl, piperidinyl, tetrahydropyranyl, tetrahydrothiopyranyl, pyrimidinyl, pyrazinyl, pyridazinyl, piperazinyl, morpholinyl, benzofuryl, dihydrobenzofuryl, benzothienyl, dihydrobenzothienyl, indolyl, indolinyl, indazolyl, benzoxazolyl, benzothiazolyl, benzimidazolyl, benzisoxazolyl, benzisothiazolyl, benzodioxolyl, quinolyl, isoquinolyl, quinazolinyl, quinoxazolinyl, dihydrobenzopyranyl, dihydrobenzothiopyranyl, or 1,4-benzodioxanyl,
each of which may be unsubstituted or further substituted with R⁶, or
C₁-C₆ alkyl may also be substituted with C₃-C₈ cycloalkyl or with phenoxy which may be unsubstituted or substituted with R⁶ or with phenyl, naphthyl, furyl, thienyl, pyrrolyl, tetrahydrofuryl, pyrrolidinyl, pyrrolinyl, tetrahydrothienyl, oxazolyl, thiazolyl, imidazolyl, pyrazolyl, isoxazolyl, isothiazolyl, triazolyl, oxadiazolyl, thiadiazolyl, tetrazolyl, pyridyl, piperidinyl, tetrahydropyranyl, tetrahydrothiopyranyl, pyrimidinyl, pyrazinyl, pyridazinyl, piperazinyl, morpholinyl, benzofuryl, dihydrobenzofuryl, benzothienyl, dihydrobenzothienyl, indolyl, indolinyl, indazolyl, benzoxazolyl, benxothiazolyl, benzimidazolyl, benzisoxazolyl, benzisothiazolyl, benzodioxolyl, quinolyl, isoquinolyl, quinazolinyl, quinoxazolinyl,
dihydrobenzopyranyl, dihydrobenzothiopyranyl, or 1,4-benzodioxanyl,
   each of which may be unsubstituted or substituted with R⁶, or

R⁴ is phenyl, naphthyl, furyl, thienyl, pyrrolyl, tetrahydrofuryl, pyrrolidinyl, pyrrolinyl, tetrahydrothienyl, oxazolyl, thiazolyl, imidazolyl, pyrazolyl, isoxazolyl, isothiazolyl, triazolyl, oxadiazolyl, thiadiazolyl, tetrazolyl, pyridyl, piperidinyl, tetrahydropyranyl, tetrahydrothiopyranyl, pyrimidinyl, pyrazinyl, pyridazinyl, piperazinyl, morpholinyl, benzofuryl, dihydrobenzofuryl, benzothienyl, dihydrobenzothienyl, indolyl, indolinyl, indazolyl, benzoxazolyl, benxothiazolyl, benzimidazolyl, benzisoxazolyl, benzisothiazolyl, benzodioxolyl, quinolyl, isoquinolyl, quinazolinyl, quinoxazolinyl, dihydrobenzopyranyl, dihydrobenzothiopyranyl, or 1,4-benzodioxanyl,
each of which may be unsubstituted or substituted with R⁶, or with phenyl, furyl, thienyl, pyrrolyl, oxazolyl, thiazolyl, imidazolyl, pyrazolyl, isoxazolyl, isothiazolyl, triazolyl, oxadiazolyl, thiadiazolyl, tetrazolyl, pyridyl, pyrrolidinyl, piperidinyl, tetrahydropyranyl, tetrahydrothiopyranyl, piperazinyl, morpholinyl, benzodioxolyl, dihydrobenzofuranyl, indolyl, pyrimidinyl or phenoxy,
each of which may be unsubstituted or substituted with R⁶;

R⁵ is H, halo or C₁-C₆ alkyl optionally substituted with oxo; and

R⁶ is halo, CF₃, C₁-C₆ alkyl optionally substituted with oxo or hydroxy, or C₁-C₆ alkoxy optionally substituted with fluoro;

or a pharmaceutically acceptable salt, ester, prodrug, stereoisomer, diastereomer, enantiomer, racemate or a combination thereof.

In one embodiment, the compound of Formula I is a meglumine, potassium or sodium salt thereof. In some embodiments, the compound of Formula I has the following structure:

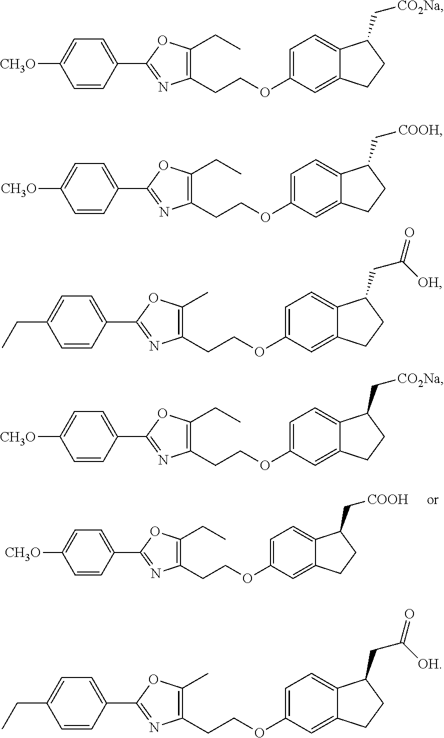

Another aspect of the present invention provides different methods of treating cognitive impairment. The methods include administering to a subject in need thereof an effective amount of a compound of Formula VI:

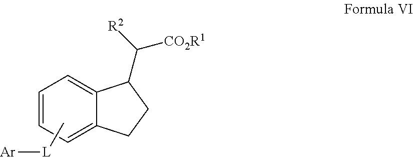

wherein

R¹ and R² are independently H, C₁-C₆ alkyl, or C₃-C₆ cycloalkyl;

L is a linker and selected from the group consisting of —(CH₂)ₘ—X—, —Y—(CH₂)ₙ—X—, and

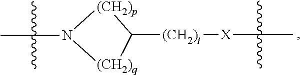

wherein
X is selected from the group O, S, S(═O), and S(═O)₂,
Y is selected from the group O, NR⁵, S, S(═O), and S(═O)₂,
m is 1, 2, or 3,
n is 2, 3, or 4,
t is 0 or 1,
p is 0, 1, 2, or 3,
q is 1, 2, 3, or 4,
   wherein the sum of p and q is 1, 2, 3, or 4;

Ar is phenyl or a 6-membered heteroaryl containing up to three N atoms,
wherein said Ar is optionally substituted at any available position by 1 to 5 independently selected R³ groups, and
optionally fused to a 5- or 6-membered saturated carbocyclic ring,
a 5- or 6-membered unsaturated carbocyclic ring, or
a 5- or 6-membered heterocyclic ring containing up to 3 additional heteroatoms selected from N, O, and S,
   wherein said fused ring may be optionally substituted at any available position by 1 to 4 independently selected R⁴ groups;

R³ is selected from the group consisting of hydroxy, SH, halo, CN, NO₂, C(═O)OH, C(═O)—OC₁-C₆ alkyl, C(═O)—OC₃-C₆ cycloalkyl, NR⁶R⁷, C(═O)NR⁶R⁷, C(═S)NR⁶R⁷, C₁-C₆ alkyl optionally substituted with halo, OH, NR⁶R⁷, or C₁-C₆ alkoxy, C₁-C₆ haloalkyl, C₁-C₆ alkoxy, C₁-C₆ thioalkyl, C₂-C₆ alkenyl, C₁-C₆ haloalkoxy, C₃-C₈ cycloalkyl, C₃-C₈ cycloalkoxy, phenoxy optionally substituted on the phenyl ring with halo, C₁-C₆ alkyl, or Cᵣ C₆ alkoxy, and
a mono or bicyclic ring radical selected from the group consisting of
   a) phenyl optionally fused to
      a 5- or 6-membered saturated or partially unsaturated carbocylic ring, or
      a 5- or 6-membered saturated or partially unsaturated heterocyclic ring containing from 1-3 heteroatoms selected from N, O, and S,
   b) a 5- or 6-membered heterocyclic ring radical containing up to 4 heteroatoms selected from N, O, or S, optionally fused to
      a 5- or 6-membered saturated or partially unsaturated carbocylic ring, or
      a 5- or 6-membered saturated or partially unsaturated heterocyclic ring containing from 1-3 heteroatoms selected from N, O, and S,
   said mono or bicyclic ring radical being optionally substituted with up to 5 groups independently selected from the group consisting of halo, hydroxy, oxo, CN, C₁-C₆ alkyl optionally substituted with halo, OH, NR⁶R⁷, C₁-C₆ alkoxy, C₁-C₆ haloalkyl, C₁-C₆ alkoxy, C₁-C₆ thioalkyl, C₁-C₆ haloalkoxy, C₃-C₈ cycloalkyl, C₃-C₈ cycloalkoxy, C₁-C₆ acyl, C(═O)OH, CH₂C(═O)OH, NR⁶R⁷, C(═O)NR⁶R⁷, C(═O)OC₃-C₆ alkyl, and C(═O)OC₃-C₆ cycloalkyl;

R⁴ is selected from the group consisting of oxo, hydroxy, halo, CN, NR⁶R⁷, C₁-C₆ alkyl optionally substituted with OH, NR⁶R⁷, or C₁-C₆ alkoxy, C₁-C₆ haloalkyl, C₁-C₆ alkoxy, C₁-C₆ thioalkyl, C₁-C₆ haloalkoxy, C₃-C₈ cycloalkyl, and C₃-C₈ cycloalkoxy;

R⁵ is selected from the group consisting of H, C₁-C₆ alkyl optionally substituted with C₃-C₆ cycloalkyl, C₁-C₆ acyl, benzyl optionally substituted with halo, C₁-C₆ alkoxy, (C₁-C₆)alkyl, CN, NH₂, N[(C₁-C₃)alkyl]₂, NO₂, or CF₃, C₃-C₆ cycloalkyl, and C(═O)OC₁-C₆ alkyl;

R⁶ and R⁷ are independently selected from the group consisting of H, C₁-C₆ alkyl optionally substituted with C₃-C₆ cycloalkyl, C₁-C₆ acyl, benzyl optionally substituted with halo, C₁-C₆ alkoxy, (C₁-C₆)alkyl, CN, NH₂, N[(C₁-C₃)alkyl]₂, NO₂, or CF₃, C₃-C₆ cycloalkyl, and phenyl optionally substituted with halo, C₁-C₆ alkoxy, (C₁-C₆)alkyl, CN, N[(C₁-C₃)alkyl]₂, NO₂, or CF₃, or

R⁶ and R⁷ may be taken together with the nitrogen atom to which they are attached to form a 5- or 6-membered heterocyclic ring optionally interrupted by NR⁵ or O;

or a pharmaceutically acceptable salt, ester prodrug, stereoisomer, diastereomer, enantiomer, racemate or a combination thereof.

In some embodiments, the compound of formula (VI) is alkali metal salt, or a basic nitrogen containing group.

In some embodiments, the compound of formula (VI) is a meglumine, caclsium, magnesium, ammonium salts, potassium or sodium salt thereof.

In one embodiment, the compound of formula (VI) has the structure:

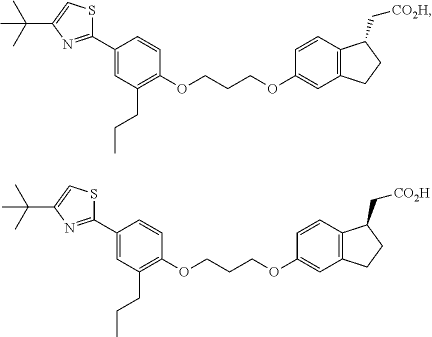 [SMILES: CCCc1cc(-c2nc(C(C)(C)C)cs2)ccc1OCCCOc1ccc2c(c1)CC[C@H]2CC(=O)O ; CCCc1cc(-c2nc(C(C)(C)C)cs2)ccc1OCCCOc1ccc2c(c1)CC[C@@H]2CC(=O)O]

or a pharmaceutically acceptable salt thereof.

In another embodiment, the methods described herein may further include administration of one or more additional therapeutic agent.

Objects of the present invention will be appreciated by those of ordinary skill in the art from a reading of the Examples and the detailed description of the embodiments, which follow, such description being merely illustrative of the present invention.

### DETAILED DESCRIPTION

The foregoing and other aspects of the present invention will now be described in more detail with respect to the description and methodologies provided herein. It should be appreciated that the invention can be embodied in different forms and should not be construed as limited to the embodiments set forth herein. Rather, these embodiments are provided so that this disclosure will be thorough and complete, and will fully convey the scope of the invention to those skilled in the art.

The terminology used in the description of the invention herein is for the purpose of describing particular embodiments only and is not intended to be limiting of the invention. As used in the description of the embodiments of the invention and the appended claims, the singular forms “a”, “an” and “the” are intended to include the plural forms as well, unless the context clearly indicates otherwise. Also, the absence of articles “a”, “an” are intended to include both the singular forms and plural forms. Also, as used herein, “and/or” refers to and encompasses any and all possible combinations of one or more of the associated listed items. Furthermore, the term “about,” as used herein when referring to a measurable value such as an amount of a compound, dose, time, temperature, and the like, is meant to encompass variations of 20%, 10%, 5%, 1%, 0.5%, or even 0.1% of the specified amount. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. Unless otherwise defined, all terms, including technical and scientific terms used in the description, have the same meaning as commonly understood by one of ordinary skill in the art to which this invention belongs.

Generally, the nomenclature used herein and the laboratory procedures in organic chemistry, medicinal chemistry, and pharmacology described herein are those well known and commonly employed in the art. Unless defined otherwise, all technical and scientific terms used herein generally have the same meaning as commonly understood by one of ordinary skill in the art to which this disclosure belongs. In the event that there is a plurality of definitions for a term used herein, those in this section prevail unless stated otherwise.

All patents, patent applications and publications referred to herein are incorporated by reference in their entirety. In case of a conflict in terminology, the present specification is controlling.

### A. Definitions

The term “halo” means F, Cl, Br, or I.

The term “C₁-C₆ alkyl” means a straight or branched saturated hydrocarbon carbon chain of from 1 to about 6 carbon atoms, respectively. Examples of such groups include methyl, ethyl, isopropyl, sec-butyl, 2-methylpentyl, n-hexyl, and the like.

The term “C₂-C₆ alkenyl” means a straight or branched unsaturated hydrocarbon carbon chain of from 2 to about 6 carbon atoms. Examples of such groups include vinyl, allyl, isopropenyl, 2-butenyl, 3-ethyl-2-butenyl, 4-hexenyl, and the like.

The term “C₁-C₆ haloalkyl” means a C₁-C₆ alkyl group substituted by 1 to 3 halogen atoms or fluorine up to the perfluoro level. Examples of such groups include trifluoromethyl, tetrafluoroethyl, 1,2-dichloropropyl, 5-bromopentyl, 6-iodohexyl, and the like.

The terms “C₃-C₆ cycloalkyl” and “C₃-C₈ cycloalkyl” mean a saturated carbocyclic ring system of from 3 to about 6 carbon atoms or from 3 to about 8 carbon atoms, respectively. Examples of such groups include cyclopropyl, cyclobutyl, cyclopentyl, cyclohexyl, and the like.

The term “C₁-C₆ acyl” means a C₁-C₆ alkyl group attached at the carbonyl carbon atom. The radical is attached to the rest of the molecule at the carbonyl bearing carbon atom. Examples of such groups include acetyl, propionyl, n-butanoyl, 2-methylpentantoyl, and the like.

The term “C₁-C₆ alkoxy” means a linear or branched saturated carbon group having from 1 to about 6 C atoms, said carbon group being attached to an O atom. The O atom is the point of attachment of the alkoxy substituent to the rest of the molecule. Such groups include, but are not limited to, methoxy, ethoxy, n-propoxy, isopropoxy, and the like.

The term “C₁-C₆ thioalkyl” means a linear or branched saturated carbon group having from 1 to about 6 C atoms, said carbon group being attached to an S atom. The S atom is the point of attachment of the thioalkyl substituent to the rest of the molecule. Such groups include, for example, methylthio, propylthio, hexylthio, and the like.

The term “C₁-C₆ haloalkoxy” means a C₁-C₆ alkoxy group further substituted on C with 1 to 3 halogen atoms or fluorine up to the perfluoro level.

The term “C₃-C₈ cycloalkoxy” means a C₃-C₈ cycloalkyl group attached to an O atom. The O atom is the point of attachment of the cycloalkoxy group with the rest of the molecule.

The term “phenoxy” means a phenyl group attached to an O atom. The O atom is the point of attachment of the phenoxy group to the rest of the molecule.

The term “6-membered heteroaryl ring” means a 6-membered monocyclic heteroaromatic ring radical containing 1-5 carbon atoms and up to the indicated number of N atoms. Examples of 6-membered heteroaryl rings are pyridyl, pyrimidyl, pyridazinyl, pyrazinyl, triazinyl, and the like.

The term “5- or 6-membered heterocyclic ring” means a 5 or 6-membered ring containing 1-5 C atoms and up to the indicated number of N, O, and S atoms, and may be aromatic, partially saturated, or fully saturated.

The term “optionally substituted” means that, unless indicated otherwise, the moiety so modified may have from one to up to the number of the substituents indicated, provided the resulting substitution is chemically feasible as recognized in the art. Each substituent may replace any H atom on the moiety so modified as long as the replacement is chemically possible and chemically stable. For example, a chemically unstable compound would be one where each of two substituents is bonded to a single C atom through each substituents heteroatom. Another example of a chemically unstable compound would be one where an alkoxy group is bonded to the unsaturated carbon of an alkene to form an enol ether. When there are two or more substituents on any moiety, each substituent is chosen independently of the other substituent so that, accordingly, the substituents can be the same or different.

When the 5- or 6-membered heterocyclic ring is attached to the rest of the molecule as a substituent, it becomes a radical. Examples of 5- or 6-membered heteroaryl ring radicals are furyl, pyrrolyl, thienyl, pyrazolyl, isoxazolyl, imidazolyl, oxazolyl, thiazolyl, isothiazolyl, triazolyl, thiadiazolyl, oxadiazolyl, pyridyl, pyrimidyl, pyridazinyl, pyrazinyl, triazinyl, and the like. Examples of partially unsaturated 5- or 6-membered heterocyclic ring radicals include dihydropyrano, pyrrolinyl, pyrazolinyl, imidazolinyl, dihydrofuryl, and the like. Examples of saturated 5- or 6-membered heterocyclic ring radicals include pyrrolidinyl, tetrahydropyridyl, piperidinyl, morpholinyl, tetrahydrofuryl, tetrahydrothienyl, piperazinyl, and the like. The point of attachment of the radical may be from any available C or N atom of the ring to the rest of the molecule. When the 5- or 6-membered heterocyclic ring is fused to another ring contained in the rest of the molecule, it forms a bicyclic ring. Examples of such 5- and 6-heterocyclic fused rings include pyrrolo, furo, pyrido, piperido, thieno, and the like. The point of fusion is at any available face of the heterocyclic ring and parent molecule.

As used herein, “subject”, as used herein, means a mammalian subject (e.g., dog, cat, horse, cow, sheep, goat, monkey, etc.), and particularly human subjects (including both male and female subjects, and including neonatal, infant, juvenile, adolescent, adult and geriatric subjects, and further including various races and ethnicities including, but not limited to, white, black, Asian, American Indian and Hispanic).

To “treat,” as used here, means to deal with medically. It includes, for example, administering a compound of the invention to prevent the onset of a cognitive impairment, to alleviate its severity, and to prevent its reoccurrence.

The term “cognitive disorder,” or “cognitive impairment” as used here, means any condition characterized by a deficit in mental activities associated with thinking, learning, or memory. Examples of such disorders include agnosias, amnesias, aphasias, apraxias, deliriums, dementias, and learning disorders.

The compounds described here may be used to treat agnosias, amnesias, aphasias, apraxias, deliriums, dementias, learning disorders and other cognitive disorders regardless of whether their cause is known or not.

The compounds described here may also be used to treat patient having deficits in mental activities that are mild or that otherwise do not significantly interfere with daily life. Mild cognitive impairment is an example of such a condition: a patient with mild cognitive impairment displays symptoms of dementia (e.g., difficulties with language or memory) but the severity of these symptoms is such that a diagnosis of dementia may not be appropriate. The compounds described here may be used to treat mild cognitive impairment and other, similarly less severe forms of cognitive disorders/impairment.

As used herein, “an effective amount” refers to an amount that causes relief of symptoms of a disorder or disease as noted through clinical testing and evaluation, patient observation, and/or the like. An “effective amount” can further designate a dose that causes a detectable change in biological or chemical activity. The detectable changes may be detected and/or further quantified by one skilled in the art for the relevant mechanism or process. Moreover, an “effective amount” can designate an amount that maintains a desired physiological state, i.e., reduces or prevents significant decline and/or promotes improvement in the condition of interest. An “effective amount” can further refer to a therapeutically effective amount.

### B. Compounds

### (I). Formula I

The present invention encompasses the compounds of Formula I,

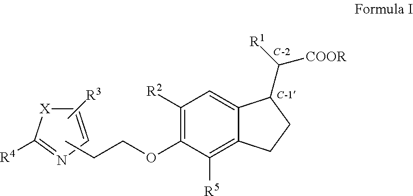

wherein in Formula I

R is H or C₁-C₆ alkyl;

R¹ is H, COOR, C₃-C₈ cycloalkyl, or C₁-C₆ alkyl, C₂-C₆ alkenyl, or C₁-C₆ alkoxy each of which may be unsubstituted or substituted with fluoro, methylenedioxyphenyl, or phenyl which may be unsubstituted or substituted with R⁶;

R² is H, halo, or C₁-C₆ alkyl which may be unsubstituted or substituted with C₁-C₆ alkoxy, oxo, fluoro, or

R² is phenyl, furyl, thienyl, pyrrolyl, oxazolyl, thiazolyl, imidazolyl, pyrazolyl, isoxazolyl, isothiazolyl, triazolyl, oxadiazolyl, thiadiazolyl, tetrazolyl, pyridyl, pyrrolidinyl, piperidinyl, tetrahydropyranyl, tetrahydrothiopyranyl, piperazinyl, or morpholinyl,
each of which may be unsubstituted or substituted with R⁶;

R³ is H, C₁-C₆ alkyl, or phenyl, which may be unsubstituted or substituted with R⁶;

X is O or S;

R⁴ is C₁-C₆ alkyl or C₃-C₈ cycloalkyl, either of which may be unsubstituted or substituted with fluoro, oxo, or C₁-C₆ alkoxy which may be unsubstituted or substituted with C₁-C₆ alkoxy, or phenyl optionally substituted with R⁶,
each of which may be substituted with phenyl, naphthyl, furyl, thienyl, pyrrolyl, tetrahydrofuryl, pyrrolidinyl, pyrrolinyl, tetrahydrothienyl, oxazolyl, thiazolyl, imidazolyl, pyrazolyl, isoxazolyl, isothiazolyl, triazolyl, oxadiazolyl, thiadiazolyl, tetrazolyl, pyridyl, piperidinyl, tetrahydropyranyl, tetrahydrothiopyranyl, pyrimidinyl, pyrazinyl, pyridazinyl, piperazinyl, morpholinyl, benzofuryl, dihydrobenzofuryl, benzothienyl, dihydrobenzothienyl, indolyl, indolinyl, indazolyl, benzoxazolyl, benzothiazolyl, benzimidazolyl, benzisoxazolyl, benzisothiazolyl, benzodioxolyl, quinolyl, isoquinolyl, quinazolinyl, quinoxazolinyl, dihydrobenzopyranyl, dihydrobenzothiopyranyl, or 1,4-benzodioxanyl,
   each of which may be unsubstituted or further substituted with R⁶, or
C₁-C₆ alkyl may also be substituted with C₃-C₈ cycloalkyl or with phenoxy which may be unsubstituted or substituted with R⁶ or with phenyl, naphthyl, furyl, thienyl, pyrrolyl, tetrahydrofuryl, pyrrolidinyl, pyrrolinyl, tetrahydrothienyl, oxazolyl, thiazolyl, imidazolyl, pyrazolyl, isoxazolyl, isothiazolyl, triazolyl, oxadiazolyl, thiadiazolyl, tetrazolyl, pyridyl, piperidinyl, tetrahydropyranyl, tetrahydrothiopyranyl, pyrimidinyl, pyrazinyl, pyridazinyl, piperazinyl, morpholinyl, benzofuryl, dihydrobenzofuryl, benzothienyl, dihydrobenzothienyl, indolyl, indolinyl, indazolyl, benzoxazolyl, benxothiazolyl, benzimidazolyl, benzisoxazolyl, benzisothiazolyl, benzodioxolyl, quinolyl, isoquinolyl, quinazolinyl, quinoxazolinyl,
dihydrobenzopyranyl, dihydrobenzothiopyranyl, or 1,4-benzodioxanyl,
   each of which may be unsubstituted or substituted with R⁶, or
R⁴ is phenyl, naphthyl, furyl, thienyl, pyrrolyl, tetrahydrofuryl, pyrrolidinyl, pyrrolinyl, tetrahydrothienyl, oxazolyl, thiazolyl, imidazolyl, pyrazolyl, isoxazolyl, isothiazolyl, triazolyl, oxadiazolyl, thiadiazolyl, tetrazolyl, pyridyl, piperidinyl, tetrahydropyranyl, tetrahydrothiopyranyl, pyrimidinyl, pyrazinyl, pyridazinyl, piperazinyl, morpholinyl, benzofuryl, dihydrobenzofuryl, benzothienyl, dihydrobenzothienyl, indolyl, indolinyl, indazolyl, benzoxazolyl, benxothiazolyl, benzimidazolyl, benzisoxazolyl, benzisothiazolyl, benzodioxolyl, quinolyl, isoquinolyl, quinazolinyl, quinoxazolinyl, dihydrobenzopyranyl, dihydrobenzothiopyranyl, or 1,4-benzodioxanyl,
   each of which may be unsubstituted or substituted with R⁶, or with phenyl, furyl, thienyl, pyrrolyl, oxazolyl, thiazolyl, imidazolyl, pyrazolyl, isoxazolyl, isothiazolyl, triazolyl, oxadiazolyl, thiadiazolyl, tetrazolyl, pyridyl, pyrrolidinyl, piperidinyl, tetrahydropyranyl, tetrahydrothiopyranyl, piperazinyl, morpholinyl, benzodioxolyl, dihydrobenzofuranyl, indolyl, pyrimidinyl or phenoxy,
each of which may be unsubstituted or substituted with R⁶;

R⁵ is H, halo or C₁-C₆ alkyl optionally substituted with oxo; and

R⁶ is halo, CF₃, C₁-C₆ alkyl optionally substituted with oxo or hydroxy, or
C₁-C₆ alkoxy optionally substituted with fluoro;
   or a pharmaceutically acceptable salt, ester prodrug, stereoisomer, diastereomer, enantiomer, racemate or a combination thereof.

R³ may be attached to the heterocyclic moiety of the compound of Formula I at either the 4 or 5 position (i.e., at either available carbon atom) and, accordingly, the remaining portion of the molecule will be attached at the remaining available carbon atom.

In some embodiments, the compound of Formula I has the following structure:

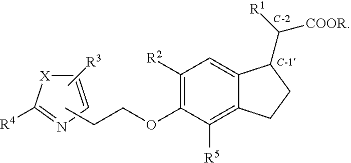

In some embodiments, the compound of Formula I is a meglumine, potassium or sodium salt thereof.

In other embodiments, for the compound of Formula I, R is H, R¹ is H, R² is H, R³ is C₁-C₆ alkyl, X is O, and R⁴ is a phenyl substituted with R⁶, wherein R⁶ is C₁-C₆ alkoxyl or C₁-C₆ alkyl, or a pharmaceutically acceptable salt thereof.

In one embodiment, the compound has the following structure:

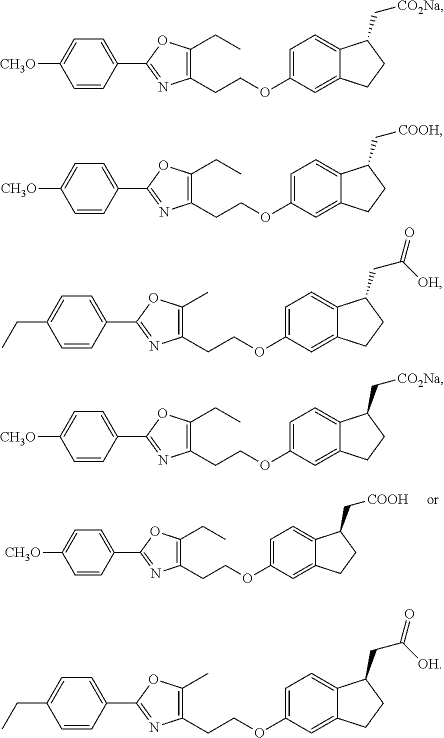

In other embodiment, the compound of Formula I is a meglumine, potassium or sodium salt of the structure

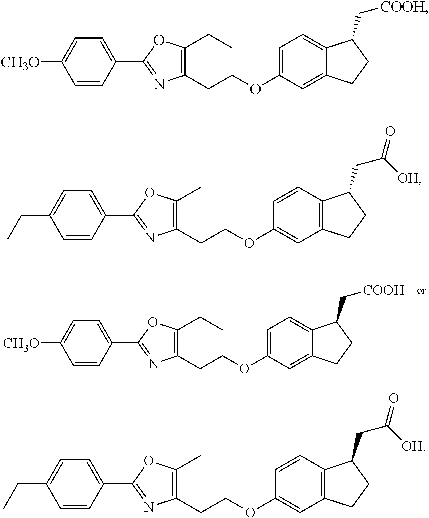

Exemplary compounds of Formula I are listed in Table 1.

TABLE 1
Illustrative Examples of Compounds of Formula I

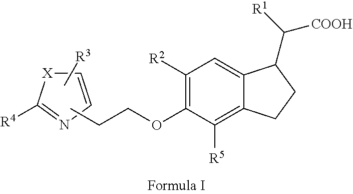

| Entry | | | | | | |
|---|---|---|---|---|---|---|
| No. | R¹ | R² | R³ | R⁴ | R⁵ | X |
| 1 | H | H | H | CH₃ | H | O |
| 2 | H | H | H | n-butyl | H | O |
| 3 | H | H | H | cyclopropyl | H | O |
| 4 | H | H | H | cyclopentyl | H | O |
| 5 | H | H | H | cyclooctyl | H | O |
| 6 | H | H | H | Ph | H | O |
| 7 | H | H | H | Ph | H | S |
| 8 | H | H | H | 2-Cl Ph | H | O |
| 9 | H | H | H | 2,3-d-F Ph | H | O |
| 10 | H | H | H | 2,4-di-CH₃ Ph | H | O |
| 11 | H | H | H | 2-thienyl | H | O |
| 12 | H | H | H | 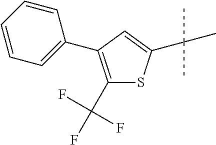 | H | O |
| 13 | H | H | H | 2-furyl | H | O |
| 14 | H | H | H | 2-furyl | H | S |
| 15 | H | H | H | 2-(4-CH₃)furyl | H | O |
| 16 | H | H | H | 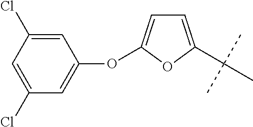 | H | O |
| 17 | H | H | H | 4-F Ph | H | O |
| 18 | H | H | H | 4-F Ph | H | S |
| 19 | H | H | CH₃ | 4-F Ph | H | O |
| 20 | H | H | Et | 4-F Ph | H | O |
| 21 | H | H | Et | 4-F Ph | H | S |
| 22 | H | H | Et | 3-pyridyl | H | O |
| 23 | H | H | Et | 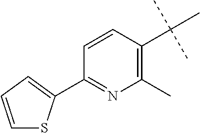 | H | O |
| 24 | H | H | isopropyl | 4-F Ph | H | O |
| 25 | H | H | isopropyl | 2,4-di-F Ph | H | O |
| 26 | H | H | n-butyl | 2,4-di-F Ph | H | O |
| 27 | H | H | n-hexyl | 2,4-di-F Ph | H | O |
| 28 | H | H | Ph | 2,4-di-F Ph | H | O |
| 29 | H | H | 4-F Ph | 2,4-di-F Ph | H | O |
| 30 | H | CH₃ | Et | Ph | H | O |
| 31 | H | CH₃ | Et | Ph | H | S |
| 32 | H | CH₃ | Et | 3-CH₃O Ph | H | O |
| 33 | H | H | Et | 3-CH₃O Ph | H | O |
| 34 | H | H | Et | 3-CH₃O Ph | H | S |
| 35 | H | H | Et | 4-CH₃O Ph | H | O |
| 36 | H | H | Et | 4-CH₃O Ph | H | S |
| 37 | H | H | Et | 4-EtO Ph | H | S |
| 38 | H | H | Et | 4-EtO Ph | H | O |
| 39 | H | H | Me | 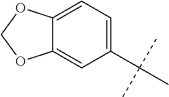 | H | O |
| 40 | H | H | Me | PhCH₂ | H | O |
| 41 | H | H | Me | 3-Cl-4-F-Ph | H | O |
| 42 | H | H | Me | 3-F-4-Me-Ph | H | O |
| 43 | H | H | Me | 3-Me-4-F-Ph | H | O |
| 44 | H | H | Me | 3-NH₂-4-Me-Ph | H | O |
| 45 | H | H | Et | 4-Et-Ph | H | O |
| 46 | H | H | Me | 4-Et-Ph | H | O |
| 47 | H | H | Et | 4-CN-Ph | H | O |
| 48 | H | H | Et | 4-(Et)₂N-Ph | H | O |
| 49 | H | H | Me | 4-i-Pr-Ph | H | O |
| 50 | H | H | Me | 4-t-Bu-Ph | H | O |
| 51 | H | H | Me | 4-Et-Ph | H | O |
| 52 | H | H | Me | 4-n-Bu-Ph | H | O |
| 53 | H | H | Et | 4-n-Pr-Ph | H | O |
| 54 | H | CH₃ | Et | 4-CH₃O Ph | H | O |
| 55 | H | CH₃ | Et | 4-CH₃O Ph | H | S |
| 56 | H | CH₃ | Et | 4-CH₃O Ph | CH₃ | O |
| 57 | H | CH₃ | Et | 3,4-di-CH₃O Ph | CH₃ | O |
| 58 | H | CH₃ | Et | 4-Ph Ph | CH₃ | O |
| 59 | H | CH₃ | Et | 4-Ph Ph | CH₃ | S |
| 60 | H | CH₃ | Et | 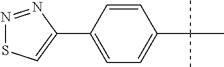 | CH₃ | O |
| 61 | H | CH₃ | Ph | cyclopropyl | H | O |
| 62 | H | CH₃ | Ph | cyclohexyl | H | O |
| 63 | H | CH₃ | Ph | cyclohexyl | H | S |
| 64 | H | CH₃ | p-F Ph | cyclohexyl | H | O |
| 65 | H | Cl | i-Pr | Ph | H | O |
| 66 | H | Cl | i-Pr | Ph | H | S |
| 67 | H | Cl | i-Pr | Ph | Cl | O |
| 68 | H | Cl | i-Pr | 4-CH₃ Ph | Cl | O |
| 69 | H | Br | CH₃ | Ph | Br | O |
| 70 | H | Br | CH₃ | 3-F Ph | Br | O |
| 71 | H | Br | CH₃ | 3-F Ph | Br | S |
| 72 | H | CH₃CO | CH₃ | n-propyl | CH₃CO | O |
| 73 | H | CH₂OCH₃ | Et | 2-thienyl | H | O |
| 74 | H | Ph | H | 2,4-di-Cl Ph | H | O |
| 75 | H | Ph | H | 2,4-di-Cl Ph | H | S |
| 76 | H | Ph | CH₃ | 2,4-di-Cl Ph | H | O |
| 77 | H | Ph | Et | 2,4-di-Cl Ph | H | O |
| 78 | H | Ph | Ph | 2,4-di-Cl Ph | H | O |
| 79 | H | Ph | Ph | 2,4-di-Cl Ph | H | S |
| 80 | H | Ph | 4-CH₃O-Ph | 2,4-di-Cl Ph | H | O |
| 81 | H | 4-F Ph | CH₃ | 4-F Ph | H | O |
| 82 | H | 4-F Ph | CH₃ | 2,4-di-Cl Ph | H | O |
| 83 | H | 3-pyridyl | CH₃ | 2,4-di-Cl Ph | H | O |
| 84 | H | 3-pyridyl | CH₃ | 2,4-di-Cl Ph | H | S |
| 85 | H | 2-thienyl | CH₃ | Ph | H | O |
| 86 | H | 2-thienyl | CH₃ | 2,4-di-Cl Ph | H | O |
| 87 | H | 2-thienyl | CH₃ | 2,4-di-Cl Ph | H | S |
| 88 | H | 2-thienyl | CH₃ | 3-pyridyl | H | O |
| 89 | H | 2-thienyl | CH₃ | cyclopentyl | H | O |
| 90 | H | 2-thienyl | CH₃ | 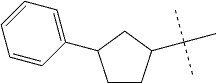 | H | O |
| 91 | H | 2-thienyl | CH₃ | Ph | 2-thienyl | O |
| 92 | CH₃ | H | H | Ph | H | O |
| 93 | CH₃ | H | H | Ph | H | S |
| 94 | CH₃ | H | H | 2-thienyl | H | O |
| 95 | CH₃ | H | H | 2-thienyl | H | S |
| 96 | CH₃ | H | H | 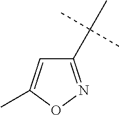 | H | O |
| 97 | CH₃ | H | H | 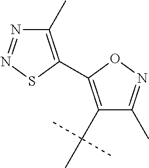 | H | O |
| 98 | CH₃ | H | H | 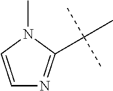 | H | O |
| 99 | CH₃ | H | H | 2-pyridyl | H | O |
| 100 | CH₃ | H | H | 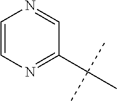 | H | O |
| 101 | CH₃ | H | CH₃ | cyclobutyl | H | O |
| 102 | CH₃ | H | CH₃ | cyclohexyl | H | O |
| 103 | CH₃ | H | CH₃ | cyclohexyl | H | S |
| 104 | CH₃ | H | CH₃ | 3,4-di-F Ph | H | O |
| 105 | CH₃ | H | CH₃ | 3,4-di-F Ph | H | S |
| 106 | CH₃ | H | CH₃ | 2-pyridyl | H | O |
| 107 | CH₃ | H | CH₃ | 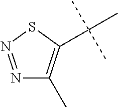 | H | O |
| 108 | CH₃ | H | CH₃ | 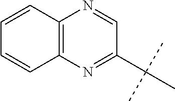 | H | O |
| 109 | CH₃ | H | Et | Ph | H | O |
| 110 | CH₃ | H | Et | Ph | H | S |
| 111 | CH₃ | H | Et | 4-CF₃ Ph | H | O |
| 112 | CH₃ | H | Et | 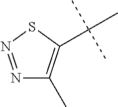 | H | O |
| 113 | CH₃ | H | Et | 2-napthyl | H | O |
| 114 | CH₃ | H | Et | 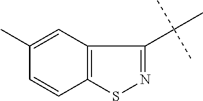 | H | O |
| 115 | CH₃ | H | Et | 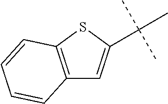 | H | O |
| 116 | CH₃ | H | Et | 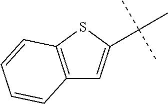 | H | S |
| 117 | CH₃ | H | Et | 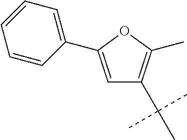 | H | O |
| 118 | CH₃ | H | Et | 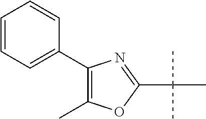 | H | O |
| 119 | CH₃ | H | i-Pr | Ph | H | O |
| 120 | CH₃ | H | i-Pr | Ph | H | S |
| 121 | CH₃ | H | i-Pr | 3,4-di-F Ph | H | O |
| 122 | CH₃ | H | i-Pr | 3,4-di-Cl Ph | H | O |
| 123 | CH₃ | H | i-Pr | 4-Ph Ph | H | O |
| 124 | CH₃ | H | i-Pr | 4-Ph Ph | H | S |
| 125 | CH₃ | H | i-Pr | 4-(4-ClPh)Ph | H | O |
| 126 | CH₃ | H | i-Pr | 4-(4-ClPh)Ph | H | S |
| 127 | CH₃ | H | i-Pr | 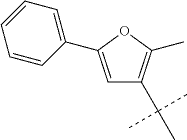 | H | O |
| 128 | CH₃ | H | i-Pr | 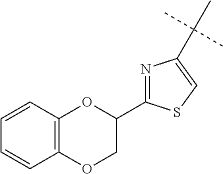 | H | O |
| 129 | CH₃ | H | i-Pr | 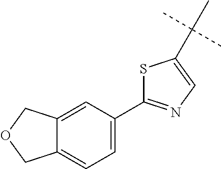 | H | O |
| 130 | CH₃ | H | i-Pr | 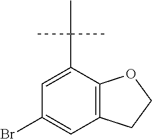 | H | O |
| 131 | CH₃ | H | i-Pr | 3-(5-CH₃) pyridyl | H | O |
| 132 | CH₃ | H | i-Pr | 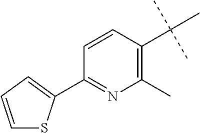 | H | O |
| 133 | CH₃ | H | i-Pr | 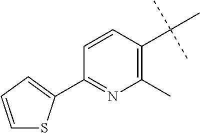 | H | S |
| 134 | CH₃ | H | i-Pr | 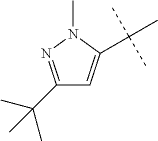 | H | O |
| 135 | CH₃ | CH₃ | i-Pr | 3,4-di-Cl Ph | CH₃ | O |
| 136 | CH₃ | n-propyl | i-Pr | 3,4-di-Cl Ph | n-propyl | O |
| 137 | CH₃ | Cl | i-Pr | 4-Cl Ph | H | O |
| 138 | CH₃ | Cl | i-Pr | 4-Cl Ph | H | S |
| 139 | CH₃ | Cl | i-Pr | 3-CH₃O Ph | H | O |
| 140 | CH₃ | Cl | i-Pr | 3-CH₃O Ph | Cl | O |
| 141 | CH₃ | Cl | i-Pr | 3-CH₃O Ph | Cl | S |
| 142 | CH₃ | Cl | i-Pr | 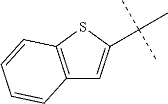 | Cl | O |
| 143 | CH₃ | Br | i-Pr | Ph | H | O |
| 144 | CH₃ | Br | i-Pr | 3-Cl Ph | H | O |
| 145 | CH₃ | Br | i-Pr | Ph | Br | O |
| 146 | CH₃ | Br | i-Pr | Ph | Br | S |
| 147 | CH₃ | CH₃ | i-Pr | Ph | H | O |
| 148 | CH₃ | CH₃ | i-Pr | Ph | H | S |
| 149 | CH₃ | CH₃ | i-Pr | 2-Cl Ph | H | O |
| 150 | CH₃ | CH₃ | i-Pr | 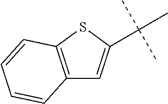 | H | O |
| 151 | CH₃ | CH₃CO | i-Pr | 3-F Ph | H | O |
| 152 | CH₃ | CH₃CO | i-Pr | 3-F Ph | H | S |
| 153 | CH₃ | n-PrCO | i-Pr | 3-F Ph | H | O |
| 154 | CH₃ | n-BuCO | i-Pr | 3-F Ph | H | O |
| 155 | CH₃ | H | n-Bu | Ph | H | O |
| 156 | CH₃ | H | n-Bu | 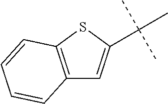 | H | O |
| 157 | CH₃ | H | n-Bu | 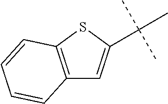 | H | S |
| 158 | CH₃ | H | n-Bu | 2-Cl Ph | H | O |
| 159 | CH₃ | H | n-Bu | 2,4 di-F Ph | H | O |
| 160 | CH₃ | H | n-Bu | 3,4 di-CH₃O Ph | H | O |
| 161 | CH₃ | H | n-Bu | 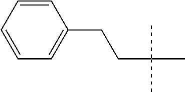 | H | O |
| 162 | CH₃ | H | n-Bu | 2-furyl | H | O |
| 163 | CH₃ | H | n-Bu | 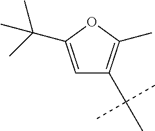 | H | O |
| 164 | CH₃ | H | n-Bu | 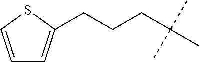 | H | O |
| 165 | CH₃ | H | n-Bu | 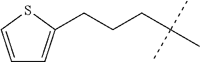 | H | S |
| 166 | CH₃ | H | n-Bu | 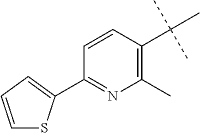 | H | O |
| 167 | CH₃ | H | n-Bu | 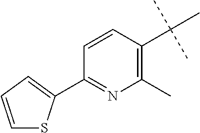 | H | S |
| 168 | CH₃ | H | n-Bu | 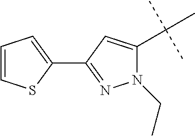 | H | O |
| 169 | CH₃ | H | n-Bu | 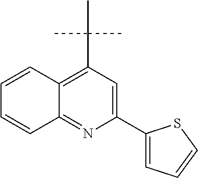 | H | O |
| 170 | CH₃ | Br | n-Bu | 2,4 di-F Ph | Br | O |
| 171 | CH₃ | Cl | n-Bu | 2,4 di-F Ph | H | O |
| 172 | CH₃ | H | n-pentyl | Ph | H | O |
| 173 | CH₃ | H | n-pentyl | 2,4 di-F Ph | H | O |
| 174 | CH₃ | H | n-pentyl | 2,4 di-F Ph | H | S |
| 175 | CH₃ | H | n-pentyl | 4-pyridyl | H | O |
| 176 | CH₃ | H | n-pentyl | 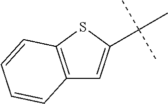 | H | O |
| 177 | CH₃ | Cl | n-pentyl | Ph | H | O |
| 178 | CH₃ | Cl | n-pentyl | Ph | H | S |
| 179 | CH₃ | H | Ph | 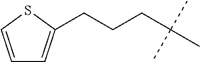 | H | O |
| 180 | CH₃ | H | 2-Cl Ph | 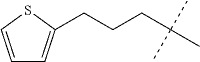 | H | O |
| 181 | CH₃ | H | 2-Cl Ph | 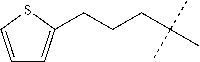 | H | S |
| 182 | CH₃ | H | H | PhOCH₂ | H | O |
| 183 | CH₃ | H | H | (4-CH₃Ph)OCH₂ | H | O |
| 184 | CH₃ | H | H | 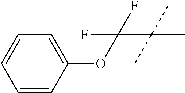 | H | O |
| 185 | CH₃ | H | CH₃ | Et | H | O |
| 186 | CH₃ | H | CH₃ | Et | H | S |
| 187 | CH₃ | H | CH₃ | CF₃CF₂ | H | O |
| 188 | CH₃ | H | CH₃ | t-butyl | H | O |
| 189 | CH₃ | H | Et | 3-(5-CH₃) pyridyl | H | O |
| 190 | CH₃ | H | Et | 4-pyridyl | H | O |
| 191 | CH₃ | H | Et | 4-pyridyl | H | S |
| 192 | CH₃ | Et | CH₃ | PhOCH₂ | H | O |
| 193 | CH₃ | Et | CH₃ | PhOCH₂ | H | S |
| 194 | CH₃ | Et | CH₃ | PhCH₂OCH₂ | H | O |
| 195 | CH₃ | n-propyl | CH₃ | PhOCH₂ | H | O |
| 196 | CH₃ | n-propyl | CH₃ | PhOCH₂ | n-propyl | O |
| 197 | CH₃ | n-butyl | CH₃ | PhOCH₂ | H | O |
| 198 | CH₃ | n-hexyl | CH₃ | PhOCH₂ | H | O |
| 199 | CH₃ | n-hexyl | CH₃ | PhOCH₂ | H | S |
| 200 | CH₃ | n-hexyl | isopropyl | 3-Cl Ph | H | O |
| 201 | CH₃ | n-hexyl | Ph | 3-Cl Ph | H | O |
| 202 | CH₃ | CH₃OCH₂ | CH₃ | PhOCH₂ | H | O |
| 203 | CH₃ | Ph | n-butyl | 3,4-di-F Ph | H | O |
| 204 | CH₃ | 3-F Ph | CH₃ | 1-napthyl | H | O |
| 205 | CH₃ | 4-pyridyl | H | 4-CF₃ Ph | H | O |
| 206 | CH₃ | 4-pyridyl | H | 4-CF₃ Ph | H | S |
| 207 | CH₃ | Cl | CH₃ | 3,5-di-F-Ph | H | O |
| 208 | CH₃ | Br | CH₃ | CF₃CF₂ | H | O |
| 209 | CH₃ | Br | n-butyl | CF₃CF₂ | H | O |
| 210 | CH₃ | Br | n-butyl | CF₃CF₂ | Br | O |
| 211 | CH₃ | Br | Ph | CF₃CF₂ | Br | O |
| 212 | CH₃ | 2-furyl | CH₃ | isobutyl | H | O |
| 213 | CH₃ | 2-furyl | CH₃ | isobutyl | H | S |
| 214 | CH₃ | 2-furyl | CH₃ | 2-F-4-CF₃ Ph | H | O |
| 215 | CH₃ | 2-furyl | CH₃ | 2-napthyl | H | O |
| 216 | CH₃ | 2-furyl | i-Pr | isobutyl | H | O |
| 217 | CH₃ | EtCO | n-propyl | 3-CH₃O Ph | EtCO | O |
| 218 | Et | H | H | cyclopropyl | H | O |
| 219 | Et | H | H | 4-F Ph | H | O |
| 220 | Et | H | H | 3,5-di-F-Ph | H | O |
| 221 | Et | H | H | 4-Cl PhCH₂ | H | O |
| 222 | Et | H | H | 2-quinolinyl | H | O |
| 223 | Et | H | CH₃ | PhCH₂ | H | O |
| 224 | Et | H | CH₃ | 4-F PhCH₂ | H | O |
| 225 | Et | H | CH₃ | 3,4-di-F-PhOCH₂ | H | O |
| 226 | Et | H | CH₃ | 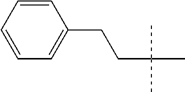 | H | O |
| 227 | Et | H | CH₃ | 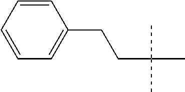 | H | S |
| 228 | Et | H | CH₃ | 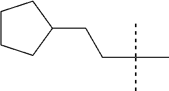 | H | O |
| 229 | Et | H | CH₃ | 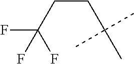 | H | O |
| 230 | Et | H | CH₃ | 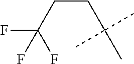 | H | S |
| 231 | Et | H | CH₃ | 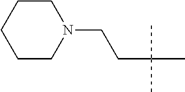 | H | O |
| 232 | Et | H | CH₃ | 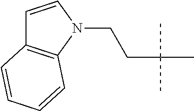 | H | O |
| 233 | Et | H | CH₃ | 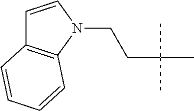 | H | S |
| 234 | Et | H | CH₃ | 2-quinolinyl | H | O |
| 235 | Et | H | CH₃ | 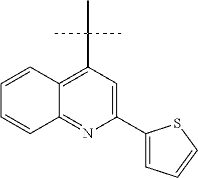 | H | O |
| 236 | Et | H | CH₃ | 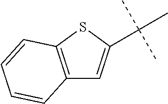 | H | O |
| 237 | Et | H | CH₃ | 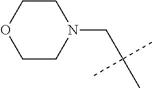 | H | O |
| 238 | Et | H | CH₃ | 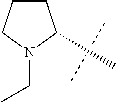 | H | O |
| 239 | Et | H | CH₃ | 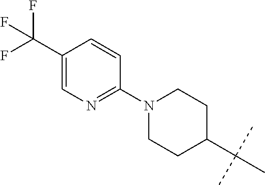 | H | O |
| 240 | Et | H | CH₃ | 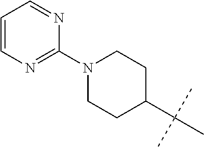 | H | O |
| 241 | Et | H | CH₃ | 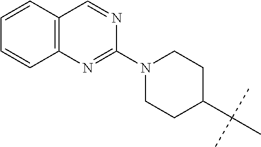 | H | O |
| 242 | Et | H | CH₃ | 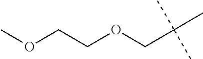 | H | O |
| 243 | Et | H | CH₃ | (4-CH₃O) | H | O |
| | | | | PhCH₂CH₂ | | |
| 244 | Et | H | CH₃ | 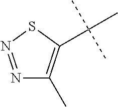 | H | O |
| 245 | Et | Cl | CH₃ | 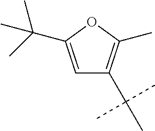 | H | O |
| 246 | Et | Br | CH₃ | 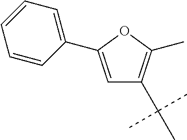 | H | O |
| 247 | Et | H | Et | 4-Ph Ph | H | O |
| 248 | Et | H | Et | 4-Ph Ph | H | S |
| 249 | Et | H | Et | 4-(4-CH₃Ph)Ph | H | O |
| 250 | Et | CH₃ | CH₃ | 2-F Ph | H | O |
| 251 | Et | CH₃ | CH₃ | 2-F Ph | CH₃ | O |
| 252 | Et | CH₃ | CH₃ | 2-F Ph | CH₃ | O |
| 253 | Et | CH₃ | CH₃ | 2-F Ph | CH₃ | S |
| 254 | Et | 3-Cl Ph | Et | 4-Ph Ph | H | O |
| 255 | Et | 3-Cl Ph | Et | 4-Ph Ph | H | S |
| 256 | Et | CH₃CO | H | 4-F Ph | H | O |
| 257 | Et | CH₃CO | isopropyl | 4-F Ph | H | O |
| 258 | Et | CH₃CO | Ph | 4-F Ph | H | O |
| 259 | Et | CH₃CO | CH₃ | cyclohexyl | CH₃CO | O |
| 260 | Et | CH₃CO | CH₃ | 4-F Ph | CH₃CO | O |
| 261 | Et | CH₃CO | Ph | 4-F Ph | CH₃CO | O |
| 262 | Et | CH₃CO | Ph | 4-F Ph | CH₃CO | S |
| 263 | Et | Cl | Et | 4-(4-CH₃Ph)Ph | H | O |
| 264 | Et | Cl | Et | 4-(4-CH₃Ph)Ph | Cl | O |
| 265 | Et | Cl | Et | 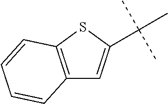 | Cl | O |
| 266 | Et | Br | Ph | 2-OCH₃ Ph | Br | O |
| 267 | CF₃CH₂ | H | H | n-butyl | H | O |
| 268 | CF₃CH₂ | H | H | Ph | H | O |
| 269 | CF₃CH₂ | H | H | 3-pyridyl | H | O |
| 270 | CF₃CH₂ | H | CH₃ | cyclopentyl | H | O |
| 271 | CF₃CH₂ | H | CH₃ | 4-(CF₃O)Ph | H | O |
| 272 | CF₃CH₂ | H | CH₃ | 4-(CF₃O)Ph | H | S |
| 273 | CF₃CH₂ | H | CH₃ | 4-(CHF₂O)Ph | H | O |
| 274 | CF₃CH₂ | H | CH₃ | 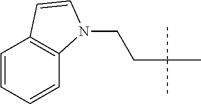 | H | O |
| 275 | CF₃CH₂ | H | n-butyl | (4-F Ph)OCH₂ | H | O |
| 276 | CF₃CH₂ | H | Ph | Ph | H | O |
| 277 | CF₃CH₂ | H | Ph | Ph | H | S |
| 278 | CF₃CH₂ | H | Ph | 2-(5-CF₃) furyl | H | O |
| 279 | CF₃CH₂ | H | Ph | 2-thienyl | H | O |
| 280 | CF₃CH₂ | H | 4-F Ph | Ph | H | O |
| 281 | CF₃CH₂ | CH₃ | H | 2-F Ph | H | O |
| 282 | CF₃CH₂ | CH₃ | H | 2-F Ph | H | S |
| 283 | CF₃CH₂ | CH₃ | H | 2-F Ph | CH₃ | O |
| 284 | CF₃CH₂ | CH₃ | Et | 3-CF₃ Ph | H | O |
| 285 | CF₃CH₂ | CH₃ | n-butyl | (4-F Ph)OCH₂ | H | O |
| 286 | CF₃CH₂ | CH₃ | n-butyl | (4-F Ph)OCH₂ | H | S |
| 287 | CF₃CH₂ | CH₃ | Ph | 2-thienyl | H | O |
| 288 | n-propyl | H | H | CH₃ | H | O |
| 289 | n-propyl | H | H | CH₃ | H | S |
| 290 | n-propyl | H | H | n-propyl | H | O |
| 291 | n-propyl | H | H | cyclobutyl | H | O |
| 292 | n-propyl | H | H | cycloheptyl | H | O |
| 293 | n-propyl | H | H | 3,4-di-CH₃ Ph | H | O |
| 294 | n-propyl | H | H | 2-thienyl | H | O |
| 295 | n-propyl | H | H | 2-thienyl | H | S |
| 296 | n-propyl | H | H | 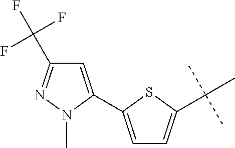 | H | O |
| 297 | n-propyl | H | CH₃ | CH₃ | H | O |
| 298 | n-propyl | H | CH₃ | CH₃ | H | S |
| 299 | n-propyl | H | CH₃ | 3-CF₃ Ph | H | O |
| 300 | n-propyl | H | CH₃ | 2-thienyl | H | O |
| 301 | n-propyl | H | CH₃ | 3-(4-(OCH₃)thienyl) | H | O |
| 302 | n-propyl | H | CH₃ | 2-(5-(CH₃)thienyl) | H | O |
| 303 | n-propyl | H | CH₃ | 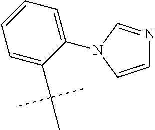 | H | O |
| 304 | n-propyl | H | CH₃ | 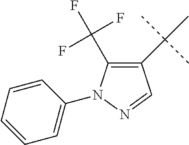 | H | O |
| 305 | n-propyl | CH₃ | CH₃ | 3-Br Ph | H | O |
| 306 | n-propyl | CH₃ | CH₃ | 3-Br Ph | H | S |
| 307 | n-propyl | CH₃ | CH₃ | 3-Br Ph | CH₃ | O |
| 308 | n-propyl | CH₃ | CH₃ | 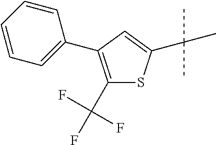 | H | O |
| 309 | n-propyl | CH₃ | CH₃ | 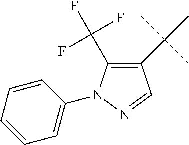 | H | O |
| 310 | n-propyl | n-propyl | CH₃ | 3-Cl Ph | H | O |
| 311 | n-propyl | n-propyl | CH₃ | 3-Cl Ph | H | S |
| 312 | n-propyl | CH₃OCH₂ | CH₃ | 3-Cl Ph | H | O |
| 313 | n-propyl | CH₃CO | CH₃ | 3-Cl Ph | H | O |
| 314 | n-propyl | PrCO | CH₃ | 3-Cl Ph | H | O |
| 315 | n-propyl | PrCO | CH₃ | 3-Cl Ph | PrCO | O |
| 316 | n-propyl | Cl | CH₃ | 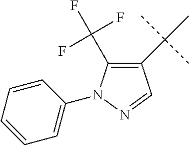 | H | O |
| 317 | n-propyl | Cl | CH₃ | 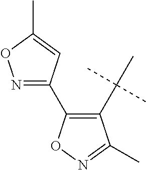 | H | O |
| 318 | n-propyl | Cl | CH₃ | 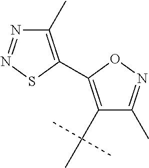 | H | O |
| 319 | n-propyl | Cl | H | Ph | Cl | O |
| 320 | n-propyl | Cl | CH₃ | Ph | Cl | O |
| 321 | n-propyl | Cl | CH₃ | Ph | Cl | S |
| 322 | n-propyl | Cl | n-propyl | 3-CH₃O Ph | Cl | O |
| 323 | n-propyl | Cl | n-propyl | 3-pyridyl | Cl | O |
| 324 | isopropyl | H | H | Ph | H | O |
| 325 | isopropyl | H | H | 2-quinolinyl | H | O |
| 326 | isopropyl | H | H | 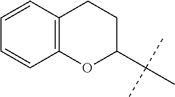 | H | O |
| 327 | isopropyl | H | CH₃ | CH₃ | H | O |
| 328 | isopropyl | H | CH₃ | t-butyl | H | O |
| 329 | isopropyl | H | CH₃ | n-heptyl | H | O |
| 330 | isopropyl | H | CH₃ | n-heptyl | H | S |
| 331 | isopropyl | H | CH₃ | 2,4-di-F Ph | H | O |
| 332 | isopropyl | H | CH₃ | 2,4-di-F Ph | H | S |
| 333 | isopropyl | H | CH₃ | 2-F-4-CF₃ Ph | H | O |
| 334 | isopropyl | H | n-propyl | 2-F-4-CF₃ Ph | H | O |
| 335 | isopropyl | H | n-propyl | 3,5-di-Cl Ph | H | O |
| 336 | isopropyl | H | Ph | 2,4-di-CF₃ Ph | H | O |
| 337 | isopropyl | H | 4-F Ph | 2-F-4-CF₃ Ph | H | O |
| 338 | isopropyl | CH₃ | Et | 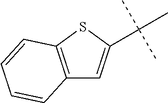 | H | O |
| 339 | isopropyl | CH₃ | Et | 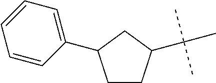 | H | O |
| 340 | isopropyl | CH₃ | Et | 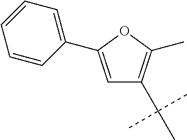 | H | O |
| 341 | isopropyl | CH₃ | Et | 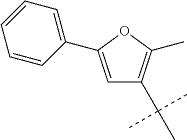 | H | S |
| 342 | isopropyl | CH₃ | Et | 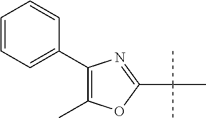 | H | O |
| 343 | isopropyl | CH₃ | Et | 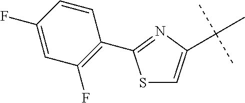 | H | O |
| 344 | isopropyl | CH₃ | Et | 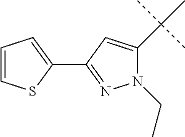 | H | O |
| 345 | isopropyl | Et | CH₃ | 3-CF₃ Ph | H | O |
| 346 | isopropyl | Et | CH₃ | 3-Et Ph | H | O |
| 347 | isopropyl | n-propyl | H | PhOCH₂ | H | O |
| 348 | isopropyl | n-propyl | H | PhOCH₂ | n-propyl | O |
| 349 | isopropyl | n-propyl | H | 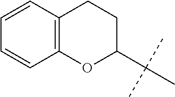 | H | O |
| 350 | isopropyl | n-propyl | H | 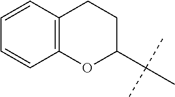 | n-propyl | O |
| 351 | isopropyl | n-propyl | H | 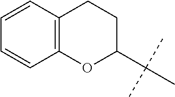 | H | S |
| 352 | isopropyl | n-propyl | H | 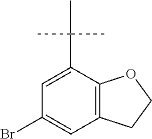 | H | O |
| 353 | isopropyl | n-propyl | n-butyl | 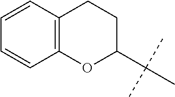 | H | O |
| 354 | isopropyl | n-propyl | Ph | 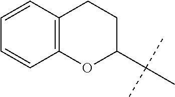 | H | O |
| 355 | isopropyl | n-butyl | H | 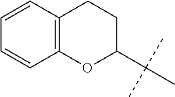 | H | O |
| 356 | isopropyl | n-hexyl | H | 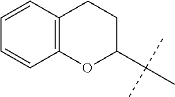 | H | O |
| 357 | isopropyl | Ph | H | CH₃ | H | O |
| 358 | isopropyl | Ph | H | n-propyl | H | O |
| 359 | isopropyl | Ph | H | n-propyl | H | S |
| 360 | isopropyl | Ph | H | 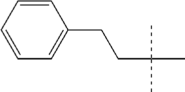 | H | O |
| 361 | isopropyl | Ph | H | 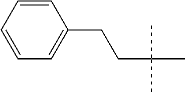 | H | S |
| 362 | isopropyl | Ph | CH₃ | 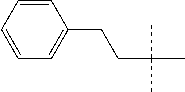 | H | O |
| 363 | isopropyl | Ph | CH₃ | 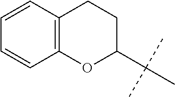 | H | O |
| 364 | isopropyl | Cl | Et | Ph | H | O |
| 365 | isopropyl | Cl | Et | Ph | H | S |
| 366 | isopropyl | Cl | Et | 2-CH₃ Ph | Cl | O |
| 367 | isopropyl | Cl | n-propyl | 3-F Ph | H | O |
| 368 | isopropyl | Cl | isopropyl | 3-F Ph | H | O |
| 369 | isopropyl | Cl | 4-F Ph | 3-F Ph | H | O |
| 370 | isopropyl | Br | Et | 2-CH₃ Ph | Br | O |
| 371 | isopropyl | Br | Et | 2-CH₃ Ph | Br | S |
| 372 | n-butyl | H | H | Cyclohexyl | H | O |
| 373 | n-butyl | H | H | Ph | H | O |
| 374 | n-butyl | H | H | 4-F Ph | H | O |
| 375 | n-butyl | H | H | 3,5-di-Cl Ph | H | O |
| 376 | n-butyl | H | H | 3,5-di-Cl Ph | H | S |
| 377 | n-butyl | H | CH₃ | 3,4-di-CH₃OPh | H | O |
| 378 | n-butyl | H | CH₃ | 4-F PhOCH₂ | H | O |
| 379 | n-butyl | H | CH₃ | 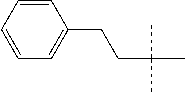 | H | O |
| 380 | n-butyl | H | CH₃ | (4-CH₃O) | H | O |
| | | | | PhCH₂CH₂F | | |
| 381 | n-butyl | H | CH₃ | 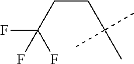 | H | O |
| 382 | n-butyl | H | CH₃ | 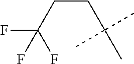 | H | S |
| 383 | n-butyl | H | Et | 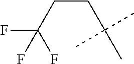 | H | O |
| 384 | n-butyl | H | n-propyl | cyclobutyl | H | O |
| 385 | n-butyl | H | n-propyl | 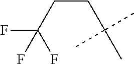 | H | O |
| 386 | n-butyl | H | isopropyl | 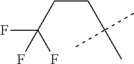 | H | O |
| 387 | n-butyl | H | Ph | n-propyl | H | O |
| 388 | n-butyl | H | Ph | 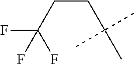 | H | O |
| 389 | n-butyl | H | Ph | Ph | H | O |
| 390 | n-butyl | H | Ph | Ph | H | S |
| 391 | n-butyl | CH₃ | CH₃ | 4-CH₃ Ph | H | O |
| 392 | n-butyl | CH₃ | CH₃ | 4-CH₃ Ph | CH₃ | O |
| 393 | n-butyl | CH₃ | Et | 4-CH₃ Ph | H | O |
| 394 | n-butyl | CH₃ | Ph | 4-CH₃ Ph | H | O |
| 395 | n-butyl | CH₃OCH₂ | CH₃ | 2,4-di-CH₃ Ph | H | O |
| 396 | n-butyl | Cl | CH₃ | 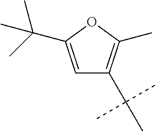 | H | O |
| 397 | n-butyl | Cl | CH₃ | 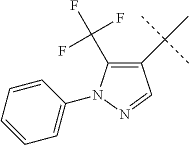 | H | O |
| 398 | n-butyl | Cl | Ph | 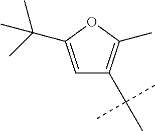 | H | O |
| 399 | n-pentyl | H | H | CH₃ | H | O |
| 400 | n-pentyl | H | H | CH₃ | H | S |
| 401 | n-pentyl | H | H | Et | H | O |
| 402 | n-pentyl | H | H | cyclopentyl | H | O |
| 403 | n-pentyl | H | H | cyclopentyl | H | S |
| 404 | n-pentyl | H | H | cycloheptyl | H | O |
| 405 | n-pentyl | H | H | Ph | H | O |
| 406 | n-pentyl | H | H | Ph | H | S |
| 407 | n-pentyl | H | H | 2-furyl | H | O |
| 408 | n-pentyl | H | H | 2-(5-CF₃) furyl | H | O |
| 409 | n-pentyl | H | H | 2-thienyl | H | O |
| 410 | n-pentyl | H | H | 3,4-di-Cl Ph | H | O |
| 411 | n-pentyl | H | CH₃ | n-butyl | H | O |
| 412 | n-pentyl | H | CH₃ | n-butyl | H | S |
| 413 | n-pentyl | H | CH₃ | 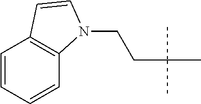 | H | O |
| 414 | n-pentyl | H | CH₃ | PhOCH₂ | H | O |
| 415 | n-pentyl | H | CH₃ | PhCH₂OCH₂ | H | O |
| 416 | n-pentyl | H | Et | 2-F Ph | H | O |
| 417 | n-pentyl | H | Et | 2-F Ph | H | S |
| 418 | n-pentyl | H | 4-CH₃ Ph | 2-F Ph | H | O |
| 419 | n-pentyl | CH₃ | Et | 4-CH₃ Ph | H | O |
| 420 | n-pentyl | Cl | CH₃ | n-butyl | H | O |
| 421 | n-pentyl | Cl | CH₃ | Ph | H | O |
| 422 | n-pentyl | Cl | CH₃ | Ph | H | S |
| 423 | n-pentyl | Cl | CH₃ | 4-Ph Ph | H | O |
| 424 | n-pentyl | Cl | CH₃ | 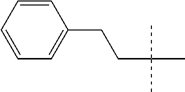 | H | O |
| 425 | n-pentyl | Cl | CH₃ | 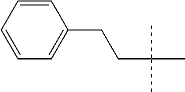 | Cl | O |
| 426 | n-pentyl | PrCO | CH₃ | 4-CH₃ Ph | PrCO | O |
| 427 | n-pentyl | Ph | CH₃ | 3-Br Ph | H | O |
| 428 | n-pentyl | 2-thienyl | CH₃ | 3-Br Ph | 2-thienyl | O |
| 429 | n-hexyl | H | H | 2-F Ph | H | O |
| 430 | n-hexyl | H | CH₃ | cyclopentyl | H | O |
| 431 | n-hexyl | H | CH₃ | cycloheptyl | H | O |
| 432 | n-hexyl | H | CH₃ | 2-F Ph | H | O |
| 433 | n-hexyl | H | CH₃ | 2-F Ph | H | S |
| 434 | n-hexyl | H | Et | 2-F Ph | H | O |
| 435 | n-hexyl | H | n-propyl | 2-F Ph | H | O |
| 436 | n-hexyl | H | isopropyl | 2-F Ph | H | O |
| 437 | n-hexyl | H | Ph | 2-F Ph | H | O |
| 438 | n-hexyl | CH₃CO | CH₃ | 2,4-di-CH₃ Ph | H | O |
| 439 | n-hexyl | CH₃OCH₂ | CH₃ | 2,4-di-CH₃ Ph | H | O |
| 440 | n-hexyl | Ph | Et | Ph | H | O |
| 441 | n-hexyl | Ph | Et | Ph | H | S |
| 442 | n-hexyl | Ph | Et | 4-pyridyl | H | O |
| 443 | n-hexyl | Br | Et | Ph | Br | O |
| 444 | n-hexyl | Br | Et | 2-F Ph | Br | O |
| 445 | cyclopropyl | H | H | cyclopentyl | H | O |
| 446 | cyclopropyl | H | H | 2,4-di-Cl Ph | H | O |
| 447 | cyclopropyl | H | H | 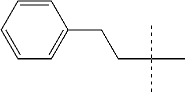 | H | O |
| 448 | cyclopropyl | H | CH₃ | 3-F Ph | H | O |
| 449 | cyclopropyl | H | CH₃ | 3-F Ph | H | S |
| 450 | cyclopropyl | H | CH₃ | 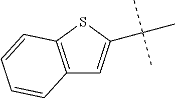 | H | O |
| 451 | cyclopropyl | H | Et | 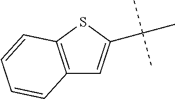 | H | O |
| 452 | cyclopropyl | H | n-propyl | 4-CF₃ Ph | H | O |
| 453 | cyclopropyl | H | isopropyl | Ph | H | O |
| 454 | cyclopropyl | H | isopropyl | 3-pyridyl | H | O |
| 455 | cyclopropyl | H | n-butyl | 4-CF₃ Ph | H | O |
| 456 | cyclopropyl | H | n-hexyl | Ph | H | O |
| 457 | cyclopropyl | H | n-hexyl | 4-CF₃ Ph | H | O |
| 458 | cyclopropyl | H | Ph | Ph | H | O |
| 459 | cyclobutyl | H | CH₃ | 4-CH₃ Ph | H | O |
| 460 | cyclobutyl | H | Et | 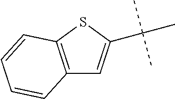 | H | O |
| 461 | cyclobutyl | H | Et | 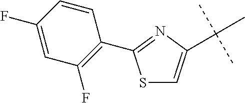 | H | O |
| 462 | cyclobutyl | H | Et | 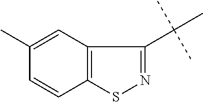 | H | O |
| 463 | cyclobutyl | H | Et | 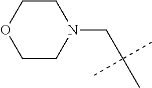 | H | O |
| 464 | cyclobutyl | H | Et | 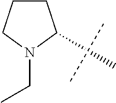 | H | O |
| 465 | cyclobutyl | H | 4-F Ph | 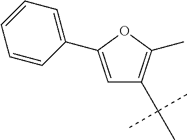 | H | O |
| 466 | cyclobutyl | Cl | CH₃ | 3-Cl Ph | Cl | O |
| 467 | cyclobutyl | Cl | CH₃ | 3-Cl Ph | Cl | S |
| 468 | cyclopentyl | H | H | 3-CF₃ Ph | H | O |
| 469 | cyclopentyl | H | CH₃ | 2,4-di-CF₃ Ph | H | O |
| 470 | cyclopentyl | H | CH₃ | 2,4-di-CF₃ Ph | H | S |
| 471 | cyclopentyl | H | n-butyl | 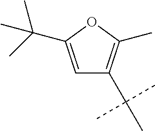 | H | O |
| 472 | cyclopentyl | H | 3-F Ph | 4-CH₃ Ph | H | O |
| 473 | cyclopentyl | CH₃ | CH₃ | Ph | H | O |
| 474 | cyclopentyl | CH₃ | CH₃ | 3,5-di-Cl Ph | H | O |
| 475 | cyclopentyl | CH₃ | CH₃ | Ph | H | S |
| 476 | cyclopentyl | Et | CH₃ | Ph | H | O |
| 477 | cyclopentyl | Cl | CH₃ | Ph | Cl | O |
| 478 | cyclopentyl | Cl | CH₃ | Ph | Cl | S |
| 479 | cyclohexyl | H | H | 3-F Ph | H | O |
| 480 | cyclohexyl | H | H | 2,4-di-CH₃ Ph | H | O |
| 481 | cyclohexyl | H | H | 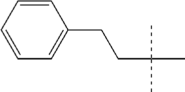 | H | O |
| 482 | cyclohexyl | H | CH₃ | n-propyl | H | O |
| 483 | cyclohexyl | H | CH₃ | n-propyl | H | S |
| 484 | cyclohexyl | H | CH₃ | 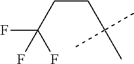 | H | O |
| 485 | cyclohexyl | H | CH₃ | 3-Cl Ph | H | O |
| 486 | cyclohexyl | H | CH₃ | 3-Cl Ph | H | S |
| 487 | cyclohexyl | H | CH₃ | 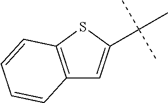 | H | O |
| 488 | cyclohexyl | H | Et | 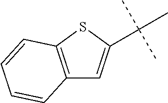 | H | O |
| 489 | cyclohexyl | H | n-propyl | 4-CF₃ Ph | H | O |
| 490 | cyclohexyl | H | n-propyl | 3-pyridyl | H | O |
| 491 | cyclohexyl | H | isopropyl | Ph | H | O |
| 492 | cyclohexyl | H | isopropyl | 3-pyridyl | H | O |
| 493 | cyclohexyl | H | n-butyl | 3-Cl Ph | H | O |
| 494 | cyclohexyl | H | n-pentyl | 3-Cl Ph | H | O |
| 495 | cyclohexyl | H | n-hexyl | 4-CF₃ Ph | H | O |
| 496 | cyclohexyl | H | 4-F Ph | Ph | H | O |
| 497 | cyclohexyl | CH₃ | CH₃ | 3-CH₃ Ph | H | O |
| 498 | cyclohexyl | CH₃ | CH₃ | 3-CH₃ Ph | H | S |
| 499 | cyclohexyl | CH₃ | Et | 3-pyridyl | CH₃ | O |
| 500 | cyclohexyl | Et | CH₃ | 2-F-4-CF₃ Ph | Et | O |
| 501 | cyclohexyl | 2-thienyl | i-Pr | 3-pyridyl | H | O |
| 502 | cyclohexyl | Cl | CH₃ | 2,3-di-CH₃ Ph | H | O |
| 503 | cyclohexyl | Cl | CH₃ | 2,3-di-CH₃ Ph | H | S |
| 504 | 2-propenyl | H | H | CH₃ | H | O |
| 505 | 2-propenyl | H | H | isopentyl | H | O |
| 506 | 2-propenyl | H | H | cyclopentyl | H | O |
| 507 | 2-propenyl | H | H | Ph | H | O |
| 508 | 2-propenyl | H | H | Ph | H | S |
| 509 | 2-propenyl | H | H | 2-quinolinyl | H | O |
| 510 | 2-propenyl | H | H | 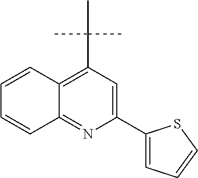 | H | O |
| 511 | 2-propenyl | H | CH₃ | 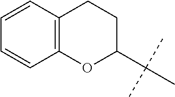 | H | O |
| 512 | 2-propenyl | H | CH₃ | 2,4-di-F Ph | H | O |
| 513 | 2-propenyl | H | CH₃ | 2,4-di-F Ph | H | S |
| 514 | 2-propenyl | H | CH₃ | 2-F-4-CF₃ Ph | H | O |
| 515 | 2-propenyl | H | Et | 2-napthyl | H | O |
| 516 | 2-propenyl | H | Et | 2-napthyl | H | S |
| 517 | 2-propenyl | H | Et | 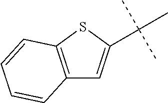 | H | O |
| 518 | 2-propenyl | H | Et | 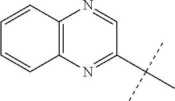 | H | O |
| 519 | 2-propenyl | H | n-propyl | 2-F-4-CF₃ Ph | H | O |
| 520 | 2-propenyl | H | Ph | 2,4-di-CF₃ Ph | H | O |
| 521 | 2-propenyl | H | 4-F Ph | 2-F-4-CF₃ Ph | H | O |
| 522 | 2-propenyl | CH₃ | Et | 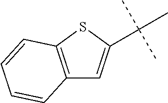 | H | O |
| 523 | 2-propenyl | Cl | CH₃ | 3-CF₃ Ph | Cl | O |
| 524 | 2-propenyl | Cl | CH₃ | 3-CF₃ Ph | Cl | S |
| 525 | 2-propenyl | Br | Et | 3-CF₃ Ph | Br | O |
| 526 | 2-isobutenyl | H | H | 3-pyridyl | H | O |
| 527 | 2-isobutenyl | H | H | 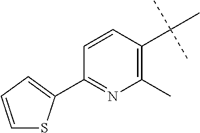 | H | O |
| 528 | 2-isobutenyl | H | CH₃ | 4-(CF₃O)Ph | H | O |
| 529 | 2-isobutenyl | H | CH₃ | 4-(CF₃O)Ph | H | O |
| 530 | 2-isobutenyl | H | CH₃ | 4-(CF₃O)Ph | H | S |
| 531 | 2-isobutenyl | H | n-butyl | 4-(CH₃O)Ph | H | O |
| 532 | 2-isobutenyl | H | n-butyl | (4-F Ph)OCH₂ | H | O |
| 533 | 2-isobutenyl | H | n-butyl | (4-CH₃O) | H | O |
| | | | | PhCH₂CH₂ | | |
| 534 | 2-isobutenyl | H | Ph | 2-thienyl | H | O |
| 535 | 2-isobutenyl | H | 4-F Ph | Ph | H | O |
| 536 | 2-isobutenyl | CH₃CO | Ph | cyclohexyl | H | O |
| 537 | 2-isobutenyl | CH₃CO | Ph | 3-F Ph | H | O |
| 538 | 4-pentenyl | H | CH₃ | Ph | H | O |
| 539 | 4-pentenyl | H | CH₃ | Ph | H | S |
| 540 | 5-hexenyl | H | H | Ph | H | O |
| 541 | 5-hexenyl | H | CH₃ | 2-F Ph | H | O |
| 542 | 5-hexenyl | H | CH₃ | 2-F Ph | H | S |
| 543 | 5-hexenyl | H | CH₃ | 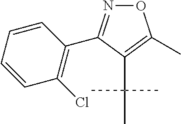 | H | O |
| 544 | 5-hexenyl | H | isopropyl | 4-(CF₃O)Ph | H | O |
| 545 | 5-hexenyl | H | Ph | 4-(CF₃O)Ph | H | O |
| 546 | 5-hexenyl | CH₃CO | CH₃ | 2-CH₃ Ph | CH₃CO | O |
| 547 | CH₃O | H | H | cyclobutyl | H | O |
| 548 | CH₃O | H | H | 2,4-di-F Ph | H | O |
| 549 | CH₃O | H | H | (4-CH₃) PhCH₂ | H | O |
| 550 | CH₃O | H | H | 2-quinolinyl | H | O |
| 551 | CH₃O | H | CH₃ | CH₃ | H | O |
| 552 | CH₃O | H | CH₃ | CH₃ | H | S |
| 553 | CH₃O | H | CH₃ | 3-CF₃ Ph | H | O |
| 554 | CH₃O | H | CH₃ | 2-furyl | H | O |
| 555 | CH₃O | H | CH₃ | 2-furyl | H | S |
| 556 | CH₃O | H | CH₃ | 2-thienyl | H | O |
| 557 | CH₃O | H | CH₃ | 3-(4-(OCH₃)thienyl) | H | O |
| 558 | CH₃O | H | CH₃ | 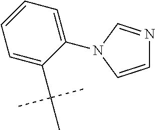 | H | O |
| 559 | CH₃O | H | n-propyl | 4-(CF₃O)Ph | H | O |
| 560 | CH₃O | H | 4-F Ph | 4-(CF₃O)Ph | H | O |
| 561 | CH₃O | Br | isobutyl | 3-CF₃ Ph | Br | O |
| 562 | CH₃O | H | CH₃ | 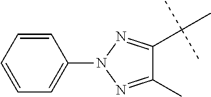 | H | O |
| 563 | EtO | 3-F Ph | Et | cyclopentyl | H | O |
| 564 | EtO | H | H | CH₃ | H | O |
| 565 | EtO | H | H | CH₃ | H | S |
| 566 | EtO | H | H | 3,4-di-CH₃ Ph | H | O |
| 567 | EtO | H | CH₃ | n-propyl | H | O |
| 568 | EtO | H | CH₃ | cyclobutyl | H | O |
| 569 | EtO | H | CH₃ | cycloheptyl | H | O |
| 570 | EtO | H | CH₃ | cycloheptyl | H | S |
| 571 | EtO | H | CH₃ | 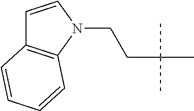 | H | O |
| 572 | EtO | H | CH₃ | 3,4-di-F Ph | H | O |
| 573 | EtO | H | CH₃ | 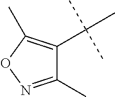 | H | O |
| 574 | EtO | H | n-butyl | 2-thienyl | H | O |
| 575 | EtO | H | Ph | 2-thienyl | H | O |
| 576 | EtO | CH3 | CH₃ | 4-Br Ph | H | O |
| 577 | EtO | Cl | CH₃ | n-hexyl | H | O |
| 578 | EtO | Cl | CH₃ | 2-Cl Ph | H | O |
| 579 | EtO | Cl | CH₃ | 2-Cl Ph | H | S |
| 580 | EtO | Cl | n-butyl | Ph | Cl | O |
| 581 | (i-Pr)O | H | H | CH₃ | H | O |
| 582 | (i-Pr)O | H | H | CH₃ | H | S |
| 583 | (i-Pr)O | H | H | 3,5-di-Cl Ph | H | O |
| 584 | (i-Pr)O | H | CH₃ | 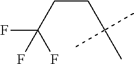 | H | O |
| 585 | (i-Pr)O | H | CH₃ | 3-Cl-5-F Ph | H | O |
| 586 | (i-Pr)O | H | CH₃ | 3-Cl-5-F Ph | H | S |
| 587 | (i-Pr)O | H | CH₃ | 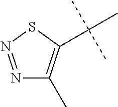 | H | O |
| 588 | (i-Pr)O | H | isopropyl | 4-Br Ph | H | O |
| 589 | (i-Pr)O | H | 4-F Ph | 3,4-di-F Ph | H | O |
| 590 | (i-Pr)O | CH₃ | Et | 2-thienyl | H | O |
| 591 | (i-Pr)O | CH₃CO | Et | 2-thienyl | CH₃CO | O |
| 592 | (i-Pr)O | Cl | 3-F Ph | 2,4-di-F Ph | Cl | O |
| 593 | n-BuO | H | H | cyclpentyl | H | O |
| 594 | n-BuO | H | H | cyclooctyl | H | O |
| 595 | n-BuO | H | H | cyclooctyl | H | S |
| 596 | n-BuO | H | Et | cyclooctyl | H | O |
| 597 | n-BuO | H | Et | Ph | H | O |
| 598 | n-BuO | H | Et | 2,4-di-F Ph | H | O |
| 599 | n-BuO | H | Et | PhOCH₂ | H | O |
| 600 | n-BuO | H | isopropyl | cyclooctyl | H | O |
| 601 | n-BuO | H | n-hexyl | cyclooctyl | H | O |
| 602 | n-BuO | CH₃ | CH₃ | 3,5-di-F Ph | H | O |
| 603 | n-BuO | PrCO | Et | 3,5-di-CH₃ Ph | H | O |
| 604 | n-BuO | Br | Ph | cyclooctyl | Br | O |
| 605 | (n-pentyl)O | H | CH₃ | 3-Br Ph | H | O |
| 606 | (n-pentyl)O | H | CH₃ | 3-Br Ph | H | S |
| 607 | (n-pentyl)O | H | CH₃ | 2-napthyl | H | O |
| 608 | (n-pentyl)O | H | CH₃ | 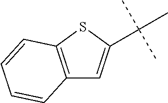 | H | O |
| 609 | (n-hexyl)O | H | CH₃ | cyclopropyl | H | O |
| 610 | (n-hexyl)O | H | CH₃ | n-pentyl | H | O |
| 611 | (n-hexyl)O | H | CH₃ | 3-Br Ph | H | O |
| 612 | (n-hexyl)O | H | CH₃ | 2-napthyl | H | O |
| 613 | (i-hexyl)O | CH₃OCH₂ | Et | Ph | H | O |
| 614 | (i-hexyl)O | CH₃OCH₂ | Et | Ph | H | S |
| 615 | CO₂H | H | H | 3,5-di-Cl Ph | H | O |
| 616 | CO₂H | H | CH₃ | 3,5-di-Cl Ph | H | O |
| 617 | CO₂H | H | propyl | Ph | H | O |
| 618 | CO₂H | H | propyl | 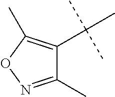 | H | O |
| 619 | CO₂H | H | CH₃ | Ph | H | O |
| 620 | CO₂H | H | CH₃ | 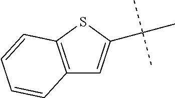 | H | O |
| 621 | CO₂H | H | CH₃ | 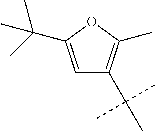 | H | O |
| 622 | CO₂H | CH₃ | CH₃ | 3,5-di-Cl Ph | H | O |
| 623 | CO₂H | CH₃ | isopropyl | 3-Br Ph | H | O |
| 624 | CO₂H | CH₃ | isopropyl | 3-Br Ph | CH₃ | O |
| 625 | CO₂H | CH₃ | 4-F Ph | propyl | H | O |
| 626 | CO₂H | Et | H | 4-F Ph | H | O |
| 627 | CO₂H | Et | H | 4-F Ph | Et | O |
| 628 | CO₂H | Et | CH₃ | 4-F Ph | Et | O |
| 629 | CO₂H | Et | propyl | Ph | H | O |
| 630 | CO₂H | Et | propyl | Ph | H | S |
| 631 | CO₂H | Ph | CH₃ | 2-furyl | H | O |
| 632 | CO₂H | Ph | CH₃ | 2-furyl | H | S |
| 633 | CO₂H | 3-Br Ph | Ph | 2-thienyl | H | O |
| 634 | CO₂H | n-PrCO | H | 3-Cl Ph | H | O |
| 635 | CO₂H | n-PrCO | H | 3-pyridyl | H | O |
| 636 | CO₂H | n-PrCO | H | 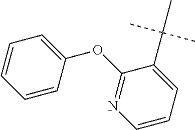 | H | O |
| 637 | CO₂H | n-PrCO | CH₃ | 3-Cl Ph | H | O |
| 638 | CO₂H | n-PrCO | CH₃ | 3-Cl Ph | n-PrCO | O |
| 639 | CO₂H | n-pentylCO | Ph | 3-Cl Ph | H | O |

The particular process to be utilized in the preparation of the compounds of this invention depends upon the specific compound desired. Such factors as the selection of the specific X moiety, and the specific substituents possible at various locations on the molecule, all play a role in the path to be followed in the preparation of the specific compounds of this invention. Those factors are readily recognized by one of ordinary skill in the art.

In general, the compounds of this invention may be prepared by standard techniques known in the art and by known processes analogous thereto. For example, the compounds may be prepared according to methods described in U.S. Pat. No. 6,828,335, which is incorporated by reference in its entirety.

For example, the compounds of Formula I may generally be synthesized according to Reaction Schemes 1, 2, and 3. Reaction Schemes 1 and 2 demonstrate how to make intermediates that are coupled in Reaction Scheme 3 to provide the compounds of Formula I.

Route (A) of Reaction Scheme 1 provides a method to prepare compounds 4 and 5 where R″ is C₁-C₆ lower alkyl or benzyl, R³ is not hydrogen, and X is O. The first step shows protection of the acid group of a commercially available aspartate derivative compound 1 by means well known in the art such as, for example, by forming a silyl ester, followed by N-acylation with the appropriate R⁴-acid derivative, R⁴COY, where Y is a leaving group such as halo. Finally, the compound is deprotected by means well known in the art such as, for example, in the case of a silyl ester, an aqueous work up, to give compound 2. Alternatively, condensation of the protected form of compound 1 with a free carboxylic acid such as R⁴COOH in the presence of a dehydrating reagent, such as DCC or EDCI, also provides compound 2. Compound 2 may then be converted to compound 3, where R³ is as defined for Formula I compounds by several methods. For example, one such method, when R³=Me, is the well known Dakin-West reaction which is typically performed using acetic anhydride and pyridine. When R³ is other than hydrogen, compound 2 may be converted to an acid chloride with a reagent such as thionyl chloride and reacted with a Grignard reagent such as R³Mg-halo, to provide compound 3. Other methods for the formation of ketones of compound 3 from acids and acid derivatives may also be employed, for example, by using Weinreb amides, which are known to those skilled in the art. Compound 3 is then cyclized under acid dehydrative conditions using, for example, phosphorus oxychloride, or a mixture of sulfuric acid and acetic anhydride, generally with heating, to provide compound 4 where X is O and the R³ group is attached at the 5 position.

It will be recognized by those skilled in the art that compound 4 and thus, compound 5, may exist in two regioisomeric forms with respect to the attachment point of the R³, CH₂CO₂R″, and CH₂CH₂OH groups. Using Route (B), one can prepare compound 4 in which the R³ is attached at the 4-position and carboxymethyl side chain is attached at the 5-position, that is, the groups are reversed from that of Route (A). In Route (B), a commercially available amino acid, compound 6, may be acylated under basic conditions, for example, with aqueous sodium hydroxide, with an appropriate R⁴-acid derivative, (e.g., R⁴COY), where Y is a leaving group such as chloro, to provide the N-acylated product 7. Compound 7 may be then coupled with an acetic acid ester in the presence of a strong non-nucleophilic base to make the keto ester 8, where R″ is C₁-C₆ alkyl or benzyl. Cyclization of compound 8 using a dehydrating reagent such as POCl₃ provides compound 4 where X=O and R³ is attached at the 4 position. Reaction of compound 8 with a nucleophilic S reagent such as P₂S₅ in solvents such as pyridine or acetonitrile/triethylamine, with heating as necessary, gives compound 4 where X=S and R³ is attached at the 4 position.

Route (C) of Reaction Scheme 1 depicts the preparation of compound 4 from ketoesters 9 or 10, where Y is a leaving group such as halo and R″ is C₁-C₆ alkyl or benzyl. Either compound 9 or 10 may be chosen as the starting material depending on whether the R³ group in the desired end product is hydrogen or is attached at the 4 or 5 position. Accordingly, compound 9 or 10 may be reacted with an amide or thioamide where X is either O or S to yield compound 4. Ketoesters 9 or 10 are commercially available, or may be prepared by methods well known in the art such as by bromination of commercially available ketoesters 9 and 10 where Y is hydrogen. Amides (R⁴C(═X)NH₂) where X is O may be commercially available carboxylic amides, or may be prepared from the corresponding available acids or acid chlorides by well known methods. Thioamides (R⁴C(═X)NH₂) where X is S may be commercially available thioamides, or may be prepared from the corresponding available amides by known methods such as through the use of Lawesson's reagent. Reaction of ketoester 9 with an amide or thioamide in the presence of a base provides compound 4 as an oxazole or a thiazole, respectively, where R³ is other than hydrogen and located at the 4-position. Reaction of ketoester 10 with an amide or thioamide in the presence of base provides compound 4 as an oxazole or thiazole, where R³ is located at the 5-position.

Routes (A), (B), and (C) each provide compound 4 where R³ and R⁴ are each as described for a compound of Formula I and where R″ is a lower alkyl or benzyl. Compound 4 may then be reduced to compound 5 using reducing agents such as lithium aluminum hydride, lithium borohydride, or other suitable hydride donors under conditions well known in the art.

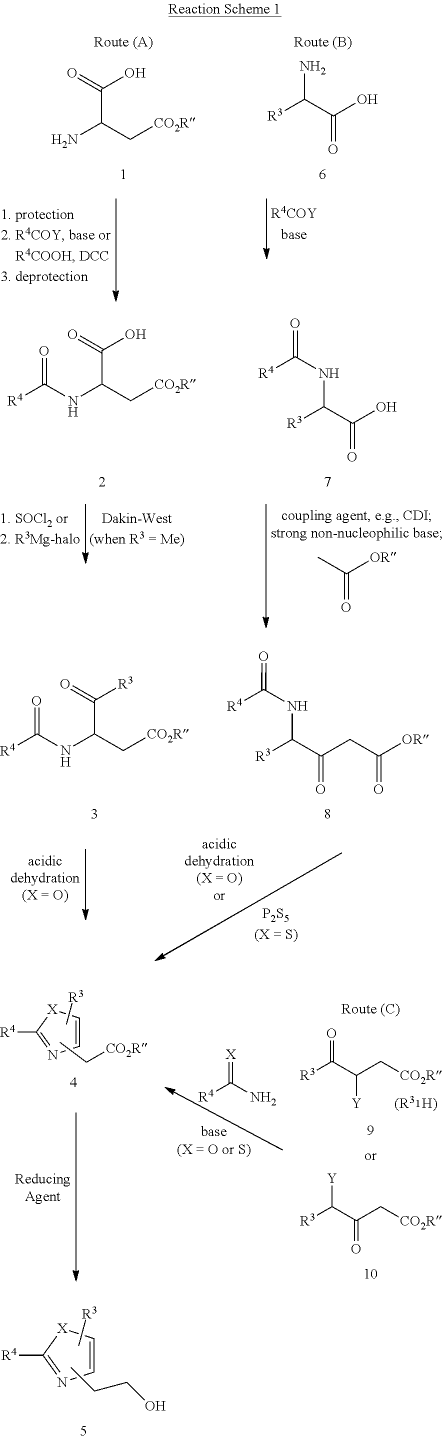

Reaction Scheme 2 depicts the conversion of commercially available hydroxy ketone 11 to a protected derivative 12, by reaction with R⁷—Y in the presence of a base, where R⁷ is C₁-C₆ alkyl optionally substituted with phenyl or oxo, C₁-C₆ trialkylsilyl, arylalkylsilyl, or COR⁸; and R⁸ is C₁-C₆ alkyl or phenyl optionally substituted with C₁-C₆ alkyl, halo, or nitro; and Y is a leaving group. “C₁-C₆ trialkylsilyl” means three independently selected straight or branched chain alkyl groups having from one to about six carbon atoms, each of which are bound to silicon and includes such groups as trimethylsilyl, tert-butyldimethyl silyl, and the like. “Arylalkylsilyl” means at least one phenyl or substituted phenyl group bound to silicon, with an appropriate number of independently selected straight or branched chain alkyl groups having from one to about six carbon atoms, each of which are also bound to silicon, and includes such groups as t-butyldiphenylsilyl methyldiphenylsilyl, dimethylpentafluorophenylsilyl, and the like. “Leaving group” includes halides such as I, Br, and Cl; carboxylates such as acetates, and trifluoroacetates; and aryl and alkyl sulfonates such as methanesulfonates (mesylates) and p-toluene sulfonates (tosylates), and the like.

Compound 12 is substituted with R² (as described in Formula I) by means of, for example, reaction with a source of electrophilic halogen, or a Friedel-Crafts reaction in the presence of a Lewis acid and R²—Y where Y is as described above, to form a substituted ketone 13. Alternatively, a halogenated compound formed in this manner (for example, substituted with bromine or iodine) may be reacted with a range of coupling partners under metal catalysis, using complexes and compounds of elements such as palladium and nickel well known to those skilled in the art, to form further substituted ketone 13. Exemplary catalysts include, but are not limited to, tetrakis(triphenylphosphine)palladium(0), [1,1′-bis(diphenylphosphino)ferrocene]dichloropalladium(II), and similar nickel(0) and nickel(II) compounds; and examples of coupling partners include boronic acids and esters (the well known Suzuki coupling, carried out in solvents such as toluene in the presence of a base such as potassium carbonate), and organometallics such as Grignard reagents, organozincs (Negishi coupling), and organotin derivatives (Stifle coupling), reaction conditions for which are widely known. Furthermore, such halogenated compounds may be coupled with secondary amines such as piperidine using similar palladium or nickel catalysts (Hartwig or Buchwald coupling) to provide further substituted ketones 13.

Further reaction of compound 13 with a halogen source or R⁵—Y, (where R⁵ is as described in Formula I), under similar conditions gives disubstituted compound 14. A Wittig reaction, or the Horner-Emmons-Wadsworth variation, each well known in the art, may be used to convert 14 to compound 15. For example, reaction of compound 14 with a trialkylphosphonoacetate, where R″ is lower alkyl and R is as described in Formula I, in the presence of a strong base such as sodium hydride, provides compound 15. In like manner, compound 13 may be converted to compound 15 where R⁵ is H.

Regardless of the isomeric mixture of isomers of 15 produced in the reaction, either isomer (E or Z) or a mixture of both, may be converted to the corresponding compound 17 by catalytic hydrogenation or reduction with a hydride reagent capable of 1,4 (conjugate) addition, which are known to those skilled in the art. This route is particularly advantageous for preparing compound 17 where R¹ is hydrogen.

Compound 17 where R¹ is COOR, may be prepared through standard condensation reactions, for example, the well known Knoevenagel reaction. In such cases, the ketone 13 or 14 may be reacted with a suitable active-hydrogen coupling partner, under the influence of acidic reagents such as titanium tetrachloride, or basic reagents such as piperidine, in appropriate solvents. The product 15b (compound 15 where R¹ is COOR), may be reduced to 17b (compound 17 where R¹ is COOR), which may be further alkylated with another R¹ group in the presence of base, hydrolyzed and decarboxylated to give 17d (compound 17 where R¹ is other than COOH and R is H). Reesterification of 17d and removal of the protecting group R⁷ would afford 17c. Reesterification may be performed using standard conditions using the well-known Fischer esterification by treatment with an acid and an alcohol or by reaction with diazoalkyl reagents or with an electrophilic species such as, for example, methyl iodide or dimethyl sulfate. Compound 17 where R¹ is alkoxy may be prepared by a similar condensation reaction of ketone 13 or 14 with a silylated enol ester of Formula R¹CH═C(OR″)O— alkylsilyl, where R¹ is alkoxy, under the influence of acidic reagents such as titanium tetrachloride, and reducing the intermediate compound 15, where R¹ is alkoxy, in the presence of hydrogen and a catalyst as described above.

A general coupling reaction of compound 13 or 14 via the Reformatsky reaction produces compound 16 (Formula II), when R¹ is alkyl, or compound 15a when R¹ is H. The ketone is condensed with an appropriate organozinc reagent prepared in situ from Zn and R¹CHYCO₂R, where Y is halo. The alpha-halo ester compounds of formula R¹CHYCO₂R, are either commercial reagents or are prepared by halogenation of commercially available R¹CH₂CO₂R compounds by methods well known to those skilled in the art.

The conversion of 16 to 17 may be accomplished by standard hydrogenation conditions, for example, Pd/C and hydrogen; and deprotection of compound 17, where R⁷ is a protecting group, to compound 17c, where R⁷ is hydrogen, may be accomplished by standard means. For example, when the R⁷ group is alkyl (e.g., methyl), the compound 17a may be generated by nucleophilic cleavage with a reagent such as an alkali metal thiolate. Alternatively, compound 17 when R⁷ is methyl, may be converted to compound 17c by reaction with a Lewis acid such as a bromoborane. When R⁷ is benzyl, the compound 17 may be converted to 17c under hydrogenation conditions, typically carried out using a catalyst such as palladium. Other conditions for the removal of the protecting group R⁷ from compound 17, where R⁷ is other than hydrogen which produces the hydroxy compound 17c, are dependent on the specific protecting group chosen from among those which are well known by those skilled in the art.

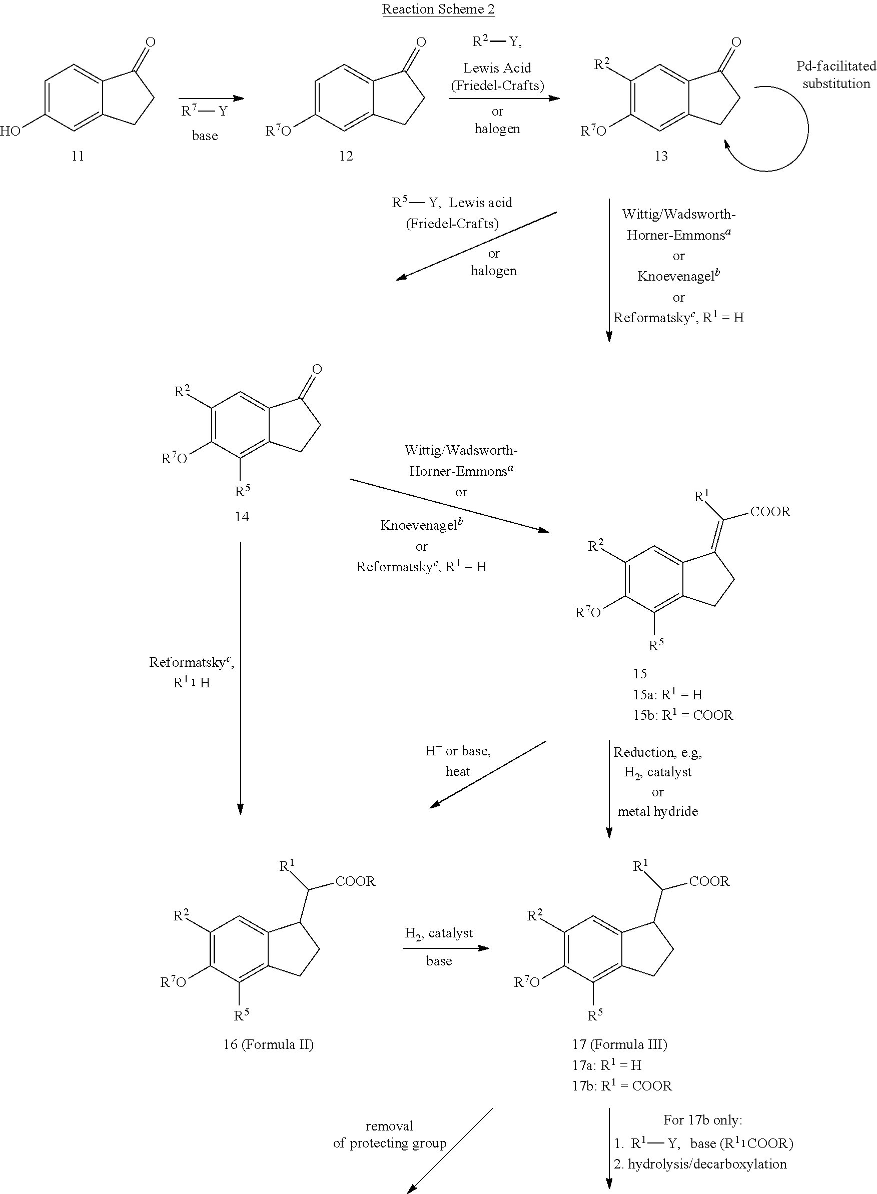

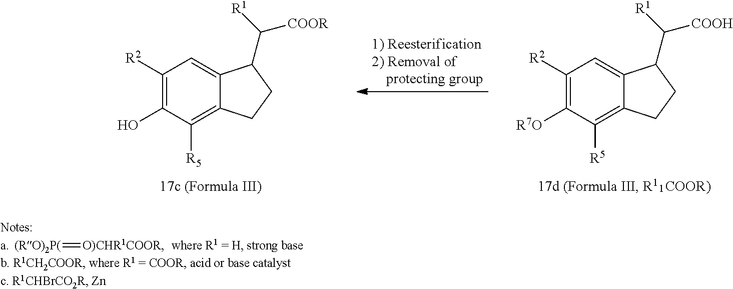

The final step in the preparation of Formula I compounds is shown in Reaction Scheme 3. The alcohol 5 (from Reaction Scheme 1) is coupled with the hydroxy indane 17c (from Reaction Scheme 2) via a Mitsunobu coupling, facilitated by an azodicarboxylate reagent such as DEAD, and a phosphine such as triphenylphosphine to make the compounds of Formula I. Alternatively, the hydroxy group of alcohol 5 is converted to a leaving group such as halo, tosylate (OTs), or mesylate (OMs), by reaction with a halogenating agent such as thionyl chloride or CCl₄/triphenylphosphine; or by reaction with a Y-halo compound, where Y is tosyl (Ts) or mesyl (Ms), in the presence of a base, providing compound 18. Compound 18 may be reacted with compound 17c in the presence of a base, providing the compounds of Formula I.

Compounds of Formula I in which R is alkyl, may be converted to compounds of Formula I in which R is H by treatment with a base (e.g., KOH) in a suitable solvent (e.g., methanol, THF, or water, or mixtures thereof) with heating. Alternatively, this conversion may be accomplished by reaction with a nucleophile such as iodide or cyanide, in a suitable solvent, such as pyridine. In addition, when R is benzyl, the cleavage to compounds of Formula I in which R is H may be affected through hydrogenolysis by means well known in the art.

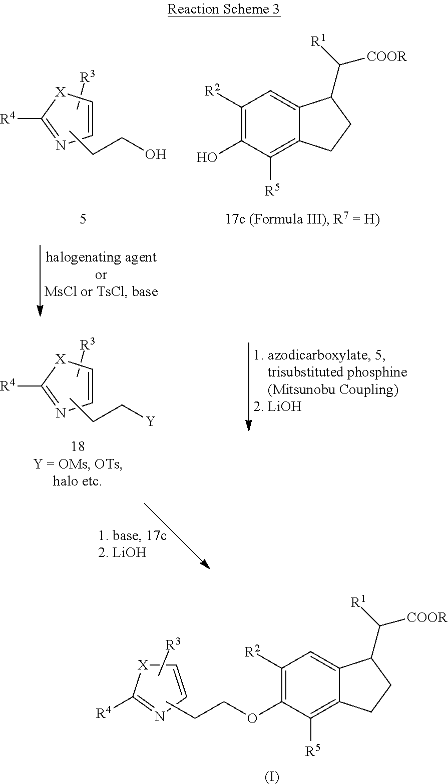

An alternative route to Formula I compounds, useful when X=S and the R⁴ group contains one or more R⁶ substituents labile to the reaction conditions of Scheme 1 or 2, is shown in Reaction Scheme 3a.

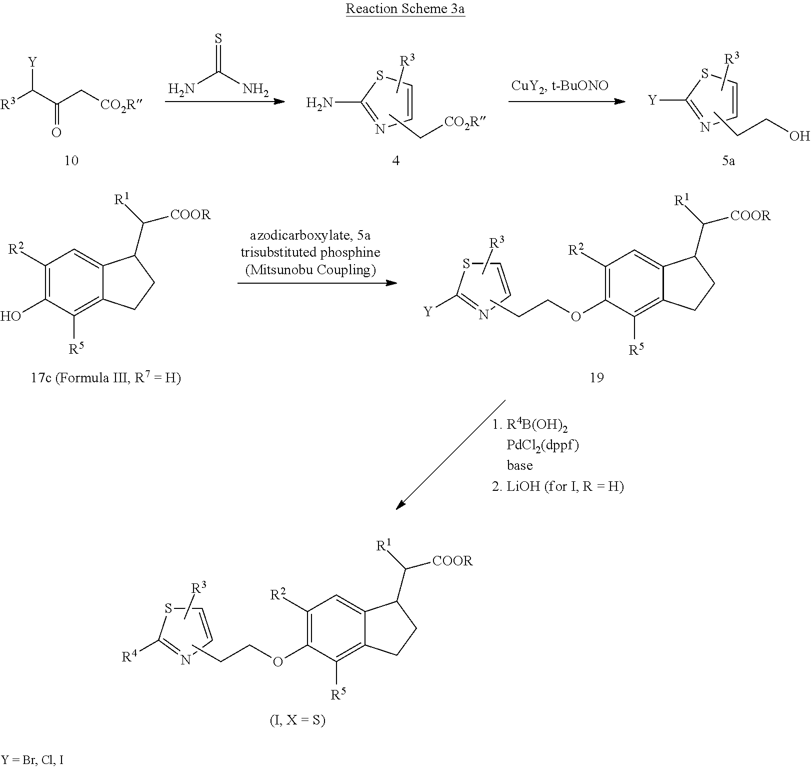

In Scheme 3a, a 2-aminothiazole 4 may be prepared using thiourea (similar to Route C, Reaction Scheme 1) and converted to a 2-halo thiazole 5a as shown above (Erlenmeyer et al., Helv. Chim. Acta 28:362-363, 1945). Mitsunobu coupling of 5a by a method analogous to Reaction Scheme 3 is then accomplished, and product 19 is further elaborated by a Palladium-catalyzed cross-coupling reaction to introduce the R⁴ substituent. Hydrolysis as described in Reaction Scheme 3 gives Formula I compounds where R=H.

The foregoing reaction schemes are further illustrated by the specific Examples described herein.

The salts and esters of this invention may be readily prepared by conventional chemical processes as described previously herein.

The invention is further directed to novel Formula II compounds (compound 16) and Formula III (compounds 17, including compounds 17a-d) compounds shown in Reaction Scheme 2. These compounds are useful in the preparation of the compounds of Formula I, and are further described as follows.

The present invention encompasses the compounds of Formula II and Formula III,

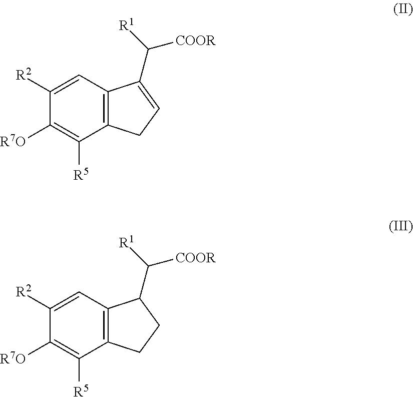

wherein
R, R¹, R², R³, R⁴, R⁵, R⁶, and X are as defined for Formula I above; and
R⁷ is H, C₁-C₆ alkyl optionally substituted with phenyl or oxo, C₁-C₆ trialkylsilyl, arylalkylsilyl, COR⁸, COOR⁸, or

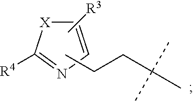

R⁸ is C₁-C₆ alkyl, or phenyl optionally substituted with C₁-C₆ alkyl, halo, or nitro; and
   the salts thereof.

C₁-C₆ trialkylsilyl means three independently selected straight or branched chain alkyl groups having from one to about six carbon atoms, each of which are bound to silicon and includes such groups as trimethylsilyl, tert-butyldimethyl silyl, and the like.

Arylalkylsilyl means at least one phenyl or substituted phenyl group bound to silicon, with an appropriate number of independently selected straight or branched chain alkyl groups having from one to about six carbon atoms, each of which are also bound to silicon, and includes such groups as t-butyldiphenylsilyl methyldiphenylsilyl, dimethylpentafluorophenylsilyl, and the like.

The salts of this invention may be readily prepared by conventional chemical processes as described previously herein.

The compounds of Formula II and Formula III may each contain one or more asymmetric centers, depending upon the location and nature of the various substituents desired. Asymmetric carbon atoms may be present in the (R) or (S) configuration. Preferred isomers are those with the absolute configuration, which produces the compound of Formula II or Formula III that will be useful in producing the compounds of Formula I having a more desirable biological activity. In certain instances, asymmetry may also be present due to restricted rotation about a given bond, for example, the central bond adjoining two aromatic rings of the specified compounds.

Substituents on a ring may also be present in either cis or trans form, and a substituent on a double bond may be present in either Z or E form.

It is intended that all isomers (including enantiomers and diastereomers), either by nature of asymmetric centers or by restricted rotation as described above, as separated, pure or partially purified isomers or racemic mixtures thereof, be included within the scope of the present invention. The purification of said isomers and the separation of said isomeric mixtures may be accomplished by standard techniques known in the art, as well as by the novel means described herein.

For example, Formula II compounds may contain an asymmetric center (labeled C-2) and Formula III compounds may contain two asymmetric centers (labeled C-2 and C-1′) which give rise to enantiomers and diastereomers. Examples of these and other compounds of Formula II and Formula III, which are illustrative of the present invention, are shown in Table 2.

TABLE 2
Illustrative Examples of Compounds II and III

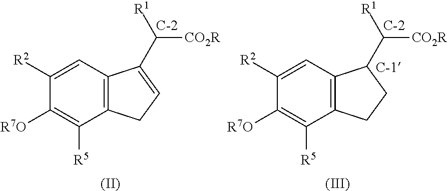

| | | absolute | | | | | |
|---|---|---|---|---|---|---|---|
| Entry | | configuration | | | | | |
| No. | Formula | C-2 | C-1′ | R¹ | R² | R⁵ | R⁷ |
| 1 | II | R | — | H | H | H | CH₃ |
| 2 | III | R | R | H | H | H | CH3 |
| 3 | II | R | — | Cl | H | H | t-Bu(CH₃)₂Si |
| 4 | III | R | S | Cl | H | H | t-Bu(CH₃)₂Si |
| 5 | II | S | — | H | H | H | CH₃ |
| 6 | III | S | S | H | H | H | CH₃ |
| 7 | II | R | — | CH₃ | H | H | CH₃ |
| 8 | III | R | R | CH₃ | H | H | CH₃ |
| 9 | II | S | — | CH₃ | H | H | CH₃ |
| 10 | III | S | R | CH₃ | H | H | CH₃ |
| 11 | II | R | — | CH₃ | H | H | PhCH₂ |
| 12 | III | R | S | CH₃ | H | H | PhCH₂ |
| 13 | II | S | — | CH₃ | H | H | PhCH₂ |
| 14 | III | S | S | CH₃ | H | H | PhCH₂ |
| 15 | II | R | — | CH₃ | H | H | t-Bu(CH₃)₂Si |
| 16 | III | R | R | CH₃ | H | H | t-Bu(CH₃)₂Si |
| 17 | II | S | — | CH₃ | H | H | t-Bu(CH₃)₂Si |
| 18 | II | R | — | CH₃ | H | H | t-BuCO |
| 19 | III | R | S | CH₃ | H | H | t-BuCO |
| 20 | II | S | — | CH₃ | H | H | t-BuCO |
| 21 | III | S | S | CH₃ | H | H | t-BuCO |
| 22 | II | R | — | CH₃ | CH₃ | H | PhCH₂ |
| 23 | II | R | — | CH₃ | CH₃CO | H | PhCH₂ |
| 24 | II | S | — | CH₃ | 2-thienyl | H | t-Bu(CH₃)₂Si |
| 25 | III | S | R | CH₃ | 2-thienyl | H | t-Bu(CH₃)₂Si |
| 26 | II | S | — | CH₃ | Ph | H | CH₃ |
| 27 | II | R | — | CH₃ | Cl | H | CH₃ |
| 28 | II | S | — | CH₃ | Cl | H | CH₃ |
| 29 | III | S | S | CH₃ | Cl | H | CH₃ |
| 30 | II | R | — | CH₃ | Br | H | Ph(CH₃)₂Si |
| 31 | III | R | R | CH₃ | Br | H | Ph(CH₃)₂Si |
| 32 | II | S | — | CH₃ | Br | H | Ph(CH₃)₂Si |
| 33 | III | S | R | CH₃ | Br | H | Ph(CH₃)₂Si |
| 34 | II | S | — | CH₃ | Cl | Cl | CH₃ |
| 35 | II | R | — | Et | H | H | CH₃ |
| 36 | III | R | R | Et | H | H | CH₃ |
| 37 | II | S | — | Et | H | H | PhCH₂ |
| 38 | III | S | S | Et | H | H | PhCH₂ |
| 39 | II | R | — | Et | H | H | t-Bu |
| 40 | II | S | — | Et | H | H | t-Bu |
| 41 | II | S | — | Et | CH₃ | H | Ph(CH₃)₂Si |
| 42 | III | S | S | Et | CH₃ | H | Ph(CH₃)₂Si |
| 43 | II | R | — | Et | n-propyl | H | CH₃ |
| 44 | II | S | — | Et | Ph | H | CH₃ |
| 45 | II | S | — | Et | 3-Cl Ph | H | t-Bu(CH₃)₂Si |
| 46 | III | S | R | Et | 3-Cl Ph | H | t-Bu(CH₃)₂Si |
| 47 | II | S | — | Et | 4-pyridyl | H | t-Bu(CH₃)₂Si |
| 48 | III | S | S | Et | 4-pyridyl | H | t-Bu(CH₃)₂Si |
| 49 | II | S | — | Et | CH₃ | H | Ph(CH₃)₂Si |
| 50 | II | R | — | Et | n-propyl | Cl | CH₃ |
| 51 | II | R | — | Et | Br | Br | t-Bu(CH₃)₂Si |
| 52 | III | R | R | Et | Br | Br | t-Bu(CH₃)₂Si |
| 53 | II | S | — | CF₃CH₂ | H | H | CH₃ |
| 54 | II | S | — | CF₃CH₂ | CH₃ | CH₃ | (4-CH₃O)PhCH₂ |
| 55 | III | S | S | CF₃CH₂ | CH₃ | CH₃ | (4-CH₃O)PhCH₂ |
| 56 | II | S | — | n-propyl | H | H | (i-Pr)₃Si |
| 57 | II | R | — | n-propyl | PrCO | PrCO | t-Bu |
| 58 | II | R | — | n-propyl | Cl | Cl | (i-Pr)₃Si |
| 59 | III | R | R | n-propyl | Cl | Cl | (i-Pr)₃Si |
| 60 | II | S | — | isopropyl | CH₃ | H | CH₃ |
| 61 | III | S | R | isopropyl | CH₃ | H | CH₃ |
| 62 | II | R | — | isopropyl | n-hexyl | H | (4-CH₃O)PhCH₂ |
| 63 | III | R | S | isopropyl | n-hexyl | H | (4-CH₃O)PhCH₂ |
| 64 | II | S | — | n-butyl | H | H | PhCH2 |
| 65 | II | S | — | n-butyl | CH₃OCH₂ | H | t-Bu(CH₃)₂Si |
| 66 | III | S | S | n-butyl | CH₃OCH₂ | H | t-Bu(CH₃)₂Si |
| 67 | II | R | — | n-butyl | Cl | H | CH₃ |
| 68 | II | R | — | n-pentyl | Cl | Cl | (4-CH₃O)PhCH₂ |
| 69 | II | S | — | n-pentyl | 2-thienyl | 2-thienyl | CH₃ |
| 70 | III | S | S | n-pentyl | 2-thienyl | 2-thienyl | CH₃ |
| 71 | II | R | — | n-hexyl | CH₃CO | H | t-Bu(CH₃)₂Si |
| 72 | III | R | S | n-hexyl | CH₃CO | H | t-Bu(CH₃)₂Si |
| 73 | II | R | — | n-hexyl | Ph | H | Ph(CH₃)₂Si |
| 74 | III | R | R | n-hexyl | Ph | H | Ph(CH₃)₂Si |
| 75 | II | R | — | cyclopropyl | H | H | t-BuCO |
| 76 | II | S | — | cyclopropyl | CH₃ | H | (i-Pr)₃Si |
| 77 | II | S | — | cyclobutyl | H | H | CH₃ |
| 78 | III | S | S | cyclobutyl | H | H | CH₃ |
| 79 | II | S | — | cyclobutyl | Cl | Cl | (4-CH₃O)PhCH₂ |
| 80 | II | R | — | cyclopentyl | CH₃ | H | t-Bu(CH₃)₂Si |
| 81 | III | R | S | cyclopentyl | CH₃ | H | t-Bu(CH₃)₂Si |
| 82 | II | S | — | cyclohexyl | Et | Et | CH₃ |
| 83 | II | R | — | cyclohexyl | 2-thienyl | H | CH₃CO |
| 84 | II | R | — | cyclohexyl | Cl | H | CH₃ |
| 85 | III | R | R | cyclohexyl | Cl | H | CH₃ |
| 86 | II | S | — | 2-propenyl | H | H | t-Bu(CH₃)₂Si |
| 87 | II | R | — | 2-propenyl | CH₃ | H | CH₃CO |
| 88 | II | S | — | 2-isobutenyl | CH₃CO | H | CH₃ |
| 89 | II | S | — | 5-hexenyl | CH₃CO | CH₃CO | CH₃ |
| 90 | II | S | — | CH₃O | H | H | PhCH₂ |
| 91 | III | S | R | CH₃O | H | H | PhCH₂ |
| 92 | II | R | — | CH₃O | 3-F Ph | H | (4-CH₃O)PhCH₂ |
| 93 | II | S | — | EtO | Cl | Cl | PhCH₂ |
| 94 | III | S | R | EtO | Cl | Cl | PhCH₂ |
| 95 | II | R | — | (i-Pr)O | H | H | PhCH₂ |
| 96 | III | R | R | (i-Pr)O | H | H | PhCH₂ |
| 97 | II | S | — | (n-pentyl)O | CH₃ | H | t-Bu(CH₃)₂Si |
| 98 | III | S | S | (n-pentyl)O | CH₃ | H | t-Bu(CH₃)₂Si |
| 99 | II | S | — | CO₂H | H | H | (4-CH₃O)PhCH₂ |

Another embodiment of the present invention is an improved process for the preparation of compounds having a specific isomeric configuration when that specific configuration is desired for the ultimate desired end product of Formula I. The improved process yields these intermediate compounds in significantly greater diastereomeric excess than was heretofore possible.

Previously, for example, in the absence of stereocontrol during the hydrogenation step of Reaction Scheme 2, hydrogenation of a Formula II compound, where R¹ is alkyl may produce an unequal mixture of diastereomeric products of Formula III, in which one pair of enantiomers is favored because of the asymmetric nature of the starting material. Separation of such compounds may be accomplished by stepwise separation of the enantiomeric pairs, then by resolution of each enantiomer by crystallization or by chiral HPLC. Prior resolution of the starting material into a single enantiomer produces mixtures with enrichment of a single enantiomer that may likewise be separated.

However, when a compound of a specific relative configuration, namely a syn form (defined below) is desired, the yield is low when R¹ is alkyl, because the conditions of the hydrogenation step described in the art may favor the other (i.e., anti) diastereomers.

The desired isomeric configurations realized from this improved process are in the syn form where, for example, in compounds of Formula Va and Vb (depicted in Reaction Schemes 4 and 5), the R⁹ group and the 2′ methylene carbon of the cyclopentane ring are both below the plane or are both above the plane. Anti diastereomers are those compounds where, for example, R⁹ is above the plane and 2′ methylene is below the plane. This is further exemplified in Examples 1 and 2 below, in which solid wedge bonds are used to indicate projection of the bond above the plane and dashed wedge bonds are used to indicate projection of the bond below the plane.

### Example 1

### Syn Diastereomers of Formula V

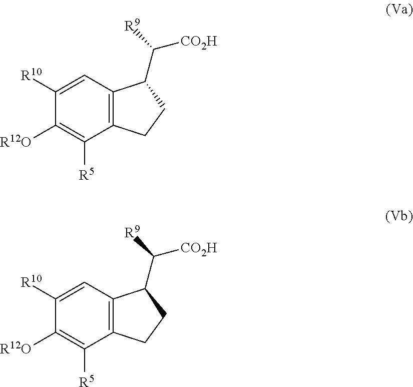

### Example 2

### Anti Diastereomers of Formula V

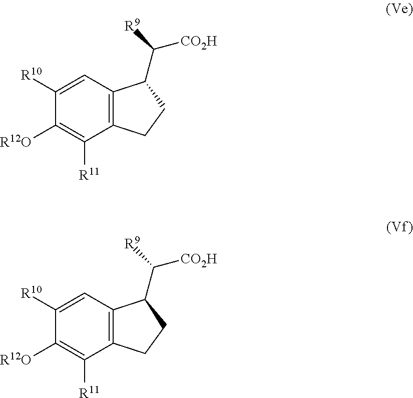

The improved process of this invention yields compounds in the syn form (Formulas Va and Vb, as drawn in Example 1 and Reaction Schemes 4 and 5) in significantly higher diastereomeric excess than was generally possible.

The intermediate compounds used as starting materials for this process (compound IV of Reaction Schemes 4 and 5) are related to the compounds of Formula II (compound 16) in Reaction Scheme 2, and may be prepared by the same or analogous methods. These intermediates may be reacted under certain conditions to yield Formula V compounds that are related to compounds of Formula III (compounds 17 and 17a of Reaction Scheme 2), or to directly yield compounds of Formula I. However, due to the constraints of the improved process, only certain substituents are appropriate for completing this process.

Accordingly, the present invention relates to an improved process for the preparation of a substantially enriched syn form of a compound of Formula V,

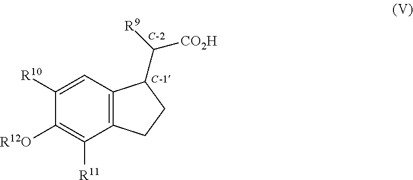

wherein
R⁹ is methoxy optionally substituted by fluoro, C₂-C₆ alkoxy, C₁-C₆ alkyl, or C₄-C₈ cycloalkyl each optionally substituted by fluoro, methylenedioxyphenyl or phenyl optionally substituted with R¹³;
R¹⁰ is hydrogen, fluoro, methyl optionally substituted with fluoro, oxo, or C₂-C₆ alkyl which may be unsubstituted or substituted with C₁-C₆ alkoxy, oxo, fluoro, or with phenyl, furyl, thienyl, pyrrolyl, oxazolyl, thiazolyl, imidazolyl, pyrazolyl, isoxazolyl, isothiazolyl, triazolyl, oxadiazolyl, thiadiazolyl, tetrazolyl, pyridyl, pyrrolidinyl, piperidinyl, tetrahydropyranyl, tetrahydrothiopyranyl, piperazinyl, or morpholinyl,

each of which may be unsubstituted or substituted with R¹³, or

R¹⁰ is phenyl, furyl, thienyl, pyrrolyl, oxazolyl, thiazolyl, imidazolyl, pyrazolyl, isoxazolyl, isothiazolyl, triazolyl, oxadiazolyl, thiadiazolyl, tetrazolyl, pyridyl, pyrrolidinyl, piperidinyl, tetrahydropyranyl, tetrahydrothiopyranyl, piperazinyl, or morpholinyl, each of which may be unsubstituted or substituted with R¹³;
R¹¹ is halo or C₁-C₆ alkyl optionally substituted with oxo;
R¹² is hydrogen, methyl optionally substituted with fluoro or oxo, C₂-C₆ alkyl optionally substituted with phenyl, fluoro, or oxo, C₁-C₆ trialkylsilyl, arylalkylsilyl, COR¹⁴, COOR¹⁴, or

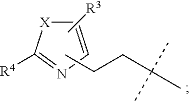

R¹³ is fluoro, CF₃, C₁-C₆ alkyl optionally substituted with oxo, or C₁-C₆ alkoxy optionally substituted with fluoro;
R¹⁴ is C₁-C₆ alkyl, or phenyl optionally substituted with C₁-C₆ alkyl or fluoro;
R¹⁵ is hydrogen, C₁-C₆ alkyl or phenyl substituted with R¹³;
R¹⁶ is methyl optionally substituted with fluoro, oxo or with phenyl, naphthyl, furyl, thienyl, pyrrolyl, tetrahydrofuryl, pyrrolidinyl, pyrrolinyl, tetrahydrothienyl, oxazolyl, thiazolyl, imidazolyl, pyrazolyl, isoxazolyl, isothiazolyl, triazolyl, oxadiazolyl, thiadiazolyl, tetrazolyl, pyridyl, piperidinyl, tetrahydropyranyl, tetrahydrothiopyranyl, pyrimidinyl, pyrazinyl, pyridazinyl, piperazinyl, morpholinyl, benzofuryl, dihydrobenzofuryl, benzothienyl, dihydrobenzothienyl, indolyl, indolinyl, indazolyl, benzoxazolyl, benxothiazolyl, benzimidazolyl, benzisoxazolyl, benzisothiazolyl, benzodioxolyl, quinolyl, isoquinolyl, quinazolinyl, quinoxazolinyl, dihydrobenzopyranyl, dihydrobenzothiopyranyl, or 1,4-benzodioxanyl,
each of which may be unsubstituted or substituted with R¹³, or C₄-C₈ cycloalkyl or C₂-C₆ alkyl, either of which may be unsubstituted or substituted with fluoro, methoxy, C₂-C₆ alkoxy optionally substituted with phenyl or C₁-C₆ alkoxy, oxo or with, phenyl, naphthyl, furyl, thienyl, pyrrolyl, tetrahydrofuryl, pyrrolidinyl, pyrrolinyl, tetrahydrothienyl, oxazolyl, thiazolyl, imidazolyl, pyrazolyl, isoxazolyl, isothiazolyl, triazolyl, oxadiazolyl, thiadiazolyl, tetrazolyl, pyridyl,
piperidinyl, tetrahydropyranyl, tetrahydrothiopyranyl, pyrimidinyl, pyrazinyl, pyridazinyl, piperazinyl, morpholinyl, benzofuryl, dihydrobenzofuryl, benzothienyl, dihydrobenzothienyl, indolyl, indolinyl, indazolyl, benzoxazolyl, benxothiazolyl, benzimidazolyl, benzisoxazolyl, benzisothiazolyl, benzodioxolyl, quinolyl, isoquinolyl, quinazolinyl, quinoxazolinyl, dihydrobenzopyranyl, dihydrobenzothiopyranyl, or 1,4-benzodioxanyl,
   each of which may be unsubstituted or substituted with R¹³, or C₂-C₆ alkyl which may also be substituted with C₄-C₈ cycloalkyl or with phenoxy which may be unsubstituted or substituted with R⁶ or with phenyl, naphthyl, furyl, thienyl, pyrrolyl, tetrahydrofuryl, pyrrolidinyl, pyrrolinyl, tetrahydrothienyl, oxazolyl, thiazolyl, imidazolyl, pyrazolyl, isoxazolyl, isothiazolyl, triazolyl, oxadiazolyl, thiadiazolyl, tetrazolyl, pyridyl, piperidinyl, tetrahydropyranyl, tetrahydrothiopyranyl, pyrimidinyl, pyrazinyl, pyridazinyl, piperazinyl, morpholinyl, benzofuryl, dihydrobenzofuryl, benzothienyl, dihydrobenzothienyl, indolyl, indolinyl, indazolyl, benzoxazolyl, benxothiazolyl, benzimidazolyl, benzisoxazolyl, benzisothiazolyl, benzodioxolyl, quinolyl, isoquinolyl, quinazolinyl, quinoxazolinyl, dihydrobenzopyranyl, dihydrobenzothiopyranyl, or 1,4-benzodioxanyl, each of which may be unsubstituted or substituted with R¹³, or
      R¹⁶ is phenyl, naphthyl, furyl, thienyl, pyrrolyl, tetrahydrofuryl, pyrrolidinyl, pyrrolinyl, tetrahydrothienyl, oxazolyl, thiazolyl, imidazolyl, pyrazolyl, isoxazolyl, isothiazolyl, triazolyl, oxadiazolyl, thiadiazolyl, tetrazolyl, pyridyl, piperidinyl, tetrahydropyranyl, tetrahydrothiopyranyl, pyrimidinyl, pyrazinyl, pyridazinyl, piperazinyl, morpholinyl, benzofuryl, dihydrobenzofuryl, benzothienyl, dihydrobenzothienyl, indolyl, indolinyl, indazolyl, benzoxazolyl, benxothiazolyl, benzimidazolyl, benzisoxazolyl, benzisothiazolyl, benzodioxolyl, quinolyl, isoquinolyl, quinazolinyl, quinoxazolinyl, dihydrobenzopyranyl, dihydrobenzothiopyranyl, or 1,4-benzodioxanyl,
each of which may be unsubstituted or substituted with R¹³, or with phenyl, furyl, thienyl, pyrrolyl, oxazolyl, thiazolyl, imidazolyl, pyrazolyl, isoxazolyl, isothiazolyl, triazolyl, oxadiazolyl, thiadiazolyl, tetrazolyl, pyridyl, pyrrolidinyl, piperidinyl, tetrahydropyranyl, tetrahydrothiopyranyl, piperazinyl, morpholinyl, pyrimidinyl or phenoxy each of which may be unsubstituted or substituted with R¹³, and
X is O or S;
   comprising hydrogenation of a racemic mixture or isolated optical isomer of a compound of Formula IV,

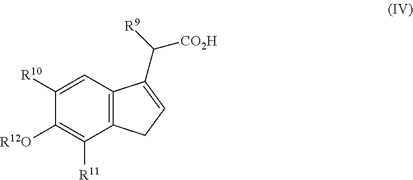

wherein the substituents are as defined above, in the presence of hydrogen source, a catalyst, optionally in the presence of a base.

Substantially enriched syn form means at least about seventy percent (70%) or greater of one or both of the compounds of the configuration of Va or Vb. This is equivalent to at least about 40% de (diastereomeric excess) of the syn diastereomer. Diastereomeric excess of the syn diastereomer is calculated from the following formula:

$$\% de ( syn ) = \frac{[ syn ] - [ anti ]}{[ syn ] + [ anti ]} \times 100 = \% syn diastereomer - \% anti diastereomer$$

in which

% de (syn) represents the diastereomeric excess of the syn diastereomer

[syn] represents the concentration of the syn diastereomer

[anti] represents the concentration of the anti diastereomer,

and where

% syn+% anti=100%.

Thus, a 40% de of the syn diastereomer is calculated from a mixture of 70% syn diastereomer and 30% anti diastereomer:

40% de(syn)=70% syn diastereomer−30% anti diastereomer

Catalyst means any of the transition metal catalysts well known in the art to effect hydrogenation reactions (P. A. Chaloner, Handbook of Co-ordination Catalysis in Organic Chemistry, Butterworth, 1986), and includes homogeneous hydrogenation catalysts. A homogeneous catalyst is a catalyst which is at least partially soluble in the reaction medium and which effects the reduction of a double bond in the presence of hydrogen. Such catalysts include, for example, ClRh[P(Ph)₃]₃ (Wilkinson's catalyst), (1,5-cyclooctadiene)tricyclohexylphosphinepyridinoiridium(I)hexafluorophosphate, (1,5-cyclooctadiene)bis(methyldiphenylphosphine)iridium(I) hexafluorophosphate (Crabtree's catalysts), and the like.

Base means a substance with a pK_{b} sufficient to form a salt in situ with a carboxylic acid (see, e.g., Advanced Organic Chemistry, 3rd Ed., Jerry March, pp 220-222). The base which is used in this reaction may be any inorganic or organic base, and may be soluble in the reaction medium. Such bases include, for example, mono, di, and tri(C₁-C₆ alkyl)amines such as isopropyl amine, diisopropyl amine, triethylamine, and the like; additional primary amines such as, for example, cyclohexane methylamine and ethanolamine; additional secondary amines such as, for example, morpholine and piperidine; and additional tertiary amines such as, for example, 1,8-diazaobicyclo[5.4.0]undec-7-ene and 1,5-diazabicyclo[4.3.0]non-5-ene as well as inorganic bases such as alkali metal and alkaline earth hydroxides, carbonates, bicarbonates, and optically active bases such as quinine, cinchonine or (+)- or (−)-alpha-methylbenzylamine.

Such bases also include, for example, the chiral bases named below that are useful for resolution. Hydrogen source refers to any means of delivering hydrogen to the reaction medium and includes the use of hydrogen gas. Hydrogenation may by performed under a broad range of hydrogen pressures, that is, from about atmospheric pressure to about 1000 psi, preferably from about 20 to about 100 psi. Suitable hydrogenation solvents include, but are not limited to, protic solvents such as ethanol, methanol, water, 2-proponal, tert-butanol, methyl cellosolve and the like, and mixtures thereof, or optionally mixtures thereof with a miscible aprotic solvent such as THF, such that the hydrogenation catalyst, the base, and the starting material are each at least partially soluble.

The resolution of the starting indene acetic acid derivatives of Formula IV or of the indane acetic acid derivatives of Formula V may be accomplished by means well known in the art, for example, by using optically active bases as resolving agents such as, for example, a readily available base such as quinine, cinchonine or (+)- or (−)-alpha-methylbenzylamine. Choice of the base will depend on the solubility properties of the salt formed, so that resolution by differential recrystallization may be readily accomplished. By selecting bases with opposite absolute configuration, separation of the salt of each enantiomer may be accomplished. For example, for the embodiment illustrated in Reaction Scheme 4, the desired enantiomer IVc or IVd may be separated, and the undesired isomer may be recycled by racemization under basic conditions to the starting material of Formula IV.

Suitable crystallization solvents refer to those solvents in which one diastereomeric salt of a mixture is more soluble than the other, enabling them to be separated by recrystallization. Such solvents include, for example, acetonitrile, acetone, t-butanol, 2-propanol, ethanol, methanol, and the like, and mixtures thereof.

Aqueous mineral acids include, for example, the commonly used inorganic acids such as hydrochloric or sulfuric acid, and the like.

The process may be carried out starting with a racemate of Formula IV (see Reaction Scheme 4), or with a Formula V compound with the configuration at one asymmetric carbon which corresponds to that of the desired end product (see Reaction Scheme 5). Starting with the generally pure configuration is preferred, although either process will yield the desired configuration of the end product (V) in substantially enriched syn form.

One embodiment of this process is shown in the example of Reaction Scheme 4 and includes the steps of
(1) formation of diastereomeric salts of IVc and IVd by treatment of IV with a suitable basic resolving agent,
(2) separation of the diastereomeric salts IVc and IVd by crystallization in a suitable crystallization solvent,
(3) optionally liberating the individual antipodes IVa and IVb from the separated salts by treatment with aqueous mineral acid, and
(4) reduction of either the separated diastereomeric salts IVc and Vd or the individual antipodes IVa and IVb by hydrogenation in the presence of a homogeneous hydrogenation catalyst, a suitable solvent and a base, wherein M+ is a cation selected from an alkali metal, alkaline earth metal, ammonium, and mono-, di-, tri- or quaternary alkylammonium or aralkylammonium, and R⁹-R¹² are as defined above.

The enantiomeric purity of the product Va and Vb will correspond to the enantiomeric purity of the isomer IVa or IVb used, respectively, but will not include any substantial amount of the other (anti) diastereoisomer.

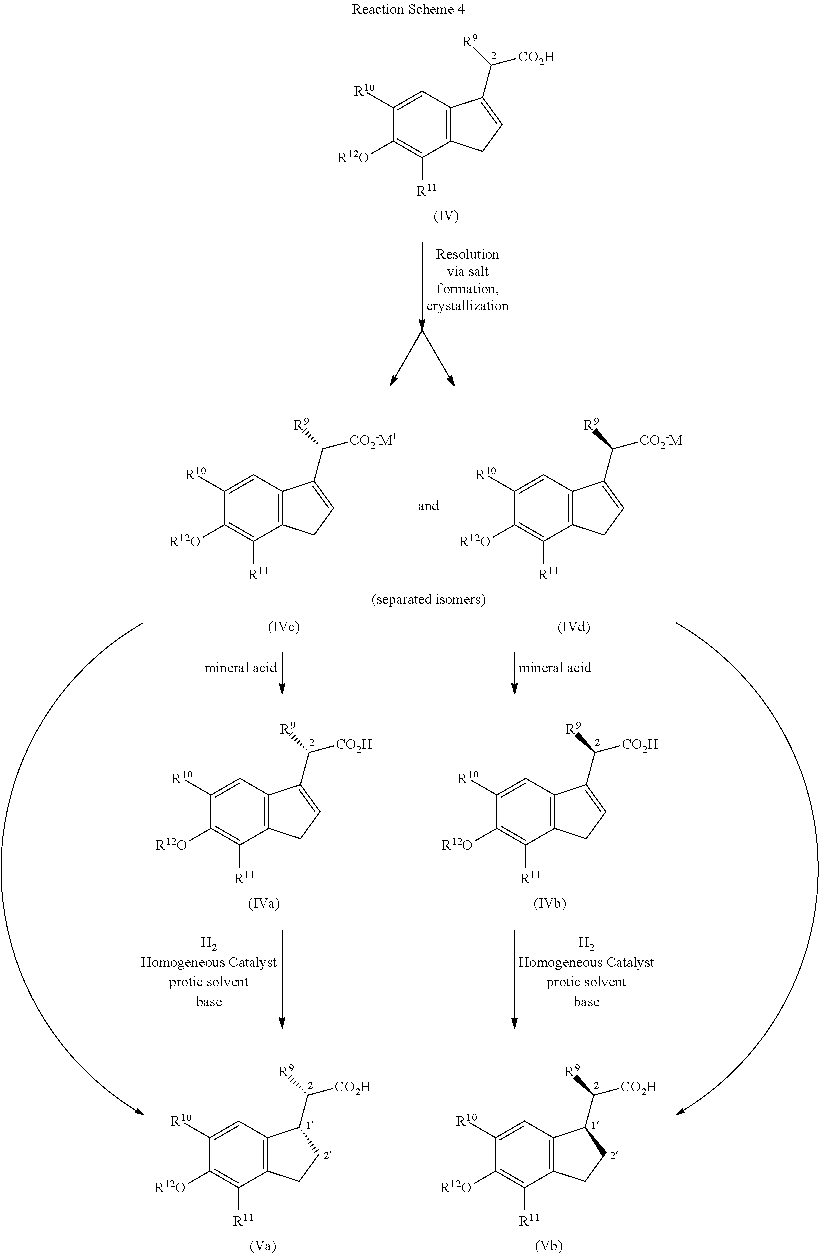

A second embodiment of this process is shown in Reaction Scheme 5 and includes the steps of
(1) reduction of the indene carboxylic acid of Formula IV by hydrogenation in the presence of a homogeneous hydrogenation catalyst, a suitable solvent, and a base,
(2) formation of diastereomeric salts of Vc and Vd by treatment of V with a suitable basic resolving agent,
(3) separation of the diastereomeric salts Vc and Vd by crystallization in a suitable crystallization solvent, and
(4) liberating the individual antipodes Va and Vb from the separated salts by treatment with aqueous mineral acid.

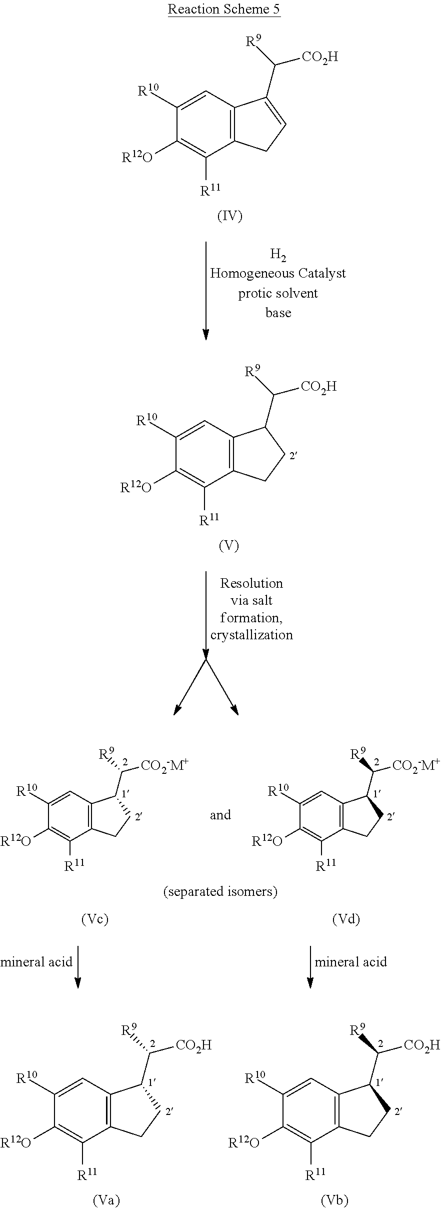

The resolution of the racemate of either Formula IV or Formula V compounds may be accomplished by means well known in the art, such as by chiral HPLC, crystallization of chiral salt derivatives, chiral ester derivatives, and the like.

The determination of absolute chirality of IVa, IVb, IVc, IVd, Va, and Vb may be accomplished by several means known to those skilled in the art. X-ray crystallographic methods may provide such information under certain well-established conditions. For example, the presence in the crystallographic unit cell of another component of known chirality, such as a chiral resolving agent or auxiliary in the form of a salt, complex, or covalently attached group, may allow such determination. Another method known in the art, heavy atom scattering technique may be utilized when the compound to be assayed contains an atom of sufficient mass (for example, bromine or iodine). Other methods involving optical properties and the use of plane-polarized light may also be employed. For example, one skilled in the art would recognize that such techniques as circular dichroism may be applicable to a given structure or structural class.

Specific examples of the intermediates that may be made with the process of the present invention are shown below by way of example, and not by way of limitation, and may be used for the preparation of compounds of Formula I of the same absolute configuration.

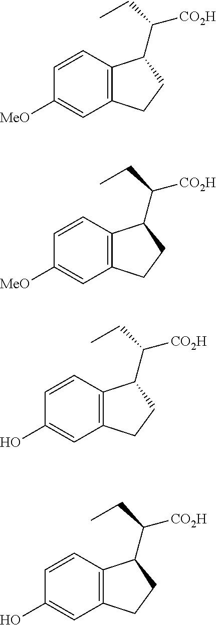 [SMILES: CC[C@H](C(=O)O)[C@@H]1CCc2cc(OC)ccc21 ; CC[C@@H](C(=O)O)[C@H]1CCc2cc(OC)ccc21 ; CC[C@H](C(=O)O)[C@@H]1CCc2cc(O)ccc21 ; CC[C@@H](C(=O)O)[C@H]1CCc2cc(O)ccc21]

Compounds of Formula III in which R¹=H may also be prepared in an optically active fashion by the methods summarized in Reaction Scheme 6. Resolution of racemic ester 17a (Formula III, where R¹ is H) may be accomplished by selective enzymatic hydrolysis using Amano Lipase PS to yield 17f. Alternatively, 17e, which may be prepared by hydrolysis of 17a, may be resolved by crystallization of the diastereomeric salts formed with an optically active amine, for example, (S)-(−)-α-methyl-benzylamine, followed by regeneration of the carboxylic acid by treating the salt with mineral acid. Further conversion of 17f to the intermediates 17g and 17h may be accomplished by means analogous to that described for the preparation of 17c in Reaction Scheme 2: reesterification and removal of the R⁷ protecting group.

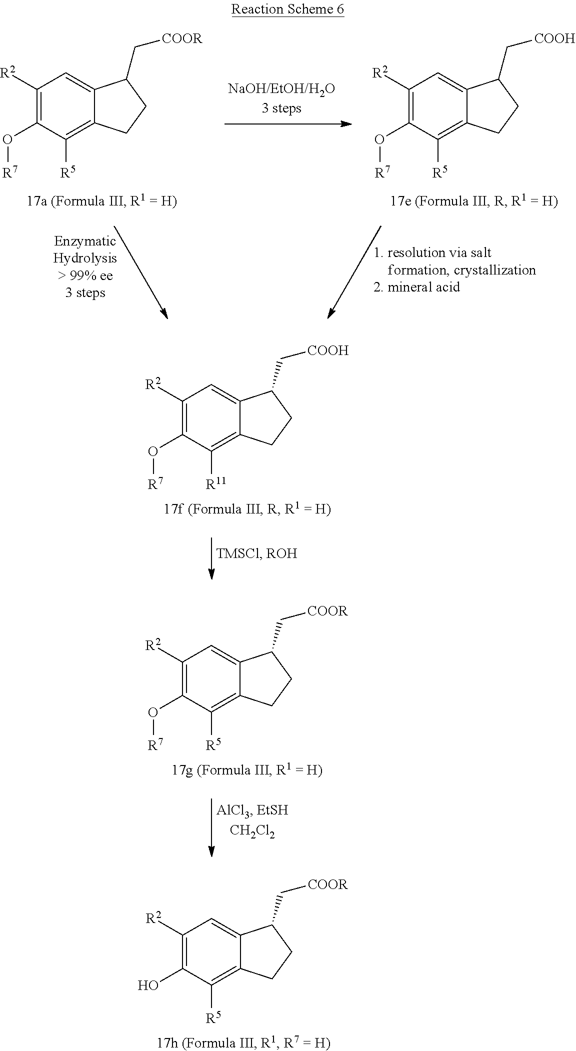

### (2) Formula VI

The present invention also encompasses compounds of Formula VI:

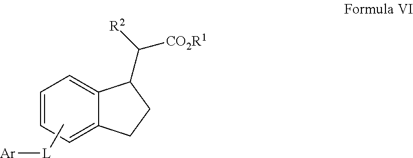

wherein

R¹ and R² are independently H, C₁-C₆ alkyl, or C₃-C₆ cycloalkyl;

L is a linker and selected from the group consisting of —(CH₂)ₘ—X—, —Y—(CH₂)ₙ—X—, and

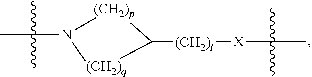

wherein
X is selected from the group O, S, S(═O), and S(═O)₂,
Y is selected from the group O, NR⁵, S, S(═O), and S(═O)₂,
m is 1, 2, or 3,
n is 2, 3, or 4,
t is 0 or 1,
p is 0, 1, 2, or 3,
q is 1, 2, 3, or 4,
   wherein the sum of p and q is 1, 2, 3, or 4;

Ar is phenyl or a 6-membered heteroaryl containing up to three N atoms,
wherein said Ar is optionally substituted at any available position by 1 to 5 independently selected R³ groups, and
optionally fused to a 5- or 6-membered saturated carbocyclic ring,
a 5- or 6-membered unsaturated carbocyclic ring, or
a 5- or 6-membered heterocyclic ring containing up to 3 additional heteroatoms selected from N, O, and S,
   wherein said fused ring may be optionally substituted at any available position by 1 to 4 independently selected R⁴ groups;

R³ is selected from the group consisting of hydroxy, SH, halo, CN, NO₂, C(═O)OH, C(═O)—OC₁-C₆ alkyl, C(═O)—OC₃-C₆ cycloalkyl, NR⁶R⁷, C(═O)NR⁶R⁷, C(═S)NR⁶R⁷, C₁-C₆ alkyl optionally substituted with halo, OH, NR⁶R⁷, or C₁-C₆ alkoxy, C₁-C₆ haloalkyl, C₁-C₆ alkoxy, C₁-C₆ thioalkyl, C₂-C₆ alkenyl, C₁-C₆ haloalkoxy, C₃-C₈ cycloalkyl, C₃-C₈ cycloalkoxy, phenoxy optionally substituted on the phenyl ring with halo, C₁-C₆ alkyl, or C₁-C₆ alkoxy, and

a mono or bicyclic ring radical selected from the group consisting of
c) phenyl optionally fused to
   a 5- or 6-membered saturated or partially unsaturated carbocylic ring, or
   a 5- or 6-membered saturated or partially unsaturated heterocyclic ring containing from 1-3 heteroatoms selected from N, O, and S,
d) a 5- or 6-membered heterocyclic ring radical containing up to 4 heteroatoms selected from N, O, or S, optionally fused to
   a 5- or 6-membered saturated or partially unsaturated carbocylic ring, or
   a 5- or 6-membered saturated or partially unsaturated heterocyclic ring containing from 1-3 heteroatoms selected from N, O, and S,
said mono or bicyclic ring radical being optionally substituted with up to 5 groups independently selected from the group consisting of halo, hydroxy, oxo, CN, C₁-C₆ alkyl optionally substituted with halo, OH, NR⁶R⁷, C₁-C₆ alkoxy, C₁-C₆ haloalkyl, C₁-C₆ alkoxy, C₁-C₆ thioalkyl, C₁-C₆ haloalkoxy, C₃-C₈ cycloalkyl, C₃-C₈ cycloalkoxy, C₁-C₆ acyl, C(═O)OH, CH₂C(═O)OH, NR⁶R⁷, C(═O)NR⁶R⁷, C(═O)OC₁-C₆ alkyl, and C(═O)OC₃-C₆ cycloalkyl;

R⁴ is selected from the group consisting of oxo, hydroxy, halo, CN, NR⁶R⁷, C₁-C₆ alkyl optionally substituted with OH, NR⁶R⁷, or C₁-C₆ alkoxy, C₁-C₆ haloalkyl, C₁-C₆ alkoxy, C₁-C₆ thioalkyl, C₁-C₆ haloalkoxy, C₃-C₈ cycloalkyl, and C₃-C₈ cycloalkoxy;

R⁵ is selected from the group consisting of H, C₁-C₆ alkyl optionally substituted with C₃-C₆ cycloalkyl, C₁-C₆ acyl, benzyl optionally substituted with halo, C₁-C₆ alkoxy, (C₁-C₆)alkyl, CN, NH₂, N[(C₁-C₃)alkyl]₂, NO₂, or CF₃, C₃-C₆ cycloalkyl, and C(═O)OC₁-C₆ alkyl;

R⁶ and R⁷ are independently selected from the group consisting of H, C₁-C₆ alkyl optionally substituted with C₃-C₆ cycloalkyl, C₁-C₆ acyl, benzyl optionally substituted with halo, C₁-C₆ alkoxy, (C₁-C₆)alkyl, CN, NH₂, N[(C₁-C₃)alkyl]₂, NO₂, or CF₃, C₃-C₆ cycloalkyl, and phenyl optionally substituted with halo, C₁-C₆ alkoxy, (C₁-C₆)alkyl, CN, N[(C₁-C₃)alkyl]₂, NO₂, or CF₃, or

R⁶ and R⁷ may be taken together with the nitrogen atom to which they are attached to form a 5- or 6-membered heterocyclic ring optionally interrupted by NR⁵ or O;

or a pharmaceutically acceptable salt, ester prodrug, stereoisomer, diastereomer, enantiomer, racemate or a combination thereof.

In some embodiments, the compound of Formula VI is a meglumine, potassium or sodium salt thereof.

In one embodiment, the compound of Formula VI, R¹ and R² are H, L is —O—(CH₂)ₙ—O, wherein n is 2, 3 or 4, Ar is a phenyl substituted with one to five R³, wherein each occurrence of R³ is independently C₁-C₆ alkyl or a 5- or 6-member heterocyclic ring containing up to 4 hetero atoms selected from the group consisting of N, O and S, wherein the heterocyclic ring is substituted with C₁-C₆ alkyl.

In some embodiments, the compound of Formula VI has a structure of

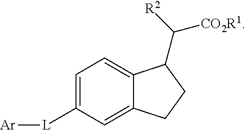

In another embodiment, the compound of Formula VI has the structure:

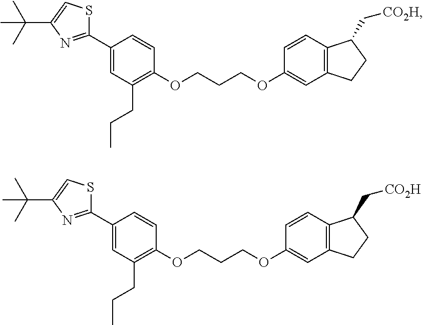 [SMILES: CCCc1cc(-c2nc(C(C)(C)C)cs2)ccc1OCCCOc1ccc2c(c1)CC[C@H]2CC(=O)O ; CCCc1cc(-c2nc(C(C)(C)C)cs2)ccc1OCCCOc1ccc2c(c1)CC[C@@H]2CC(=O)O]

or a pharmaceutically acceptable salt thereof. In one embodiment, the pharmaceutically acceptable salt is a meglumine, potassium or sodium salt of the above two structures.

In some embodiments, the linker L is substituted at either the 4- or 5-carbon atom (as shown above) of the indane ring in Formula (VI), replacing H atom.

Exemplary compounds of Formula (VI), wherein R² and R¹ are H, L is —Y—(CH₂)ₙ—X—, X and Y are O, and n is 2, are shown in Table 3a below.

TABLE 3a

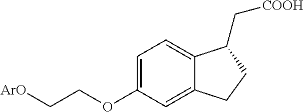

| Ex. | | HPLC RT | LC-MS |
|---|---|---|---|
| No | Ar | (min) | [M + H]⁺ |
| 1 | 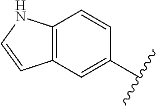 [SMILES: *c1ccc2[nH]ccc2c1] | 2.87 | 352.2 |
| 2 | 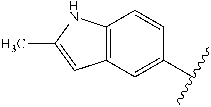 [SMILES: *c1ccc2[nH]c(C)cc2c1] | 3.00 | 366.2 |
| 3 | 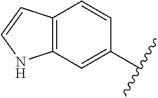 [SMILES: *c1ccc2cc[nH]c2c1] | 2.95 | 352.1 |
| 4 | 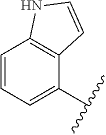 [SMILES: *c1cccc2[nH]ccc12] | 2.91 | 352.1 |
| 5 | 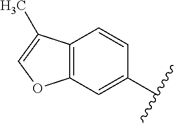 [SMILES: *c1ccc2c(C)coc2c1] | 3.33 | 367.5 |
| 6 | 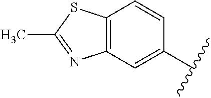 [SMILES: *c1ccc2sc(C)nc2c1] | 2.93 | 384.3 |
| 7 | 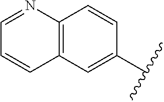 [SMILES: *c1ccc2ncccc2c1] | 2.02 | 364.2 |
| 8 | 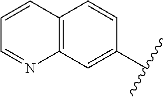 [SMILES: *c1ccc2cccnc2c1] | 2.08 | 364.3 |

TABLE 3b
IUPAC Names for Compounds in Table 3a

| Ex. | |
|---|---|
| No. | IUPAC Name |
| 37 | 2-[(1S)-5-(2-indol-5-yloxyethoxy)indanyl]acetic acid |
| 38 | 2-{(1S)-5-[2-(2-methylindol-5-yloxy)ethoxy]indanyl}acetic acid |
| 39 | 2-[(1S)-5-(2-indol-6-yloxyethoxy)indanyl]acetic acid |
| 40 | 2-[(1S)-5-(2-indol-4-yloxyethoxy)indanyl]acetic acid |
| 41 | 2-{(1S)-5-[2-(3-methylbenzo[3,4-b]furan-6- |
| | yloxy)ethoxy]indanyl}acetic acid |
| 42 | 2-{(1S)-5-[2-(2-methylbenzothiazol-5-yloxy)ethoxy]indanyl}acetic |
| | acid |
| 43 | 2-[(1S)-5-(2-(6-quinolyloxy)ethoxy)indanyl]acetic acid |
| 44 | 2-[(1S)-5-(2-(7-quinolyloxy)ethoxy)indanyl]acetic acid |

Examples of compounds of Formula (Imm) [Formula (II), where R² and R¹ are H, L is —Y—(CH₂)ₙ—X—, X and Y are O, and n is 3], as shown in Table 4a below.

TABLE 4a

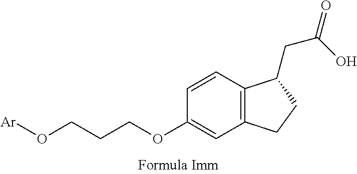

| | | HPLC | LC-MS |
|---|---|---|---|
| Ex. | | RT | [M + H]⁺ |
| No | Ar | (min) | or NMR data |
| 9 | 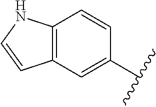 [SMILES: *c1ccc2[nH]ccc2c1] | 3.13 | 366.0 |
| 10 | 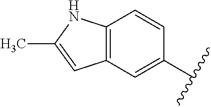 [SMILES: *c1ccc2[nH]c(C)cc2c1] | 3.16 | 380.2 |
| 11 | 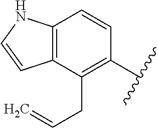 [SMILES: *c1ccc2[nH]ccc2c1CC=C] | 3.40 | 406.0 |
| 12 | 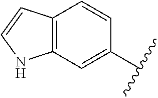 [SMILES: *c1ccc2cc[nH]c2c1] | 3.19 | 366.2 |
| 13 | 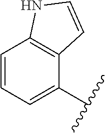 [SMILES: *c1cccc2[nH]ccc12] | 3.07 | 366.2 |
| 14 | 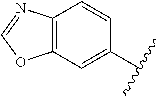 [SMILES: *c1ccc2ncoc2c1] | 3.28 | 368.1 |
| 15 | 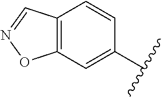 [SMILES: *c1ccc2cnoc2c1] | 2.76 | 367.9 |
| 16 | 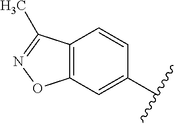 [SMILES: *c1ccc2c(C)noc2c1] | 2.97 | 382.1 |
| 17 | 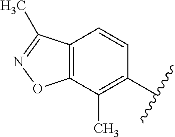 [SMILES: *c1ccc2c(C)noc2c1C] | 3.84 | 396.3 |
| 18 | 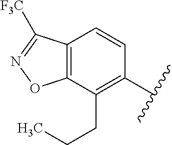 [SMILES: *c1ccc2c(C(F)(F)F)noc2c1CCC] | 4.18 | 477.9 |
| 19 | 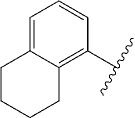 [SMILES: *c1cccc2c1CCCC2] | 4.51 | [a] |
| 20 | 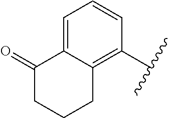 [SMILES: *c1cccc2c1CCCC2=O] | 3.90 | [b] |

TABLE 4b
IUPAC Names for Compounds in Table 4a

| Ex. | |
|---|---|
| No. | IUPAC Name |
| 74 | 2-[(1S)-5-(3-indol-5-yloxypropoxy)indanyl]acetic acid |
| 75 | 2-{(1S)-5-[3-(2-methylindol-5-yloxy)propoxy]indanyl}acetic acid |
| 76 | 2-{(1S)-5-[3-(4-prop-2-enylindol-5-yloxy)propoxy]indanyl}acetic |
| | acid |
| 77 | 2-[(1S)-5-(3-indol-6-yloxypropoxy)indanyl]acetic acid |
| 78 | 2-[(1S)-5-(3-indol-4-yloxypropoxy)indanyl]acetic acid |
| 79 | 2-[(1S)-5-(3-benzoxazol-6-yloxypropoxy)indanyl]acetic acid |
| 80 | 2-[(1S)-5-(3-benzo[d]isoxazol-6-yloxypropoxy)indanyl]acetic acid |
| 81 | 2-{(1S)-5-[3-(3-methylbenzo[d]isoxazol-6- |
| | yloxy)propoxy]indanyl}acetic acid |
| 82 | 2-{(1S)-5-[3-(3,7-dimethylbenzo[d]isoxazol-6- |
| | yloxy)propoxy]indanyl}acetic acid |
| 83 | ((1S)-5-{3-[(3-methyl-7-propyl-1,2-benzisoxazol-6- |
| | yl)oxy]propoxy}-2,3-dihydro-1H-inden-1-yl)acetic acid |
| 84 | 2-[(1S)-5-(3-(5,6,7,8- |
| | tetrahydronaphthyloxy)propoxy)indanyl]acetic acid |
| 85 | 2-{(1S)-5-[3-(5-oxo(6,7,8- |
| | trihydronaphthyloxy))propoxy]indanyl}acetic acid |

The compounds of Formula (Inn) [Formula (II) where R¹ and R² are H, L is —Y—(CH₂)ₙ—X—, X and Y are O, Ar is substituted phenyl, and n is 3] are shown below in Table 5a.

TABLE 5a

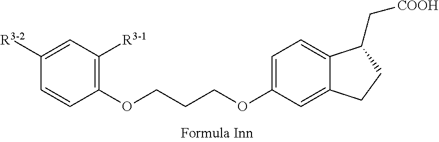

| Ex. | | | HPLC RT | LC-MS |
|---|---|---|---|---|
| No | R³⁻¹ | R³⁻² | (min) | [M + H]⁺ |
| 21 | H | H | 3.98 | * |
| 22 | n-Pr | H | 4.44 | * |
| 23 | H | Me | 4.14 | * |
| 24 | Me | Me | 3.80 | * |
| 25 | OMe | Me | 3.25 | 371.0 |
| 26 | OEt | Me | 3.42 | 385.0 |
| 27 | Br | Me | 4.29 | * |
| 28 | —NH(C(═O)C₃H₇) | Me | 3.35 | 426.2 |
| 29 | 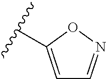 [SMILES: *c1ccno1] | Me | 3.22 | 407.8 |
| 30 | H | Et | 4.26 | 355.0 |
| 31 | OMe | Et | 3.47 | 384.9 |
| 32 | H | i-Pr | 3.96 | 369.2 |
| 33 | H | CF₃ | 3.67 | * |
| 34 | H | CN | 3.31 | 351.8 |
| 35 | n-Pr | CN | 3.70 | 393.8 |
| 36 | OMe | CN | 3.06 | 381.8 |
| 37 | H | OMe | 3.18 | * |
| 38 | n-Pr | OPh | 4.21 | * |
| 39 | H | OEt | 3.47 | 370.9 |
| 40 | H | OCF₃ | 3.75 | * |
| 41 | OMe | Br | 5.00 | 435.2 |
| | | | | (M − H)− |
| 42 | H | 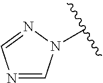 [SMILES: *n1cncn1] | 3.51 | 394.3 |
| 43 | —NH(C(═O)CH₃ | 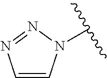 [SMILES: *n1ccnn1] | 3.25 | 451.2 |
| 44 | Cl | 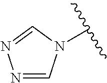 [SMILES: *n1cnnc1] | 2.73 | 428.1 |
| 45 | Me | 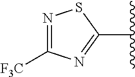 [SMILES: *c1nc(C(F)(F)F)ns1] | 4.16 | 492.9** |
| 46 | H | 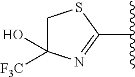 [SMILES: *C1=NC(O)(C(F)(F)F)CS1] | 3.34 | 496.0 |
| 47 | H | 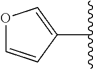 [SMILES: *c1ccoc1] | 3.55 | 393.0 |
| 48 | OMe | 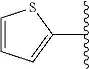 [SMILES: *c1cccs1] | 5.49 | 437.2 (M − H)− |
| 49 | OMe | 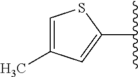 [SMILES: *c1cc(C)cs1] | 3.75 | 453.0 |
| 50 | H | Ph | 3.84 | * |
| 51 | OMe | 4-MeO-Ph- | 5.44 | 461.3 |
| | | | | (M − H)− |
| 52 | OMe | 4-F-Ph- | 5.57 | 449.3 |
| | | | | (M − H)− |
| 53 | H | 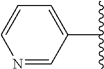 [SMILES: *c1cccnc1] | 2.42 | 404.2 |
| 54 | OMe | 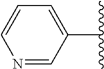 [SMILES: *c1cccnc1] | 2.36 | 434.0 |
| 55 | H | 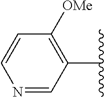 [SMILES: *c1cnccc1OC] | 3.47 | 434.2 |
| 56 | H | 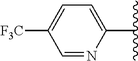 [SMILES: *c1ccc(C(F)(F)F)cn1] | 3.82 | 472.1 |
| 57 | H | 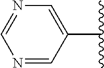 [SMILES: *c1cncnc1] | 2.95 | 405.1 |
| 58 | H | 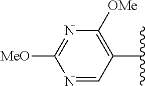 [SMILES: *c1cnc(OC)nc1OC] | 3.31 | 465.2 |
| 59 | H | 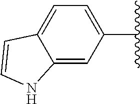 [SMILES: *c1ccc2cc[nH]c2c1] | 3.57 | 492.0 |

*These compounds did not ionize under ESI-MS conditions.

TABLE 5b
IUPAC Names for Compounds in Table 5a

| Ex. | |
|---|---|
| No. | IUPAC Name |
| 104 | 2-[(1S)-5-(3-phenoxypropoxy)indanyl]acetic acid |
| 105 | 2-{(1S)-5-[3-(2-propylphenoxy)propoxy]indanyl}acetic acid |
| 106 | 2-{(1S)-5-[3-(4-methylphenoxy)propoxy]indanyl}acetic acid |
| 107 | 2-{(1S)-5-[3-(2,4-dimethylphenoxy)propoxy]indanyl}acetic acid |
| 108 | 2-{(1S)-5-[3-(2-methoxy-4-methylphenoxy)propoxy]indanyl}acetic acid |
| 109 | 2-{(1S)-5-[3-(2-ethoxy-4-methylphenoxy)propoxy]indanyl}acetic acid |
| 110 | 2-{(1S)-5-[3-(2-bromo-4-methylphenoxy)propoxy]indanyl}acetic acid |
| 111 | 2-((1S)-5-{3-[2-(butanoylamino)-4- |
| | methylphenoxy]propoxy}indanyl)acetic acid |
| 112 | 2-{(1S)-5-[3-(2-isoxazol-5-yl-4-methylphenoxy)propoxy]indanyl}acetic |
| | acid |
| 113 | 2-{(1S)-5-[3-(4-ethylphenoxy)propoxy]indanyl}acetic acid |
| 114 | 2-{(1S)-5-[3-(4-ethyl-2-methoxyphenoxy)propoxy]indanyl}acetic acid |
| 115 | 2-((1S)-5-{3-[4-(methylethyl)phenoxy]propoxy}indanyl)acetic acid |
| 116 | 2-((1S)-5-{3-[4-(trifluoromethyl)phenoxy]propoxy}indanyl)acetic acid |
| 117 | 2-{(1S)-5-[3-(4-cyanophenoxy)propoxy]indanyl}acetic acid |
| 118 | 2-{(1S)-5-[3-(4-cyano-2-propylphenoxy)propoxy]indanyl}acetic acid |
| 119 | 2-{(1S)-5-[3-(4-cyano-2-methoxyphenoxy)propoxy]indanyl}acetic acid |
| 120 | 2-{(1S)-5-[3-(4-methoxyphenoxy)propoxy]indanyl}acetic acid |
| 121 | 2-{(1S)-5-[3-(4-phenoxy-2-propylphenoxy)propoxy]indanyl}acetic acid |
| 122 | 2-{(1S)-5-[3-(4-ethoxyphenoxy)propoxy]indanyl}acetic acid |
| 123 | 2-((1S)-5-{3-[4-(trifluoromethoxy)phenoxy]propoxy}indanyl)acetic acid |
| 124 | 2-{(1S)-5-[3-(4-bromo-2-methoxyphenoxy)propoxy]indanyl}acetic acid |
| 125 | 2-{(1S)-5-[3-(4-(1,2,4-triazolyl)phenoxy)propoxy]indanyl}acetic acid |
| 126 | 2-((1S)-5-{3-[2-(acetylamino)-4-(1,2,3- |
| | triazolyl)phenoxy]propoxy}indanyl) acetic acid |
| 127 | 2-{(1S)-5-[3-(2-chloro-4-(1,2,4-triazol-4- |
| | yl)phenoxy)propoxy]indanyl}acetic acid |
| 128 | 2-[(1S)-5-(3-{2-methyl-4-[3-(trifluoromethyl)(1,2,4-thiadiazol-5- |
| | yl)]phenoxy}propoxy)indanyl]acetic acid |
| 129 | 2-[(1S)-5-(3-{4-[4-hydroxy-4-(trifluoromethyl)(1,3-thiazolin-2- |
| | yl)]phenoxy} propoxy)indanyl]acetic acid |
| 130 | 2-{(1S)-5-[3-(4-(3-furyl)phenoxy)propoxy]indanyl}acetic acid |
| 131 | 2-{(1S)-5-[3-(2-methoxy-4-(2-thienyl)phenoxy)propoxy]indanyl}acetic |
| | acid |
| 132 | 2-((1S)-5-{3-[2-methoxy-4-(4-methyl(2- |
| | thienyl))phenoxy]propoxy}indanyl) acetic acid |
| 133 | {(1S)-5-[3-(1,1′-biphenyl-4-yloxy)propoxy]-2,3-dihydro-1H-inden-1- |
| | yl}acetic acid |
| 134 | 2-((1S)-5-{3-[2-methoxy-4-(4- |
| | methoxyphenyl)phenoxy]propoxy}indanyl) acetic acid |
| 135 | 2-((1S)-5-{3-[4-(4-fluorophenyl)-2- |
| | methoxyphenoxy]propoxy}indanyl)acetic acid |
| 136 | 2-{(1S)-5-[3-(4-(3-pyridyl)phenoxy)propoxy]indanyl}acetic acid |
| 137 | 2-{(1S)-5-[3-(2-methoxy-4-(3-pyridyl)phenoxy)propoxy]indanyl}acetic |
| | acid |
| 138 | 2-((1S)-5-{3-[4-(4-methoxy-(3-pyridyl))phenoxy]propoxy}indanyl)acetic |
| | acid |
| 139 | 2-[(1S)-5-(3-{4-[5-(trifluoromethyl)(2- |
| | pyridyl)]phenoxy}propoxy)indanyl]acetic acid |
| 140 | 2-{(1S)-5-[3-(4-pyrimidin-5-ylphenoxy)propoxy]indanyl}acetic acid |
| 141 | 2-((1S)-5-{3-[4-(2,4-dimethoxypyrimidin-5- |
| | yl)phenoxy]propoxy}indanyl)acetic acid |
| 142 | 2-{(1S)-5-[3-(4-indol-6-ylphenoxy)propoxy]indanyl}acetic acid |

Compounds of Formula (Ioo)) [Formula (II), where R¹ and R² are H, L is —Y—(CH₂)ₙ—X—, X and Y are O, Ar is heterocyclyl substituted phenyl, and n is 3], and (Ipp) [Formula (II), where R¹ and R² are H, L is —Y—(CH₂)ₙ—X—, X and Y are O, Ar is substituted phenyl, and n is 3], are listed in Table 6a and Table 7a, respectively, below.

TABLE 6a

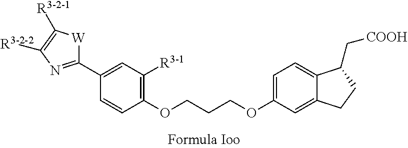

| Ex. | | | | | HPLC RT | LC-MS |
|---|---|---|---|---|---|---|
| No | W | R³⁻²⁻¹ | R³⁻²⁻² | R³⁻¹ | (min) | [M + H]⁺ |
| 60 | S | H | H | n-Pr | 3.73 | 452.1 |
| 61 | S | H | Me | OMe | 3.18 | 454.3 |
| 62 | S | H | Et | H | 3.56 | 438.3 |
| 63 | O | H | Et | H | 3.35 | 422.3 |
| 64 | O | H | Et | n-Pr | 3.82 | 464.2 |
| 65 | S | H | t-Bu | n-Pr | 4.64 | 508.3 |
| 66 | O | H | t-Bu | H | 3.77 | 450.2 |
| 67 | O | H | t-Bu | OMe | 3.69 | 480.2 |
| 68 | S | H | CF₃ | n-Pr | 4.18 | 520.2 |
| 69 | S | H | CF₃ | OMe | 3.63 | 507.9 |
| 70 | O | H | CF₃ | H | 3.58 | 462.1 |
| 71 | O | H | CF₃ | OMe | 3.52 | 491.9 |
| 72 | S | Me | Me | H | 3.31 | 438.3 |
| 73 | S | Me | Me | OMe | 3.19 | 468.3 |
| 74 75 76 | S S S | 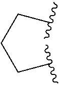 [SMILES: *CCC*] | | H n-Pr OMe | 3.66 4.12 3.51 | 450.3 492.4 480.4 |
| 77 78 79 80 81 82 83 | S S O O S S O | 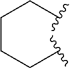 [SMILES: *CCCC*] | | H OMe H n-Pr OEt O-Pr OMe | 3.61 3.49 3.47 3.98 3.59 3.80 3.39 | 464.4 494.2 448.4 490.3 508.3 522.3 478.2 |
| 84 | S | 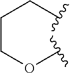 [SMILES: *CCCO*] | | OMe | 3.41 | 496.4 |
| 85 | S | 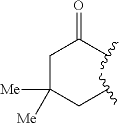 [SMILES: *CC(C)(C)CC(*)=O] | | n-Pr | 4.12 | 548.3 |
| 86 | S | H | OMe | H | 3.41 | 440.2 |
| 87 | S | H | OMe | OMe | 3.27 | 470.3 |
| 88 | S | H | OEt | H | 3.60 | 454.2 |
| 89 | S | H | OEt | n-Pr | 4.10 | 496.2 |
| 90 | S | H | OEt | OMe | 3.46 | 484.3 |
| 91 | S | H | O-i-Pr | n-Pr | 4.24 | 510.1 |
| 92 | S | Me | OEt | n-Pr | 4.51 | 510.2 |
| 93 | S | Me | OEt | OMe | 3.90 | 498.2 |
| 94 | S | Et | OEt | OMe | 4.07 | 512.1 |
| 95 | S | C(═O)CH₃ | Me | H | 3.50 | 466.1 |
| 96 | S | C(═O)CH₃ | Me | n-Pr | 3.99 | 508.2 |
| 97 | S | C(═O)CH₃ | Me | OMe | 3.30 | 496.3 |
| 98 | O | C(═O)CH₃ | Me | H | 3.21 | 450.3 |
| 99 | O | C(═O)CH₃ | Me | n-Pr | 3.74 | 492.1 |
| 100 | O | C(═O)CH₃ | Me | OMe | 3.08 | 480.3 |
| 101 | S | C(═O)NMe₂ | Me | n-Pr | 3.42 | 537.5 |
| 102 | S | C(═O)NMe₂ | Me | OMe | 2.96 | 525.1 |
| 103 | S | C(═O)OH | Me | H | 3.13 | 468.3 |
| 104 | S | C(═O)OH | Me | n-Pr | 3.58 | 510.2 |

TABLE 6b
IUPAC Names for Compounds in Table 6a

| Ex. | |
|---|---|
| No. | IUPAC Name |
| 175 | 2-{(1S)-5-[3-(2-propyl-4-(1,3-thiazol-2-yl)phenoxy)propoxy]indanyl}acetic |
| | acid |
| 176 | 2-((1S)-5-{3-[2-methoxy-4-(4-methyl(1,3-thiazol-2- |
| | yl))phenoxy]propoxy}indanyl) acetic acid |
| 177 | 2-((1S)-5-{3-[4-(4-ethyl(1,3-thiazol-2-yl))phenoxy]propoxy}indanyl)acetic |
| | acid |
| 178 | 2-((1S)-5-{3-[4-(4-ethyl(1,3-oxazol-2-yl))phenoxy]propoxy}indanyl)acetic |
| | acid |
| 179 | 2-((1S)-5-{3-[4-(4-ethyl(1,3-oxazol-2-yl))-2- |
| | propylphenoxy]propoxy}indanyl) acetic acid |
| 180 | 2-[(1S)-5-(3-{4-[4-(tert-butyl)(1,3-thiazol-2-yl)]-2-propylphenoxy}propoxy) |
| | indanyl]acetic acid |
| 181 | 2-[(1S)-5-(3-{4-[4-(tert-butyl)(1,3-oxazol-2- |
| | yl)]phenoxy}propoxy)indanyl]acetic acid |
| 182 | 2-[(1S)-5-(3-{4-[4-(tert-butyl)(1,3-oxazol-2-yl)]-2- |
| | methoxyphenoxy}propoxy) indanyl]acetic acid |
| 183 | 2-[(1S)-5-(3-{2-propyl-4-[4-(trifluoromethyl)(1,3-thiazol-2- |
| | yl)]phenoxy}propoxy) indanyl]acetic acid |
| 184 | 2-[(1S)-5-(3-{2-methoxy-4-[4-(trifluoromethyl)(1,3-thiazol-2-yl)]phenoxy} |
| | propoxy)indanyl]acetic acid |
| 185 | 2-[(1S)-5-(3-{4-[4-(trifluoromethyl)(1,3-oxazol-2-yl)]phenoxy}propoxy) |
| | indanyl]acetic acid |
| 186 | 2-[(1S)-5-(3-{2-methoxy-4-[4-(trifluoromethyl)(1,3-oxazol-2-yl)]phenoxy} |
| | propoxy)indanyl]acetic acid |
| 187 | 2-((1S)-5-{3-[4-(4,5-dimethyl(1,3-thiazol-2- |
| | yl))phenoxy]propoxy}indanyl)acetic acid |
| 188 | 2-((1S)-5-{3-[4-(4,5-dimethyl(1,3-thiazol-2-yl))-2- |
| | methoxyphenoxy]propoxy} indanyl)acetic acid |
| 189 | 2-{(1S)-5-[3-(4-(4,5,6-trihydrocyclopenta[1,2-d]1,3-thiazol-2-yl)phenoxy) |
| | propoxy]indanyl}acetic acid |
| 190 | 2-{(1S)-5-[3-(2-propyl-4-(4,5,6-trihydrocyclopenta[1,2-d]1,3-thiazol-2-yl) |
| | phenoxy)propoxy]indanyl}acetic acid |
| 191 | 2-{(1S)-5-[3-(2-methoxy-4-(4,5,6-trihydrocyclopenta[1,2-d]1,3-thiazol-2- |
| | yl)phenoxy)propoxy]indanyl}acetic acid |
| 192 | 2-{(1S)-5-[3-(4-(4,5,6,7-tetrahydrobenzothiazol-2-yl)phenoxy)propoxy] |
| | indanyl}acetic acid |
| 193 | 2-{(1S)-5-[3-(2-methoxy-4-(4,5,6,7-tetrahydrobenzothiazol-2-yl)phenoxy) |
| | propoxy]indanyl}acetic acid |
| 194 | 2-{(1S)-5-[3-(4-(4,5,6,7-tetrahydrobenzoxazol-2- |
| | yl)phenoxy)propoxy]indanyl} acetic acid |
| 195 | 2-{(1S)-5-[3-(2-propyl-4-(4,5,6,7-tetrahydrobenzoxazol-2- |
| | yl)phenoxy)propoxy] indanyl}acetic acid |
| 196 | 2-{(1S)-5-[3-(2-ethoxy-4-(4,5,6,7-tetrahydrobenzothiazol-2-yl)phenoxy) |
| | propoxy]indanyl}acetic acid |
| 197 | 2-{(1S)-5-[3-(2-propoxy-4-(4,5,6,7-tetrahydrobenzothiazol-2-yl)phenoxy) |
| | propoxy]indanyl}acetic acid |
| 198 | 2-{(1S)-5-[3-(2-methoxy-4-(4,5,6,7-tetrahydrobenzoxazol-2-yl)phenoxy) |
| | propoxy]indanyl}acetic acid |
| 199 | 2-{(1S)-5-[3-(2-methoxy-4-(5,6,7-trihydro-2H-pyrano[2,3-d]1,3-thiazol-2- |
| | yl)phenoxy)propoxy]indanyl}acetic acid |
| 200 | 2-((1S)-5-{3-[4-(5,5-dimethyl-7-oxo(4,5,6-trihydrobenzothiazol-2-yl))-2- |
| | propylphenoxy]propoxy}indanyl)acetic acid |
| 201 | 2-((1S)-5-{3-[4-(4-methoxy(1,3-thiazol-2- |
| | yl))phenoxy]propoxy}indanyl)acetic acid |
| 202 | 2-((1S)-5-{3-[2-methoxy-4-(4-methoxy(1,3-thiazol-2- |
| | yl))phenoxy]propoxy} indanyl)acetic acid |
| 203 | 2-((1S)-5-{3-[4-(4-ethoxy(1,3-thiazol-2- |
| | yl))phenoxy]propoxy}indanyl)acetic acid |
| 204 | 2-((1S)-5-{3-[4-(4-ethoxy(1,3-thiazol-2-yl))-2- |
| | propylphenoxy]propoxy}indanyl) acetic acid |
| 205 | 2-((1S)-5-{3-[4-(4-ethoxy(1,3-thiazol-2-yl))-2-methoxyphenoxy]propoxy} |
| | indanyl)acetic acid |
| 206 | 2-[(1S)-5-(3-{4-[4-(methylethoxy)(1,3-thiazol-2-yl)]-2- |
| | propylphenoxy}propoxy) indanyl]acetic acid |
| 207 | 2-((1S)-5-{3-[4-(4-ethoxy-5-methyl(1,3-thiazol-2-yl))-2- |
| | propylphenoxy]propoxy} indanyl)acetic acid |
| 208 | 2-((1S)-5-{3-[4-(4-ethoxy-5-methyl(1,3-thiazol-2-yl))-2-methoxyphenoxy] |
| | propoxy}indanyl)acetic acid |
| 209 | 2-((1S)-5-{3-[4-(4-ethoxy-5-ethyl(1,3-thiazol-2-yl))-2-methoxyphenoxy] |
| | propoxy}indanyl)acetic acid |
| 210 | 2-((1S)-5-{3-[4-(5-acetyl-4-methyl(1,3-thiazol-2- |
| | yl))phenoxy]propoxy}indanyl) acetic acid |
| 211 | 2-((1S)-5-{3-[4-(5-acetyl-4-methyl(1,3-thiazol-2-yl))-2- |
| | propylphenoxy]propoxy} indanyl)acetic acid |
| 212 | 2-((1S)-5-{3-[4-(5-acetyl-4-methyl(1,3-thiazol-2-yl))-2-methoxyphenoxy] |
| | propoxy}indanyl)acetic acid |
| 213 | 2-((1S)-5-{3-[4-(5-acetyl-4-methyl(1,3-oxazol-2- |
| | yl))phenoxy]propoxy}indanyl) acetic acid |
| 214 | 2-((1S)-5-{3-[4-(5-acetyl-4-methyl(1,3-oxazol-2-yl))-2- |
| | propylphenoxy]propoxy} indanyl)acetic acid |
| 215 | 2-((1S)-5-{3-[4-(5-acetyl-4-methyl(1,3-oxazol-2-yl))-2-methoxyphenoxy] |
| | propoxy}indanyl)acetic acid |
| 216 | 2-[(1S)-5-(3-{4-[5-(N,N-dimethylcarbamoyl)-4-methyl(1,3-thiazol-2-yl)]-2- |
| | propylphenoxy}propoxy)indanyl]acetic acid |
| 217 | 2-[(1S)-5-(3-{4-[5-(N,N-dimethylcarbamoyl)-4-methyl(1,3-thiazol-2-yl)]-2- |
| | methoxyphenoxy}propoxy)indanyl]acetic acid |
| 218 | 2-(4-{3-[(1S)-1-(carboxymethyl)indan-5-yloxy]propoxy}phenyl)-4-methyl- |
| | 1,3-thiazole-5-carboxylic acid |
| 219 | 2-(4-{3-[(1S)-1-(carboxymethyl)indan-5-yloxy]propoxy}-3-propylphenyl)- |
| | 4-methyl-1,3-thiazole-5-carboxylic acid |

TABLE 7a
Formula Ipp

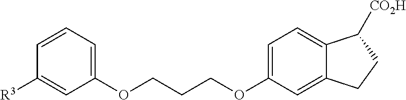

| | | HPLC | |
|---|---|---|---|
| Ex. | | RT | LC-MS |
| No | R³ | (min) | [M + H]⁺ |
| 105 | 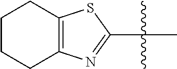 [SMILES: *c1nc2c(s1)CCCC2] | 3.79 | 464.3 |
| 106 | 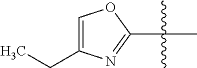 [SMILES: *c1nc(CC)co1] | 3.49 | 422.2 |
| 107 | 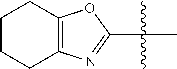 [SMILES: *c1nc2c(o1)CCCC2] | 3.64 | 448.3 |
| 108 | 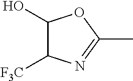 | 3.17 | 480.1 * |

* Elimination of water did not occur in this case.

TABLE 7b
IUPAC Names for Compounds in Table 7a

| Ex. | |
|---|---|
| No. | IUPAC Name |
| 220 | ((1S)-5-{3-[3-(4,5,6,7-tetrahydro-1,3-benzothiazol-2- |
| | yl)phenoxy] propoxy}-2,3-dihydro-1H-inden-1-yl)acetic acid |
| 221 | ((1S)-5-{3-[3-(4-ethyl-1,3-oxazol-2-yl)phenoxy]propoxy}-2,3- |
| | dihydro-1H-inden-1-yl)acetic acid |
| 222 | ((1S)-5-{3-[3-(4,5,6,7-tetrahydro-1,3-benzoxazol-2-yl)phenoxy] |
| | propoxy}-2,3-dihydro-1H-inden-1-yl)acetic acid |
| 223 | ((1S)-5-{3-[3-(4-hydroxy-5-methyl-4,5-dihydro-1,3-oxazol-2- |
| | yl)phenoxy]propoxy}-2,3-dihydro-1H-inden-1-yl)acetic acid |

Compounds of Formula (Iqq) [Formula (II), where R¹ and R² are H, L is —Y—(CH₂)ₙ—X—, X and Y are O, Ar is substituted phenyl, and n is 3], appear in Table 8a below.

TABLE 8a
Formula Iqq

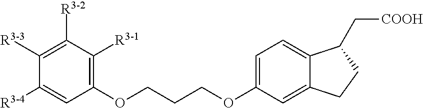

| | | | | | HPLC | |
|---|---|---|---|---|---|---|
| Ex. | | | | | RT | LC-MS |
| No | R³⁻¹ | R³⁻² | R³⁻³ | R³⁻⁴ | (min) | [M + H]⁺ |
| 109 | H | Me | H | H | 3.45 | * |
| 110 | H | OMe | H | H | 3.23 | 357.0 |
| 111 | H | Ph | H | H | 3.53 | * |
| 112 | OMe | OMe | H | H | 3.07 | 387.0 |
| 113 | H | H | NHC(═O)CH₃ | OMe | 3.38 | 414.1 |
| 114 | H | H | Me | Me | 4.24 | * |
| 115 | H | OMe | OMe | OMe | 3.73 | 417.2 |

*These compounds did not ionize under ESI-MS conditions.

TABLE 8b
IUPAC Names for Compounds in Table 8a

| Ex. | |
|---|---|
| No. | IUPAC Name |
| 227 | 2-{(1S)-5-[3-(3-methylphenoxy)propoxy]indanyl}acetic acid |
| 228 | 2-{(1S)-5-[3-(3-methoxyphenoxy)propoxy]indanyl}acetic acid |
| 229 | 2-{(1S)-5-[3-(3-phenylphenoxy)propoxy]indanyl}acetic acid |
| 230 | 2-{(1S)-5-[3-(2,3-dimethoxyphenoxy)propoxy]indanyl}acetic |
| | acid |
| 231 | 2-((1S)-5-{3-[4-(acetylamino)-3-methoxyphenoxy]propoxy} |
| | indanyl)acetic acid |
| 232 | 2-{(1S)-5-[3-(3,4-dimethylphenoxy)propoxy]indanyl}acetic |
| | acid |
| 233 | 2-{(1S)-5-[3-(3,4,5- |
| | trimethoxyphenoxy)propoxy]indanyl}acetic acid |

Exemplary compounds of Formula (Irr)) [Formula (II), where R¹ is H, R² is methyl, L is —Y—(CH₂)ₙ—X—, X and Y are O, and n is 3] is shown in Table 9a below.

TABLE 9a
Formula Irr

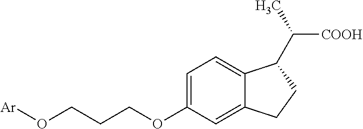

| | | HPLC | |
|---|---|---|---|
| Ex. | | RT | LC-MS |
| No | Ar | (min) | [M + H]⁺ |
| 116 | 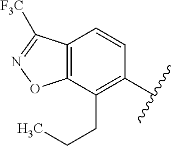 [SMILES: *c1ccc2c(C(F)(F)F)noc2c1CCC] | 4.21 | * |
| 117 | 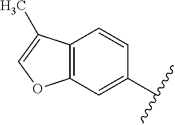 [SMILES: *c1ccc2c(C)coc2c1] | 3.79 | 395.0 |
| 118 | 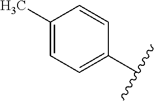 [SMILES: *c1ccc(C)cc1] | 3.70 | * |
| 119 | 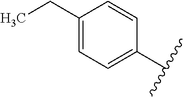 [SMILES: *c1ccc(CC)cc1] | 3.87 | 369.1 |
| 120 | 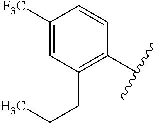 [SMILES: *c1ccc(C(F)(F)F)cc1CCC] | 4.06 | * |

TABLE 9b
IUPAC Names for Compounds in Table 9a

| Ex. | |
|---|---|
| No. | IUPAC Name |
| 237 | (2S)-2-((1S)-5-{3-[7-propyl-3-(trifluoromethyl)benzo[d]isoxazol- |
| | 6-yloxy] propoxy}indanyl)propanoic acid |
| 238 | (2S)-2-{(1S)-5-[3-(3-methylbenzo[3,4-b]furan-6- |
| | yloxy)propoxy]indanyl} propanoic acid |
| 239 | (2S)-2-{(1S)-5-[3-(4-methylphenoxy)propoxy]indanyl}propanoic |
| | acid |
| 240 | (2S)-2-{(1S)-5-[3-(4-ethylphenoxy)propoxy]indanyl}propanoic |
| | acid |
| 241 | (2S)-2-((1S)-5-{3-[2-propyl-4- |
| | (trifluoromethyl)phenoxy]propoxy}indanyl) propanoic acid |

TABLE 10a

| | | HPLC | |
|---|---|---|---|
| Ex. | | RT | LC-MS |
| No | Structure | (min) | [M + H]⁺ |
| 121 | 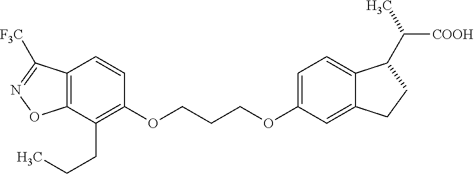 | 4.21 | * |
| 122 | 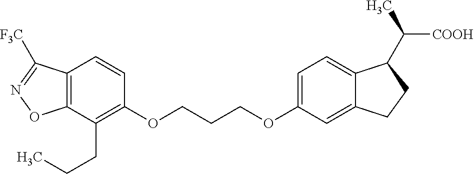 [SMILES: CCCc1c(OCCCOc2ccc3c(c2)CC[C@@H]3[C@@H](C)C(=O)O)ccc2c(C(F)(F)F)noc12] | 4.20 | * |

TABLE 10b
IUPAC Names for Compounds in Table 10a

| Ex. | |
|---|---|
| No. | IUPAC Name |
| 244 | (2S)-2-[(1S)-5-(3-{[7-propyl-3-(trifluoromethyl)-1,2-benzisoxazol- |
| | 6-yl]oxy} propoxy)-2,3-dihydro-1H-inden-1-yl]propanoic acid |
| 245 | (2R)-2-[(1R)-5-(3-{[7-propyl-3-(trifluoromethyl)-1,2-benzisoxazol- |
| | 6-yl]oxy} propoxy)-2,3-dihydro-1H-inden-1-yl]propanoic acid |

TABLE 11
Formula Iss

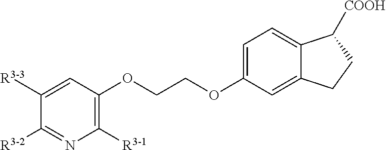

| | | | | LC-MS | |
|---|---|---|---|---|---|
| Ex. | | | | RT | LC-MS |
| No | R³⁻¹ | R³⁻² | R³⁻³ | (min) | [M + H]⁺ |
| 123 | H | H | H | 1.66 | 314.3 |
| 124 | H | CH₃ | H | 1.73 | 328.2 |
| 125 | CH₃ | H | H | 2.15 | 328.3 |
| 126 | H | H | Cl | 3.46 | 348.2 |
| 127 | H | H | C(═O)OH | 2.79 | 358.2 |

TABLE 11b
IUPAC Names for Compounds in Table 11a

| Ex. | |
|---|---|
| No. | IUPAC Name |
| 252 | 2-[(1S)-5-(2-(3-pyridyloxy)ethoxy)indanyl]acetic acid |
| 253 | 2-{(1S)-5-[2-(6-methyl(3-pyridyloxy))ethoxy]indanyl}acetic acid |
| 254 | 2-{(1S)-5-[2-(2-methyl(3-pyridyloxy))ethoxy]indanyl}acetic acid |
| 255 | 2-{(1S)-5-[2-(5-chloro(3-pyridyloxy))ethoxy]indanyl}acetic acid |
| 256 | 5-{2-[(1S)-1-(carboxymethyl)indan-5-yloxy]ethoxy}pyridine-3- |
| | carboxylic acid |

TABLE 12a
Formula Itt

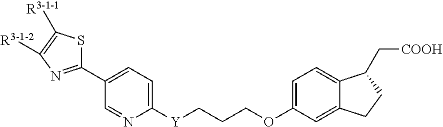

| Ex. | | | | LC-MS | LC-MS |
|---|---|---|---|---|---|
| No. | R³⁻¹⁻¹ | R³⁻¹⁻² | Y | RT (min) | [M + H]⁺ |
| 128 | H | Et | O | 3.55 | 439.1 |
| 129 | CH₃C(═O) | CH₃ | O | 3.30 | 467.1 |
| 130 | —CH₂CH₂CH₂CH₂— | | O | 3.67 | 465.1 |
| 131 | H | EtO | O | 3.40 | 455.1 |
| 132 | H | Et | NH | 2.31 | 438.2 |
| 133 | CH₃C(═O) | CH₃ | NH | 2.35 | 466.2 |
| 134 | CH₃ | CH₃ | NH | 2.27 | 438.2 |
| 135 | H | Et | NCH₃ | 2.40 | 452.4 |
| 136 | CH₃C(═O) | CH₃ | NCH₃ | 2.52 | 480.4 |
| 137 | CH₃ | CH₃ | NCH₃ | 2.32 | 452.4 |
| 138 | H | Et | N—n-Pr | 2.84 | 480.2 |
| 139 | CH₃C(═O) | CH₃ | N—n-Pr | 3.03 | 508.2 |

TABLE 12b
IUPAC Names for Compounds in Table 12a

| Ex. | |
|---|---|
| No. | IUPAC Name |
| 269 | 2-((1S)-5-{3-[5-(4-ethyl(1,3-thiazol-2-yl))(2- |
| | pyridyloxy)]propoxy}indanyl)acetic acid |
| 270 | 2-((1S)-5-{3-[5-(5-acetyl-4-methyl(1,3-thiazol-2-yl))(2- |
| | pyridyloxy)]propoxy} indanyl)acetic acid |
| 271 | 2-{(1S)-5-[3-(5-(4,5,6,7-tetrahydrobenzothiazol-2-yl)(2- |
| | pyridyloxy))propoxy] indanyl}acetic acid |
| 272 | 2-((1S)-5-{3-[5-(4-ethoxy(1,3-thiazol-2-yl))(2- |
| | pyridyloxy)]propoxy}indanyl) acetic acid |
| 273 | 2-[(1S)-5-(3-{[5-(4-ethyl(1,3-thiazol-2-yl))(2- |
| | pyridyl)]amino}propoxy)indanyl] acetic acid |
| 274 | 2-[(1S)-5-(3-{[5-(5-acetyl-4-methyl(1,3-thiazol-2-yl))(2- |
| | pyridyl)]amino}propoxy) indanyl]acetic acid |
| 275 | 2-[(1S)-5-(3-{[5-(4,5-dimethyl(1,3-thiazol-2-yl))(2- |
| | pyridyl)]amino}propoxy) indanyl]acetic acid |
| 276 | 2-[(1S)-5-(3-{[5-(4-ethyl(1,3-thiazol-2-yl))(2- |
| | pyridyl)]methylamino}propoxy) indanyl]acetic acid |
| 277 | 2-[(1S)-5-(3-{[5-(5-acetyl-4-methyl(1,3-thiazol-2-yl))(2- |
| | pyridyl)]methylamino} propoxy)indanyl]acetic acid |
| 278 | 2-[(1S)-5-(3-{[5-(4,5-dimethyl(1,3-thiazol-2-yl))(2- |
| | pyridyl)]methylamino} propoxy)indanyl]acetic acid |
| 279 | 2-[(1S)-5-(3-{[5-(4-ethyl(1,3-thiazol-2-yl))(2- |
| | pyridyl)]propylamino}propoxy) indanyl]acetic acid |
| 280 | 2-[(1S)-5-(3-{[5-(5-acetyl-4-methyl(1,3-thiazol-2-yl))(2- |
| | pyridyl)]propylamino} propoxy)indanyl]acetic acid |

TABLE 13a
Formula Iuu

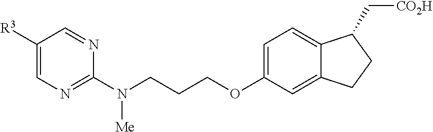

| | | LCMS | RT |
|---|---|---|---|
| Example | R³ | (M + H) | (min) |
| 140 | 3,4-dioxolane-Ph | 462.2 | 3.02 |
| 141 | 4-F—Ph | 436.2 | 3.18 |
| 142 | 4-MeO—Ph | 448.3 | 3.01 |
| 143 | 4-t-Bu | 474.2 | 3.70 |
| 144 | 3-thienyl | 424.1 | 3.07 |
| 145 | 2-benzothienyl | 474.2 | 3.72 |

TABLE 13b
IUPAC Names for Compounds in Table 13a

| Ex. | |
|---|---|
| No. | IUPAC Name |
| 284 | ((1S)-5-{3-[[5-(1,3-benzodioxol-5-yl)-2- |
| | pyrimidinyl](methyl)amino]propoxy}-2,3-dihydro-1H- |
| | inden-1-yl)acetic acid |
| 285 | 2-[(1S)-5-(3-{[5-(4-fluorophenyl)pyrimidin- |
| | 2yl]methylamino}propoxy)indanyl] acetic acid |
| 286 | 2-[(1S)-5-(3-{[5-(4-methoxyphenyl)pyrimidin-2- |
| | yl]methylamino}propoxy)indanyl] acetic acid |
| 287 | 2-{(1S)-5-[3-({5-[4-(tert-butyl)phenyl]pyrimidin-2- |
| | yl}methylamino)propoxy] indanyl}acetic acid |
| 288 | 2-((1S)-5-{3-[methyl(5-(3-thienyl)pyrimidin-2- |
| | yl)amino]propoxy}indanyl)acetic acid |
| 289 | 2-((1S)-5-{3-[(5-benzo[b]thiophen-2-ylpyrimidin-2- |
| | yl)methylamino]propoxy} indanyl)acetic acid |

TABLE 14a
Formula Ivv

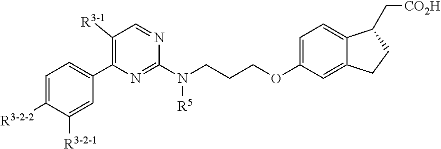

| Ex. | | | | | LCMS | RT |
|---|---|---|---|---|---|---|
| No. | R³⁻¹ | R⁵ | R³⁻²⁻¹ | R³⁻²⁻² | (M + H) | (min) |
| 146 | H | Me | H | CF₃ | 3.28 | 486.4 |
| 147 | H | n-Pr | H | CF₃ | 3.73 | 514.4 |
| 148 | H | n-Pr | —O—CH₂—O— | | 2.94 | 490.2 |
| 149¹ | CF₃ | Me | H | Et | 4.04 | 514.3 |
| 150¹ | CF₃ | Me | H | MeO | 3.73 | 516.3 |
| 151¹ | CF₃ | Me | H | Cl | 3.96 | 520.3 |

| | | | | | |
|---|---|---|---|---|---|
| 152¹ | CF₃ | Me | —O—CH₂—O— | 3.68 | 530.3 |

TABLE 14b
IUPAC Names for Compounds in Table 14a

| Ex. | |
|---|---|
| No. | IUPAC Name |
| 294 | 2-{(1S)-5-[3-(methyl{4-[4-(trifluoromethyl)phenyl]pyrimidin-2- |
| | yl}amino) propoxy]indanyl}acetic acid |
| 295 | 2-{(1S)-5-[3-(propyl{4-[4-(trifluoromethyl)phenyl]pyrimidin-2- |
| | yl}amino) propoxy]indanyl}acetic acid |
| 296 | ((1S)-5-{3-[[4-(1,3-benzodioxol-5-yl)-2- |
| | pyrimidinyl](propyl)amino]propoxy}-2,3-dihydro-1H-inden-1- |
| | yl)acetic acid |
| 297 | 2-[(1S)-5-(3-{[4-(4-ethylphenyl)-5-(trifluoromethyl)pyrimidin-2- |
| | yl]methyl amino}propoxy)indanyl]acetic acid |
| 298 | 2-[(1S)-5-(3-{[4-(4-methoxyphenyl)-5-(trifluoromethyl)pyrimidin- |
| | 2-yl]methyl amino}propoxy)indanyl]acetic acid |
| 299 | 2-[(1S)-5-(3-{[4-(4-chlorophenyl)-5-(trifluoromethyl)pyrimidin-2- |
| | yl]methyl amino}propoxy)indanyl]acetic acid |
| 300 | 2-[(1S)-5-(3-{[4-(2H-benzo[3,4-d]1,3-dioxolan-5-yl)-5- |
| | (trifluoromethyl) pyrimidin-2- |
| | yl]methylamino}propoxy)indanyl]acetic acid |

TABLE 15a
Exemplary Compounds of Formula (Iww)
Formula Iww

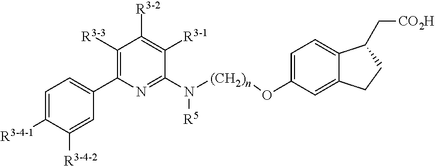

| | | | | | | | | LC- | LC- |
|---|---|---|---|---|---|---|---|---|---|
| | | | | | | | | MS | MS |
| Ex. | | | | | | | | RT | [M + |
| No. | R³⁻¹ | R³⁻² | R³⁻³ | R³⁻⁴⁻¹ | R³⁻⁴⁻² | R⁵ | n | (min) | H]⁺ |
| 153 | CF₃ | H | H | CH₃O | F | H | 3 | 3.80 | 519.3 |
| 154 | CF₃ | H | H | CH₃O | CH₃O | H | 3 | 3.61 | 531.3 |
| 155 | CF₃ | H | H | —OCH₂O— | | H | 3 | 3.76 | 515.3 |
| 156 | CF₃ | H | H | F | H | H | 3 | 4.02 | 489.1 |
| 157 | CF₃ | H | H | CH₃ | H | H | 3 | 4.69 | 485.3 |
| 158 | CF₃ | H | H | H | H | H | 3 | 4.00 | 471.1 |
| 159 | CF₃ | H | H | Et | H | H | 2 | 4.04 | 485.3 |
| 160 | CF₃ | H | H | Et | H | CH₃ | 3 | 4.50 | 513.2 |
| 161 | CF₃ | H | H | Et | H | CH₃ | 2 | 4.44 | 499.1 |
| 162 | H | H | CF₃ | CH₃O | F | H | 3 | 3.38 | 519.1 |
| 163 | H | H | CF₃ | CH₃O | CH₃O | H | 3 | 3.02 | 531.1 |
| 164 | H | H | CF₃ | —OCH₂O— | | H | 3 | 3.23 | 515.1 |
| 165 | H | H | CF₃ | F | H | H | 3 | 3.46 | 489.1 |
| 166 | H | H | CF₃ | CH₃ | H | H | 3 | 3.37 | 485.2 |
| 167 | H | H | CF₃ | H | H | H | 3 | 3.31 | 471.2 |
| 168 | F | H | CN | Et | H | CH₃ | 3 | 3.86 | 488.3 |
| 169 | H | CH₃ | CN | Et | H | CH₃ | 3 | 3.78 | 484.4 |
| 170 | H | CH₃ | CN | CH₃O | H | CH₃ | 3 | 3.54 | 486.4 |

TABLE 15b
IUPAC Names for Compounds in Table 15a

| Ex. | |
|---|---|
| No. | IUPAC Name |
| 321 | 2-[(1S)-5-(3-{[6-(3-fluoro-4-methoxyphenyl)-3-(trifluoromethyl)(2- |
| | pyridyl)] amino}propoxy)indanyl]acetic acid |
| 322 | 2-[(1S)-5-(3-{[6-(3,4-dimethoxyphenyl)-3-(trifluoromethyl)(2- |
| | pyridyl)]amino} propoxy)indanyl]acetic acid |
| 323 | 2-[(1S)-5-(3-{[6-(2H-benzo[3,4-d]1,3-dioxolan-5-yl)-3- |
| | (trifluoromethyl)(2-pyridyl)]amino}propoxy)indanyl]acetic acid |
| 324 | 2-[(1S)-5-(3-{[6-(4-fluorophenyl)-3-(trifluoromethyl)(2- |
| | pyridyl)]amino}propoxy) indanyl]acetic acid |
| 325 | 2-[(1S)-5-(3-{[6-(4-methylphenyl)-3-(trifluoromethyl)(2- |
| | pyridyl)]amino}propoxy) indanyl]acetic acid |
| 326 | 2-[(1S)-5-(3-{[6-phenyl-3-(trifluoromethyl)(2- |
| | pyridyl)]amino}propoxy)indanyl] acetic acid |
| 327 | 2-[(1S)-5-(2-{[6-(4-ethylphenyl)-3-(trifluoromethyl)(2- |
| | pyridyl)]amino}ethoxy) indanyl]acetic acid |
| 328 | 2-[(1S)-5-(3-{[6-(4-ethylphenyl)-3-(trifluoromethyl)(2- |
| | pyridyl)]methylamino} propoxy)indanyl]acetic acid |
| 329 | 2-[(1S)-5-(2-{[6-(4-ethylphenyl)-3-(trifluoromethyl)(2- |
| | pyridyl)]methylamino} ethoxy)indanyl]acetic acid |
| 330 | 2-[(1S)-5-(3-{[6-(3-fluoro-4-methoxyphenyl)-5-(trifluoromethyl)(2- |
| | pyridyl)] amino}propoxy)indanyl]acetic acid |
| 331 | 2-[(1S)-5-(3-{[6-(3,4-dimethoxyphenyl)-5-(trifluoromethyl)(2- |
| | pyridyl)]amino} propoxy)indanyl]acetic acid |
| 332 | 2-[(1S)-5-(3-{[6-(2H-benzo[3,4-d]1,3-dioxolan-5-yl)-5- |
| | (trifluoromethyl)(2-pyridyl)]amino}propoxy)indanyl]acetic acid |
| 333 | 2-[(1S)-5-(3-{[6-(4-fluorophenyl)-5-(trifluoromethyl)(2- |
| | pyridyl)]amino}propoxy) indanyl]acetic acid |
| 334 | 2-[(1S)-5-(3-{[6-(4-methylphenyl)-5-(trifluoromethyl)(2- |
| | pyridyl)]amino}propoxy) indanyl]acetic acid |
| 335 | 2-[(1S)-5-(3-{[6-phenyl-5-(trifluoromethyl)(2- |
| | pyridyl)]amino}propoxy)indanyl] acetic acid |
| 336 | 2-[(1S)-5-(3-{[5-cyano-6-(4-ethylphenyl)-3-fluoro(2- |
| | pyridyl)]methylamino} propoxy)indanyl]acetic acid |
| 337 | 2-[(1S)-5-(3-{[5-cyano-6-(4-ethylphenyl)-4-methyl(2- |
| | pyridyl)]methylamino} propoxy)indanyl]acetic acid |
| 338 | 2-[(1S)-5-(3-{[5-cyano-6-(4-methoxyphenyl)-4-methyl(2- |
| | pyridyl)]methyl amino}propoxy)indanyl]acetic acid |

Exemplary compounds of Formula (Ixx) and (Iyy) were listed in Table 16a and Table 17a below.

TABLE 16a
Formula Ixx

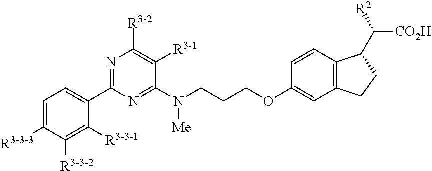

| Ex. | | | | | | | LCMS | RT |
|---|---|---|---|---|---|---|---|---|
| No. | R² | R³⁻¹ | R³⁻² | R³⁻³⁻¹ | R³⁻³⁻² | R³⁻³⁻³ | (M + H) | (min) |
| 171 | H | H | H | H | H | Me | 432.2 | 2.41 |
| 172 | H | H | H | H | H | Et | 446.4 | 2.27 |
| 173 | H | H | H | H | H | F | 436.3 | 2.27 |
| 174 | H | H | H | H | —O—CH₂—O— | | 462.3 | 2.25 |
| 175 | H | H | H | H | H | EtO | 462.3 | 2.50 |
| 176 | H | H | H | H | H | MeO | 448.4 | 2.30 |
| 177 | H | H | H | H | MeO | MeO | 478.4 | 2.20 |
| 178 | H | H | H | H | H | Ac | 460.3 | 2.31 |
| 179 | H | Me | H | H | H | F | 450.2 | 2.44 |
| 180 | H | Me | H | H | —O—CH₂—O— | | 476.3 | 2.43 |
| 181 | H | Me | H | H | H | MeO | 462.3 | 2.44 |
| 182 | H | Me | H | H | H | Me | 446.4 | 2.38 |
| 183 | H | Me | H | H | H | t-Bu | 488.5 | 2.64 |
| 184 | H | Me | H | H | F | Me | 464.4 | 2.43 |
| 185 | H | Me | H | H | EtO | H | 476.4 | 2.41 |
| 186 | H | Me | H | H | MeO | MeO | 492.4 | 2.27 |
| 187 | H | Me | H | H | Me | Me | 460.3 | 2.46 |
| 188 | H | Me | H | H | H | i-Pr | 474.5 | 2.56 |
| 189 | H | Me | H | H | H | EtO | 476.4 | 2.43 |
| 190 | H | Me | H | H | H | Ac | 474.3 | 2.25 |
| 191 | H | Me | H | H | H | H | 432.4 | 2.27 |
| 192 | H | Me | H | H | Me | H | 446.3 | 2.38 |
| 193 | H | Me | H | H | Cl | H | 466.4 | 3.18 |
| 194 | H | Me | H | H | H | Cl | 466.3 | 2.43 |
| 195 | Me | Me | H | H | H | Et | 474.5 | 2.59 |
| 196 | Me | Me | H | H | H | MeO | 476.5 | 2.44 |
| 197 | Me | Me | H | H | H | Cl | 480.4 | 2.55 |
| 198 | Me | Me | H | H | —O—CH₂—O— | | 490.5 | 2.40 |
| 199 | H | F | H | H | H | MeO | 466.4 | 2.57 |
| 200 | H | F | H | H | H | CF₃ | 504.4 | 3.58 |
| 201 | H | F | H | H | H | i-Pr | 478.4 | 3.01 |
| 202 | H | F | H | H | H | Ac | 478.4 | 3.00 |
| 203 | H | F | H | H | H | Cl | 470.3 | 3.28 |
| 204 | H | F | H | H | H | H | 436.2 | 2.88 |
| 205 | H | F | H | H | H | CF₃O | 520.2 | 3.64 |
| 206 | H | F | H | H | H | EtO | 480.3 | 2.83 |
| 207 | H | F | H | H | H | Me | 450.2 | 2.93 |
| 208 | H | F | H | H | H | F | 454.2 | 3.20 |
| 209 | H | F | H | H | H | Et | 464.3 | 3.06 |
| 210 | H | F | H | H | —O—CH₂—O— | | 480.4 | 2.66 |
| 211 | H | Et | H | H | H | F | 464.3 | 2.49 |
| 212 | H | Et | H | H | H | Et | 474.5 | 2.61 |
| 213 | H | Et | H | H | —O—CH₂—O— | | 490.4 | 2.43 |
| 214 | H | H | Me | H | H | Et | 460.3 | 2.56 |
| 215 | H | H | Me | H | H | i-Pr | 474.3 | 2.62 |
| 216 | H | H | Me | H | H | EtO | 476.3 | 2.53 |
| 217 | H | H | Me | H | H | Cyclohexyl | 514.4 | 2.97 |
| 218 | H | H | Me | H | H | n-butyl | 488.6 | 2.69 |
| 219 | H | H | Me | H | H | Me | 448.3 | 2.46 |
| 220 | H | H | Me | H | H | t-Bu | 448.3 | 2.30 |
| 221 | H | H | Me | H | H | Ac | 474.3 | 2.30 |
| 222 | H | H | Me | H | —O—CH₂—O— | | 476.3 | 2.36 |
| 223 | H | H | Me | H | H | F | 450.4 | 2.29 |
| 224 | H | H | Me | F | H | H | 450.4 | 2.22 |

TABLE 16b
IUPAC Names for Compounds in Table 16a

| Ex. | |
|---|---|
| No. | IUPAC Name |
| 347 | 2-[(1S)-5-(3-{[2-(4-methylphenyl)pyrimidin-4-yl]methylamino}propoxy) |
| | indanyl]acetic acid |
| 348 | 2-[(1S)-5-(3-{[2-(4-ethylphenyl)pyrimidin-4-yl]methylamino}propoxy) |
| | indanyl]acetic acid |
| 349 | 2-[(1S)-5-(3-{[2-(4-fluorophenyl)pyrimidin-4-yl]methylamino}propoxy) |
| | indanyl]acetic acid |
| 350 | 2-((1S)-5-{3-[(2-(2H-benzo[3,4-d]1,3-dioxolan-5-yl)pyrimidin-4-yl)methyl |
| | amino]propoxy}indanyl)acetic acid |
| 351 | 2-[(1S)-5-(3-{[2-(4-ethoxyphenyl)pyrimidin-4-yl]methylamino}propoxy) |
| | indanyl]acetic acid |
| 352 | 2-[(1S)-5-(3-{[2-(4-methoxyphenyl)pyrimidin-4-yl]methylamino}propoxy) |
| | indanyl]acetic acid |
| 353 | 2-[(1S)-5-(3-{[2-(3,4-dimethoxyphenyl)pyrimidin-4- |
| | yl]methylamino}propoxy) indanyl]acetic acid |
| 354 | 2-[(1S)-5-(3-{[2-(4-acetylphenyl)pyrimidin-4-yl]methylamino}propoxy) |
| | indanyl]acetic acid |
| 355 | 2-[(1S)-5-(3-{[2-(4-fluorophenyl)-5-methylpyrimidin-4- |
| | yl]methylamino}propoxy) indanyl]acetic acid |
| 356 | 2-((1S)-5-{3-[(2-(2H-benzo[3,4-d]1,3-dioxolan-5-yl)-5-methylpyrimidin-4- |
| | yl) methylamino]propoxy}indanyl)acetic acid |
| 357 | 2-[(1S)-5-(3-{[2-(4-methoxyphenyl)-5-methylpyrimidin-4-yl]methylamino} |
| | propoxy)indanyl]acetic acid |
| 358 | 2-[(1S)-5-(3-{methyl[5-methyl-2-(4-methylphenyl)pyrimidin-4-yl]amino} |
| | propoxy)indanyl]acetic acid |
| 359 | 2-{(1S)-5-[3-({2-[4-(tert-butyl)phenyl]-5-methylpyrimidin-4- |
| | yl}methylamino) propoxy]indanyl}acetic acid |
| 360 | 2-[(1S)-5-(3-{[2-(3-fluoro-4-methylphenyl)-5-methylpyrimidin-4- |
| | yl]methylamino} propoxy)indanyl]acetic acid |
| 361 | 2-[(1S)-5-(3-{[2-(3-ethoxyphenyl)-5-methylpyrimidin-4-yl]methylamino} |
| | propoxy)indanyl]acetic acid |
| 362 | 2-[(1S)-5-(3-{[2-(3,4-dimethoxyphenyl)-5-methylpyrimidin-4- |
| | yl]methylamino} propoxy)indanyl]acetic acid |
| 363 | 2-[(1S)-5-(3-{[2-(3,4-dimethylphenyl)-5-methylpyrimidin-4- |
| | yl]methylamino} propoxy)indanyl]acetic acid |
| 364 | 2-{(1S)-5-[3-(methyl{5-methyl-2-[4-(methylethyl)phenyl]pyrimidin-4- |
| | yl}amino) propoxy]indanyl}acetic acid |
| 365 | 2-[(1S)-5-(3-{[2-(4-ethoxyphenyl)-5-methylpyrimidin-4-yl]methylamino} |
| | propoxy)indanyl]acetic acid |
| 366 | 2-[(1S)-5-(3-{[2-(4-acetylphenyl)-5-methylpyrimidin-4-yl]methylamino} |
| | propoxy)indanyl]acetic acid |
| 367 | 2-((1S)-5-{3-[methyl(5-methyl-2-phenylpyrimidin-4- |
| | yl)amino]propoxy}indanyl) acetic acid |
| 368 | 2-[(1S)-5-(3-{methyl[5-methyl-2-(3-methylphenyl)pyrimidin-4-yl]amino} |
| | propoxy)indanyl]acetic acid |
| 369 | 2-[(1S)-5-(3-{[2-(3-chlorophenyl)-5-methylpyrimidin-4-yl]methylamino} |
| | propoxy)indanyl]acetic acid |
| 370 | 2-[(1S)-5-(3-{[2-(4-chlorophenyl)-5-methylpyrimidin-4-yl]methylamino} |
| | propoxy)indanyl]acetic acid |
| 371 | (2S)-2-[(1S)-5-(3-{[2-(4-ethylphenyl)-5-methylpyrimidin-4- |
| | yl]methylamino} propoxy)indanyl]propanoic acid |
| 372 | (2S)-2-[(1S)-5-(3-{[2-(4-methoxyphenyl)-5-methylpyrimidin-4- |
| | yl]methylamino} propoxy)indanyl]propanoic acid |
| 373 | (2S)-2-[(1S)-5-(3-{[2-(4-chlorophenyl)-5-methylpyrimidin-4- |
| | yl]methylamino} propoxy)indanyl]propanoic acid |
| 374 | (2S)-2-((1S)-5-{3-[(2-(2H-benzo[3,4-d]1,3-dioxolan-5-yl)-5- |
| | methylpyrimidin-4-yl)methylamino]propoxy}indanyl)propanoic acid |
| 375 | 2-[(1S)-5-(3-{[5-fluoro-2-(4-methoxyphenyl)pyrimidin-4-yl]methylamino} |
| | propoxy)indanyl]acetic acid |
| 376 | 2-{(1S)-5-[3-({5-fluoro-2-[4-(trifluoromethyl)phenyl]pyrimidin-4-yl}methyl |
| | amino)propoxy]indanyl}acetic acid |
| 377 | 2-{(1S)-5-[3-({5-fluoro-2-[4-(methylethyl)phenyl]pyrimidin-4- |
| | yl}methylamino) propoxy]indanyl}acetic acid |
| 378 | 2-[(1S)-5-(3-{[2-(4-acetylphenyl)-5-fluoropyrimidin-4- |
| | yl]methylamino}propoxy) indanyl]acetic acid |
| 379 | 2-[(1S)-5-(3-{[2-(4-chlorophenyl)-5-fluoropyrimidin-4-yl]methylamino} |
| | propoxy)indanyl]acetic acid |
| 380 | ((1S)-5-{3-[(5-fluoro-2-phenyl-4-pyrimidinyl)(methyl)amino]propoxy}-2,3- |
| | dihydro-1H-inden-1-yl)acetic acid |
| 381 | 2-{(1S)-5-[3-({5-fluoro-2-[4-(trifluoromethoxy)phenyl]pyrimidin-4- |
| | yl}methyl amino)propoxy]indanyl}acetic acid |
| 382 | 2-[(1S)-5-(3-{[2-(4-ethoxyphenyl)-5-fluoropyrimidin-4-yl]methylamino} |
| | propoxy)indanyl]acetic acid |
| 383 | 2-[(1S)-5-(3-{[5-fluoro-2-(4-methylphenyl)pyrimidin-4-yl]methylamino} |
| | propoxy)indanyl]acetic acid |
| 384 | 2-[(1S)-5-(3-{[5-fluoro-2-(4-fluorophenyl)pyrimidin-4-yl]methylamino} |
| | propoxy)indanyl]acetic acid |
| 385 | 2-[(1S)-5-(3-{[2-(4-ethylphenyl)-5-fluoropyrimidin-4-yl]methylamino} |
| | propoxy)indanyl]acetic acid |
| 386 | 2-((1S)-5-{3-[(2-(2H-benzo[3,4-d]1,3-dioxolan-5-yl)-5-fluoropyrimidin-4- |
| | yl) methylamino]propoxy}indanyl)acetic acid |
| 387 | ((1S)-5-{3-[[5-ethyl-2-(4-fluorophenyl)-4- |
| | pyrimidinyl](methyl)amino]propoxy}-2,3-dihydro-1H-inden-1-yl)acetic |
| | acid |
| 388 | 2-[(1S)-5-(3-{[5-ethyl-2-(4-ethylphenyl)pyrimidin-4- |
| | yl]methylamino}propoxy) indanyl]acetic acid |
| 389 | 2-((1S)-5-{3-[(2-(2H-benzo[3,4-d]1,3-dioxolan-5-yl)-5-ethylpyrimidin-4- |
| | yl) methylamino]propoxy}indanyl)acetic acid |
| 390 | ((1S)-5-{3-[[2-(4-ethylphenyl)-6-methyl-4- |
| | pyrimidinyl](methyl)amino]propoxy}-2,3-dihydro-1H-inden-1-yl)acetic |
| | acid |
| 391 | 2-{(1S)-5-[3-(methyl{6-methyl-2-[4-(methylethyl)phenyl]pyrimidin-4- |
| | yl}amino) propoxy]indanyl}acetic acid |
| 392 | 2-[(1S)-5-(3-{[2-(4-ethoxyphenyl)-6-methylpyrimidin-4-yl]methylamino} |
| | propoxy)indanyl]acetic acid |
| 393 | 2-[(1S)-5-(3-{[2-(4-cyclohexylphenyl)-6-methylpyrimidin-4- |
| | yl]methylamino} propoxy)indanyl]acetic acid |
| 394 | 2-[(1S)-5-(3-{[2-(4-butylphenyl)-6-methylpyrimidin-4-yl]methylamino} |
| | propoxy)indanyl]acetic acid |
| 395 | 2-[(1S)-5-(3-{methyl[6-methyl-2-(4-methylphenyl)pyrimidin-4-yl]amino} |
| | propoxy)indanyl]acetic acid |
| 396 | 2-{(1S)-5-[3-({2-[4-(tert-butyl)phenyl]-6-methylpyrimidin-4- |
| | yl}methylamino) propoxy]indanyl}acetic acid |
| 397 | 2-[(1S)-5-(3-{[2-(4-acetylphenyl)-6-methylpyrimidin-4-yl]methylamino} |
| | propoxy)indanyl]acetic acid |
| 398 | 2-((1S)-5-{3-[(2-(2H-benzo[3,4-d]1,3-dioxolan-5-yl)-6-methylpyrimidin-4- |
| | yl) methylamino]propoxy}indanyl)acetic acid |
| 399 | 2-[(1S)-5-(3-{[2-(4-fluorophenyl)-6-methylpyrimidin-4-yl]methylamino} |
| | propoxy)indanyl]acetic acid |
| 400 | 2-[(1S)-5-(3-{[2-(2-fluorophenyl)-6-methylpyrimidin-4- |
| | yl]methylamino}propoxy) indanyl]acetic acid |

TABLE 17a
Formula Iyy

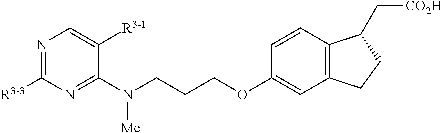

| Ex. | | | LCMS | RT |
|---|---|---|---|---|
| No. | R³⁻¹ | R³⁻³ | (M + H) | (min) |
| 225 | H | Cl | 390.3 | 3.46 |
| 226 | Me | 3-thienyl | 438.3 | 2.25 |
| 227 | Me | 4-MeO—Ph—O | 478.5 | 2.35 |
| 228 | Me | 4-F—Ph—O | 466.4 | 2.41 |
| 229 | Me | 3-F—Ph—O | 478.5 | 2.39 |
| 230 | H | 2-benzofuryl | 458.3 | 2.45 |
| 231 | F | 2-benzofuryl | 476.4 | 3.10 |

TABLE 17b
IUPAC Names for Compounds in Table 17a

| Ex. | |
|---|---|
| No. | IUPAC Name |
| 401 | ((1S)-5-{3-[(2-chloro-4-pyrimidinyl)(methyl)amino]propoxy}- |
| | 2,3-dihydro-1H-inden-1-yl)acetic acid |
| 402 | 2-((1S)-5-{3-[methyl(5-methyl-2-(3-thienyl)pyrimidin-4- |
| | yl)amino]propoxy} indanyl)acetic acid |
| 403 | ((1S)-5-{3-[[2-(4-methoxyphenoxy)-5-methyl-4- |
| | pyrimidinyl](methyl)amino] propoxy}-2,3-dihydro-1H-inden- |
| | 1-yl)acetic acid |
| 404 | ((1S)-5-{3-[[2-(4-fluorophenoxy)-5-methyl-4- |
| | pyrimidinyl](methyl)amino] propoxy}-2,3-dihydro-1H-inden- |
| | 1-yl)acetic acid |
| 405 | ((1S)-5-{3-[[2-(3-methoxyphenoxy)-5-methyl-4- |
| | pyrimidinyl](methyl) amino]propoxy}-2,3-dihydro-1H-inden- |
| | 1-yl)acetic acid |
| 406 | 2-((1S)-5-{3-[(2-benzo[d]furan-2-ylpyrimidin-4- |
| | yl)methylamino]propoxy} indanyl)acetic acid |
| 407 | 2-((1S)-5-{3-[(2-benzo[d]furan-2-yl-5-fluoropyrimidin- |
| | 4-yl)methylamino] propoxy}indanyl)acetic acid |

Exemplary compounds of Formula (Izz) is listed in Table 18a.

TABLE 18a
Formula Izzz

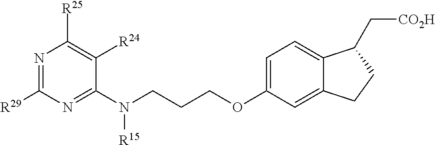

| Ex. | | | | | LCMS | RT |
|---|---|---|---|---|---|---|
| No. | R⁵ | R³⁻¹ | R³⁻² | R³⁻³ | (M + H) | (min) |
| 232 | H | H | Ph | H | 404.3 | 2.11 |
| 233 | H | H | H | 4-MePh | 418.4 | 3.02 |
| 234 | H | Me | H | 4-Et—Ph | 446.3 | 2.46 |
| 235 | H | Me | H | 4-MePh | 432.3 | 2.46 |
| 236 | H | Me | H | 4-MeOPh | 448.4 | 2.30 |
| 237 | H | Me | H | 3,4-dioxolane- | 462.3 | 2.25 |
| | | | | Ph | | |
| 238 | H | Me | H | 3-thienyl | 424.3 | 2.20 |
| 239 | H | Me | H | 4-F—Ph | 436.3 | 2.28 |
| 240 | H | Me | H | 3-MePh | 432.3 | 2.34 |
| 241 | H | Me | H | 3-MeO—Ph | 448.3 | 2.29 |
| 242 | H | Me | H | 4-CF₃—Ph | 486.3 | 2.48 |
| 243 | n-Pr | Me | H | 4-Me—Ph | 474.4 | 3.29 |
| 244 | n-Pr | Me | H | 4-MeO—Ph | 490.4 | 3.24 |
| 245 | n-Pr | Me | H | 3,4-dioxolane- | 504.4 | 3.20 |
| | | | | Ph | | |
| 246 | n-Pr | Me | H | 3-thienyl | 466.3 | 3.17 |
| 247 | n-Pr | Me | H | 4-F—Ph | 478.4 | 3.24 |
| 248 | n-Pr | Me | H | 3-Me—Ph | 474.4 | 3.29 |
| 249 | n-Pr | H | H | 4-Me—Ph | 460.3 | 2.65 |
| 250 | n-Pr | H | H | 4-Et—Ph | 474.3 | 2.77 |
| 251 | n-Pr | H | H | 3,4-dioxolane- | 490.3 | 2.53 |
| | | | | Ph | | |
| 252 | n-Pr | H | H | 4-MeO—Ph | 476.5 | 2.46 |
| 253 | 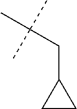 | H | H | 4-Et | 500.5 | 2.74 |
| 254 | Et | Me | H | 4-Et—Ph | 474.5 | 2.61 |
| 255 | Et | Me | H | 4-Me—Ph | 460.4 | 2.52 |
| 256 | Et | Me | H | 3,4-dioxolane- | 490.4 | 2.42 |
| | | | | Ph | | |
| 257 | Ac | Me | H | 4-Et—Ph | 488.1 | 3.35 |
| 258 | Ac | Me | H | 3,4-dioxolane- | 504.2 | 2.92 |
| | | | | Ph | | |

TABLE 18b
IUPAC Names for Compounds in Table 18a

| Ex. | |
|---|---|
| No. | IUPAC Name |
| 411 | ((1S)-5-{3-[(6-phenyl-4-pyrimidinyl)amino]propoxy}-2,3-dihydro-1H- |
| | inden-1-yl)acetic acid |
| 412 | 2-[(1S)-5-(3-{[2-(4-methylphenyl)pyrimidin-4- |
| | yl]amino}propoxy)indanyl]acetic acid |
| 413 | 2-[(1S)-5-(3-{[2-(4-ethylphenyl)-5-methylpyrimidin-4-yl]amino}propoxy) |
| | indanyl]acetic acid |
| 414 | 2-[(1S)-5-(3-{[5-methyl-2-(4-methylphenyl)pyrimidin-4-yl]amino}propoxy) |
| | indanyl]acetic acid |
| 415 | 2-[(1S)-5-(3-{[2-(4-methoxyphenyl)-5-methylpyrimidin-4- |
| | yl]amino}propoxy) indanyl]acetic acid |
| 416 | 2-((1S)-5-{3-[(2-(2H-benzo[3,4-d]1,3-dioxolan-5-yl)-5-methylpyrimidin-4- |
| | yl)amino]propoxy}indanyl)acetic acid |
| 417 | 2-((1S)-5-{3-[(5-methyl-2-(3-thienyl)pyrimidin-4- |
| | yl)amino]propoxy}indanyl) acetic acid |
| 418 | 2-[(1S)-5-(3-{[2-(4-fluorophenyl)-5-methylpyrimidin-4-yl]amino}propoxy) |
| | indanyl]acetic acid |
| 419 | 2-[(1S)-5-(3-{[5-methyl-2-(3-methylphenyl)pyrimidin-4-yl]amino}propoxy) |
| | indanyl]acetic acid |
| 420 | 2-[(1S)-5-(3-{[2-(3-methoxyphenyl)-5-methylpyrimidin-4- |
| | yl]amino}propoxy) indanyl]acetic acid |
| 421 | 2-{(1S)-5-[3-({5-methyl-2-[4-(trifluoromethyl)phenyl]pyrimidin-4- |
| | yl}amino) propoxy]indanyl}acetic acid |
| 422 | 2-[(1S)-5-(3-{[5-methyl-2-(4-methylphenyl)pyrimidin-4-yl]propylamino} |
| | propoxy)indanyl]acetic acid |
| 423 | ((1S)-5-{3-[[2-(4-methoxyphenyl)-5-methyl-4-pyrimidinyl](propyl)amino] |
| | propoxy}-2,3-dihydro-1H-inden-1-yl)acetic acid |
| 424 | 2-((1S)-5-{3-[(2-(2H-benzo[3,4-d]1,3-dioxolan-5-yl)-5-methylpyrimidin-4- |
| | yl)propylamino]propoxy}indanyl)acetic acid |
| 425 | 2-((1S)-5-{3-[(5-methyl-2-(3-thienyl)pyrimidin-4-yl)propylamino]propoxy} |
| | indanyl)acetic acid |
| 426 | 2-[(1S)-5-(3-{[2-(4-fluorophenyl)-5-methylpyrimidin-4-yl]propylamino} |
| | propoxy)indanyl]acetic acid |
| 427 | 2-[(1S)-5-(3-{[5-methyl-2-(3-methylphenyl)pyrimidin-4-yl]propylamino} |
| | propoxy)indanyl]acetic acid |
| 428 | 2-[(1S)-5-(3-{[2-(4-methylphenyl)pyrimidin-4-yl]propylamino}propoxy) |
| | indanyl]acetic acid |
| 429 | 2-[(1S)-5-(3-{[2-(4-ethylphenyl)pyrimidin-4-yl]propylamino}propoxy) |
| | indanyl]acetic acid |
| 430 | 2-((1S)-5-{3-[(2-(2H-benzo[3,4-d]1,3-dioxolan-5-yl)pyrimidin-4- |
| | yl)propylamino] propoxy}indanyl)acetic acid |
| 431 | 2-[(1S)-5-(3-{[2-(4-methoxyphenyl)pyrimidin-4-yl]propylamino}propoxy) |
| | indanyl]acetic acid |
| 432 | 2-[(1S)-5-(3-{(cyclopropylmethyl)[2-(4-ethylphenyl)-5-methylpyrimidin-4- |
| | yl]amino}propoxy)indanyl]acetic acid |
| 433 | 2-[(1S)-5-(3-{ethyl[2-(4-ethylphenyl)-5-methylpyrimidin-4- |
| | yl]amino}propoxy) indanyl]acetic acid |
| 434 | [(1S)-5-(3-{ethyl[5-methyl-2-(4-methylphenyl)-4- |
| | pyrimidinyl]amino}propoxy)-2,3-dihydro-1H-inden-1-yl]acetic acid |
| 435 | 2-((1S)-5-{3-[(2-(2H-benzo[3,4-d]1,3-dioxolan-5-yl)-5-methylpyrimidin-4- |
| | yl)ethylamino]propoxy}indanyl)acetic acid |
| 436 | 2-[(1S)-5-(3-{N-[2-(4-ethylphenyl)-5-methylpyrimidin-4-yl]acetylamino} |
| | propoxy)indanyl]acetic acid |
| 437 | [(1S)-5-(3-{acetyl[2-(1,3-benzodioxol-5-yl)-5-methyl-4- |
| | pyrimidinyl]amino} propoxy)-2,3-dihydro-1H-inden-1-yl]acetic acid |

Exemplary compounds of Formula (Iaaa) are listed in Table 19a below.

TABLE 19a
Formula Iaaa

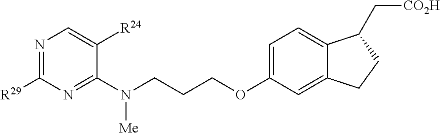

| Ex. | | | LCMS | RT |
|---|---|---|---|---|
| No. | R³⁻¹ | R³⁻² | (M + H) | (min) |
| 259 | 4-Ac—Ph | 4-Ac—Ph | 578.2 | 2.75 |
| 260 | 4-CF₃—Ph | 4-CF₃—Ph | 630.5 | 3.61 |
| 261 | 4-F—Ph | 4-F—Ph | 530.3 | 2.78 |
| 262 | 4-Et—Ph | Cl | 480.6 | 3.34 |
| 263 | 4-CF₃O—Ph | Cl | 536.5 | 3.90 |
| 264 | 4-Ac—Ph | Cl | 494.5 | 3.37 |
| 265 | 4-CF₃—Ph | Cl | 520.5 | 3.96 |
| 266 | 3,4-dioxolane-Ph | Cl | 496.3 | 3.06 |
| 267 | 4-F—Ph | Cl | 470.5 | 3.41 |
| 268 | 4-Me—Ph | Cl | 466.2 | 3.16 |
| 269 | 3,4-diF—Ph | Cl | 488.2 | 3.81 |

TABLE 19b
IUPAC Names for Compounds in Table 19a

| Ex. | |
|---|---|
| No. | IUPAC Name |
| 447 | 2-[(1S)-5-(3-{[2,5-bis(4-acetylphenyl)pyrimidin-4- |
| | yl]methylamino}propoxy) indanyl]acetic acid |
| 448 | 2-{(1S)-5-[3-({2,5-bis[4-(trifluoromethyl)phenyl]pyrimidin-4- |
| | yl}methylamino) propoxy]indanyl}acetic acid |
| 449 | 2-[(1S)-5-(3-{[2,5-bis(4-fluorophenyl)pyrimidin-4- |
| | yl]methylamino}propoxy) indanyl]acetic acid |
| 450 | 2-[(1S)-5-(3-{[2-chloro-5-(4-ethylphenyl)pyrimidin-4-yl]methylamino} |
| | propoxy)indanyl]acetic acid |
| 451 | 2-{(1S)-5-[3-({2-chloro-5-[4-(trifluoromethoxy)phenyl]pyrimidin-4- |
| | yl}methyl amino)propoxy]indanyl}acetic acid |
| 452 | 2-[(1S)-5-(3-{[5-(4-acetylphenyl)-2-chloropyrimidin-4-yl]methylamino} |
| | propoxy)indanyl]acetic acid |
| 453 | 2-{(1S)-5-[3-({2-chloro-5-[4-(trifluoromethyl)phenyl]pyrimidin-4-yl}methyl |
| | amino)propoxy]indanyl}acetic acid |
| 454 | 2-((1S)-5-{3-[(5-(2H-benzo[3,4-d]1,3-dioxolan-5-yl)-2-chloropyrimidin-4- |
| | yl) methylamino]propoxy}indanyl)acetic acid |
| 455 | 2-[(1S)-5-(3-{[2-chloro-5-(4-fluorophenyl)pyrimidin-4-yl]methylamino} |
| | propoxy)indanyl]acetic acid |
| 456 | ((1S)-5-{3-[[2-chloro-5-(4-methylphenyl)-4-pyrimidinyl](methyl)amino] |
| | propoxy}-2,3-dihydro-1H-inden-1-yl)acetic acid |
| 457 | ((1S)-5-{3-[[2-chloro-5-(3,4-difluorophenyl)-4-pyrimidinyl](methyl)amino] |
| | propoxy}-2,3-dihydro-1H-inden-1-yl)acetic acid |

Exemplary compounds of Formula (Ibbb) are shown in Table 20a below.

TABLE 20a
Formula Ibbb

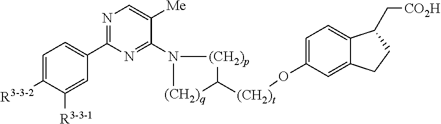

| Ex. | | | | | | LCMS | RT |
|---|---|---|---|---|---|---|---|
| No. | R³⁻³⁻² | R³⁻³⁻¹ | p | q | t | (M + H) | (min) |
| 270¹ | Et | H | 0 | 3 | 1 | 472.5 | 2.57 |
| 271 | F | H | 2 | 2 | 0 | 462.3 | 2.52 |
| 272 | i-Pr | H | 2 | 2 | 0 | 486.4 | 2.76 |
| 273 | MeO | H | 2 | 2 | 0 | 474.3 | 2.47 |
| 274 | Cl | H | 2 | 2 | 0 | 478.3 | 2.70 |

| | | | | | | |
|---|---|---|---|---|---|---|
| 275 | —O—CH₂—O— | 2 | 2 | 0 | 488.3 | 2.45 |

¹The absolute configuration at carbon * is S.

TABLE 20b
IUPAC Names for Compounds in Table 20a

| Ex. | |
|---|---|
| No. | IUPAC Name |
| 461 | [(1S)-5-({(2S)-1-[2-(4-ethylphenyl)-5-methyl-4-pyrimidinyl]-2- |
| | pyrrolidinyl}methoxy)-2,3-dihydro-1H-inden-1-yl]acetic acid |
| 462 | [(1S)-5-({1-[2-(4-fluorophenyl)-5-methyl-4-pyrimidinyl]-4- |
| | piperidinyl} oxy)-2,3-dihydro-1H-inden-1-yl]acetic acid |
| 463 | [(1S)-5-({1-[2-(4-i-propylphenyl)-5-methyl-4-pyrimidinyl]-4- |
| | piperidinyl} oxy)-2,3-dihydro-1H-inden-1-yl]acetic acid |
| 464 | [(1S)-5-({1-[2-(4-methoxyphenyl)-5-methyl-4-pyrimidinyl]-4- |
| | piperidinyl} oxy)-2,3-dihydro-1H-inden-1-yl]acetic acid |
| 465 | [(1S)-5-({1-[2-(4-chlorophenyl)-5-methyl-4-pyrimidinyl]-4- |
| | piperidinyl} oxy)-2,3-dihydro-1H-inden-1-yl]acetic acid |
| 466 | [(1S)-5-({1-[2-(1,3-benzodioxol-5-yl)-5-methyl-4-pyrimidinyl]-4- |
| | piperidinyl}oxy)-2,3-dihydro-1H-inden-1-yl]acetic acid |

More exemplary compounds of Formula (Iccc), is listed in Table 21a below.

TABLE 21a
Formula Iccc

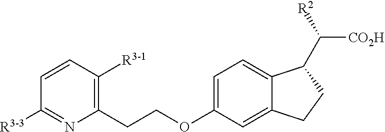

| Ex. | | | | LCMS | RT |
|---|---|---|---|---|---|
| No. | R² | R³⁻¹ | R³⁻³ | (M + H) | (min) |
| 276 | H | Me | H | 312.2 | 1.75 |
| 277 | H | H | 4-Me—Ph | 388.2 | 2.64 |
| 278 | H | H | 4-Ac—Ph | 416.2 | 2.94 |
| 279 | H | H | 4-MeO—Ph | 404.1 | 2.55 |
| 280 | H | H | 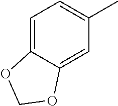 | 418.1 | 2.67 |
| 281 | H | H | 4-Cl—Ph | 408.1 | 3.24 |
| 282 | H | H | 4-F—Ph | 392.1 | 2.93 |
| 283 | H | H | H | 374.2 | 2.69 |
| 284 | H | H | 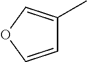 | 364.1 | 2.43 |
| 285 | H | H | 4-CF₃—Ph | 442.2 | 4.13 |
| 286 | H | H | 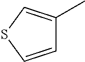 | 380.3 | 3.19 |
| 287 | H | H | 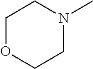 | 383.3 | 2.68 |
| 288 | H | H | 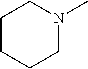 | 381.3 | 2.10 |
| 289 | H | H | 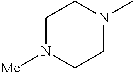 | 396.3 | 1.91 |
| 290 | Et | H | 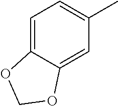 | 446.3 | 3.74 |
| 291 | Et | H | 4-Et—Ph | 430.4 | 3.74 |
| 292 | Et | H | 4-CF₃—Ph | 470.4 | 4.35 |
| 293 | H | Me | 4-Et—Ph | 416.2 | 2.85 |
| 294 | H | Me | 4-CF₃—Ph | 456.2 | 3.57 |
| 295 | Me | H | Et | 340.2 | 2.07 |
| 296 | H | H | Et | 326.2 | 1.94 |

TABLE 21b
IUPAC Names for Compounds in Table 21a

| Ex. | |
|---|---|
| No. | IUPAC Name |
| 483 | 2-{(1S)-5-[2-(3-methyl(2-pyridyl))ethoxy]indanyl}acetic acid, |
| | trifluoromethanane acetic acid salt |
| 484 | 2-(5-{2-[6-(4-methylphenyl)-2-pyridyl]ethoxy}indanyl)acetic acid |
| 485 | 2-((1S)-5-{2-[6-(4-acetylphenyl)(2-pyridyl)]ethoxy}indanyl)acetic acid |
| 486 | 2-((1S)-5-{2-[6-(4-methoxyphenyl)(2-pyridyl)]ethoxy}indanyl)acetic acid |
| 487 | 2-{5-[2-(6-(2H-benzo[3,4-d]1,3-dioxolan-5-yl)(2-pyridyl))ethoxy] |
| | (1S)indanyl}acetic acid |
| 488 | 2-((1S)-5-{2-[6-(4-chlorophenyl)(2-pyridyl)]ethoxy}indanyl)acetic acid |
| 489 | 2-((1S)-5-{2-[6-(4-fluorophenyl)(2-pyridyl)]ethoxy}indanyl)acetic acid |
| 490 | 2-{(1S)-5-[2-(6-phenyl(2-pyridyl))ethoxy]indanyl}acetic acid |
| 491 | 2-{(1S)-5-[2-(6-(3-furyl)(2-pyridyl))ethoxy]indanyl}acetic acid |
| 492 | 2-((1S)-5-{2-[6-(4-trifluoromethylphenyl)(2-pyridyl)]ethoxy}indanyl)acetic |
| | acid |
| 493 | 2-{(1S)-5-[2-(6-(3-thienyl)(2-pyridyl))ethoxy]indanyl}acetic acid |
| 494 | 2-{(1S)-5-[2-(6-morpholin-4-yl(2-pyridyl))ethoxy]indanyl}acetic acid |
| 495 | ((1S)-5-{2-[6-(1-piperidinyl)-2-pyridinyl]ethoxy}-2,3-dihydro-1H-inden-1- |
| | yl) acetic acid |
| 496 | 2-((1S)-5-{2-[6-(4-methylpiperazinyl)(2-pyridyl)]ethoxy}indanyl)acetic |
| | acid |
| 497 | 2-{5-[2-(6-(2H-benzo[3,4-d]1,3-dioxolan-5-yl)(2-pyridyl))ethoxy] |
| | (1S)indanyl}(2S)butanoic acid |
| 498 | (2S)-2-((1S)-5-{2-[6-(4-ethylphenyl)(2-pyridyl)]ethoxy}indanyl)butanoic |
| | acid |
| 499 | (2S)-2-[(1S)-5-(2-{6-[4-(trifluoromethyl)phenyl](2-pyridyl)}ethoxy) |
| | indanyl]butanoic acid |
| 500 | 2-((1S)-5-{2-[6-(4-ethylphenyl)-3-methyl(2-pyridyl)]ethoxy}indanyl)acetic |
| | acid, chloride |
| 501 | 2-[(1S)-5-(2-{3-methyl-6-[4-(trifluoromethyl)phenyl](2-pyridyl)}ethoxy) |
| | indanyl]acetic acid |
| 502 | (2S)-2-{(1S)-5-[2-(5-ethyl(2-pyridyl))ethoxy]indanyl}propanoic acid |
| 503 | 2-{(1S)-5-[2-(5-ethyl(2-pyridyl))ethoxy]indanyl}acetic acid |

In general, the compounds of Formula VI of this invention may be prepared by standard techniques known in the art and by known processes analogous thereto. For example, the compounds may be prepared according to methods described in U.S. Patent Application Publication No. 2006/0084680, which is incorporated by reference in its entirety.

The present invention also encompasses indane acetic acid compounds and derivatives described in U.S. Pat. No. 7,476,742 and U.S. Patent Application Publication No. 2006/0264486, which are incorporated by references in their entirety.

The compounds described in Tables 1-20 are intended to be representative examples of the invention, and it will be understood that the scope of the invention is not limited by the scope of the examples. Those skilled in the art will recognize that the invention may be practiced with variations on the disclosed structures, materials, compositions and methods, and such variations are regarded as within the ambit of the invention.

A salt of a compound described in the present invention may be prepared in situ during the final isolation and purification of a compound or by separately reacting the purified compound in its free base form with a suitable organic or inorganic acid and isolating the salt thus formed. Likewise, when the compound described in the present invention contain a carboxylic acid moiety, (e.g., R=H), a salt of said compound may be prepared by separately reacting it with a suitable inorganic or organic base and isolating the salt thus formed. The term “pharmaceutically acceptable salt” refers to a relatively non-toxic, inorganic or organic acid addition salt of a compound of the present invention (see, e.g., Berge et al., J. Pharm. Sci. 66:1-19, 1977).

Representative salts of the compounds described in the present invention include the conventional non-toxic salts and the quaternary ammonium salts, which are formed, for example, from inorganic or organic acids or bases by means well known in the art. For example, such acid addition salts include acetate, adipate, alginate, ascorbate, aspartate, benzoate, benzenesulfonate, bisulfate, butyrate, citrate, camphorate, camphorsulfonate, cinnamate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, itaconate, lactate, maleate, mandelate, methanesulfonate, 2-naphthalenesulfonate, nicotinate, nitrate, oxalate, pamoate, pectinate, persulfate, 3-phenylpropionate, picrate, pivalate, propionate, succinate, sulfonate, tartrate, thiocyanate, tosylate, undecanoate, and the like.

Base salts include, for example, alkali metal salts such as potassium and sodium salts, alkaline earth metal salts such as calcium and magnesium salts, and ammonium salts with organic bases such as dicyclohexylamine and N-methyl-D-glucamine. Additionally, basic nitrogen containing groups in the conjugate base may be quaternized with alkyl halides, e.g., C₁₋₉ alkyl halides such as methyl, ethyl, propyl, and butyl chlorides, bromides and iodides; dialkyl sulfates like dimethyl, diethyl, and dibutyl sulfate; and diamyl sulfates, C₁₀₋₄₀ alkyl halides such as decyl, lauryl, myristyl and strearyl chlorides, bromides and iodides; or aralkyl halides like benzyl and phenethyl bromides. In some embodiments, the salts are alkali salt such as sodium or potassium salt or an adduct with an acceptable nitrogen base such as meglumine (N-Methyl-d-glucamine) salt.

The esters of the compounds described in the present invention are non-toxic, pharmaceutically acceptable esters, for example, alkyl esters such as methyl, ethyl, propyl, isopropyl, butyl, isobutyl, or pentyl esters. Additional esters such as, for example, methyl ester or phenyl-C₁-C₅ alkyl may be used. The compound described in the present invention may be esterified by a variety of conventional procedures including reacting the appropriate anhydride, carboxylic acid, or acid chloride with the alcohol group of the compounds described in the present invention compound. The appropriate anhydride may be reacted with the alcohol in the presence of a base to facilitate acylation such as 1,8-bis[dimethylamino]naphthalene or N,N-dimethylaminopyridine. An appropriate carboxylic acid may be reacted with the alcohol in the presence of a dehydrating agent such as dicyclohexylcarbodiimide, 1-[3-dimethylaminopropyl]-3-ethylcarbodiimide, or other water soluble dehydrating agents which are used to drive the reaction by the removal of water, and optionally, an acylation catalyst. Esterification may also be effected using the appropriate carboxylic acid in the presence of trifluoroacetic anhydride and optionally, pyridine, or in the presence of N,N-carbonyldiimidazole with pyridine. Reaction of an acid chloride with the alcohol may be carried out with an acylation catalyst such as 4-DMAP or pyridine.

One skilled in the art would readily know how to successfully carry out these as well as other methods of esterification of alcohols.

Additionally, sensitive or reactive groups on the compound described in the present invention may need to be protected and deprotected during any of the above methods for forming esters. Protecting groups in general may be added and removed by conventional methods well known in the art (see, e.g., T. W. Greene and P. G. M. Wuts, Protective Groups in Organic Synthesis; Wiley: New York, (1999)).

The compounds described in the present invention may contain one or more asymmetric centers, depending upon the location and nature of the various substituents desired. Asymmetric carbon atoms may be present in the (R) or (S) configuration. Preferred isomers are those with the absolute configuration, which produces the compound of described in the present invention with the more desirable biological activity. In certain instances, asymmetry may also be present due to restricted rotation about a given bond, for example, the central bond adjoining two aromatic rings of the specified compounds.

Substituents on a ring may also be present in either cis or trans form, and a substituent on a double bond may be present in either Z or E form.

It is intended that all isomers (including enantiomers and diastereomers), either by nature of asymmetric centers or by restricted rotation as described above, as separated, pure or partially purified isomers or racemic mixtures thereof, be included within the scope of the instant invention. The purification of said isomers and the separation of said isomeric mixtures may be accomplished by standard techniques known in the art.

As described herein, compounds of the invention may optionally be substituted with one or more substituents, such as are illustrated generally above, or as exemplified by particular classes, subclasses, and species of the invention. In general, the term “substituted” refers to the replacement of hydrogen radicals in a given structure with the radical of a specified substituent. Unless otherwise indicated, a substituted group may have a substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds.

### C. Evaluation of Biological Activity of Compounds

Recent research indicates that PPAR agonists may be used to treat cognitive impairment in Alzheimer's disease. (See Escribano et al., Rosiglitazone reverses memory decline and hippocampal glucocorticoid receptor down-regulation in an Alzheimer's disease mouse model, Biochemical and Biophysical Research Communications, 379, 406-410 (2009)).

PPAR receptor agonist activity may be determined by conventional screening methods known to the skilled in the art. For example, methods described in U.S. Patent Application Publication No. 2007/0054907, 2008/0262047 and U.S. Pat. No. 7,314,879, which are incorporated by reference in their entireties. Exemplary screening tests are described below:

### (1) Binding Assay

Compounds may be tested for their ability to bind to hPPAR gamma, hPPAR alpha or hPPAR delta using a Scintillation Proximity Assay (SPA). The PPAR ligand binding domain (LBD) may be expressed in E. coli as polyHis tagged fusion proteins and purified. The LBD may then be labelled with biotin and immobilized on streptavidin-modified scintillation proximity beads. The beads may then be incubated with a constant amount of the appropriate radioligand (5-{4-[2-(Methyl-pyridin-2-yl-amino)-ethoxy]-benzyl}-thiazolidine-2,4-dio-ne (J. Med. Chem. 1994, 37(23), 3977), for PPAR gamma), and labelled GW 2433 (see Brown, P. J et al. Chem. Biol. 1997 4: 909-918), for the structure and synthesis of this ligand) for PPAR alpha and PPAR delta) and variable concentrations of test compound, and after equilibration the radioactivity bound to the beads may be measured by a scintillation counter. The amount of nonspecific binding, as assessed by control wells containing 50 μM of the corresponding unlabeled ligand, is subtracted from each data point. For each compound tested, plots of ligand concentration vs. CPM of radioligand bound may be constructed and apparent Ki values are estimated from nonlinear least squares fit of the data assuming simple competitive binding. The details of this assay have been reported elsewhere (see, Blanchard, S. G. et. al. Anal. Biochem., 257 112-119 (1998)).

### (2). Functional Assays

(a) Functional cell based assays are developed to discriminate agonists and antagonists.

Agonist Assay: HEK 293 cells stably expressing a human melanocortin receptor (see e.g., Yang, et al., Mol-Endocrinol., 11(3): 274-80, 1997) are dissociated from tissue culture flasks using a trypsin/EDTA solution (0.25%; Life Technologies, Rockville, Md.). Cells are collected by centrifugation and resuspended in DMEM (Life Technologies, Rockville, Md.) supplemented with 1% L-glutamine and 0.5% fetal bovine serum. Cells are counted and diluted to 4.5×10⁵/ml.

A compound of the present invention is diluted in dimethylsulfoxide (DMSO) (3×10⁻⁵ to 3×10⁻¹⁰ M final concentrations) and 0.05 volume of compound solution is added to 0.95 volumes of cell suspension; the final DMSO concentration is 0.5%. After incubation at 37° C./5% CO₂ for 5 hours, cells are lysed by addition of luciferin solution (50 mM Tris, 1 mM MgCl₂, 0.2% Triton-X100, 5 mM DTT, 500 micromolar Coenzyme A, 150 micromolar ATP, and 440 micromolar luciferin) to quantify the activity of the reporter gene luciferase, an indirect measurement of intracellular cAMP production.

Luciferase activity is measured from the cell lysate using a Wallac Victor 2 luminometer. The amount of lumen production which results from a compound of present invention is compared to that amount of lumens produced in response to NDP-alpha-MSH, defined as a 100% agonist, to obtain the relative efficacy of a compound. The EC₅₀ is defined as the compound concentration that results in half maximal stimulation, when compared to its own maximal level of stimulation.

(b) Melanocortin Receptor Whole Cell cAMP Accumulation Assay Compound preparation:

In the agonist assay, compounds are prepared as 10 mM and NDP-aMSH (control) as 33.3 μM stock solutions in 100% DMSO. These are serially diluted in 100% DMSO. The compound plate is further diluted 1:200 in compound dilution buffer (HBSS-092, 1 mM Ascorbic Acid, 1 mM IBMX, 0.6% DMSO, 0.1% BSA). The final concentration range being 10 μM-100 pM for compound and 33.33 nM-0.3 pM for control in 0.5% DMSO. Transfer 20 μl from this plate into four PET 96-well plates (all assays are performed in duplicate for each receptor).

(c) Cell Culture and Cell Stimulation:

HEK 293 cells stably transfected with the MC3R and MC4R are grown in DMEM containing 10% FBS and 1% Antibiotic/Antimycotic Solution. On the day of the assay the cells are dislodged with enzyme free cell dissociation solution and resuspended in cell buffer (HBSS-092, 0.1% BSA, 10 mM HEPES) at 1×e6 cells/ml. Add 40 μl of cells/well to the PET 96-well plates containing 20 ul diluted compound and control. Incubate @ 37° C. in a water bath for 20 minutes. Stop the assay by adding 50 μl Quench Buffer (50 mM Na Acetate, 0.25% Triton X-100).

### (3) Radioligand Binding Assays

Radioligand binding assays are run in SPA buffer (50 mM Sodium Acetate, 0.1% BSA). The beads, antibody and radioligand are diluted in SPA buffer to provide sufficient volume for each 96-well plate. To each quenched assay well is added 100 ul cocktail containing 33.33 μl of beads, 33.33 μl antibody and 33.33 μl ¹²⁵I-cAMP. This is based on a final concentration of 6.3 mg/ml beads, 0.65% anti-goat antibody and 61 pM of ¹²⁵I-cAMP (containing 25000-30000 CPM) in a final assay volume of 210 μl. The plates are counted in a Wallac MicroBeta counter after a 12-hour incubation.

The data is converted to pmoles cAMP using a standard curve assayed under the same conditions. The data is analyzed using Activity Base software to generate agonist potencies (EC₅₀) and percent relative efficacy data to NDP-aMSH.

### (4) Transfection Assay

Compounds may be screened for functional potency in transient transfection assays in CV-1 cells for their ability to activate the PPAR subtypes (transactivation assay). A previously established chimeric receptor system may be utilized to allow comparison of the relative transcriptional activity of the receptor subtypes on the same target gene and to prevent endogenous receptor activation from complicating the interpretation of results. See, for example, Lehmann, J. M et al J. Biol. Chem., 1995 270:12953-6. The ligand binding domains for murine and human PPAR alpha, PPAR gamma and PPAR delta are each fused to the yeast transcription factor GAL4 DNA binding domain. CV-1 cells are transiently transfected with expression vectors for the respective PPAR chimera along with a reporter construct containing five copies of the GAL4 DNA binding site driving expression of secreted placental alkaline phosphatase (SPAP) and beta-galactosidase. After 16 h, the medium are exchanged to DME medium supplemented with 10% delipidated fetal calf serum and the test compound at the appropriate concentration. After an additional 24 h, cell extracts are prepared and assayed for alkaline phosphatase and beta-galactosidase activity. Alkaline phosphatase activity is corrected for transfection efficiency using the beta-galactosidase activity as an internal standard (see, for example, Kliewer, S. A., et. al. Cell 1995 83: 813-819). Rosiglitazone (BRL 49653) may be used as a positive control in the hPPAR gamma assay. The positive control in the hPPAR alpha assays may be 2-4-[2-(3-[4-fluorophenyl]-1-heptylureido)ethyl]-phenoxy-(2-methyl propionic acid (WO 97/36579). The positive control for PPAR delta assays may be 2-{2-methyl-4-[({4-methyl-2-{trifluoromethyl)phenyl]-1,3-thiazol-5-yl}methyl)sulfanyl]phenoxy}acetic acid (WO 01/00603). An EC50 may be determined as the concentration at which a compound achieves 50% activation relative to the appropriate positive control.

An “agonist” will typically have a pKi of at least 6.0 preferably at least 7.0 to the relevant PPAR in the Binding Assay described above, and achieves at least 50% activation of the relevant PPAR relative to the appropriate indicated positive control in the Transfection Assay described above at concentrations of 10^{-5 M} or less.

### (5) Cross Curve PPAR Transactivation Test

The activation of receptors with an agonist (activator) in HeLN cells leads to the expression of a reporter gene, luciferase, which, in the presence of a substrate, generates light. The modulation of the receptors is measured as quantity of luminescence produced after incubating the cells in the presence of a reference agonist. The ligands will displace the agonist from its site. The measurement of the activity is performed by quantification of the light produced. This measurement makes it possible to determine the modulatory activity of the compounds according to the invention by determining the constant, which is the affinity of the molecule for the receptor. Since this value can fluctuate according to the basal activity and the expression of the receptor, it is called apparent Kd (Kd app in nM).

To determine this constant, the cells are in contact with a concentration of the product to be tested and a concentration of the reference agonist, 2-(4-{2-[3-(2,4-difluorophenyl)-1-heptylureido]ethyl}phenylsulfanyl)-2-methylpropionic acid for PPARα, {2-methyl-4-[4-methyl-2-(4-trifluoromethylphenyl)thiazol-5-ylmethylsulfanyl]phenoxy}acetic acid for PPARδ and 5-{4-[2-(methylpyridin-2-ylamino)ethoxy]benzyl}thiazolidine-2,4-dione for PPARγ. Measurements are also carried out for the controls total agonist with the same products.

The HeLN cell lines used are stable transfectants containing the plasmids ERE-βGlob-Luc-SV-Neo (reporter gene) and PPAR (α, δ, γ) Gal-hPPAR. These cells are inoculated into 96-well plates in an amount of 10 000 cells per well in 100 μl of DMEM medium free of phenol red and supplemented with 10% lipid-free calf serum. The plates are then incubated at 37° C., 7% CO₂ for 16 hours.

The various dilutions of the test products and of the reference ligand are added in an amount of 5 μl per well. The plates are then incubated for 18 hours at 37° C., 7% CO₂. The culture medium is removed by turning over and 100 μl of a 1:1 PBS/Luciferin mixture are added to each well. After 5 minutes, the plates are read by the luminescence reader.

These cross curves make it possible to determine the AC₅₀ values (concentrations at which 50% activation is observed) for the reference ligand at various concentrations of test product. These AC₅₀ values are used to calculate the Schild regression by plotting a straight line corresponding to the Schild equation (“Quantitation in Receptor Pharmacology” Terry P. Kenakin, Receptors and Channels, 2001, 7, 371-385) which leads to Kd app values being obtained (in nM).

### (6) Animal Model

(a) Cognitive Impairment

The compounds described in the present invention may be tested in any animal model known to those skilled in the art. Exemplary animal models include, but are not limited to, transgenic mouse models of cognitive impairment diseases; aged rats; rats with induced damage to the entorhinal cortex; aged rhesus monkeys, and monkeys with entorhinal cortex damage.

For each model, the test result is compared with a control group that is not treated with the compounds described in the present invention. The treated animals are expected to demonstrate significant improvement in the performance of a variety of learning and memory tests (e.g. rat water maze test). For example, it is expected to observe that the brains of the treated animals also exhibit enhanced cell size, improved cell signaling, improved neural connectivity and/or activation of function in neurons that would otherwise have degenerated, compared to untreated animals. These benefits may extend to the degenerating hippocampus where short-term memory is processed, one of the first regions of the brain to suffer damage in disease, as well as the mediobasal hypothalamus, posterior cingulate cortex, parietotemporal areas and mediotemporal lobe.

### D. Pharmaceutical Compositions

According to another aspect of the present invention, pharmaceutical compositions of compounds described herein are provided. In some embodiments, the pharmaceutical compositions further include a pharmaceutically acceptable carrier.

In some embodiments, the pharmaceutical compositions described herein may further include one or more additional therapeutic agents.

In one embodiment, the additional therapeutic agents are used to treat or prevent Alzheimer's disease or other cognitive disorders. Exemplary additional therapeutic agents include, but are not limited to, cholinesterase inhibitors (for example tacrine, galantamine, rivastigamine or donepezil) and NMDA inhibitors (for example memantine). In one embodiment the additional therapeutic agent is one used to regulate treat or prevent disease such as:

Therapeutic agents that regulate beta amyloid plaque disease pathology including; (1) Beta-amyloid peptide synthesis inhibitors including beta secretase and gamma secretase inhibitors (2) Amyloid plaque inhibitors that block beta amyloid peptide aggregation through passive immunization, i.e. with antibodies, or active immunization, i.e. with vaccines, (3) muscarinic receptor modulators, (4) phosphodiesterase-4 inhibitors, and (5) chelating agents. These would include selected from the group consisting of bapineuzumab, solanezumab, gammagaard, MABT5102A, AN-1792, ACC-001, Affitope AD02, CAD-106, MK-8931, HPP854, RG7129, E2609, LY2886721.

Therapeutic agents that regulate tau disease pathology including; (1) tau aggregation inhibitors, (2) tau protease inhibitors, (3) tau kinase inhibitors. Included are Rember, epothilone D.

Therapeutic agents that regulate neurodegeneration pathology including; (1) nicotinic acetylcholine receptor agonists including α4β2 receptor agonists, α7 receptor agonists (2) ion channel modulators (3) 5HT receptor modulators including the group consisting of group consisting of Lu AE58054, EVP-6124, A-582941, GTS-21, AZD3480, MEM 3454, ABT-560, ABT-894.

Therapeutic agents that regulate neuro-inflammation including cox inhibitors and anti-oxidants including agents selected from the group consisting of naproxen, ibuprofen, diclofenac, indomethacin, nabumetone, piroxicam, celecoxib, aspirin.

Therapeutic agents that inhibit neuronal cell death via inhibition of caspases, Par-4, FAS, Bax, Bad, p53; or are neurotrophic factors including BDNF, NGF, bFGF; or activate telomerase, Bcl2, Bcl-X_{L}, Mn-SOD, inhibitor of apoptosis proteins, NCKAP1.

Therapeutic agents that are anti-hypertensives which include angiotensin-converting enzyme (ACE) inhibitors and angiotensin II receptor blockers (ARBs) including from the group consisting of enalapril, ramipril, quinapril, perindopril, lisinopril, benazepril, imidapril, zofenopril, trandolapril, valsartan, telmisartan, losartan, irbesartan, azilsartan, olmesartan.

Therapeutic agents that are an anti-diabetic agent such as the agents selected from the group consisting of insulin, metformin, rosiglitazone, pioglitazone, MSDC-0160, GLP-1 receptor agonists, GLP-1, GLP-1 analogues, DPP-IV inhibitors, sulfonylureas.

Therapeutic agents that are an RXR nuclear receptor agonists and partial agonists, such as bexarotene.

Based on well known assays used to determine the efficacy for treatment of conditions identified above in mammals, and by comparison of these results with the results of known medicaments that are used to treat these conditions, the effective dosage of the compounds of this invention can readily be determined for treatment of each desired indication. The amount of the active ingredient (e.g., compounds) to be administered in the treatment of one of these conditions can vary widely according to such considerations as the particular compound and dosage unit employed, the mode of administration, the period of treatment, the age and sex of the patient treated, and the nature and extent of the condition treated.

The total amount of the active ingredient to be administered may generally range from about 0.0001 mg/kg to about 200 mg/kg, and preferably from about 0.001 mg/kg to about 2 mg/kg body weight per day. A unit dosage may contain from about 0.05 mg to about 150 mg of active ingredient, and may be administered one or more times per day. The daily dosage for administration by injection, including intravenous, intramuscular, subcutaneous, and parenteral injections, and use of infusion techniques may be from about 0.01 to about 20 mg/kg. The daily rectal dosage regimen may be from 0.01 to 20 mg/kg of total body weight. The transdermal concentration may be that required to maintain a daily dose of from 0.01 to 20 mg/kg.

Of course, the specific initial and continuing dosage regimen for each patient will vary according to the nature and severity of the condition as determined by the attending diagnostician, the activity of the specific compound employed, the age of the patient, the diet of the patient, time of administration, route of administration, rate of excretion of the drug, drug combinations, and the like. The desired mode of treatment and number of doses of a compound of the present invention may be ascertained by those skilled in the art using conventional treatment tests.

The compounds of this invention may be utilized to achieve the desired pharmacological effect by administration to a patient in need thereof in an appropriately formulated pharmaceutical composition. A patient, for the purpose of this invention, is a mammal, including a human, in need of treatment for a particular condition or disease. Therefore, the present invention includes pharmaceutical compositions which include a pharmaceutically acceptable carrier and a therapeutically effective amount of a compound. A pharmaceutically acceptable carrier is any carrier which is relatively non-toxic and innocuous to a patient at concentrations consistent with effective activity of the active ingredient so that any side effects ascribable to the carrier do not vitiate the beneficial effects of the active ingredient. A therapeutically effective amount of a compound is that amount which produces a result or exerts an influence on the particular condition being treated. The compounds described herein may be administered with a pharmaceutically-acceptable carrier using any effective conventional dosage unit forms, including, for example, immediate and timed release preparations, orally, parenterally, topically, or the like.

For oral administration, the compounds may be formulated into solid or liquid preparations such as, for example, capsules, pills, tablets, troches, lozenges, melts, powders, solutions, suspensions, or emulsions, and may be prepared according to methods known to the art for the manufacture of pharmaceutical compositions. The solid unit dosage forms may be a capsule which can be of the ordinary hard- or soft-shelled gelatin type containing, for example, surfactants, lubricants, and inert fillers such as lactose, sucrose, calcium phosphate, and corn starch.

In another embodiment, the compounds of this invention may be tableted with conventional tablet bases such as lactose, sucrose, and cornstarch in combination with binders such as acacia, cornstarch, or gelatin; disintegrating agents intended to assist the break-up and dissolution of the tablet following administration such as potato starch, alginic acid, corn starch, and guar gum; lubricants intended to improve the flow of tablet granulation and to prevent the adhesion of tablet material to the surfaces of the tablet dies and punches, for example, talc, stearic acid, or magnesium, calcium or zinc stearate; dyes; coloring agents; and flavoring agents intended to enhance the aesthetic qualities of the tablets and make them more acceptable to the patient. Suitable excipients for use in oral liquid dosage forms include diluents such as water and alcohols, for example, ethanol, benzyl alcohol, and polyethylene alcohols, either with or without the addition of a pharmaceutically acceptable surfactant, suspending agent, or emulsifying agent. Various other materials may be present as coatings or to otherwise modify the physical form of the dosage unit. For instance tablets, pills or capsules may be coated with shellac, sugar or both.

Dispersible powders and granules are suitable for the preparation of an aqueous suspension. They provide the active ingredient in admixture with a dispersing or wetting agent, a suspending agent, and one or more preservatives. Suitable dispersing or wetting agents and suspending agents are exemplified by those already mentioned above. Additional excipients, for example, those sweetening, flavoring and coloring agents described above, may also be present.

The pharmaceutical compositions of this invention may also be in the form of oil-in-water emulsions. The oily phase may be a vegetable oil such as liquid paraffin or a mixture of vegetable oils. Suitable emulsifying agents may be (1) naturally occurring gums such as gum acacia and gum tragacanth, (2) naturally occurring phosphatides such as soy bean and lecithin, (3) esters or partial esters derived from fatty acids and hexitol anhydrides, for example, sorbitan monooleate, and (4) condensation products of said partial esters with ethylene oxide, for example, polyoxyethylene sorbitan monooleate. The emulsions may also contain sweetening and flavoring agents.

Oily suspensions may be formulated by suspending the active ingredient in a vegetable oil such as, for example, arachis oil, olive oil, sesame oil, or coconut oil; or in a mineral oil such as liquid paraffin. The oily suspensions may contain a thickening agent such as, for example, beeswax, hard paraffin, or cetyl alcohol. The suspensions may also contain one or more preservatives, for example, ethyl or n-propyl p-hydroxybenzoate; one or more coloring agents; one or more flavoring agents; and one or more sweetening agents such as sucrose or saccharin.

Syrups and elixirs may be formulated with sweetening agents such as, for example, glycerol, propylene glycol, sorbitol, or sucrose. Such formulations may also contain a demulcent, and preservative, flavoring and coloring agents.

The compounds of this invention may also be administered parenterally, that is, subcutaneously, intravenously, intramuscularly, or interperitoneally, as injectable dosages of the compound in a physiologically acceptable diluent with a pharmaceutical carrier which may be a sterile liquid or mixture of liquids such as water, saline, aqueous dextrose and related sugar solutions; an alcohol such as ethanol, isopropanol, or hexadecyl alcohol; glycols such as propylene glycol or polyethylene glycol; glycerol ketals such as 2,2-dimethyl-1,1-dioxolane-4-methanol, ethers such as poly(ethyleneglycol) 400; an oil; a fatty acid; a fatty acid ester or glyceride; or an acetylated fatty acid glyceride with or without the addition of a pharmaceutically acceptable surfactant such as a soap or a detergent, suspending agent such as pectin, carbomers, methycellulose, hydroxypropylmethylcellulose, or carboxymethylcellulose, or emulsifying agent and other pharmaceutical adjuvants.

Illustrative of oils which can be used in the parenteral formulations of this invention are those of petroleum, animal, vegetable, or synthetic origin, for example, peanut oil, soybean oil, sesame oil, cottonseed oil, corn oil, olive oil, petrolatum, and mineral oil. Suitable fatty acids include oleic acid, stearic acid, and isostearic acid. Suitable fatty acid esters are, for example, ethyl oleate and isopropyl myristate. Suitable soaps include fatty alkali metal, ammonium, and triethanolamine salts and suitable detergents include cationic detergents, for example, dimethyl dialkyl ammonium halides, alkyl pyridinium halides, and alkylamine acetates; anionic detergents, for example, alkyl, aryl, and olefin sulfonates, alkyl, olefin, ether, and monoglyceride sulfates, and sulfosuccinates; nonionic detergents, for example, fatty amine oxides, fatty acid alkanolamides, and polyoxyethylenepolypropylene copolymers; and amphoteric detergents, for example, alkyl-beta-aminopropionates, and 2-alkylimidazoline quarternary ammonium salts, as well as mixtures.

The parenteral compositions of this invention may typically contain from about 0.5% to about 25% by weight of the active ingredient in solution. Preservatives and buffers may also be used advantageously. In order to minimize or eliminate irritation at the site of injection, such compositions may contain a non-ionic surfactant having a hydrophile-lipophile balance (HLB) of from about 12 to about 17. The quantity of surfactant in such formulation ranges from about 5% to about 15% by weight. The surfactant can be a single component having the above HLB or can be a mixture of two or more components having the desired HLB.

Illustrative of surfactants used in parenteral formulations are the class of polyethylene sorbitan fatty acid esters, for example, sorbitan monooleate and the high molecular weight adducts of ethylene oxide with a hydrophobic base, formed by the condensation of propylene oxide with propylene glycol.

The pharmaceutical compositions may be in the form of sterile injectable aqueous suspensions. Such suspensions may be formulated according to known methods using suitable dispersing or wetting agents and suspending agents such as, for example, sodium carboxymethylcellulose, methylcellulose, hydroxypropylmethyl-cellulose, sodium alginate, polyvinylpyrrolidone, gum tragacanth and gum acacia; dispersing or wetting agents which may be a naturally occurring phosphatide such as lecithin, a condensation product of an alkylene oxide with a fatty acid, for example, polyoxyethylene stearate, a condensation product of ethylene oxide with a long chain aliphatic alcohol, for example, heptadecaethyleneoxycetanol, a condensation product of ethylene oxide with a partial ester derived form a fatty acid and a hexitol such as polyoxyethylene sorbitol monooleate, or a condensation product of an ethylene oxide with a partial ester derived from a fatty acid and a hexitol anhydride, for example polyoxyethylene sorbitan monooleate.

The sterile injectable preparation may also be a sterile injectable solution or suspension in a non-toxic parenterally acceptable diluent or solvent. Diluents and solvents that may be employed are, for example, water, Ringer's solution, and isotonic sodium chloride solution. In addition, sterile fixed oils are conventionally employed as solvents or suspending media. For this purpose, any bland, fixed oil may be employed including synthetic mono or diglycerides. In addition, fatty acids such as oleic acid may be used in the preparation of injectables.

A composition of the invention may also be administered in the form of suppositories for rectal administration of the drug. These compositions may be prepared by mixing the drug (e.g., compound) with a suitable non-irritation excipient which is solid at ordinary temperatures but liquid at the rectal temperature and will therefore melt in the rectum to release the drug. Such material are, for example, cocoa butter and polyethylene glycol.

Another formulation employed in the methods of the present invention employs transdermal delivery devices (“patches”). Such transdermal patches may be used to provide continuous or discontinuous infusion of the compounds of the present invention in controlled amounts. The construction and use of transdermal patches for the delivery of pharmaceutical agents is well known in the art (see, e.g., U.S. Pat. No. 5,023,252, incorporated herein by reference). Such patches may be constructed for continuous, pulsatile, or on demand delivery of pharmaceutical agents.

It may be desirable or necessary to introduce the pharmaceutical composition to the patient via a mechanical delivery device. The construction and use of mechanical delivery devices for the delivery of pharmaceutical agents is well known in the art. For example, direct techniques for administering a drug directly to the brain usually involve placement of a drug delivery catheter into the patient's ventricular system to bypass the blood-brain barrier. One such implantable delivery system, used for the transport of agents to specific anatomical regions of the body, is described in U.S. Pat. No. 5,011,472, incorporated herein by reference.

The compositions of the invention may also contain other conventional pharmaceutically acceptable compounding ingredients, generally referred to as carriers or diluents, as necessary or desired. Any of the compositions of this invention may be preserved by the addition of an antioxidant such as ascorbic acid or by other suitable preservatives. Conventional procedures for preparing such compositions in appropriate dosage forms can be utilized.

Commonly used pharmaceutical ingredients which may be used as appropriate to formulate the composition for its intended route of administration include: acidifying agents, for example, but are not limited to, acetic acid, citric acid, fumaric acid, hydrochloric acid, nitric acid; and alkalinizing agents such as, but are not limited to, ammonia solution, ammonium carbonate, diethanolamine, monoethanolamine, potassium hydroxide, sodium borate, sodium carbonate, sodium hydroxide, triethanolamine, trolamine.

Other pharmaceutical ingredients include, for example, but are not limited to, adsorbents (e.g., powdered cellulose and activated charcoal); aerosol propellants (e.g., carbon dioxide, CCl₂F₂, F₂ClC—CClF₂ and CClF₃); air displacement agents (e.g., nitrogen and argon); antifungal preservatives (e.g., benzoic acid, butylparaben, ethylparaben, methylparaben, propylparaben, sodium benzoate); antimicrobial preservatives (e.g., benzalkonium chloride, benzethonium chloride, benzyl alcohol, cetylpyridinium chloride, chlorobutanol, phenol, phenylethyl alcohol, phenylmercuric nitrate and thimerosal); antioxidants (e.g., ascorbic acid, ascorbyl palmitate, butylated hydroxyanisole, butylated hydroxytoluene, hypophosphorus acid, monothioglycerol, propyl gallate, sodium ascorbate, sodium bisulfite, sodium formaldehyde sulfoxylate, sodium metabisulfite); binding materials (e.g., block polymers, natural and synthetic rubber, polyacrylates, polyurethanes, silicones and styrene-butadiene copolymers); buffering agents (e.g., potassium metaphosphate, potassium phosphate monobasic, sodium acetate, sodium citrate anhydrous and sodium citrate dihydrate); carrying agents (e.g., acacia syrup, aromatic syrup, aromatic elixir, cherry syrup, cocoa syrup, orange syrup, syrup, corn oil, mineral oil, peanut oil, sesame oil, bacteriostatic sodium chloride injection and bacteriostatic water for injection); chelating agents (e.g., edetate disodium and edetic acid); colorants (e.g., FD&C Red No. 3, FD&C Red No. 20, FD&C Yellow No. 6, FD&C Blue No. 2, D&C Green No. 5, D&C Orange No. 5, D&C Red No. 8, caramel and ferric oxide red); clarifying agents (e.g., bentonite); emulsifying agents (but are not limited to, acacia, cetomacrogol, cetyl alcohol, glyceryl monostearate, lecithin, sorbitan monooleate, polyethylene 50 stearate); encapsulating agents (e.g., gelatin and cellulose acetate phthalate); flavorants (e.g., anise oil, cinnamon oil, cocoa, menthol, orange oil, peppermint oil and vanillin); humectants (e.g., glycerin, propylene glycol and sorbitol); levigating agents (e.g., mineral oil and glycerin); oils (e.g., arachis oil, mineral oil, olive oil, peanut oil, sesame oil and vegetable oil); ointment bases (e.g., lanolin, hydrophilic ointment, polyethylene glycol ointment, petrolatum, hydrophilic petrolatum, white ointment, yellow ointment, and rose water ointment); penetration enhancers (transdermal delivery) (e.g., monohydroxy or polyhydroxy alcohols, saturated or unsaturated fatty alcohols, saturated or unsaturated fatty esters, saturated or unsaturated dicarboxylic acids, essential oils, phosphatidyl derivatives, cephalin, terpenes, amides, ethers, ketones and ureas); plasticizers (e.g., diethyl phthalate and glycerin); solvents (e.g., alcohol, corn oil, cottonseed oil, glycerin, isopropyl alcohol, mineral oil, oleic acid, peanut oil, purified water, water for injection, sterile water for injection and sterile water for irrigation); stiffening agents (e.g., cetyl alcohol, cetyl esters wax, microcrystalline wax, paraffin, stearyl alcohol, white wax and yellow wax); suppository bases (e.g., cocoa butter and polyethylene glycols (mixtures)); surfactants (e.g., benzalkonium chloride, nonoxynol 10, oxtoxynol 9, polysorbate 80, sodium lauryl sulfate and sorbitan monopalmitate); suspending agents (e.g., agar, bentonite, carbomers, carboxymethylcellulose sodium, hydroxyethyl cellulose, hydroxypropyl cellulose, hydroxypropyl methylcellulose, kaolin, methylcellulose, tragacanth and veegum); sweetening e.g., aspartame, dextrose, glycerin, mannitol, propylene glycol, saccharin sodium, sorbitol and sucrose); tablet anti-adherents (e.g., magnesium stearate and talc); tablet binders (e.g., acacia, alginic acid, carboxymethylcellulose sodium, compressible sugar, ethylcellulose, gelatin, liquid glucose, methylcellulose, povidone and pregelatinized starch); tablet and capsule diluents (e.g., dibasic calcium phosphate, kaolin, lactose, mannitol, microcrystalline cellulose, powdered cellulose, precipitated calcium carbonate, sodium carbonate, sodium phosphate, sorbitol and starch); tablet coating agents (e.g., liquid glucose, hydroxyethyl cellulose, hydroxypropyl cellulose, hydroxypropyl methylcellulose, methylcellulose, ethylcellulose, cellulose acetate phthalate and shellac); tablet direct compression excipients (e.g., dibasic calcium phosphate); tablet disintegrants (e.g., alginic acid, carboxymethylcellulose calcium, microcrystalline cellulose, polacrillin potassium, sodium alginate, sodium starch glycollate and starch); tablet glidants (e.g., colloidal silica, corn starch and talc); tablet lubricants (e.g., calcium stearate, magnesium stearate, mineral oil, stearic acid and zinc stearate); tablet/capsule opaquants (e.g., titanium dioxide); tablet polishing agents (e.g., carnuba wax and white wax); thickening agents (e.g., beeswax, cetyl alcohol and paraffin); tonicity agents (e.g., dextrose and sodium chloride); viscosity increasing agents (e.g., alginic acid, bentonite, carbomers, carboxymethylcellulose sodium, methylcellulose, povidone, sodium alginate and tragacanth); and wetting agents (e.g., heptadecaethylene oxycetanol, lecithins, polyethylene sorbitol monooleate, polyoxyethylene sorbitol monooleate, and polyoxyethylene stearate).

The compounds described herein may be administered as the sole pharmaceutical agent or in combination with one or more other pharmaceutical agents where the combination causes no unacceptable adverse effects. For example, the compounds of this invention can be combined with known anti-obesity, or with known antidiabetic or other indication agents, and the like, as well as with admixtures and combinations thereof.

The compounds described herein may also be utilized, in free base form or in compositions, in research and diagnostics, or as analytical reference standards, and the like. Therefore, the present invention includes compositions which include an inert carrier and an effective amount of a compound identified by the methods described herein, or a salt or ester thereof. An inert carrier is any material which does not interact with the compound to be carried and which lends support, means of conveyance, bulk, traceable material, and the like to the compound to be carried. An effective amount of compound is that amount which produces a result or exerts an influence on the particular procedure being performed.

The compounds may be administered to subjects by any suitable route, including orally (inclusive of administration via the oral cavity), parenterally, by inhalation spray, topically, transdermally, rectally, nasally, sublingually, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. In some embodiments, the compositions are administered orally, parenterally, transdermally or by inhalation spray.

It should also be understood that a specific dosage and treatment regimen for any particular patient will depend upon a variety of factors, including the activity of the specific compound employed, the age, body weight, general health, gender, diet, time of administration, rate of excretion, drug combination, and the judgment of the treating physician and the severity of the particular disease being treated. The amount of a compound of the present invention in the composition will also depend upon the particular compound in the composition.

The following examples are presented to illustrate the invention described herein, but should not be construed as limiting the scope of the invention in any way.

Capsule Formulation

A capsule formula is prepared from:

| | | |
|---|---|---|
| Compound of this invention | 10 | mg |
| Starch | 109 | mg |
| Magnesium stearate | 1 | mg |

The components are blended, passed through an appropriate mesh sieve, and filled into hard gelatin capsules.

Tablet Formulation

A tablet is prepared from:

| | | |
|---|---|---|
| Compound of this invention | 25 | mg |
| Cellulose, microcrystalline | 200 | mg |
| Colloidal silicon dioxide | 10 | mg |
| Stearic acid | 5.0 | mg |

The ingredients are mixed and compressed to form tablets. Appropriate aqueous and non-aqueous coatings may be applied to increase palatability, improve elegance and stability or delay absorption.

Sterile IV Solution

A mg/mL solution of the desired compound of this invention is made using sterile, injectable water, and the pH is adjusted if necessary. The solution is diluted for administration with sterile 5% dextrose and is administered as an IV infusion.

Intramuscular Suspension

The following intramuscular suspension is prepared:

| | | |
|---|---|---|
| Compound of this invention | 50 | μg/mL |
| Sodium carboxymethylcellulose | 5 | mg/mL |
| TWEEN 80 | 4 | mg/mL |
| Sodium chloride | 9 | mg/mL |
| Benzyl alcohol | 9 | mg/mL |

The suspension is administered intramuscularly.

Hard Shell Capsules

A large number of unit capsules are prepared by filling standard two-piece hard galantine capsules each with powdered active ingredient, 150 mg of lactose, 50 mg of cellulose, and 6 mg of magnesium stearate.

Soft Gelatin Capsules

A mixture of active ingredient in a digestible oil such as soybean oil, cottonseed oil, or olive oil is prepared and injected by means of a positive displacement pump into molten gelatin to form soft gelatin capsules containing the active ingredient. The capsules are washed and dried. The active ingredient can be dissolved in a mixture of polyethylene glycol, glycerin and sorbitol to prepare a water miscible medicine mix.

Immediate Release Tablets/Capsules

These are solid oral dosage forms made by conventional and novel processes. These units are taken orally without water for immediate dissolution and delivery of the medication. The active ingredient is mixed in a liquid containing ingredient such as sugar, gelatin, pectin, and sweeteners. These liquids are solidified into solid tablets or caplets by freeze drying and solid state extraction techniques. The drug compounds may be compressed with viscoelastic and thermoelastic sugars and polymers or effervescent components to produce porous matrices intended for immediate release, without the need of water.

### F. Methods of use

### Cognitive Impairment

According to one aspect of the present invention, methods of preventing or treating a cognitive impairment are provided. The methods include administering to a subject in need of such treatment an effective amount of a compound of the present invention. In some embodiments, the compound is administered intravenously, orally, buccally, transdermally, rectally, nasally, optically, intrathecally or intra-cranially.

In another embodiment, the compounds of the present invention may be administered in combination with one or more additional therapeutic agent. Exemplary additional therapeutic agents include, but are not limited to, an anti-diabetic insulin-sensitizing agent, a beta secretase inhibitor, a neurotrophic agent, an acetyl cholinesterase inhibitor, an anti-hypertensive, an amyloid-beta peptide, and an anti-amyloid-beta peptide antibody.

The compounds described herein may be administered in combination with one or more further medicaments of use for the treatment or prevention of a cognitive disorder such as Alzheimer's disease. Further medicaments for the treatment or prevention of Alzheimer's disease include cholinesterase inhibitors (for example tacrine, galantamine, rivastigamine or donepezil) and NMDA inhibitors (for example memantine). The compounds described herein may be administered in combination with one or more further medicaments of use for the treatment or prevention of other cognitive disorders. Other further medicaments include non-steroidal anti-inflammatory drugs (NSAIDs) such as such as naproxen, ibuprofen, diclofenac, indomethacin, nabumetone, piroxicam, celecoxib and aspirin. Other medicaments that may be combined with compounds described herein include HMG-CoA reductase inhibitors such as statins (eg simvastatin (Zocor), atovastatin (Lipitor), rosuvastatin (Crestor), fluvastatin (Lescol)).

Depending on the individual medicaments utilized in a combination therapy for simultaneous administration, they may be formulated in combination (where a stable formulation may be prepared and where desired dosage regimes are compatible) or the medicaments may be formulated separately (for concomitant or separate administration through the same or alternative routes).

In some embodiments, the subject of the present invention possesses one or more risk factors for developing a cognitive disorder selected from a family history of the disease; low cerebral glucose metabolism a genetic predisposition for the disease; elevated serum cholesterol; adult-onset diabetes mellitus; elevated baseline hippocampal volume; elevated or lowered cerebrospinal fluid levels of total tau; elevated cerebrospinal fluid levels of phospho-tau; and lowered cerebrospinal fluid levels of A β(1-42).

### G. Examples

The present invention will now be described in more detail with reference to the following examples. However, these examples are given for the purpose of illustration and are not to be construed as limiting the scope of the invention

### Example 1

### Preparation of methyl 2-(6-methoxy-1H-inden-3-yl)butanoate

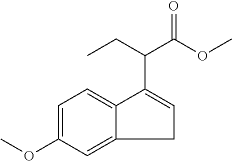

An oven dried 5-L four-necked round-bottomed flask was fitted with a thermometer, a condenser, an addition funnel, and a mechanical stirrer. Under Ar protection, a suspension of 5-methoxy-1-indanone (80.0 g, 494 mmol), Zn powder (Lancaster, 56.2 g, 865 mmol) in 2 L anhydrous THF was stirred at 60° C. (internal temperature), while a solution of methyl bromobutyrate (134.1 g, 741 mmol) in 400 mL anhydrous THF was added slowly through an addition funnel. After completion of the addition, the reaction mixture was stirred at 60° C. (internal temperature) for 1 hour. The reaction was followed by TLC analysis of aliquots following 1N aqueous HCl work-up. After the reaction was completed, it was cooled in an ice-water bath followed by slow addition of 3 L of 1N HCl solution. The pot temperature was kept below 20° C. The mixture was then extracted with 1 L EtOAc. The organic layer was washed with water until pH 6.0-7.0, then saturated NaCl solution, and dried over Na₂SO₄. The product (127 g, >99%), a yellow oil, was obtained after solvent removal and drying under vacuum. ¹H NMR (DMSO-d₆) δ 7.28 (d, 1H), 7.05 (d, 1H), 6.82 (dd, 1H), 6.22 (s, 1H), 3.72 (s, 3H), 3.60 (m, 1H), 3.58 (s, 3H), 3.28 (s, 2H), 1.95 (m, 1H), 1.80 (m, 1H), 0.88 (t, 3H).

### Example 2a

### Preparation of 2-(6-methoxy-1H-inden-3-yl) butanoic Acid

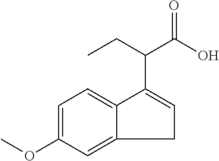 [SMILES: CCC(C(=O)O)C1=CCc2cc(OC)ccc21]

To a solution of the ester prepared in Example 1 (14.0 g, 58.9 mmol) in 140 mL MeOH, was added a solution of KOH (6.4 g, 113.8 mmol) in 5 mL water. The reaction mixture was stirred at 60° C. (pot temperature) for 2 hours. TLC showed 70% conversion. A solution of KOH (3.0 g, 53.6 mmol) in 100 mL water was then slowly added to the pot. After 1 hour, the reaction was completed. After cooling to room temperature, the solvents were removed at a reduced pressure. The residue was dissolved in 500 mL water, and then washed with EtOAc. The aqueous layer was cooled in an ice-water bath, and then acidified with conc. HCl to pH<3.0. The product was extracted into 300 mL CH₂Cl₂, washed with water (2×100 mL), then dried over Na₂SO₄. After Na₂Sa₄ was filtered off, the CH₂Cl₂ solution was stirred with 3.0 g of charcoal for 2 hours. The charcoal was removed by filtration through a pad of Celite®. The title product (12.5 g, 95%) was obtained as a light brown solid after solvent removal and vacuum drying. ¹H NMR (DMSO-d₆) δ 12.20 (b, 1H), 7.30 (d, 1H), 7.06 (d, 1H), 6.82 (dd, 1H), 6.22 (s, 1H), 3.75 (s, 3H), 3.45 (t, 1H), 3.30 (s, 2H), 1.90 (m, 1H), 1.78 (m, 1H), 0.90 (t, 3H).

### Example 2b

### Preparation of 2-(6-methoxy-1H-inden-3-yl) propanoic Acid

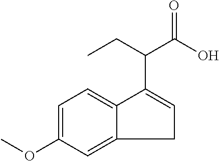 [SMILES: CCC(C(=O)O)C1=CCc2cc(OC)ccc21]

This substrate was prepared using the same procedures as described for Examples 1 and 2a, starting with 5-methoxyl-1-indanone and methyl 2-bromopropionate. Yield: 68%. ¹H NMR (CD₂Cl₂) δ 7.34 (d, J=9, 1H), 7.07 (d, J=2, 1H), 6.85 (dd, J=9, J=2, 1H), 6.32 (m, 1H), 3.82 (m, 4H), 3.36 (m, 2H), 1.56 (d, J=7, 3H).

### Example 3

### Preparation of (2S)-2-(6-methoxy-1H-inden-3-yl)butanoic Acid

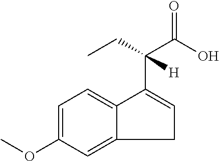

To a solution of the racemic indene acid prepared in Example 2a (300 g, 1.29 mol) in 4.5 L CH₃CN, was added quinine (324 g, 1.0 mol) at rt. The mixture was stirred for 1 hour, and became a solution. A small amount of insoluble particles was removed by filtration through a microfiber filter under vacuum. The filtrate was then mechanically stirred under Ar over night. After 24 hours, a small sample of solid was taken and analyzed, showing 76.2% ee. The agitation was stopped after two more days. The suspension was filtered. The filter cake was washed with CH₃CN (3×200 mL), and then dried under vacuum at 40° C. for 3 hours. This solid was stirred with 4.5 L CH₃CN at 70° C. until all solids went into solution. The solution was allowed to cool down to rt slowly. The resulting suspension was stirred at rt for 24 hours. The suspension was filtered. The filter cake was washed with CH₃CN (3×250 mL), and then dried under vacuum at 40° C. for 24 hours. This quinine salt was collected as a white solid (254.6 g, 35.4% yield, 96.8% ee).

The quinine salt (544.3 g, 0.98 mol) was dissolved in 4.0 L CH₂Cl₂ to obtain a clear solution. It was stirred vigorously with 4.0 L of 2N HCl solution in a 22-L round-bottomed flask with a bottom valve. After 30 minutes, the mixture was allowed to settle. The bottom layer was separated and top aqueous layer was extracted with 1 L CH₂Cl₂. The combined CH₂Cl₂ layers were washed with water (3×2.0 L) until pH 5.0-6.0, and then dried over Na₂SO₄. The product (230.8 g, 99%, 96.8% ee) was obtained as an off white solid after solvent removal and vacuum drying. ¹H NMR was identical to that of the racemic material described in Example 2a.

Treatment of the mother liquor in similar fashion gave the (R) isomer. Alternatively, the mother liquor may be subjected to aqueous basic conditions in order to effect racemization and recovery of racemic starting material.

### Example 4

### Preparation of (2S)-2-[(1S)-5-methoxy-2,3-dihydro-1H-inden-1-yl]butanoic Acid

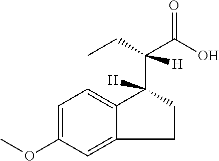 [SMILES: CC[C@@H](C(=O)O)[C@H]1CCc2cc(OC)ccc21]

A solution of the product obtained in Example 3 (105 g, 453 mmol), ClRh(PPh₃)₃ (21.0 g, 5% eq.) and triethylamine (68.8 g, 679.5 mmol) in EtOH (945 mL) and THF (105 mL) was shaken in a 2-L pressure bottle under 60 psi H₂ for 16 hours. The solvents were removed at a reduced pressure. The resulting mixture was stirred in 1.5 L of 1N HCl solution and 1.5 L CH₂Cl₂. The aqueous layer was extracted with CH₂Cl₂ (2×250 mL). The combined CH₂Cl₂ layers were washed with 1 L of 1N HCl solution and stirred with 1 L of 1N NaOH solution. The organic layer was extracted with 1N NaOH solution (2×0.5 L). The combined aqueous layer was washed with CH₂Cl₂ (2×250 mL), and acidified (pH 2.0-3.0) by a slow addition of conc. HCl solution at below 15° C. The acidic mixture was extracted with CH₂Cl₂ (2×1.5 L), and washed with water (2×0.5 L) until pH 5.0-6.0. After washing with brine and drying over anhydrous Na₂SO₄, solvent was evaporated under a reduced pressure. The product (101.0 g, 95% yield, 96.8% ee) was obtained as a light yellow oil. ¹H NMR (DMSO-d₆) δ 12.20 (s, 1H), 7.04 (d, 1H), 6.78 (d, 1H), 6.66 (dd, 1H), 3.70 (s, 3H), 3.28 (m, 1H), 2.72 (m, 2H), 2.32 (m, 1H), 2.06 (m, 1H), 1.80 (m, 1H), 1.50 (m, 1H), 1.36 (m, 1H), 0.82 (t, 3H).

### Example 5a

### Preparation of syn-2-[5-methoxy-2,3-dihydro-1H-inden-1-yl]butanoic Acid

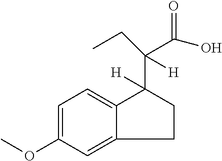 [SMILES: CCC(C(=O)O)C1CCc2cc(OC)ccc21]

A suspension of racemic indene acid (Example 2, 980 mg, 4.2 mmol), ClRh(PPh₃)₃ (139 mg, 0.15 mmol), NaHCO₃ (378 mg, 4.5 mmol) in EtOH (20 mL), and H₂O (10 mL) was shaken in a 500 mL pressure bottle under 60 psi H₂ for 30 hours. Additional ClRh(PPh₃)₃ (300 mg, 0.33 mmol) was added to the reaction mixture and hydrogenation was continued for 3 more days. After this time, EtOH was removed at a reduced pressure and the residue was diluted with 200 mL water. The black solid was removed by filtration and the filtrate was washed with EtOAc (2×200 mL). The aqueous solution was then acidified with conc. HCl, and extracted with CH₂Cl₂ (2×100 mL). The combined CH₂Cl₂ layer was washed with brine and dried over Na₂SO₄. Removal of the solvent in vacuum afforded the indane acid as light yellow oil (600 mg, 60%). The product mixture resulted a diastereomeric mixture (87:13) in favor of the syn isomers as determined by NMR analysis, using the ratio of integration of NMR peaks δ 7.11 (d, 1H) for the anti, and δ 7.03 (d, 1H) for the syn isomers.

Resolution of the product into optical isomers may be accomplished as follows: to a mechanically stirred solution of the syn indane acetic acid [(2R,1R) and (2S,1S), 14.69 g, 62.7 mmol] in acetonitrile (290 mL) at rt, was added (R)-(+)-α-methylbenzylamine (8.49 mL, 65.9 mmol) in one portion. The resulting mixture was stirred overnight. Little solid formation was observed. The reaction mixture was concentrated to dryness and the residue was redissolved in acetonitrile (200 mL) with heating. Magnetic stirring was begun to initiate precipitation. The mixture was stirred overnight. The solids were collected by filtration, and washed three times with a small amount of cold acetonitrile. The solids were then dried under vacuum for 1.5 hours (8.1 g, 86% ee). The slightly wet solids were recrystallized in acetonitrile (120 mL) to give 6.03 g of the (2S)-2-[(1S)-5-methoxy-2,3-dihydro-1H-inden-1-yl]butanoic acid, (R)-α-methylbenzylamine salt (94.4% ee). A second crop was collected from various filtrates (0.89 g, 97.6% ee). The overall yield of resolution was 31% (62% based on the maximum content of (2S,1S) acid in the racemate). The material was identical to that obtained in Example 4.

Optical purity for this Example and that of Example 4 may also be analyzed by chiral HPLC; Column: Chiracel AD, 4.6 (I.D.)×250 mm; Mobile Phase, A: 0.1% TFA (trifluoroacetic acid) in hexanes, B: 0.1% TFA in IPA (isopropyl alcohol); Method, Isocratic 95% A (5% B), 20 min.; Flow Rate, 1.5 mL/min.; Detector (UV), 284 nm. Retention times for the four possible diastereomers are 5.163 min. (2S,1R), 6.255 min. (2R,1S), 10.262 min. (2R,1R) and 14.399 min. (2S,1S). The first locator (2S or 2R) denotes the absolute configuration of the carbon adjacent to the carboxyl group (the 2-positon); the second locator (1S or 1R) denotes the absolute configuration of the indane ring carbon (its 1-position).

The stereochemical assignment for each peak was determined by chiral HPLC analysis of a non-equal (syn/anti) racemic diastereomeric mixture of compound 5, which provided four baseline-resolved peaks. Peaks 3 and 4, and peaks 1 and 2 represented enantiomer pairs, based on UV integration. The absolute configuration of the compound of peak 4 was determined to be 2S,1S by X-ray structural analysis. Peak 3, the corresponding enantiomer, was then assigned a 2R,1R configuration with certainty. Peak 1 was assigned to the (2S,1R)-diastereomer (retention time: 5.363 min., ca. 0.97% area) by comparison to the minor product obtained from the reduction of the chiral acid (Example 3) as described in Example 4. The remaining peak 2, could then be assigned with certainty to the compound with 2R,1S configuration.

### Example 5b

### Preparation of syn-2-[5-methoxy-2,3-dihydro-1H-inden-1-yl]propanoic Acid

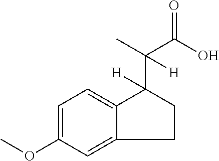 [SMILES: COc1ccc2c(c1)CCC2C(C)C(=O)O]

The compound was prepared in 71% yield and >99% de using the same procedure as described for Example 4 starting with (racemic) Example 2b: ¹H NMR (DMSO-d₆) δ 12.18 (s, 1H), 7.03 (d, J=8, 1H), 6.75 (d, J=2, 1H), 6.67 (dd, J=8, J₂=2, 1H), 3.68 (s, 3H), 3.37 (m, 1H), 2.72 (m, 3H), 2.03 (m, 1H), 1.75 (m, 1H), 0.89 (d, J=7, 3H); ¹³C NMR (CD₂Cl₂) δ 12.626, 28.228, 31.950, 43.300, 46.445, 55.607, 110.054, 112.510, 124.552, 136.702, 146.411, 159.464, 182.330.

### Example 6

### Preparation of methyl (2S)-2-[(1S)-5-methoxy-2,3-dihydro-1H-inden-1-yl]butanoate

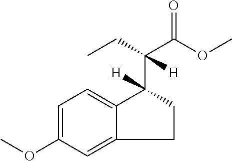

A suspension of acid prepared in Example 4 (220.0 g, 0.94 mol), NaHCO₃ (237.0 g, 2.82 mol), CH₃I (200 g, 1.41 mol) in 2.0 L DMF was stirred under Ar at rt for 18 hours.

NMR analysis showed 95% reaction. Adding CH₃I (100 g), and stirring for additional 24 hours at rt caused completion of the reaction. The reaction mixture was poured into 4.0 L water, and extracted with EtOAc (2×2 L). The organic layer was sequentially washed with water (2×1 L), 1 L of 1N NaOH solution, water (2×1 L), and 500 mL brine, and dried over Na₂SO₄. The product (233 g, 99%) was obtained as a light yellow oil after solvent removal and vacuum drying. ¹H NMR (DMSO-d₆) δ 6.90 (d, 1H), 6.78 (d, 1H), 6.66 (dd, 1H), 3.70 (s, 3H), 3.60 (s, 3H), 3.20 (m, 1H), 2.80 (m, 2H), 2.40 (m, 1H), 2.08 (m, 1H), 1.80 (m, 1H), 1.58 (m, 1H), 1.40 (m, 1H), 0.80 (t, 3H).

### Example 7

### Preparation of methyl (2S)-2-[(1S)-5-hydroxy-2,3-dihydro-1H-inden-1-yl]butanoate

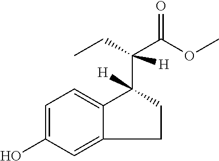

To a cold solution (ice water bath) of the compound prepared in Example 6 (233 g, 0.94 mol) in 2.5 L CH₂Cl₂, was added AlCl₃ (630 g, 4.7 mol) slowly under Ar. The pot temperature was kept below 20° C., and the color of the reaction turned purple. EtSH (345 mL, 4.7 mol) was added slowly via an addition funnel to the reaction mixture, and the inside temperature was kept below 15° C. After 2 hours of stirring at below 20° C., the reaction went to completion by NMR analysis. The pot mixture was slowly poured into 2.5 L ice water with a strong agitation. The organic layer was separated, and the aqueous layer was extracted with 1 L CH₂Cl₂. The combined CH₂Cl₂ layers were washed with water (4×1 L) until pH 6.0-7.0, and then dried over Na₂SO₄. The product (216 g, 98%) was obtained as a white solid after solvent removal and vacuum drying. ¹H NMR (DMSO-d₆) δ 9.10 (s, 1H), 6.78 (d, 1H), 6.58 (d, 1H), 6.50 (dd, 1H), 3.60 (s, 3H), 3.20 (q, 1H), 2.70 (m, 2H), 2.40 (m, 1H), 2.08 (m, 1H), 1.80 (m, 1H), 1.50 (m, 2H), 0.80 (t, 3H).

### Example 8

### Preparation of methyl 3-[(4-methylbenzoyl)amino]-4-oxopentanoate

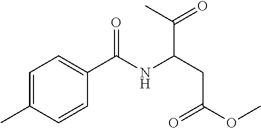

To a suspension of L-aspartic acid -methyl ester hydrochloride (250 g, 1.36 mol) in chilled (<5° C.) CH₂Cl₂ (4 L) was added Et₃N (440 g, 4.35 mol) in a steady flow followed by a slow addition of Me₃SiCl (324 g, 2.99 mol). The mixture was warmed to 25° C. and stirred for one hour, cooled again (<10° C.), and p-toluoyl chloride (205 g, 1.36 mol) was added dropwise. The mixture was allowed to warm to ambient slowly with stirring for 16 hours. The reaction mixture was then diluted with CH₂Cl₂ (500 mL) and washed with 1N HCl (500 mL), brine (500 mL), and dried over Na₂SO₄. The resultant amide product (310 g, 91%), a white solid, was obtained after solvent removal and drying under vacuum. It was then dissolved in pyridine (1.25 L) and DMAP (5 g) was added. Acetic anhydride (840 mL) was added slowly and then the reaction was heated at 90° C. for 2 hours. The cooled solution was poured into 7 L ice water and extracted with 6 L EtOAc. The organic layer was washed with 2N HCl (3×1 L) and 1N NaOH (1 L), dried over MgSO₄ and concentrated to afford the title compound as a white solid (301 g, 93%).

### Example 9

### Preparation of methyl [5-methyl-2-(4-methylphenyl)-1,3-oxazol-4-yl]acetate

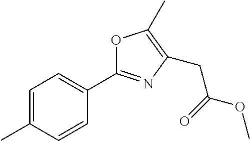

The intermediate prepared in Example 8 (280 g, 1.06 mol) was dissolved in acetic anhydride (650 mL) followed by slow addition of conc. H₂SO₄ (60 mL). The pot temperature reached 80° C. The reaction was then held at 85° C. for 1 hour, cooled, and the acetic anhydride removed in vacuo. The residue was poured into ice water (2 L) and extracted with EtOAc (4 L total). The organic layer was then stirred with 1 N NaOH (500 mL) for 1 hour, separated, then dried with MgSO₄ and concentrated to afford the title ester as a clear oil (223 g, 87%), which slowly solidified to a white solid.

### Example 10

### Preparation of 2-[5-methyl-2-(4-methylphenyl)-1,3-oxazol-4-yl]ethanol

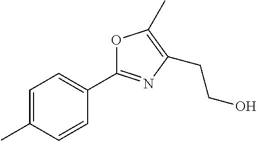 [SMILES: Cc1ccc(-c2nc(CCO)c(C)o2)cc1]

The oxazole ester prepared in Example 9 (300 g, 1.22 mol) was dissolved in THF (2.7 L) and solid LiBH₄ (26.6 g, 1.22 mol) was added in 5-g portions while maintaining temperature below 45° C. Reaction was complete within an hour after addition. Solvent was reduced to half volume and then poured into ice water (3 L). The mixture was then acidified by slowly adding 1 N HCl (1 L). A white precipitate formed and was collected by filtration and oven dried over P₂O₅ to give the desired oxazole ester (214 g, 83%).

### Example 11

### Preparation of methyl (2S)-2-(1S)-5-{2-[5-methyl-2-(4-methylphenyl)-1,3-oxazol-4-yl]ethoxy}-2,3-dihydro-1H-inden-1-yl)butanoate

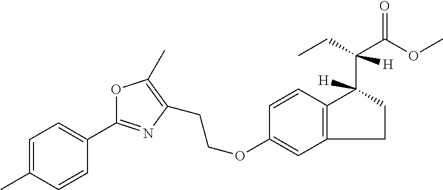

A suspension of the hydroxyindane carboxylate prepared in Example 7 (208 g, 889 mmol), oxazole alcohol prepared in Example 10 (212 g, 977 mmol), ADDP (335 g, 1.33 mol), Ph₃P (348 g, 1.33 mol) in 6.0 L anhydrous THF was stirred at rt under Ar. The reaction was followed by ¹H NMR. No further progress was observed after 2 days. After solids were removed by filtration, THF was removed under reduced pressure. The remaining mixture was stirred in 3 L of 50/50 mixture EtOAc/hexane for 10 minutes, and more solids were formed and removed by filtration. The filtrate was concentrated and subjected to the same procedure with 25/75 mixture of EtOAc/hexane. After solvents were removed, the resulting oily mixture was purified on a silica gel (3.0 kg) column using CH₂Cl₂ (10.0 L) and 20% CH₃CN/CH₂Cl₂ (10.0 L) as solvent. Fractions containing product were collected, and then concentrated. The crude mixture was dissolved in 4.0 L CH₂Cl₂, and the unreacted hydroxy compound was removed by washing with 1N NaOH (3×1 L). The CH₂Cl₂ layer was dried over Na₂SO₄. The product (358 g, 93%) was obtained as a light yellow oil after solvent removal and vacuum drying. ¹H NMR (DMSO-d₆) δ 7.78 (d, 2H), 7.30 (d, 2H), 6.90 (d, 1H), 6.75 (d, 1H), 6.65 (dd, 1H), 4.15 (t, 2H), 3.60 (s, 3H), 3.25 (q, 1H), 2.90 (t, 2H), 2.75 (m, 2H), 2.40 (m, 1H), 2.35 (s, 3H), 2.32 (s, 3H), 2.05 (m, 1H), 1.80 (m, 1H), 1.50 (m, 2H), 0.80 (t, 3H).

### Example 12

### Preparation of (2S)-2-((1S)-5-{2-[5-methyl-2-(4-methylphenyl)-1,3-oxazol-4-yl]ethoxy}-2,3-dihydro-1H-inden-1-yl)butanoic Acid

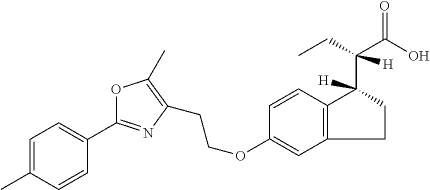 [SMILES: CC[C@@H](C(=O)O)[C@H]1CCc2cc(OCCc3nc(-c4ccc(C)cc4)oc3C)ccc21]

To a solution of LiOH (90.4 g, 3.76 mol) in 1.3 L water and 1.3 L MeOH, was added a solution of the ester prepared in Example 11 (325 g, 0.75 mol) in 3.9 L THF at rt. The solution turned cloudy. This mixture was heated at 60° C. (pot temperature) for 4 hours, and TLC (50% EtOAc/hexane) analysis showed ca. 50% conversion. A solution of LiOH (30.1 g, 1.25 mol) in water (200 mL) was added to the reaction mixture. After 2 hours, TLC analysis showed ca. 85% reaction. Again, a solution of LiOH (30.1 g, 1.25 mol) in water (200 mL) was added to the reaction mixture. After 2 hours, TLC analysis showed very little starting ester left. After the reaction mixture was cooled to rt, THF and MeOH were removed at a reduced pressure. The residue was diluted with water until the solids dissolved (a total of 60 L of water used). Conc. HCl solution was slowly added to this aqueous solution until pH 2.0-3.0. The solid was collected by filtration, and dried under house vacuum overnight. This solid was stirred with 15 L EtOAc and 2 L of 1N HCl solution for 30 minutes. The EtOAc layer was separated and washed with 1N HCl solution (2×1 L). The organic phase was then washed with water (4×2 L) until pH=5.0-6.0. Under Ar protection, the EtOAc solution was reduced to 2.5 L by normal pressure distillation, then cooled to rt without disturbance. White solid precipitated out. After further cooling in an ice water bath for 2 hours, the solid was filtrated and washed with 500 mL cold EtOAc. After drying under high vacuum at 35° C. to a constant weight, the final product (266 g, 81%, 98% ee,) was collected as a white crystal. ¹H NMR (CDCl₃) δ 7.82 (d, 2H), 7.20 (d, 2H), 7.05 (d, 1H), 6.75 (d, 1H), 6.70 (dd, 1H), 4.20 (t, 2H), 3.42 (q, 1H), 2.95 (t, 2H), 2.80 (m, 2H), 2.50 (m, 1H), 2.35 (s, 3H), 2.32 (s, 3H), 2.20 (m, 1H), 1.90 (m, 1H), 1.65 (m, 1H), 1.45 (m, 1H), 0.90 (t, 3H). Chiral purity, 99% ee, [ ]_{D}=+16.11 (CHCl₃), mp 149.5-150.5° C.

### Example 13

### Preparation of 2-{5-[2-(5-methyl-2-phenyl-1,3-oxazol-4-yl)ethoxy]-2,3-dihydro-1H-inden-1-yl}butanoic Acid

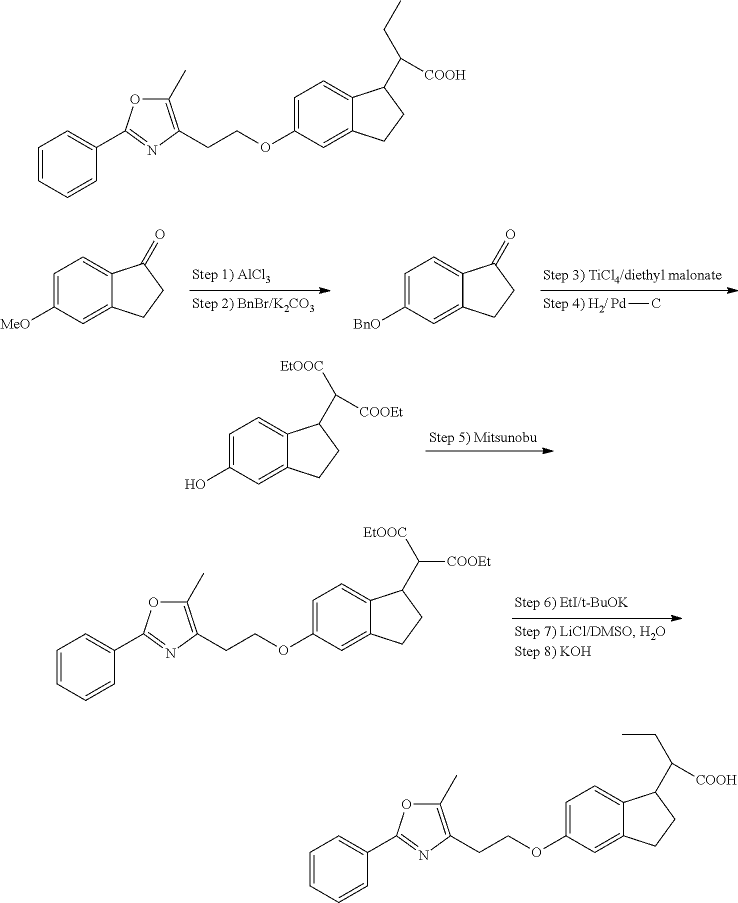

Step 1. To a solution of 5-methoxy-indanone (10 g) dissolved in toluene (150 mL) was added AlCl₃ (15 g). The mixture was refluxed for 4 hours until a precipitate appeared. The resulting mixture was cooled and poured into ice water (150 mL). The precipitate was filtered and washed with water, then air-dried to give the desired product (8.5 g, 90%).

Step 2. Benzyl bromide (17 g), 5-hydroxyl-indanone (15 g), K₂CO₃ (20 g), and 200 mL acetone were mixed in a round-bottom flask (500 mL). The mixture was refluxed for 1 hour. The K₂CO₃ was filtered off, and the filtrate was evaporated. The resulting residue was crystallized from EtOAc to give 18 g product (75%).

Step 3. A solution of 5-benzyloxyl-indanone (1.14 g, 4.79 mmol) and diethyl malonate (0.844 g, 5.29 mmol) in THF (20 mL) was cooled to 0° C. under argon, and TiCl₄ (10 mL, 1M in CH₂Cl₂) was added dropwise. Pyridine (2 mL) was added finally. The resulting mixture was stirred overnight at rt. After filtration, EtOAc (30 mL) was added into the filtrate. The organic layer was washed with brine (20 mL×3), dried with Na₂SO₄, and evaporated. The residue was separated by silica gel chromatography to give 800 mg product (50%).

Step 4, The product of step 3 (1.7 g) was dissolved in MeOH (25 mL), and Pd—C (300 mg) was added as a slurry in MeOH, and placed under 60 psi H₂ in a Parr shaker for 6 hours. After filtration and concentration, 1.2 g product was obtained (92%).

Step 5. P(Ph)₃ (420 mg) and ADDP (420 mg) were dissolved in THF (5 mL) at 0° C., and stirred for 10 minutes. A THF solution of oxazole (300 mg) and phenol (430 mg) was added to the flask. The resulting mixture was stirred for 24 hours, and filtered. The filtrate was evaporated and the resulting residue was separated by silica gel chromatography to give product (320 mg, 45%).

Step 6. The intermediate prepared in step 5 (160 mg) was dissolved in THF (5 mL), and iodoethane (0.5 mL) and t-BuOK (50 mg) were added to the solution and stirred overnight. After filtration, the product was separated by using TLC, providing 100 mg (65%).

Step 7. The intermediate prepared in step 6 (30 mg) was dissolved in DMSO (1 mL). LiCl (160 mg) was added into the flask. The mixture was refluxed for 5 hours. From the resulting mixture, the product was separated by TLC, giving 13 mg (52%).

Step 8. The intermediate prepared in step 7 was subjected to hydrolysis in aqueous KOH as described for Example 2 to obtain the product: LC-MS, RT 3.57 min., M+1 406; ¹H NMR (CD₂Cl₂): δ 0.93 (t, 3H), 1.40-1.70 (m, 2H), 1.80-2.20 (m, 2H), 2.30 (s, 3H), 2.40 (m, 1H), 2.60-2.80 (m, 2H), 2.90 (t, 2H), 3.20-3.40 (m, 1H), 4.10 (t, 2H), 6.60 (dd, 1H), 6.70 (d, 1H), 7.00 (d, 1H), 7.30 (m, 3H), 7.90 (m, 2H).

By using the procedures from Examples 1-13 together in some cases with the chiral HPLC separation method described in the general section, and by using the appropriate starting materials, the following were prepared and characterized in a similar manner:

### Example 14

### 2-(5-{2-[5-methyl-2-(4-methylphenyl)-1,3-oxazol-4-yl]ethoxy}-2,3-dihydro-1H-inden-1-yl)butanoic Acid

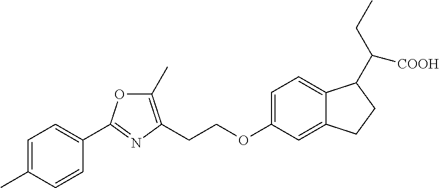 [SMILES: CCC(C(=O)O)C1CCc2cc(OCCc3nc(-c4ccc(C)cc4)oc3C)ccc21]

LC-MS, RT 3.70 min., M+1 420; ¹H NMR (CD₂Cl₂): δ 0.93 (t, 3H), 1.40-1.70 (m, 2H), 1.80-2.20 (m, 2H), 2.30 (s, 3H), 2.35 (s, 3H), 2.40 (m, 1H), 2.60-2.80 (m, 2H), 2.90 (t, 2H), 3.20-3.40 (m, 1H), 4.10 (t, 2H), 6.60 (dd, 1H), 6.70 (d, 1H), 7.00 (d, 1H), 7.20 (m, 3H), 7.80 (m, 2H).

### Example 15

### (2S)-2-{(1S)-5-[2-(5-methyl-2-phenyl-1,3-oxazol-4-yl)ethoxy]-2,3-dihydro-1H-inden-1-yl}butanoic Acid

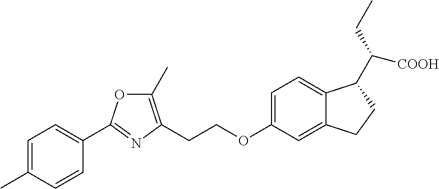 [SMILES: CC[C@H](C(=O)O)[C@@H]1CCc2cc(OCCc3nc(-c4ccc(C)cc4)oc3C)ccc21]

The enantiomer was isolated by chiral HPLC. LC-MS, RT 3.57 min., M+1 406; ¹H NMR (CD₂Cl₂): δ 0.93 (t, 3H), 1.48 (ddq, 1H), 1.68 (ddq, 1H), 1.93 (dddd, 1H), 2.18 (dddd, 1H), 2.34 (s, 3H), 2.50 (ddd, 1H), 2.77 (ddd, 1H), 2.87 (ddd, 1H), 2.96 (t, 2H), 3.42 (ddd, 1H), 4.19 (t, 2H), 6.68 (dd, 1H) 6.77 (d, 1H). 7.08 (d, 1H), 7.42 (m, 2H), 7.44 (m, 1H), 7.97 (dd, 2H). ¹³C NMR: δ 10.4, 12.4, 22.4, 26.6, 29.5, 31.8, 46.5, 51.8, 67.2, 110.9, 113.0, 124.7, 126.2, 128.1, 129.1, 130.2, 133.2, 137.1, 145.6, 146.3, 158.7, 159.7, 180.4.

### Example 16

### (2S)-2-{(1R)-5-[2-(5-methyl-2-phenyl-1,3-oxazol-4-yl)ethoxy]-2,3-dihydro-1H-inden-1-yl}butanoic Acid

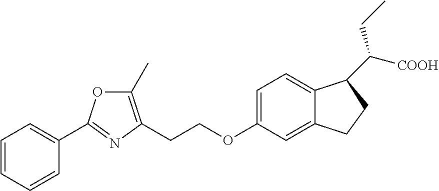 [SMILES: CC[C@H](C(=O)O)[C@H]1CCc2cc(OCCc3nc(-c4ccccc4)oc3C)ccc21]

The enantiomer was isolated by chiral HPLC. LC-MS, RT 3.57 min., M+1 406; ¹H NMR (CD₂Cl₂): δ 0.93 (t, 3H), 1.61 (ddq, 1H), 1.69 (ddq, 1H), 1.99 (dddd, 1H), 2.19 (dddd, 1H), 2.47 (s, 3H), 2.52 (ddd, 1H), 2.73 (ddd, 1H), 2.89 (ddd, 1H), 3.11 (t, 2H), 3.31 (ddd, 1H), 4.21 (t, 2H), 6.66 (dd, 1H) 6.74 (d, 1H). 7.13 (d, 1H), 7.55 (m, 2H), 7.61 (m, 1H), 8.05 (dd, 2H). ¹³C NMR: δ 10.5, 12.2, 23.8, 24.8, 30.3, 31.5, 46.4, 50.9, 66.1, 110.8, 112.6, 125.9, 127.4, 123.6, 129.8, 133.3, 129.7, 137.0, 148.4, 146.5, 158.2, 160.5, 181.0.

### Example 17

### (2R)-2-{(1R)-5-[2-(5-methyl-2-[4-methylphenyl]-1,3-oxazol-4-yl)ethoxy]-2,3-dihydro-1H-inden-1-yl}butanoic Acid

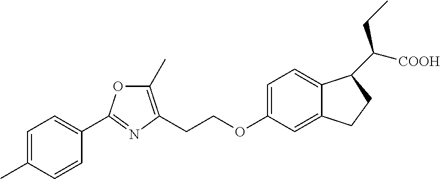 [SMILES: CC[C@@H](C(=O)O)[C@H]1CCc2cc(OCCc3nc(-c4ccc(C)cc4)oc3C)ccc21]

The enantiomer was isolated by chiral HPLC. LC-MS, RT 3.70 min., M+1 420; ¹H NMR (CD₂Cl₂): δ 0.95 (t, 3H), 1.40 (m, 1H), 1.70 (m, 1H), 1.90 (m, 1H), 2.20 (m, 1H), 2.30 (s, 3H), 2.35 (s, 3H), 2.50 (m, 1H), 2.60-2.80 (m, 2H), 2.90 (t, 2H), 3.40 (dd, 1H), 4.20 (t, 2H), 6.60 (dd, 1H), 6.70 (d, 1H), 7.10 (d, 1H), 7.20 (m, 3H), 7.80 (m, 2H).

### Example 18

### 2-(5-{2-[5-methyl-2-phenyl-1,3-oxazol-4-yl]ethoxy}-2,3-dihydro-1H-inden-1-yl)propanoic Acid

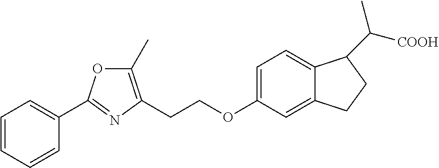 [SMILES: Cc1oc(-c2ccccc2)nc1CCOc1ccc2c(c1)CCC2C(C)C(=O)O]

LC-MS, RT 3.41 min., M+1 392; ¹H NMR (CD₂Cl₂): δ 1.10 (d, 3H), 1.90 (m, 2H), 2.20 (m, 1H), 2.40 (s, 3H), 2.70-3.00 (m, 2H), 2.95 (t, 2H), 3.45 (m, 1H), 4.20 (t, 2H), 6.70 (dd, 1H), 6.80 (d, 1H), 7.10 (d, 1H), 7.45 (m, 3H), 8.00 (m, 2H).

### Example 19

### 2-{5-[2-(5-methyl-2-phenyl-1,3-oxazol-4-yl)ethoxy]-2,3-dihydro-1H-inden-1-yl}malonic Acid

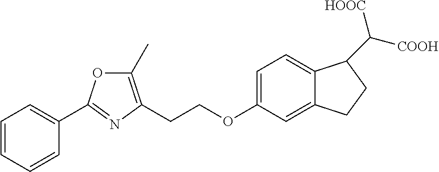 [SMILES: Cc1oc(-c2ccccc2)nc1CCOc1ccc2c(c1)CCC2C(C(=O)O)C(=O)O]

LC-MS, RT 3.00 min., M+1 422; ¹H NMR (CD₂Cl₂): δ 1.90 (m, 2H), 2.40 (t, 3H), 2.60-3.00 (m, 3H), 3.40 (t, 2H), 3.70 (m, 1H), 4.20 (t, 2H), 6.60 (dd, 1H), 6.80 (d, 1H), 7.10 (d, 1H), 7.50 (m, 3H), 7.95 (m, 2H).

### Example 20

### 3-ethoxy-2-{5-[2-(5-methyl-2-phenyl-1,3-oxazol-4-yl)ethoxy]-2,3-dihydro-1H-inden-1-yl}-3-oxopropanoic Acid

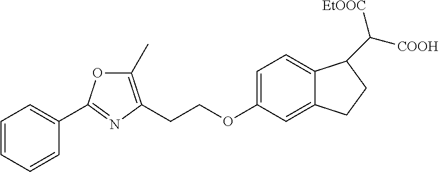 [SMILES: CCOC(=O)C(C(=O)O)C1CCc2cc(OCCc3nc(-c4ccccc4)oc3C)ccc21]

LC-MS, RT 3.39 min., M+1 450; ¹H NMR (CD₂Cl₂): δ 1.20 (t, 3H), 2.00 (m, 1H), 2.30 (m, 1H), 2.40 (s, 3H), 2.90 (m, 2H), 3.10 (t, 2H), 3.80 (m, 1H), 4.20 (t & q, 4H), 6.70 (dd, 1H), 6.80 (d, 1H), 7.10 (d, 1H), 7.50 (m, 3H), 8.00 (m, 2H).

### Example 21

### 2-{5-[2-(5-methyl-2-phenyl-1,3-oxazol-4-yl)ethoxy]-2,3-dihydro-1H-inden-1-yl}-5-phenylpentanoic Acid

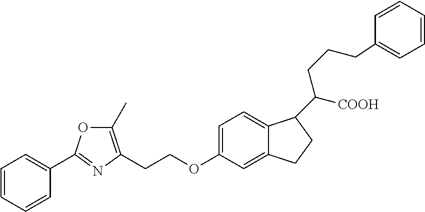 [SMILES: Cc1oc(-c2ccccc2)nc1CCOc1ccc2c(c1)CCC2C(CCCc1ccccc1)C(=O)O]

LC-MS, RT 3.98 min., M+1 396; ¹H NMR (CD₂Cl₂): δ 1.40-1.80 (m, 4H), 1.90-2.20 (m, 2H), 2.35 (s, 3H), 2.40-3.00 (m, 5H), 2.90 (t, 2H), 3.35 (m, 1H), 4.10 (t, 2H), 6.60 (dd, 1H), 6.70 (d, 1H), 6.90-7.20 (m, 6H), 7.30 (m, 3H), 7.95 (m, 2H).

### Example 22

### 2-(5-{2-[5-methyl-2-(4-methylphenyl)-1,3-oxazol-4-yl]ethoxy}-2,3-dihydro-1H-inden-1-yl)propanoic Acid

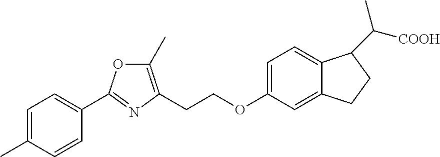 [SMILES: Cc1ccc(-c2nc(CCOc3ccc4c(c3)CCC4C(C)C(=O)O)c(C)o2)cc1]

LC-MS, RT 3.52 min., M+1 406; ¹H NMR (CD₂Cl₂): δ 1.10 (d, 3H), 1.90 (m, 2H), 2.20 (m, 1H), 2.30 (s, 3H), 2.31 (s, 3H), 2.70-3.00 (m, 2H), 2.95 (t, 2H), 3.40 (m, 1H), 4.10 (t, 2H), 6.60 (dd, 1H), 6.70 (d, 1H), 7.00 (d, 1H), 7.20 (d, 2H), 7.80 (d, 2H).

### Example 23

### 2-(5-{2-[5-methyl-2-(4-methylphenyl)-1,3-oxazol-4-yl]ethoxy}-2,3-dihydro-1H-inden-1-yl)hexanoic Acid

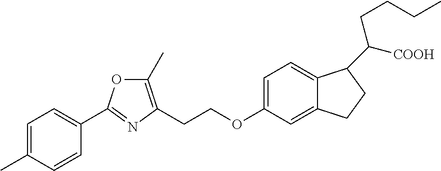 [SMILES: CCCCC(C(=O)O)C1CCc2cc(OCCc3nc(-c4ccc(C)cc4)oc3C)ccc21]

LC-MS, RT 3.92 min., M+1 448; ¹H NMR (CD₂Cl₂): δ 0.93 (t, 3H), 1.10-1.30 (m, 4H), 1.40-1.70 (m, 2H), 1.80-2.20 (m, 2H), 2.30 (s, 3H), 2.31 (s, 3H), 2.40 (m, 1H), 2.60-2.80 (m, 2H), 2.90 (t, 2H), 3.20-3.40 (m, 1H), 4.10 (t, 2H), 6.60 (dd, 1H), 6.70 (d, 1H), 7.00 (d, 1H), 7.20 (d, 2H), 7.80 (d, 2H).

### Example 24

### 4-methyl-2-(5-{2-[5-methyl-2-(4-methylphenyl)-1,3-oxazol-4-yl]ethoxy}-2,3-dihydro-1H-inden-1-yl)pentanoic Acid

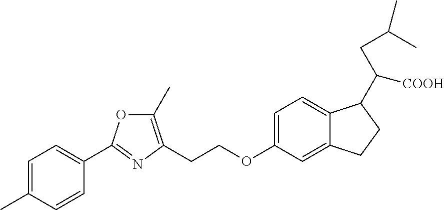 [SMILES: Cc1ccc(-c2nc(CCOc3ccc4c(c3)CCC4C(CC(C)C)C(=O)O)c(C)o2)cc1]

LC-MS, RT 4.00 min., M+1 448; ¹H NMR (CD₂Cl₂): δ 0.93 (m, 6H), 1.20 (m, 1H), 1.40-1.70 (m, 2H), 1.80-2.20 (m, 2H), 2.30 (s, 3H), 2.31 (s, 3H), 2.40 (m, 1H), 2.60-2.80 (m, 2H), 2.90 (t, 2H), 3.20-3.40 (m, 1H), 4.10 (t, 2H), 6.60 (dd, 1H), 6.70 (d, 1H), 7.00 (d, 1H), 7.40 (d, 2H), 8.40 (d, 2H).

### Example 25

### 4-methyl-2-(5-{2-[5-methyl-2-(4-methylphenyl)-1,3-oxazol-4-yl]ethoxy}-2,3-dihydro-1H-inden-1-yl)-4-pentenoic Acid

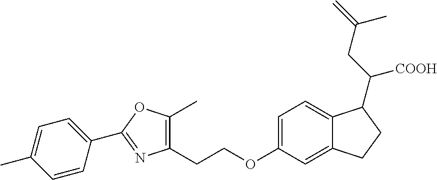 [SMILES: C=C(C)CC(C(=O)O)C1CCc2cc(OCCc3nc(-c4ccc(C)cc4)oc3C)ccc21]

LC-MS, RT 3.74 min., M+1 446; ¹H NMR (CD₂Cl₂): δ 1.60 (s, 3H), 1.70 (m, 2H), 1.80-2.20 (m, 2H), 2.30 (s, 3H), 2.31 (s, 3H), 2.40 (m, 1H), 2.60-2.80 (m, 2H), 2.90 (t, 2H), 3.20-3.40 (m, 1H), 4.10 (t, 2H), 5.60 (m, 2H), 6.60 (dd, 1H), 6.70 (d, 1H), 7.00 (d, 1H), 7.20 (d, 2H), 7.80 (d, 2H).

### Example 26

### Preparation of 2-{6-chloro-5-[2-(5-methyl-2-phenyl-1,3-oxazol-4-yl)ethoxy]-2,3-dihydro-1H-inden-1-yl}butanoic Acid Via 2-(5-methyl-2-phenyl-1,3-oxazol-4-yl)ethyl methanesulfonate and methyl 2-(6-chloro-5-hydroxy-2,3-dihydro-1H-inden-1-yl)butanoate

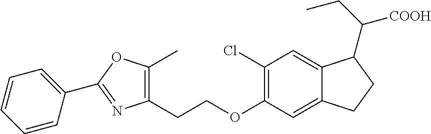 [SMILES: CCC(C(=O)O)C1CCc2cc(OCCc3nc(-c4ccccc4)oc3C)c(Cl)cc21]

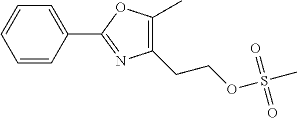 [SMILES: Cc1oc(-c2ccccc2)nc1CCOS(C)(=O)=O]

Step 1. To a solution of 2-phenyl-4-methyl-5-hydroxyethyloxazole (500 mg, 2.5 mmol) in 12.5 mL THF, was added methanesulfonyl chloride (0.21 mL, 2.75 mmol) and triethylamine (0.42 mL, 3 mmol). The reaction solution was stirred at rt under argon for two hours then concentrated in vacuo. The resulting residue was taken up in ethyl acetate, washed with 1% aqueous hydrochloric acid (three times) and brine. It was then dried over sodium sulfate, filtered, and concentrated in vacuo to provide (617 mg, 88%): ES-MS m/z 282 ((M+H)⁺); HPLC RT 2.67; ¹H NMR (d₆-DMSO) δ 2.33 (s, 3H), 2.89 (t, 2H), 3.13 (s, 3H), 4.41 (t, 2H), 7.47-7.51 (m, 3H), 7.88-7.91 (m, 2H).

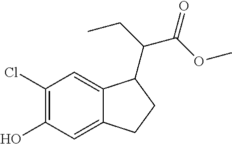 [SMILES: CCC(C(=O)OC)C1CCc2cc(O)c(Cl)cc21]

Step 2. Sulfuryl chloride (0.035 mL, 0.43 mmol) was added to a solution of methyl-5-hydroxy-2,3-dihydro-1-(2-butanoate) (100 mg, 0.43 mmol) in 2.15 mL acetic acid. The reaction solution was stirred at rt for 30 minutes, then concentrated in vacuo. The resulting residue was taken up in ethyl acetate and washed with water, saturated aqueous sodium bicarbonate, and brine. It was then dried over sodium sulfate, filtered, and concentrated in vacuo to provide 63 mg of the desired intermediate as a crude yellow oil which was carried on without further purification: GC-MS 269, ((M+H)⁺); GC RT (min.) 8.71; ¹H NMR (d₆-DMSO) δ 0.81 (t, 3H), 1.40-1.63 (m, 2H), 1.77-1.88 (m, 1H), 2.00-2.15 (m, 1H), 2.40-2.80 (m, 3H), 3.15-3.22 (m, 1H), 3.50 (s, 3H), 6.76 (s, 1H), 7.13 (s, 1H), 9.84 (s, 1H).

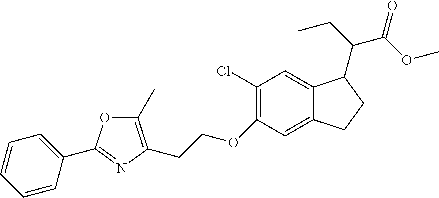 [SMILES: CCC(C(=O)OC)C1CCc2cc(OCCc3nc(-c4ccccc4)oc3C)c(Cl)cc21]

Step 3. A solution of the product obtained in step 2 (30.5 mg, 0.12 mmol) in 0.6 mL DMF was cooled to 0° C. in an ice bath. A 60% dispersion of sodium hydride in oil (5.2 mg, 0.13 mmol) was then added and the ice bath was removed. After stirring the reaction mixture for 1 hour at rt, the mesylate from step 1 (34 mg, 0.12 mmol) was added. The reaction mixture was heated at 50° C. for 24 hours, then cooled to 0° C. An additional 9.6 mg NaH (60% dispersion in oil) was added and heating was resumed for two hours, after which the reaction mixture was cooled to rt and stirred for 48 hours. At this time, ethyl acetate was added and the organic solution was washed with water and brine (three times), dried over sodium sulfate, filtered, and concentrated in vacuo. The resulting residue was purified through silica gel flash chromatography by using 5:1 hexane:ethyl acetate as the eluant to provide product (19 mg, 35%) as a mixture of diastereomers (3:1): ES-MS m/z 454 ((M+H)⁺); HPLC RT (min.) 4.21; ¹H NMR (d₆-DMSO) δ 0.80 (t, 3H), 1.38-1.63 (m, 2H), 1.79-1.90 (m, 1H), 2.02-2.14 (m, 1H), 2.34 (s, 3H), 2.51-2.57 (m, 1H), 2.63-2.84 (m, 2H), 2.91 (t, 2H), 3.19-3.25 (m, 1H), 3.49 (s, 2.3H), 3.58 (s, 0.7H), 4.22 (t, 2H), 7.00 (s, 1H), 7.21 (s, 1H), 7.43-7.51 (m, 3H), 7.85-7.90 (m, 2H).

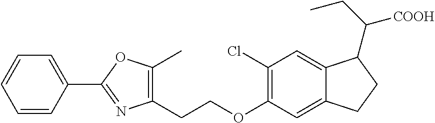 [SMILES: CCC(C(=O)O)C1CCc2cc(OCCc3nc(-c4ccccc4)oc3C)c(Cl)cc21]

Step 4. Under the standard hydrolysis conditions, the ester from step 3 was converted to the acid (a mixture of diastereomers 3:2): ES-MS m/z 440 ((M+H)⁺); HPLC RT (min.) 3.69; ¹H NMR (d₆-DMSO) δ 0.83 (t, 3H), 2.34 (s, 3H), 2.92 (t, 2H), 4.21 (t, 2H), 7.00-7.02 (d, 1H), 7.12 (s, 0.24H), 7.21 (s, 0.37H), 7.47-7.48 (m, 3H), 7.87-7.90 (m, 2H).

### Example 27

### Preparation of ethyl 2-{5-[2-(5-methyl-2-phenyl-1,3-oxazol-4-yl)ethoxy]-2,3-dihydro-1H-inden-1-yl}pentanoate

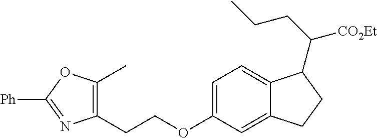 [SMILES: CCCC(C(=O)OCC)C1CCc2cc(OCCc3nc(-c4ccccc4)oc3C)ccc21]

An oven dried 15 mL round-bottom flask and stir bar, cooled under a stream of Ar(g), was charged with ethyl 2-{5-[2-(5-methyl-2-phenyl-1,3-oxazol-4-yl)ethoxy]-2,3-dihydro-1H-inden-1-yl}acetate (0.070 g, 0.17 mmol) followed by addition of 0.2 mL THF. The stirred solution was then cooled to −78° C. followed by dropwise addition of lithium bis(trimethylsilyl)amide (1.0 M hexane solution, 0.86 mL, 0.86 mmol). Upon complete addition of base, the solution was allowed to stir at −78° C. for 1 hour, then iodopropane (0.142 g, 0.86 mmol) was added via syringe. The contents were then slowly warmed to rt and maintained for 1 hour. The contents of the flask were poured into 5 mL NH₄Cl(aq), then extracted with ethyl acetate (3×10 mL). The organic layers were combined and dried over Na₂SO₄ and concentrated in vacuo yielding 3.0 mg (4.0% yield) of a colorless film. The product had: ¹H NMR (300 MHz, d6-acetone) δ 7.96 (dd, 8.1, 1.5 Hz, 2H), 7.48 (m, 3H), 6.99 (d, 8.4 Hz, 1H), 6.79 (d, 2.7 Hz, 1H), 6.70 (dd, 8.1, 2.7 Hz, 1H), 4.22 (t, 6.9 Hz, 2H), 4.11 (q, 7.2 Hz, 2H), 3.33 (q, 6.6 Hz, 1H), 2.94 (t, 6.9 Hz, 2H), 2.78 (m, 3H), 2.54 (m, 1H), 2.39 (s, 3H), 2.14 (m, 2H), 1.91 (m, 1H), 1.63 (qt, 10.2, 3.9 Hz, 2H), 1.21 (t, 7.2 Hz, 3H), 0.852 (t, 7.5 Hz, 3H); mass spectroscopy gave MH⁺ of 448.2 (calc'd molecular weight for C₂₈H₃₃NO₄=447.57).

### Example 28

### Preparation of 2-{5-[2-(5-methyl-2-phenyl-1,3-oxazol-4-yl)ethoxy]-2,3-dihydro-1H-inden-1-yl}pentanoic Acid

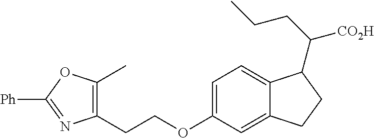 [SMILES: CCCC(C(=O)O)C1CCc2cc(OCCc3nc(-c4ccccc4)oc3C)ccc21]

Hydrolysis of the product of Example 27 by the method described above for Example 2 gave a product with the following ¹H NMR (300 MHz, d6-acetone); δ 7.96 (dd, 8.1, 1.5 Hz, 2H), 7.48 (m, 3H), 7.10 (d, 8.4 Hz, 1H), 6.79 (d, 2.7 Hz, 1H), 6.71 (dd, 8.1, 2.7 Hz, 1H), 4.22 (t, 6.9 Hz, 2H), 3.40 (m, 1H), 2.91 (t, 6.9 Hz, 2H), 2.74 (m, 1H), 2.58 (m, 1H), 2.39 (s, 3H), 2.26 (m, 1H), 2.11 (m, 1H), 1.95 (m, 2H), 1.84 (m, 1H), 1.62 (m, 2H), 0.859 (td, 6.9, 1.5 Hz, 3H); mass spectroscopy gave MH⁺ of 420.1 (calc'd molecular weight for C₂₆H₂₉NO₄=419.51).

### Example 29

### Preparation of 2-{6-bromo-5-[2-(5-methyl-2-phenyl-1,3-oxazol-4-yl)ethoxy]-2,3-dihydro-1H-inden-1-yl}butanoic acid via methyl 2-(6-bromo-5-hydroxy-2,3-dihydro-1H-inden-1-yl)butanoate

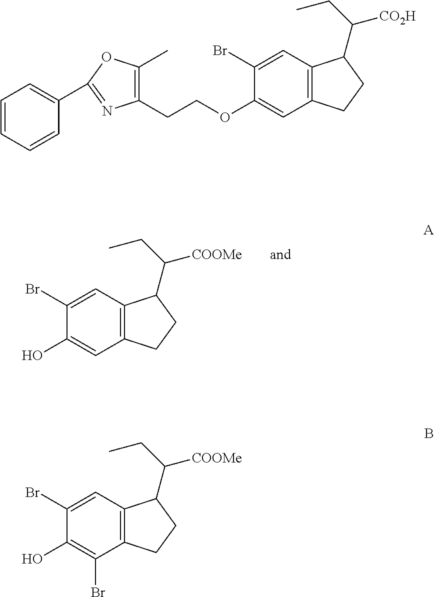 [SMILES: CCC(C(=O)O)C1CCc2cc(OCCc3nc(-c4ccccc4)oc3C)c(Br)cc21 ; CCC(C(=O)OC)C1CCc2cc(O)c(Br)cc21 ; CCC(C(=O)OC)C1CCc2c1cc(Br)c(O)c2Br]

Step 1. A solution of bromine (0.032 mL, 0.60 mmol) in dioxane (3 mL) was cooled to 0° C. for 15 minutes after which a solution of 2-(5-hydroxy-indan-1-yl)-butyric acid methyl ester (141 mg, 0.60 mmol) in dioxane (3 mL) was added. After 5 minutes, the ice bath was removed and the reaction was stirred at rt for 4 hours. Solvent was removed by rotary evaporation. The residue was purified by column chromatography (8% EtOAc in hexane) to obtain a colorless oil of mono-bromo intermediate (A) (145 mg, 77%) and dibromo intermediate (B) (20 mg).

A: R_{f}=0.46 (4:1 hexane:EtOAc); GC-MS (+Cl): m/z=313 (M⁺); ¹H NMR (DMSO-d₆): δ 0.840 (m, 3H), 1.511 (m, 2H), 1.905 (m, 1H), 2.091 (m, 1H), 2.410-2.793 (m, 3H), 3.212 (m, 1H), 3.505 and 3.512 (s, 3H), 6.713 and 6.753 (s, 1H), 7.034 and 7.274 (s, 1H), 9.932 and 9.934 (s, OH).
B: R_{f}=0.30 (4:1 hexane:EtOAc); GC-MS (+Cl): m/z=393 (M⁺); ¹H NMR (DMSO-d₆): δ 0.817 (m, 3H), 1.459-1.596 (m, 2H), 1.910 (m, 1H), 2.101 (m, 1H), 2.433-2.768 (m, 3H), 3.371 (m, 1H), 3.400 and 3.596 (s, 3H), 7.168 and 7.357 (s, 1H), 9.535 and 9.542 (s, OH).

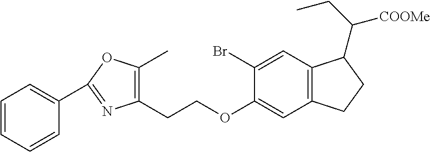 [SMILES: CCC(C(=O)OC)C1CCc2cc(OCCc3nc(-c4ccccc4)oc3C)c(Br)cc21]

Step 2. To a solution of (A) from step 1 above (118 mg, 0.38 mmol) in DMF (3.8 mL) at 0° C., was added NaH (60% in mineral oil, 30 mg). After 1 hour, the mesylate as prepared in step 1, Example 26 was added. The mixture was heated to 50° C. for 30 hours. The solution was diluted with water, and then extracted with ethyl acetate three times. The combined organic layer was washed with water and brine, then dried (Na₂SO₄) and concentrated. The residue was purified by column chromatography (10% ethyl acetate in hexane) to give product (63 mg, 34%); R_{f}=0.46 (2:1 hexane:EtOAc); ESLC-MS: m/z=498 (MH⁺); ¹H NMR (DMSO-d₆): δ 0.847 (m, 3H), 1.468 (m, 2H), 1.812 (m, 1H), 2.146 (m, 1H), 2.340 (s, 3H), 2.525-2.788 (m, 3H), 2.902 (m, 2H), 3.236 (m, 1H), 3.481 and 3.586 (s, 3H), 4.211 (m, 2H), 6.969 (s, 1H), 7.347 and 7.386 (s, 1H), 7.452 (m, 3H), 7.833 (m, 2H).

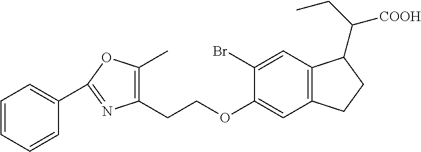 [SMILES: CCC(C(=O)O)C1CCc2cc(OCCc3nc(-c4ccccc4)oc3C)c(Br)cc21]

Step 3. To a solution of product from step 2 (5.6 mg) in methanol, was added 3 N KOH (1 mL) followed by addition of THF until the cloudy solution became clear. The mixture was refluxed overnight. Conc. HCl was added to adjust the pH to 2, then extracted three times with ethyl acetate. The organic layers were combined, dried, and concentrated to give white solid (4 mg). R_{f}=0.18 (2:1 hexane:EtOAc); ESLC-MS: m/z=484 (MH⁺); ¹H NMR (DMSO-d₆): δ 0.832 (m, 3H), 1.468 (m, 2H), 1.812 (m, 1H), 2.146 (m, 1H), 2.405 (m, 1H), 2.788 (m, 2H), 2.904 (m, 2H), 3.015 (m, 1H), 3.136 and 3.138 (s, 3H), 4.209 (m, 2H), 6.987 and 7.344 (s, 1H), 6.972 and 7.251 (s, 1H), 7.487 (m, 3H), 7.882 (m, 2H).

### Example 30

### Preparation of 2-{5-[2-(5-methyl-2-phenyl-1,3-oxazol-4-yl)ethoxy]-6-phenyl-2,3-dihydro-1H-inden-1-yl}butanoic Acid Via methyl 2-{5-[2-(5-methyl-2-phenyl-1,3-oxazol-4-yl)ethoxy]-6-phenyl-2,3-dihydro-1H-inden-1-yl}butanoate

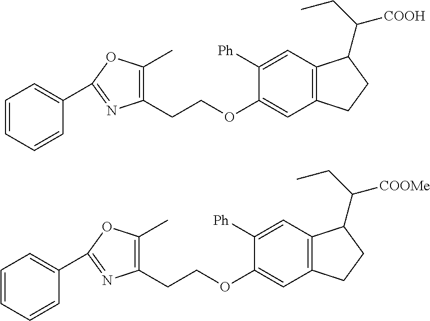 [SMILES: CCC(C(=O)O)C1CCc2cc(OCCc3nc(-c4ccccc4)oc3C)c(-c3ccccc3)cc21 ; CCC(C(=O)OC)C1CCc2cc(OCCc3nc(-c4ccccc4)oc3C)c(-c3ccccc3)cc21]

Step 1. A mixture of the product of step 2, Example 29 and Pd(PPh₃)₄ in THF (1.5 mL) was stirred at rt for 30 minutes. Phenylboronic acid (13.2 mg, 0.108 mmol) and 2 N NaOH were then added into the solution. The mixture was heated to reflux for 14 hours. The solution was allowed to cool down, diluted with water, and extracted with ethyl acetate three times. The combined organic layers were washed with brine and dried over sodium sulfate. The crude product was purified by column chromatography eluting with 5% ethyl acetate in hexane to obtain the desired product (8.6 mg). R_{f}=0.48 (2:1 hexane:EtOAc); ESLC-MS: m/z=496 (MH⁺); ¹H NMR (DMSO-d₆): δ 0.804 (m, 3H), 1.541 (m, 2H), 1.880 (m, 1H), 1.987 (m, 1H), 2.090 (s, 3H), 2.247-2.698 (m, 3H), 2.791 (m, 2H), 3.199 (m, 1H), 3.524 and 3.537 (s, 3H), 4.190 (m, 2H), 6.970 (s, 1H), 7.062 (s, 1H), 7.275 (m, 5H), 7.472 (m, 3H), 7.868 (m, 2H).

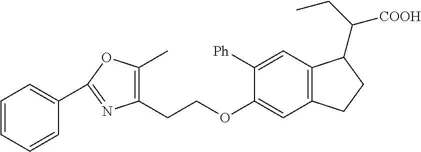 [SMILES: CCC(C(=O)O)C1CCc2cc(OCCc3nc(-c4ccccc4)oc3C)c(-c3ccccc3)cc21]

Step 2. The ester was hydrolyzed by methods described above to give product: R_{f}=0.16 (2:1 hexane:EtOAc); ESLC-MS: m/z=482 (MH⁺); ¹H NMR (DMSO-d₆): δ 0.923 (m, 3H), 1.504 (m, 2H), 1.812 (m, 1H), 2.146 (m, 1H), 2.188 (s, 3H), 2.334 (m, 2H), 2.432 (m, 2H), 2.539 (m, 1H), 2.625 (m, 1H), 4.287 (m, 2H), 7.059 (s, 1H), 7.160 (s, 1H), 7.351 (m, 5H), 7.544 (m, 3H), 7.971 (m, 2H).

### Example 31

### Preparation of methyl 2-{6-(4-chlorophenyl)-5-[2-(5-methyl-2-phenyl-1,3-oxazol-4-yl)ethoxy]-2,3-dihydro-1H-inden-1-yl}butanoate

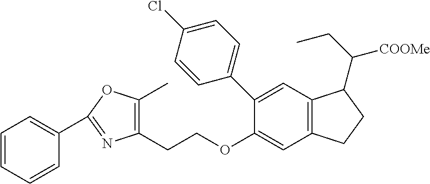 [SMILES: CCC(C(=O)OC)C1CCc2cc(OCCc3nc(-c4ccccc4)oc3C)c(-c3ccc(Cl)cc3)cc21]

A mixture of the product prepared in step 2, Example 29 (71.4 mg, 0.14 mmol), NaHCO₃ (14.3 mg, 0.17 mmol), 4-chlorophenylboronic acid (26.8 mg, 0.17 mmol) in ethylene glycol dimethyl ether (1.5 mL) and water (0.4 mL) was degassed for 20 minutes. Pd(dppf)Cl₂ was then added to the solution. The mixture was heated to reflux for 2 days. The mixture was then concentrated and purified with column chromatography (10% EtOAc in hexane) to obtain desired product (25 mg). R_{f}=0.51 (2:1 hexane:EtOAc); ESLC-MS: m/z=530 (MH⁺); ¹H NMR (DMSO-d₆): δ 0.841 (m, 3H), 1.557 (m, 2H), 1.888 (m, 1H), 1.987 (m, 1H), 2.146 (s, 3H), 2.247-2.698 (m, 3H), 2.791 (m, 2H), 3.214 (m, 1H), 3.487 and 3.5538 (s, 3H), 4.189 (m, 2H), 6.993 (s, 1H), 7.080 (s, 1H), 7.308 (s, 4H), 7.493 (m, 3H), 7.868 (m, 2H).

By using the above described methods for Examples 26-31 and substituting the appropriate starting materials, the following were made and characterized:

### Example 32

### 2-{6-chloro-5-[2-(5-methyl-2-phenyl-1,3-oxazol-4-yl)ethoxy]-2,3-dihydro-1H-inden-1-yl}butanoic Acid

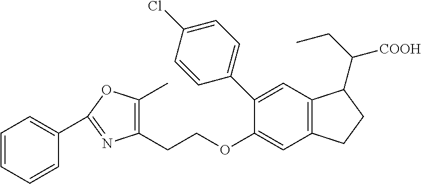 [SMILES: CCC(C(=O)O)C1CCc2cc(OCCc3nc(-c4ccccc4)oc3C)c(-c3ccc(Cl)cc3)cc21]

ESLC-MS: m/z=516 (MH⁺); ¹H NMR (DMSO-d₆): δ 0.847 (m, 3H), 1.557 (m, 2H), 1.888 (m, 1H), 1.987 (m, 1H), 2.137 (s, 3H), 2.247-2.687 (m, 3H), 2.819 (m, 2H), 3.234 (m, 1H), 4.187 (m, 2H), 6.994 (s, 1H), 7.089 (s, 1H), 7.298 and 7.308 (m, 4H), 7.484 (m, 3H), 7.869 (m, 2H).

### Example 33

### Methyl 2-{6-methyl-5-[2-(5-methyl-2-phenyl-1,3-oxazol-4-yl)ethoxy]-2,3-dihydro-1H-inden-1-yl}butanoate

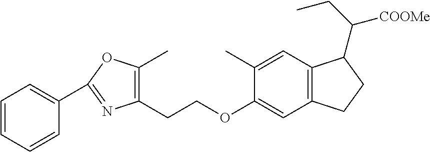 [SMILES: CCC(C(=O)OC)C1CCc2cc(OCCc3nc(-c4ccccc4)oc3C)c(C)cc21]

R_{f}=0.23 (2:1 hexane:EtOAc); ESLC-MS: m/z=434 (MH⁺); ¹H NMR (DMSO-d₆): δ 0.804 (m, 3H), 1.522 (m, 2H), 1.830 (m, 1H), 1.987 (m, 1H), 2.037 (s, 3H), 2.335 (s, 3H), 2.410-2.550 (m, 3H), 2.901 (m, 2H), 3.146 (m, 1H), 3.507 (s, 3H), 4.163 (m, 2H), 6.777 (s, 1H), 6.939 (s, 1H), 7.483 (m, 3H), 7.875 (m, 2H).

### Example 34

### 2-{6-methyl-5-[2-(5-methyl-2-phenyl-1,3-oxazol-4-yl)ethoxy]-2,3-dihydro-1H-inden-1-yl}butanoic Acid

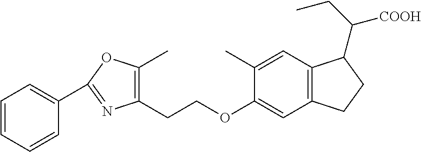 [SMILES: CCC(C(=O)O)C1CCc2cc(OCCc3nc(-c4ccccc4)oc3C)c(C)cc21]

R_{f}=0.31 (2:1 hexane:EtOAc); ESLC-MS: m/z=420 (MH⁺); ¹H NMR (DMSO-d₆): δ 0.827 (m, 3H), 1.508 (m, 2H), 1.828 (m, 1H), 1.987 (m, 1H), 2.017 (s, 3H), 2.333 (s, 3H), 2.410-2.550 (m, 3H), 2.894 (m, 2H), 3.146 (m, 1H), 4.116 (m, 2H), 6.773 (s, 1H), 6.942 (s, 1H), 7.467 (m, 3H), 7.880 (m, 2H).

### Example 35

### Methyl 2-[5-[2-(5-methyl-2-phenyl-1,3-oxazol-4-yl)ethoxy]-6-(2-thienyl)-2,3-dihydro-1H-inden-1-yl]butanoate

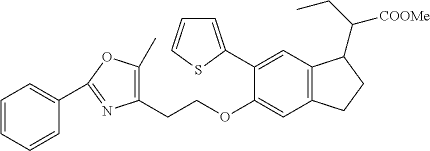 [SMILES: CCC(C(=O)OC)C1CCc2cc(OCCc3nc(-c4ccccc4)oc3C)c(-c3cccs3)cc21]

R_{f}=0.60 (2:1 hexane:EtOAc); ESLC-MS: m/z=502 (MH⁺); ¹H NMR (DMSO-d₆): δ 0.801 (m, 3H), 1.535 (m, 2H), 1.891 (m, 1H), 1.987 (m, 1H), 2.299 (s, 3H), 2.410-2.550 (m, 3H), 2.988 (m, 2H), 3.146 (m, 1H), 3.506 (s, 3H), 4.337 (m, 2H), 7.011-7.041 (m, 2H), 7.405-7.493 (m, 5H), 7.884 (m, 2H).

### Example 36

### 2-[5-[2-(5-methyl-2-phenyl-1,3-oxazol-4-yl)ethoxy]-6-(2-thienyl)-2,3-dihydro-1H-inden-1-yl]butanoic Acid

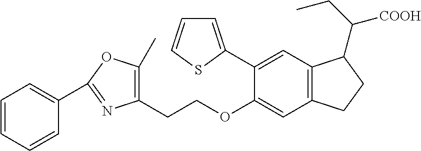 [SMILES: CCC(C(=O)O)C1CCc2cc(OCCc3nc(-c4ccccc4)oc3C)c(-c3cccs3)cc21]

R_{f}=0.18 (2:1 hexane:EtOAc); ESLC-MS: m/z=488 (MH⁺); ¹H NMR (DMSO-d₆): δ 0.801 (m, 3H), 1.535 (m, 2H), 1.891 (m, 1H), 1.987 (m, 1H), 2.299 (s, 3H), 2.410-2.550 (m, 3H), 2.988 (m, 2H), 3.146 (m, 1H), 4.337 (m, 2H), 7.078 (m, 2H), 7.472 (m, 5H), 7.896 (m, 2H).

### Example 37

### Methyl 2-{4,6-dibromo-5-[2-(5-methyl-2-phenyl-1,3-oxazol-4-yl)ethoxy]-2,3-dihydro-1H-inden-1-yl}butanoate

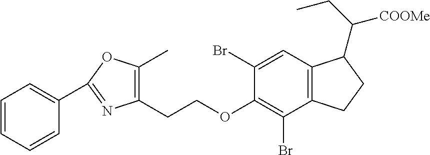 [SMILES: CCC(C(=O)OC)C1CCc2c1cc(Br)c(OCCc1nc(-c3ccccc3)oc1C)c2Br]

R_{f}=0.35 (4:1 hexane:EtOAc); ESLC-MS: m/z=578 (MH⁺); ¹H NMR (DMSO-d₆): δ 0.847 (m, 3H), 1.468 (m, 2H), 1.812 (m, 1H), 2.146 (m, 1H), 2.350 (s, 3H), 2.407-2.788 (m, 3H), 2.982 (m, 2H), 3.225 (m, 1H), 3.480 and 3.588 (s, 3H), 4.145 (m, 2H), 7.276 (s, 1H), 7.458 (m, 3H), 7.866 (m, 2H).

### Example 38

### 2-{4,6-dibromo-5-[2-(5-methyl-2-phenyl-1,3-oxazol-4-yl)ethoxy]-2,3-dihydro-1H-inden-1-yl}butanoic Acid

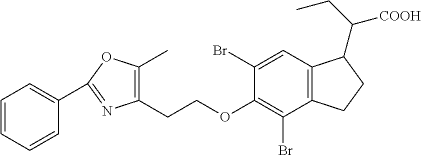 [SMILES: CCC(C(=O)O)C1CCc2c1cc(Br)c(OCCc1nc(-c3ccccc3)oc1C)c2Br]

R_{f}=0.17 (2:1 hexane:EtOAc); ESLC-MS: m/z=564 (MH⁺); ¹H NMR (DMSO-d₆): δ 0.847 (m, 3H), 1.468 (m, 2H), 1.812 (m, 1H), 2.146 (m, 1H), 2.361 (s, 3H), 2.414-2.781 (m, 3H), 2.995 (m, 2H), 3.123 (m, 1H), 4.125 (m, 2H), 7.345 (s, 1H), 7.437 (m, 3H), 7.886 (m, 2H).

### Example 39

### Preparation of 2-{6-acetyl-5-[2-(5-methyl-2-phenyl-1,3-oxazol-4-yl)ethoxy]-2,3-dihydro-1H-inden-1-yl}butanoic Acid Via methyl 2-(6-acetyl-5-methoxy-2,3-dihydro-1H-inden-1-yl)butanoate

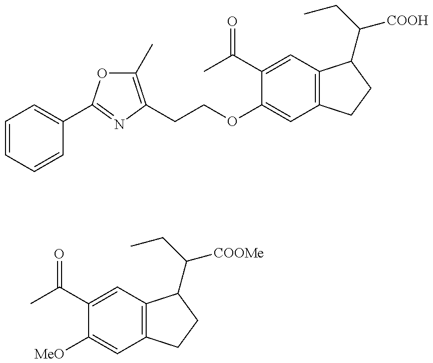 [SMILES: CCC(C(=O)O)C1CCc2cc(OCCc3nc(-c4ccccc4)oc3C)c(C(C)=O)cc21 ; CCC(C(=O)OC)C1CCc2cc(OC)c(C(C)=O)cc21]

Step 1. To a solution of AlCl₃ (103 mg, 0.78 mmol) in methylene chloride (2.5 mL) at 0° C., was added acetyl chloride (0.044 mL, 0.63 mmol), followed by the addition of a solution of methyl 5-methoxy-2,3-dihydro-1H-indene-1-yl-butanoate (130 mg, 0.52 mmol) in methylene chloride (2.7 mL) dropwise. The mixture was stirred at 0° C. for 15 minutes. The ice bath was removed and the mixture stirred at rt for 16 hours. The mixture was poured over ice and 4 drops of conc. HCl were added. This mixture was extracted with methylene chloride twice. The combined organic layers were washed with water, 0.05 N NaOH and water. The organic layer was dried, concentrated, and purified by chromatography with 10% EtOAc:hexane to give desired product (103 mg, 68%). R_{f}=0.28 (4:1 hexane:EtOAc); GC-MS (+Cl): m/z=291 (M⁺); ¹H NMR (DMSO-d₆): δ 0.840 (m, 3H), 1.536 (m, 2H), 1.876 (m, 1H), 2.108 (m, 1H), 2.505 (s, 3H), 2.521 (m, 1H), 2.760-2.889 (m, 2H), 3.236 (m, 1H), 3.511 and 3.589 (s, 3H), 3.836 (s, 3H), 7.012 and 7.253 (s, 1H), 7.440 (s, 1H).

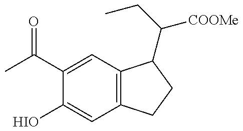

Step 2. To a solution of AlCl₃ (238 mg, 1.77 mmol) in CH₂Cl₂ (1 mL), was added the product of step 1 (103 mg, 0.35 mmol) in CH₂Cl₂ (2 mL). The mixture was cooled to 0° C. for 5 minutes, then EtSH (0.13 mL, 1.77 mmol) was added slowly. The mixture was stirred at this temperature for 4.5 hours. The mixture was then poured over ice water, stirred for 10 minutes, and extracted with CH₂Cl₂ twice. The combined organic layers were washed with water, dried over sodium sulfate, and concentrated to give product (86 mg, 89%). R_{f}=0.51 (4:1 hexane:EtOAc); GC-MS (+Cl): m/z=276 (M⁺); ¹H NMR (DMSO-d₆): δ 0.841 (m, 3H), 1.574 (m, 2H), 1.888 (m, 1H), 2.094 (m, 1H), 2.585 (s, 3H), 2.639 (m, 1H), 2.729-2.847 (m, 2H), 3.244 (m, 1H), 3.513 and 3.628 (s, 3H), 6.774 and 7.503 (s, 1H), 6.792 and 7.715 (s, 1H), 12.117 and 12.143 (s, 1H).

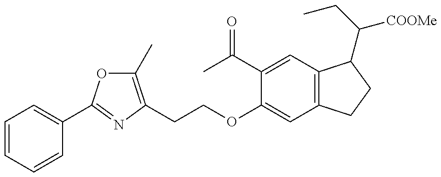 [SMILES: CCC(C(=O)OC)C1CCc2cc(OCCc3nc(-c4ccccc4)oc3C)c(C(C)=O)cc21]

Step 3. The coupling of the hydroxy indene acetic acid ester of step 2 with the mesylate of step 2, Example 26. ESLC-MS: m/z=462 (MH⁺);

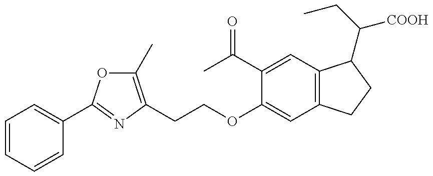 [SMILES: CCC(C(=O)O)C1CCc2cc(OCCc3nc(-c4ccccc4)oc3C)c(C(C)=O)cc21]

Step 4. The hydrolysis of the product from step 3 was carried out in similar fashion as described above to give product: R_{f}=0.08 (2:1 hexane:EtOAc); ESLC-MS: m/z=448 (MH⁺); ¹H NMR (DMSO-d₆): δ 0.848 (m, 3H), 1.468 (m, 2H), 1.812 (m, 1H), 2.146 (m, 1H), 2.305 (s, 3H), 2.368 (s, 3H), 2.405 (m, 1H), 2.788 (m, 2H), 2.971 (m, 2H), 3.015 (m, 1H), 4.332 (m, 2H), 7.039 and 7.441 (s, 1H), 7.446 (s, 1H), 7.465 (m, 3H), 7.875 (m, 2H).

Using a combination of the above described procedures and substituting the appropriate starting materials, a variety of compounds were prepared and are described below.

### Example 40

### Methyl 2-{5-[2-(2,5-diphenyl-1,3-oxazol-4-yl)ethoxy]-2,3-dihydro-1H-inden-1-yl}butanoate

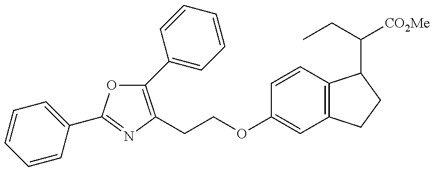 [SMILES: CCC(C(=O)OC)C1CCc2cc(OCCc3nc(-c4ccccc4)oc3-c3ccccc3)ccc21]

Yield: 0.09 g, 46%; ¹H NMR (CDCl₃, 400 MHz) δ 0.83-0.93 (t, 3H), 1.55-1.78 (m, 2H), 1.87-1.97 (m, 1H), 2.10-2.22 (m, 1H), 2.44-2.52 (m, 1H), 2.67-2.80 (m, 1H), 2.81-2.93 (m, 1H), 3.21-3.29 (m, 1H), 3.23-3.33 (t, 2H), 3.62 (s, 3H), 4.34-4.43 (t, 2H), 6.66-6.72 (m, 1H), 6.76 (s, 1H), 7.05-7.14 (d, 1H), 7.33-7.39 (t, 1H), 7.43-7.51 (m, 5H), 7.78-7.84 (d, 2H), 8.06-8.12 (m, 2H).

### Example 41

### 2-{5-[2-(2,5-diphenyl-1,3-oxazol-4-yl)ethoxy]-2,3-dihydro-1H-inden-1-yl}butanoic Acid

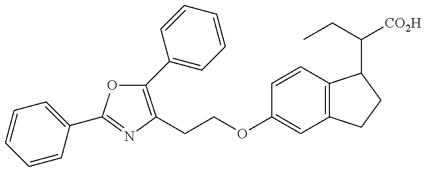 [SMILES: CCC(C(=O)O)C1CCc2cc(OCCc3nc(-c4ccccc4)oc3-c3ccccc3)ccc21]

Yield: 0.07 g, 70%; ¹H NMR (CDCl₃, 400 MHz) δ 0.85-0.98 (m, 3H), 1.23-1.47 (m, 1H), 1.57-1.78 (m, 1H), 1.88-2.07 (m, 1H), 2.12-2.27 (m, 1H), 2.43-2.56 (m, 1H), 2.68-2.97 (m, 2H), 3.27-3.35 (t, 2H), 3.42-3.50 (m, 1H), 4.34-4.41 (t, 2H), 6.66-6.73 (d, 1H), 6.77 (s, 1H), 7.02-7.16 (d, 1H), 7.34-7.40 (t, 1H), 7.43-7.52 (m, 5H), 7.78-7.83 (d, 2H), 8.05-8.12 (m, 2H).

### Example 42

### Methyl 2-{5-[2-(5-isopropyl-2-phenyl-1,3-oxazol-4-yl)ethoxy]-2,3-dihydro-1H-inden-1-yl}butanoate

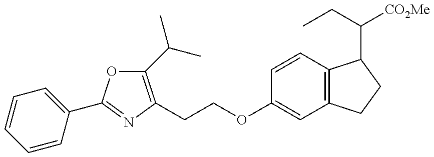 [SMILES: CCC(C(=O)OC)C1CCc2cc(OCCc3nc(-c4ccccc4)oc3C(C)C)ccc21]

Yield: 0.09 g, 45%; ¹H NMR (CDCl₃, 400 MHz) δ 0.78-0.96 (t, 3H), 1.26-1.32 (d, 6H), 1.51-1.62 (m, 1H), 1.64-1.75 (m, 1H), 1.81-1.93 (m, 1H), 2.07-2.21 (m, 1H), 2.40-2.51 (m, 1H), 2.65-2.75 (m, 1H), 2.77-2.98 (m, 1H), 2.91-2.98 (t, 2H), 3.09-3.16 (m, 1H), 3.21-3.28 (m, 1H), 3.62 (s, 3H), 4.10-4.17 (t, 2H), 6.60-6.68 (d, 1H), 6.72 (s, 1H), 7.01-7.13 (d, 1H), 7.33-7.45 (m, 3H), 7.94-8.00 (d, 2H).

### Example 43

### 2-{5-[2-(5-isopropyl-2-phenyl-1,3-oxazol-4-yl)ethoxy]-2,3-dihydro-1H-inden-1-yl}butanoic Acid

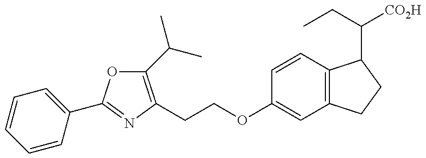 [SMILES: CCC(C(=O)O)C1CCc2cc(OCCc3nc(-c4ccccc4)oc3C(C)C)ccc21]

Yield: 0.08 g, 97%; ¹H NMR (CDCl₃, 400 MHz) δ 0.91-0.98 (t, 3H), 1.30-1.36 (d, 6H), 1.58-1.79 (m, 2H), 1.89-2.05 (m, 1H), 2.12-2.27 (m, 1H), 2.44-2.57 (m, 1H), 2.69-2.80 (m, 1H), 2.83-2.96 (m, 1H), 2.97-3.02 (t, 2H), 3.10-3.21 (m, 1H), 3.24-3.32 (m, 1H), 4.14-4.21 (t, 2H), 6.63-6.71 (d, 1H), 6.75 (s, 1H), 7.04-7.16 (d, 1H), 7.36-7.45 (m, 3H), 7.94-8.00 (d, 2H).

### Example 44

### Methyl 2-{5-[2-(5-ethyl-2-phenyl-1,3-oxazol-4-yl)ethoxy]-2,3-dihydro-1H-indenyl}butanoate

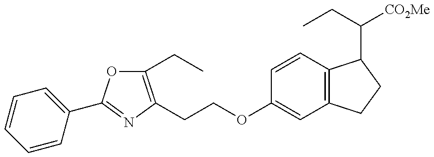 [SMILES: CCc1oc(-c2ccccc2)nc1CCOc1ccc2c(c1)CCC2C(CC)C(=O)OC]

Yield: 0.14 g, 60%; ¹H NMR (CDCl₃, 400 MHz) δ 0.85-0.91 (t, 3H), 1.25-1.35 (t, 3H), 1.58-1.77 (m, 2H), 1.85-1.97 (m, 1H), 2.10-2.22 (m, 1H), 2.44-2.64 (m, 2H), 2.68-2.80 (q, 2H), 2.82-2.93 (m, 1H), 2.95-3.01 (t, 2H), 3.25-3.34 (m, 1H), 3.62 (s, 3H), 4.16-4.25 (t, 2H), 6.66-6.71 (d, 1H), 6.75 (s, 1H), 7.08-7.14 (d, 1H), 7.38-7.46 (m, 3H), 7.95-8.01 (m, 2H).

### Example 45

### 2-{5-[2-(5-ethyl-2-phenyl-1,3-oxazol-4-yl)ethoxy]-2,3-dihydro-1H-inden-1-yl}butanoic Acid

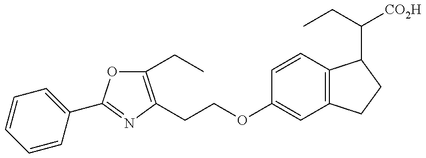 [SMILES: CCc1oc(-c2ccccc2)nc1CCOc1ccc2c(c1)CCC2C(CC)C(=O)O]

Yield: 0.05 g, 60%; ¹H NMR (CDCl₃, 400 MHz) δ 0.85-0.98 (m, 3H), 1.21-1.33 (m, 3H), 1.37-1.54 (m, 1H), 1.56-1.78 (m, 2H), 1.87-2.29 (m, 2H), 2.45-2.60 (m, 1H), 2.69-2.79 (q, 2H), 2.85-2.95 (m, 1H), 2.96-3.01 (t, 2H), 3.27-3.49 (m, 1H), 4.14-4.23 (t, 2H), 6.65-6.71 (d, 1H), 6.75 (s, 1H), 7.03-7.17 (d, 1H), 7.38-7.46 (m, 3H), 7.95-8.01 (d, 2H).

### Example 46

### Methyl 2-{5-[2-(2-phenyl-1,3-oxazol-4-yl)ethoxy]-2,3-dihydro-1H-indenyl}butanoate

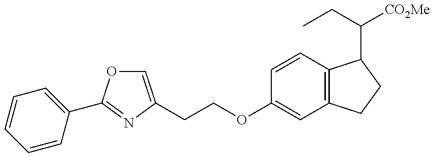 [SMILES: CCC(C(=O)OC)C1CCc2cc(OCCc3coc(-c4ccccc4)n3)ccc21]

Yield: 0.18 g, 80%; ¹H NMR (CDCl₃, 400 MHz) δ 0.82-0.92 (t, 3H), 1.56-1.66 (m, 1H), 1.67-1.77 (m, 1H), 1.88-1.99 (m, 1H), 2.12-2.23 (m, 1H), 2.43-2.52 (m, 1H), 2.68-2.81 (m, 1H), 2.84-2.97 (m, 1H), 3.02-3.11 (t, 2H), 3.25-3.33 (m, 1H), 3.63 (s, 3H), 4.21-4.30 (t, 2H), 6.69-6.74 (d, 1H), 6.79 (s, 1H), 7.11-7.16 (d, 1H), 7.41-7.47 (m, 3H), 7.55-7.58 (m, 1H), 7.99-8.05 (m, 2H).

### Example 47

### 2-{5-[2-(2-phenyl-1,3-oxazol-4-yl)ethoxy]-2,3-dihydro-1H-inden-1-yl}butanoic Acid

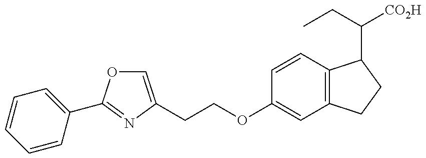 [SMILES: CCC(C(=O)O)C1CCc2cc(OCCc3coc(-c4ccccc4)n3)ccc21]

Yield: 0.07 g, 46%; ¹H NMR (CDCl₃, 400 MHz) δ 0.84-1.01 (m, 3H), 1.36-1.51 (m, 1H), 1.59-1.81 (m, 1H), 1.88-2.00 (m, 1H), 2.11-2.29 (m, 1H), 2.43-2.64 (m, 1H), 2.68-2.81 (m, 1H), 2.82-3.00 (m, 2H), 3.02-3.11 (t, 2H), 3.23-3.37 (m, 1H), 4.17-4.28 (t, 2H), 6.66-6.74 (d, 1H), 6.78 (s, 1H), 7.04-7.19 (m, 1H), 7.39-7.47 (m, 2H), 7.55 (s, 1H), 7.98-8.05 (m, 2H).

### Example 48

### Methyl 2-(5-{2-[2-(2,3-dihydro-1-benzofuran-6-yl)-5-methyl-1,3-oxazol-4-yl]ethoxy}-2,3-dihydro-1H-inden-1-yl)butanoate

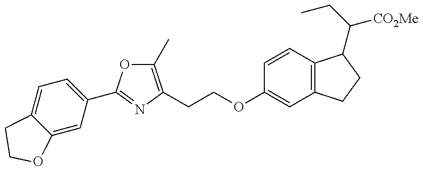 [SMILES: CCC(C(=O)OC)C1CCc2cc(OCCc3nc(-c4ccc5c(c4)OCC5)oc3C)ccc21]

Yield: 0.17 g, 58%; ¹H NMR (CDCl₃, 400 MHz) δ 0.86-0.97 (t, 3H), 1.41-1.53 (m, 1H), 1.61-1.77 (m, 1H), 1.92-2.01 (m, 1H), 2.04-2.20 (m, 1H), 2.40 (s, 3H), 2.49-2.56 (m, 1H), 2.71-2.92 (m, 2H), 3.93-3.00 (t, 2H), 3.21-3.32 (t, 2H), 3.34-3.49 (m, 1H), 3.75 (s, 3H), 4.18-4.24 (t, 2H), 4.54-4.70 (t, 2H), 6.70-6.76 (d, 1H), 6.79 (s, 1H), 6.82-6.89 (d, 1H), 6.92-7.01 (d, 1H), 7.75-7.80 (d, 1H), 7.87 (s, 1H).

### Example 49

### 2-(5-{2-[2-(2,3-dihydro-1-benzofuran-6-yl)-5-methyl-1,3-oxazol-4-yl]ethoxy}-2,3-dihydro-1H-inden-1-yl)butanoic Acid

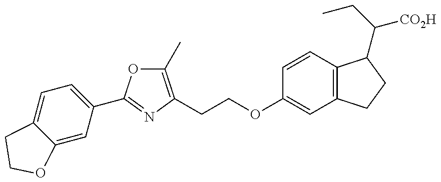 [SMILES: CCC(C(=O)O)C1CCc2cc(OCCc3nc(-c4ccc5c(c4)OCC5)oc3C)ccc21]

Yield: 0.10 g, 99%; ¹H NMR (CDCl₃, 400 MHz) δ 0.90-1.04 (t, 3H), 1.41-1.54 (m, 1H), 1.60-1.76 (m, 1H), 1.83-1.97 (m, 1H), 2.12-2.23 (m, 1H), 2.35 (s, 3H), 2.48-2.60 (m, 1H), 2.69-2.90 (m, 2H), 2.92-3.01 (t, 2H), 3.18-3.28 (t, 2H), 3.39-3.50 (m, 1H), 4.08-4.12 (t, 2H), 4.46-4.64 (t, 2H), 6.76-6.71 (d, 1H), 6.73 (s, 1H), 6.77-6.84 (d, 1H), 7.01-7.09 (d, 1H), 7.71-7.78 (d, 1H), 7.83 (s, 1H).

### Example 50

### Preparation of ethoxy{5-[2-(5-methyl-2-phenyl-1,3-oxazol-4-yl)ethoxy]-2,3-dihydro-1H-inden-1-yl}acetic Acid Via ethyl [5-(benzyloxy)-2,3-dihydro-1H-inden-1-ylidene](ethoxy)ethanoate

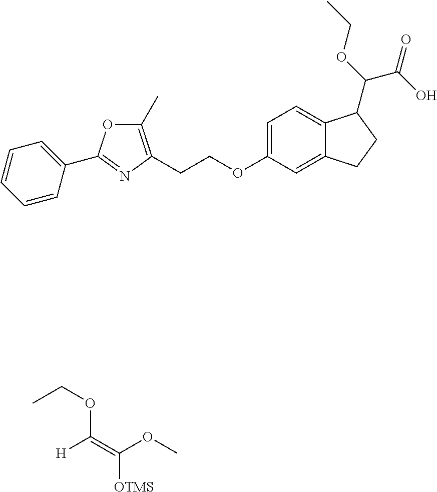

Step 1. LDA (prepared from 11 mmol DIA and 11 mmole BuLi) was added to methyl 2-ethoxyacetate (10 mmol) in 50 mL THF at −78° C., stirred for 1 hour, then TMSCl (30 mmol) was added. The mixture was concentrated in vacuo, and was carried to the next step directly without purification.

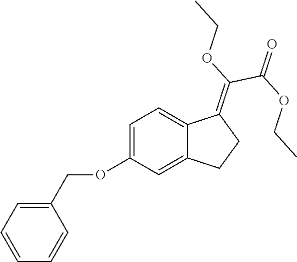 [SMILES: CCOC(=O)/C(OCC)=C1\CCc2cc(OCc3ccccc3)ccc21]

Step 2. 5-Benzyloxy-1-indanone in CH₂Cl₂ (5 mL) was slowly added to TiClₐ in CH₂Cl₂ (10 mL) at −78° C., stirred at −60° C. for 10 minutes, and cooled to −78° C. The product of step 1 in CH₂Cl₂ (5 mL) was slowly added and stirred for 10 minutes. The reaction was quenched with saturated K₂CO₃, filtered, extracted with ethyl acetate, and dried over sodium sulfate. Column chromatography yielded a colorless oil as product. LC-MSMH⁺=353.1, RT=4.00 min.; NMR (CDCl₃, 400 MHz) δ 7.9 (1H, d), 7.25 (5H, m), 6.78 (2H, m), 4.93 (2H, s), 4.15 (2H, q), 3.75 (2H, q), 3.05 (2H, m), 2.85 (2H, m), 1.22 (6H, m)

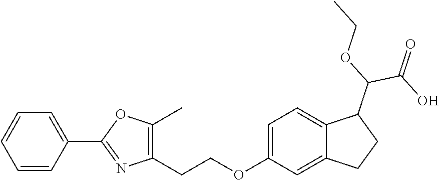 [SMILES: CCOC(C(=O)O)C1CCc2cc(OCCc3nc(-c4ccccc4)oc3C)ccc21]

Step 3. Using the product of step 2 as starting material and procedures similar to that described for Example 13, steps 4-8, the desired final product was prepared and characterized: LC-MS [MH⁺]=422.2, RT=3.25 min.; NMR (CDCl₃, 400 MHz) δ 8.26 (1H, d), 7.55 (2H, m), 7.16 (2H, d), 6.70 (3H, m), 4.16 (2H, q), 3.63 (2H, t) 3.5 (2H, m), 3.30 (1H, m), 3.20 (1H, m), 2.50 (3H, s), 1.10 (3H, m).

### Example 51

### Preparation of 2-{5-[2-(5-methyl-2-phenyl-1,3-oxazol-4-yl)ethoxy]-2,3-dihydro-1H-inden-1-yl}butanoic Acid Via 2-(4-methyl-2-phenyl-1,3-oxazol-5-yl)ethanol

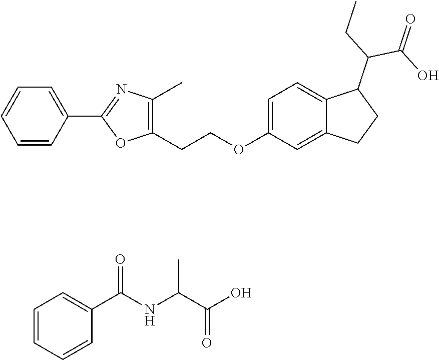 [SMILES: CCC(C(=O)O)C1CCc2cc(OCCc3oc(-c4ccccc4)nc3C)ccc21 ; CC(NC(=O)c1ccccc1)C(=O)O]

Step 1. To a solution of sodium hydroxide (8.98 g, 224.49 mmol) in water (112.25 mL), was added at rt DL-Alanine (10 g, 112.25 mmol). The resulting solution was heated at 75° C. and the benzoyl chloride (15.77 g, 112.25 mmol) was slowly added. The reaction was heated for 30 minutes, and cooled down to 0° C. with an ice bath. Conc. HCl was added to adjust the pH to 1, then the white solid was filtrated through a fritted glass funnel and vacuum dried with P₂O₅ overnight. No purification was needed. This gave N-benzoylalanine (19.6 g, 90.4% yield) as white solid. ¹H NMR (DMSO-d₆) δ 12.61 (s br, 1H), 8.64 (d, 1H), 7.87-7.85 (m, 2H), 7.52-7.43 (m, 3H), 4.40 (q, 1H), 1.39 (d, 3H).

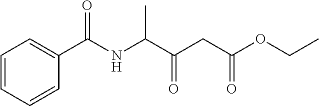 [SMILES: CCOC(=O)CC(=O)C(C)NC(=O)c1ccccc1]

Step 2. In the first flask, N-benzoylalanine (2 g, 10.35 mmol) was dissolved in THF (20 mL), and carbonyl diimidazole (CDI) (1.84 g, 11.39 mmol) was added. The resulting mixture was stirred 1 hour at rt and cooled down to −78° C. Into a second flask, ethyl acetate (3.83 g, 43.48 mmol) in THF (40 mL) was cooled down to −78° C. and LDA (24.3 mL, 48.51 mmol, 2 M in THF) pre-cooled to −78° C. was added. The resulting solution was stirred 30 minutes at −78° C., and the lithium enolate generated was cannulated into the first flask. The resulting white slurry was stirred 30 minutes at −78° C. and warmed up to −10° C. The reaction was quenched with a saturated aqueous solution of NH₄Cl. Phases were separated and the organics were dried over MgSO₄ and solvents removed under reduced pressure. The crude product was carried to the next step without purification. This gave ethyl 4-(benzoylamino)-3-oxopentanoate (2.6 g, 95.5% yield) as a white solid. ES-MS m/z 263.4 ((MH)⁺); HPLC RT (min.) 1.53; ¹H NMR (Acetone-d₆) δ 8.13 (s br, 1H), 7.93-7.91 (m, 2H), 7.58-7.43 (m, 3H), 4.72 (m, 1H), 4.19-4.01 (q, 2H), 3.67 (s, 2H), 1.47 (d, 3H), 1.15 (t, 3H).

Step 3. To a crude mixture of ethyl 4-(benzoylamino)-3-oxopentanoate (0.6 g, 2.28 mmol) in DMF (4 mL) at rt, was added POCl₃ (1.04 g, 6.84 mmol) The resulting solution was heated at 90° C. for 1 hour, then cooled down to rt, and poured into ice for 30 minutes. The aqueous solution was carefully added to a saturated aqueous solution of NaHCO₃. Phases were separated with EtOAc and the combined organic extracts were dried over MgSO4 and solvent removed under reduced pressure. The crude material was purified on Biotage small column using a solvent gradient of 0 to 50% EtOAc/Hexane. This gave ethyl (4-methyl-2-phenyl-1,3-oxazol-5-yl)acetate (0.269 g 48% yield) as yellowish oil. ES-MS m/z 246.2 ((MH)⁺); HPLC RT (min.) 2.77; ¹H NMR (CDCl₃) δ 8.01-7.98 (m, 2H), 7.45-7.41 (m, 3H), 4.20 (q, 2H), 3.71 (s, 2H), 2.21 (s, 3H), 1.28 (t, 3H).

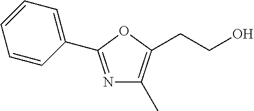

Step 4. Ethyl (4-methyl-2-phenyl-1,3-oxazol-5-yl)acetate (0.922 g, 3.76 mmol) in THF (6 mL) at rt, was added LiBH₄ 2M/THF (9.41 mL, 4.70 mmol). The reaction was stirred overnight at rt, then treated with 2 N HCl until pH 7. The solvent THF was removed under reduced pressure, EtOAc was added, and phases separated. The combined organic extracts were dried over MgSO₄ and solvent concentrated in vacuo. The crude material was purified by Biotage using a gradient of 10 to 100% EtOAc/Hexane as solvent mixture. This gave 2-(4-methyl-2-phenyl-1,3-oxazol-5-yl)ethanol (0.193 g, 25% yield) as colorless oil. ES-MS m/z 204.2 (MH)⁺); HPLC RT (min.) 2.02; ¹H NMR (Acetone-d₆) δ 7.98-7.95 (m, 2H), 7.52-7.42 (m, 3H), 3.95 (s br, 1H), 3.82 (t, 2H)ₘ, 2.90 (t, 2H), 2.13 (s, 3H).

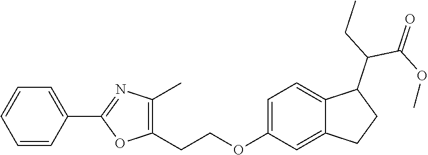 [SMILES: CCC(C(=O)OC)C1CCc2cc(OCCc3oc(-c4ccccc4)nc3C)ccc21]

Step 5. DEAD (0.84 mL, 5.28 mmol) in THF (1.5 mL) was slowly added to a solution of the product of step 3 (4.95 mmol), methyl 5-hydroxy-2,3-dihydro-inden-1-yl-2-butanoate (0.78 g, 3.3 mmol), PPh₃ (1.4 g, 5.28 mmol) in THF (13 mL). The mixture was stirred at rt overnight. The mixture was filtered, washed with water, brine, dried over sodium sulfate, and concentrated. Column chromatography yielded a colorless oil as product. LC-MS [C₂₆H₂₉NO₄H]⁺=420.4, RT=4.00 min.; ¹H NMR (CDCl₃): δ 7.9 (2H, d), 7.45 (2H, dd), 7.1 (d), 6.6-6.8 (3H, m), 4.2 (2H, t), 3.62 (3H, s), 3.3 (1H, m), 3.15 (2H, t), 2.6-3.0 (2H, m, br), 2.5 (1H, m), 2.21 (3H, s), 1.95 (1H, m), 1.56-1.6 (3H, br, m), 0.88 (3H, t).

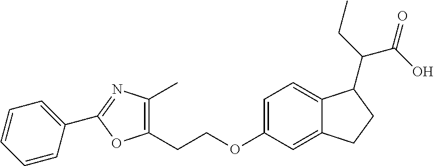 [SMILES: CCC(C(=O)O)C1CCc2cc(OCCc3oc(-c4ccccc4)nc3C)ccc21]

Step 6. KOH (0.5 mL, 3 N) was added to a solution of the product of step 4 (42 mg, 0.1 mmol) in THF/MeOH (1 mL, THF:MeOH 8:2). The mixture was stirred at 70° C. for 6 hours, then cooled down. The pH was adjusted to 4 with 1 N HCl. The mixture was extracted with ethyl acetate (3×2 mL). The combined organic layers were dried over sodium sulfate and concentrated in vacuo. Column chromatography (2:8/hexane:ethyl acetate) gave a white solid as the product (33 mg, 81%). LC-MS [C₂₅H₂₇Na₄H]⁺=406.3, RT=3.37 min.; ¹H NMR (CDCl₃): δ 8.0 (2H, d), 7.45 (2H, dd), 7.15 (1H, d), 6.7-6.8 (3H, m), 4.2 (2H, t), 3.3 (1H, m), 3.15 (2H, t), 2.6-3.0 (2H, m, br), 2.5 (1H, m), 2.21 (3H, s), 1.95 (1H, m), 1.56-1.6 (3H, br, m), 0.88 (3H, t)

By using the procedure described above for Example 51 and substituting the appropriate starting materials, the following were similarly prepared and characterized.

### Example 52

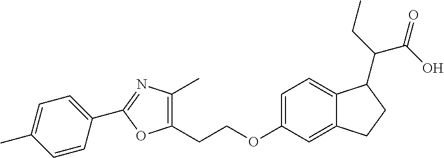 [SMILES: CCC(C(=O)O)C1CCc2cc(OCCc3oc(-c4ccc(C)cc4)nc3C)ccc21]

LC-MS [C₂₆H₂₉Na₄H]⁺=420.3, RT=3.52 min.; ¹H NMR (CDCl₃): δ 7.87 (2H, d), 7.25 (2H, dd), 7.1 (1H, d), 6.6-6.8 (3H, m), 4.2 (2H, t), 3.45 (1H, m), 3.30 (1H, m), 3.15 (2H, t), 2.7-3.0 (2H, m, br), 2.5 (1H, m), 2.4 (3H, s) 1.95 (1H, m), 1.56-1.60 (3H, br, m), 0.88 (3H, t)

### Example 53

### 2-{5-[2-(4-methyl-2-propyl-1,3-oxazol-5-yl)ethoxy]-2,3-dihydro-1H-inden-1-yl}butanoic Acid

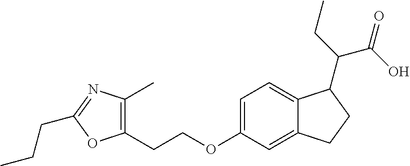 [SMILES: CCCc1nc(C)c(CCOc2ccc3c(c2)CCC3C(CC)C(=O)O)o1]

LC-MS [C₂₂H₂₉Na₄H]⁺=372.3, RT=3.16 min.; ¹H NMR (CDCl₃): δ 7.1 (1H, d), 6.6 (2H, d), 4.2 (2H, t), 3.3 (1H, m), 3.3 (1H, m), 2.8 (2H, t), 2.7 (1H, m), 2.6 (2H, t), 2.4 (2H, m), 2.2 (3H, s), 2.0-1.8 (2H, br, m), 0.88 (3H, t)

By using the methods described above for Examples 1-53 and by substituting the appropriate starting materials, compounds of Formula Ia, listed in Table 3 below, were similarly prepared.

TABLE 3
Preparative Examples of Compounds of Formula (Ia)
(Ia)

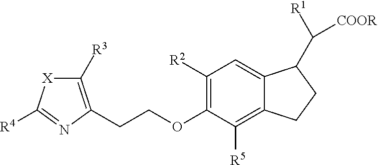

| | | | | | | | | LC-MS |
|---|---|---|---|---|---|---|---|---|
| Ex. | | | | | | | | [M + H]⁺ |
| No. | R | R¹ | R² | R³ | R⁴ | R⁵ | X | or NMR |
| 54 | H | Et | H | Me | PhOCH₂— | H | O | 436.2 |
| 55 | H | Et | H | Me | PhCH₂— | H | O | 420 |
| 56 | H | H | H | Me | Ph | H | O | 378.2 |
| 57 | Me | Ph(CH₂)₃— | H | Me | Ph | H | O | 3.45/3.52 (t, |
| | | | | | | | | 3H), 4.10 (t, |
| | | | | | | | | 2H), 7.3 (m, |
| | | | | | | | | 3H), 7.83 |
| | | | | | | | | (d, 2H) |
| 58 | Et | EtO₂C— | H | Me | Ph | H | O | 478.2 |
| 59 | Et | Et | H | Me | Ph | H | O | 434.3 |
| 60 | H | MeO | H | Me | Ph | H | O | 3.30 (s, 3H), |
| | | | | | | | | 4.04 (d, 1H), |
| | | | | | | | | 7.98 (m, 2H) |
| 61 | Et | EtO | H | Me | Ph | H | O | 450.3 |
| 62 | H | CF₃CH₂— | H | Me | Ph | H | O | 2.51 (s, 3H), |
| | | | | | | | | 4.36 (m, 2H), |
| | | | | | | | | 8.32 (m, 2H) |
| 63 | Et | CF₃CH₂— | H | Me | Ph | H | O | 1.18 (t, 3H), |
| | | | | | | | | 4.21 (t, 2H), |
| | | | | | | | | 7.98 (d, 2H) |
| 64 | Me | cyc-Pr | H | Me | Ph | H | O | 432.3 |
| 65 | H | cyc-Pr | H | Me | Ph | H | O | 0.02 (m, 1H), |
| | | | | | | | | 0.12 (m, 1H), |
| | | | | | | | | 4.18 (m, 2H), |
| | | | | | | | | 7.94 (m, 2H) |
| 66 | H | 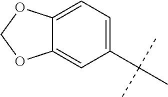 | H | Me | Ph | H | O | 512.3 |
| 67 | H | Et | H | Me | Ph | H | S | 422.3 |
| 68 | H | 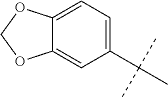 | H | Me | Ph | H | O | 526.4 |
| 69 | H | Et | H | Me | Ph | H | S | 422.3 |
| 70 | 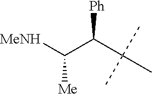 | Et | H | Me | Ph | H | S | |
| 71 | Me | Et | H | Me | Ph | H | O | 0.82 (t, 3H), |
| | | | | | | | | 3.54 (s, 3H), |
| | | | | | | | | 4.16 (t, 2H), |
| | | | | | | | | 7.90 (m, 2H) |
| 72 | H | Et | H | i-Pr | Ph | H | O | 434.3 |
| 73 | H | Et | H | Ph | Ph | H | O | 468.3 |
| 74 | H | Me | H | Me | Ph | H | S | 422.3 |
| 75 | Me | Me | H | Me | Ph | H | S | |
| 76 | Me | Et | MeC(O)— | Me | Ph | H | O | 462.4 |
| 77 | Me | Et | 4-MeO—Ph | Me | Ph | H | O | 526.4 |
| 78 | H | Et | 4-MeO—Ph | Me | Ph | H | O | 512.3 |
| 79 | Me | Et | 4-pyridyl | Me | Ph | H | O | 497.3 |
| 80 | H | Et | H | Me | cyc-Pentyl | H | O | 398 |
| 81 | H | Et | H | Me | cyc-Hexyl | H | O | 412 |
| 82 | H | Et | H | Me | 4-Ph—Ph— | H | O | 482 |
| 83 | Et | EtO₂C— | H | Me | 4-Me—Ph— | H | O | 492.3 |
| 84 | H | PhCH₂— | H | Me | 4-Me—Ph— | H | O | 482.4 |
| 85 | Et | n-Bu | H | Me | 4-Me—Ph— | H | O | 476.3 |
| 86 | Et | Me | H | Me | 4-Me—Ph— | H | O | 434.3 |
| 87 | Et | PhCH₂— | H | Me | 4-Me—Ph— | H | O | 510.4 |
| 88 | H | Et | H | Me | 4-MeO—Ph | H | O | 436.1 |
| 89 | H | Et | H | Me | 4-i-Pr—Ph | H | O | 448.2 |
| 90 | H | Et | H | Me | 4-F—PhCH₂— | H | O | 438.3 |
| 91 | H | Et | H | Me | 4-F—Ph | H | O | 424.3 |
| 92 | H | Et | H | Me | 4-Et—Ph | H | O | 434.3 |
| 93 | H | Et | H | Me | 4-Cl—PhOCH₂— | H | O | 470.2 |
| 94 | H | Et | H | Me | 4-Cl—Ph | H | 0 | 440 |
| 95 | Me | Et | H | Me | 4-Cl—Ph | H | S | 470.3 |
| 96 | Me | Et | H | Me | 4-Cl—Ph | H | S | 470.3 |
| 97 | H | Et | H | Me | 4-CF₃—Ph | H | S | 490.3 |
| 98 | Me | Et | H | Me | 4-CF₃—Ph | H | S | 504.3 |
| 99 | H | Et | H | Me | 4-CF₃—Ph | H | O | 474.3 |
| 100 | H | Et | H | Me | 4-(n-Bu)—Ph | H | O | 462.3 |
| 101 | H | Et | H | Me | 4-(t-Bu)—Ph | H | O | 462.3 |
| 102 | H | Et | H | Me | 3-Me—Ph | H | O | 420.4 |
| 103 | H | Et | H | Me | 3-MeO—Ph | H | O | 436.3 |
| 104 | H | Et | H | Me | 3-Me-5-isoxazolyl | H | O | 411.3 |
| 105 | H | Et | H | Me | 3-F—Ph | H | O | 424.2 |
| 106 | H | Et | H | Me | 3-F-4-Me—Ph | H | O | 438.2 |
| 107 | H | Et | H | Me | 4-F-3-Me—Ph | H | O | 438.3 |
| 108 | Me | Et | H | Me | 3-Cl—Ph | H | S | 470.3 |
| 109 | H | Et | H | Me | 3-Cl—Ph | H | O | 440.3 |
| 110 | H | Et | H | Me | 3-Cl—Ph | H | S | 456.3 |
| 111 | H | Et | H | Me | 3-CF₃—Ph | H | O | 474.2 |
| 112 | H | Et | H | Me | 3,5-(CF₃)₂—Ph | H | O | 542.1 |
| 113 | H | Et | H | Me | 3,4-Me₂—Ph | H | O | 434.3 |
| 114 | H | Et | H | Me | 3,4-Cl₂—Ph | H | O | 474.2 |
| 115 | H | Et | H | Me | 2,3-Cl₂—Ph | H | O | 474.1 |
| 116 | H | Et | H | Me | 3,4-(MeO)₂—Ph | H | O | 466.3 |
| 117 | H | Et | H | Me | 3,4-methylenedioxy-Ph | H | O | 466.3 |
| 118 | H | Et | H | Me | 2-thienyl | H | O | 412 |
| 119 | H | Et | H | Me | 2-naphthyl | H | O | 456.3 |
| 120 | H | Et | H | Me | 2-Me—Ph | H | O | 420.3 |
| 121 | H | Et | H | Me | 2-furyl | H | O | 396 |
| 122 | H | Et | H | Me | 2-F—Ph | H | O | 424.1 |
| 123 | H | Et | H | Me | 2-benzothienyl | H | O | 462.2 |
| 124 | H | Et | H | Me | 2,6-F₂—Ph | H | O | 442.2 |
| 125 | H | Et | H | Me | 3,4-F₂—Ph | H | O | 442.2 |
| 126 | H | Et | H | Me | 2,4-Cl₂—Ph | H | O | 473 |
| 127 | H | Et | H | Me | 1-naphthyl | H | O | 456.3 |
| 128 | Me | Et | H | Me | 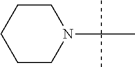 | H | O | 0.90 (t, 3H), 3.45 (bs, 4H), 3.74 (s, 3H) |

### Example 129

### Preparation of ethyl (5-methoxy-2,3-dihydro-1H-inden-1-ylidene)ethanoate

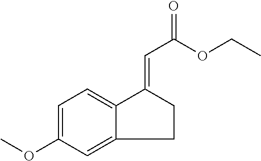 [SMILES: CCOC(=O)/C=C1\CCc2cc(OC)ccc21]

To a solution of 5-methoxyindanone (150 g, 0.91 mol) in anhydrous tetrahydrofuran (4.5 L), was added zinc (30 mesh, 103.64 g, 1.59 mol) and copper(I) chloride (4.53 g, 0.045 mol). The suspension was stirred under Ar atmosphere and refluxed for 15 minutes; approximately a 25% portion of ethyl bromoacetate (133 mL, 1.18 mol) was added to the refluxing mixture in a slow dropwise fashion. After allowing to cool and stirring overnight at rt, TLC showed the presence of desired product, indicating the formation of reactive zinc species. The remainder of ethyl bromoacetate was added dropwise; an exotherm was observed (internal temperature increased to 35° C.). After 4 hours, TLC showed complete reaction. After the solids settled to the bottom of the flask, the liquid was siphoned off leaving a small amount behind to cover the solids. The flask was re-charged with 5-methoxyindanone (157.6 g, 1.86 mol total), anhydrous tetrahydrofuran (4.5 L), and zinc (80.92 g, 2.73 mol total). Ethyl bromoacetate (140 mL, 2.36 mol total) was added dropwise. An exotherm was observed (internal temperature increased to 35° C.). When the stirred mixture cooled to rt, TLC showed the reaction to be complete. The solids were allowed to settle and the liquid was siphoned off. The combined reaction solutions were concentrated in vacuo to a volume of ˜2 L. The liquid was then poured into sufficient 1N aqueous hydrochloric acid (cooled in ice water) to bring the pH to 1. The product was extracted with ethyl acetate (2×1 L, 1×500 mL). The combined extracts were washed with water, brine (1 L each), dried over sodium sulfate, filtered, and concentrated in vacuo to afford a dark red oil which solidified gradually (438.3 g; theoretical yield=432 g). ¹H NMR (CDCl₃): δ 7.5 (d, 1H), 6.8 (m, 2H), 6.2 (t, 1H), 4.2 (q, 2H), 3.8 (s, 3H), 3.3 (m, 2H), 3.0 (t, 2H), 1.3 (t, 3H). MS (Cl) m/z 233 [M+H]⁺.

### Example 130

### Preparation of ethyl (5-methoxy-2,3-dihydro-1H-inden-1-yl)acetate

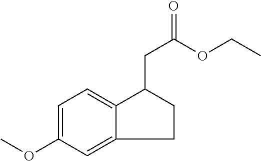 [SMILES: CCOC(=O)CC1CCc2cc(OC)ccc21]

The crude product of Example 129 was dissolved in absolute ethanol (2.6 L) and hydrogenated at 40 psi of hydrogen over 10% palladium on carbon (21.6 g). Filtration through Celite and concentration of the filtrate afforded 433.3 g of brown oil (99% yield for 2 steps). ¹H NMR (CDCl₃): δ 7.1 (dd, 1H), 6.8 (d, 1H), 6.7 (dd, 1H), 4.2 (q, 2H), 3.8 (s, 3H), 3.5 (m, 1H), 2.9 (m, 2H), 2.7 (dd, 1H), 2.4 (m, 2H), 1.7 (m, 1H), 1.3 (t, 3H). MS (Cl) m/z 235 [M+H]⁺.

### Example 131

### Preparation of (5-methoxy-2,3-dihydro-1H-inden-1-yl)acetic Acid

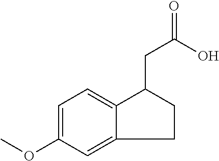

To a solution of the crude ester (416 g, 1.77 mol) prepared in Example 130 in 1 L EtOH, was added a solution of NaOH (142 g, 3.54 mol) in 1.5 L water. The cloudy reaction mixture was heated to reflux, during which time the color changed to dark red, and the reaction became homogeneous. After 1 hour, the reaction was cooled to rt, and the EtOH was removed under reduced pressure. The basic aqueous layer was washed with Et₂O (3×500 mL), then acidified with conc. HCl to pH ˜4 upon which an oil residue formed. The mixture was extracted with Et₂O (4×500 mL). The combined extracts were washed with water (2×300 mL), brine, then dried over Na₂SO₄. Filtration and evaporation of solvent under reduced pressure gave the title compound (305 g, 83%) as a yellow solid after overnight drying under vacuum. ¹H NMR (CDCl₃) δ 7.34 (d, 1H), 6.71 (s, 1H), 6.65 (dd, 1H), 3.71 (s, 3H), 3.47 (m, 1H), 2.80 (m, 3H), 2.35 (m, 2H), 1.71 (m, 1H). MS (Cl) m/z 207 [M+H]⁺.

### Example 132

### Preparation of [(1S)-5-methoxy-2,3-dihydro-1H-inden-1-yl]acetic Acid

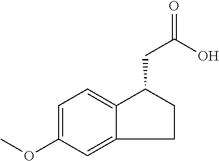

To a solution of the acid (341.0 g, 1.65 mol) prepared in Example 131 in 8.2 L reagent grade acetone, was added (S)-(−)-α-methylbenzylamine (223.8 mL, 1.74 mol) dropwise at rt with stirring. A thick white precipitate formed during the addition. An additional 500 mL acetone was added and stirring continued for 1 hour. The solids were collected by filtration, washed with 300 mL acetone, and dried under suction. The solids were then suspended in acetone (8.2 L) and warmed to reflux until all solids dissolved. The solution was cooled slowly overnight, during which time a white precipitate formed. The suspension was cooled to 0° C., then filtered, and the solids were washed with 500 mL acetone. After drying under suction, a sample analyzed by HPLC showed 95% ee. The recrystallization process was repeated as above using 6.7 L acetone. HPLC analysis showed 99% ee. After drying under suction, 192 g salt were obtained. The salt was suspended in 2 L EtOAc and 1 L of 1 N HCl solution, and shaken in a separatory funnel, whereupon the salt dissolved. The organic layer was separated, washed with 1 N HCl (500 mL), water (2×300 mL), and brine, then dried over Na₂SO₄. The solvent was evaporated under reduced pressure, giving an oil which soon solidified. The title product (120.5 g, 35%) was obtained as an off-white solid after vacuum drying. ¹H NMR (CDCl₃) δ 7.10 (d, 1H), 6.79 (d, 1H), 6.73 (dd, 1H), 3.79 (s, 3H), 3.55 (m, 1H), 2.89 (m, 2H), 2.79 (dd, 1H), 2.46 (dd, 1H), 2.43 (m, 1H), 1.80 (m, 1H). MS (ESI) m/z 207 [M+H]⁺.

### Example 133

### Preparation of [(1S)-5-methoxy-2,3-dihydro-1H-inden-1-yl]acetic Acid

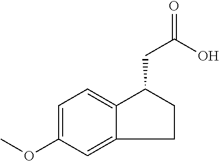

As an alternative to Example 132, the title compound may also be prepared via an enzymatic process. Thus, a cloudy mixture of the crude ester (500.0 g, 2.13 mol; 87% pure as determined by HPLC) prepared in Example 130, in 1 L reagent grade acetone, 2.5 L Phosphate Buffer (pH 7.0, 0.05 M) and 2.5 L deionized water was treated in one portion with Amano Lipase PS (150 g), and the mixture stirred efficiently at rt overnight. HPLC analysis of an aliquot (homogeneous aliquot prepared by dissolving aliquot in IPA followed by filtration) showed one peak corresponding to unreacted R-ester and another peak corresponding to desired S-acid. Trace amounts of S-ester and R-acid were noted. 2 N HCl (500 mL, ensure a pH ˜2) was added in one portion to the reaction and stirred for 20 minutes. The mixture was filtered and the solids were washed with EtOAc (2×500 mL), then water (500 mL). The combined filtrates were further diluted with 1 L EtOAc, and the layers stirred together vigorously. Stirring was stopped and the layers allowed to separate. Emulsions were noted, but could be broken with the addition of solid NaCl and stirring. The aqueous layer was removed, then extracted with EtOAc (3×1 L) in the same fashion. The combined organic extractions were washed with water (4×500 mL), then with brine. The resulting organic layer was extracted with a 5% Na₂CO₃ solution (8×500 mL). HPLC analysis of the organic layer showed that it contained none of the S-enantiomer acid. The combined Na₂CO₃ extracts were washed with EtOAc (2×1 L), then acidified to pH ˜2 by the addition of 2N HCl. A white solid precipitated, accompanied by

CO₂ evolution. The mixture was extracted with EtOAc (3×1 L). The combined extracts were washed with water (2×1 L) and brine, then dried over Na₂SO₄. HPLC analysis of this solution showed the material was 98% ee. The solvent was evaporated under reduced pressure, giving an oil which soon solidified. The title product (172.9 g) was obtained as an off-white solid after vacuum drying. This material was recrystallized from boiling hexanes (8.8 L). After overnight cooling, light yellow needles were collected via filtration, washed with hexanes (200 mL), and dried under suction. The title product (146.9 g, 38% from crude starting ester) was obtained as light yellow needles after vacuum drying. ¹H NMR results as above.

### Example 134

### Preparation of ethyl [(1S)-5-methoxy-2,3-dihydro-1H-inden-1-yl]acetate

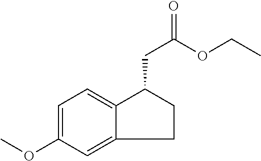 [SMILES: CCOC(=O)C[C@@H]1CCc2cc(OC)ccc21]

To a solution of the acid (305 g, 1.48 mol) prepared in either Example 132 or 133 in 4.8 L absolute EtOH at rt under argon, was added chlorotrimethylsilane (413 mL, 3.25 mol) dropwise. An approximate 5° C. rise in temperature was noted during the addition. The reaction was stirred overnight. EtOH was evaporated under reduced pressure, giving a bi-phasic liquid mixture. This was diluted in 500 mL ice-water, then extracted with EtOAc (2×750 mL). The combined extracts were washed with water (3×300 mL), then with saturated NaHCO₃ (200 mL). The organic was washed once more with water (300 mL), then brine, and dried over Na₂SO₄. The title compound (354 g, 102%) was obtained as a light yellow oil after solvent removal and vacuum drying. ¹H NMR (CDCl₃) δ 7.07 (d, 1H), 6.78 (d, 1H), 6.71 (dd, 1H), 4.18 (q, 2H), 3.78 (s, 3H), 3.52 (m, 1H), 2.89 (m, 2H), 2.72 (dd, 1H), 2.37 (o, 2H), 1.74 (m, 1H), 1.28 (t, 3H). MS (Cl) m/z 235 [M+H]⁺.

### Example 135

### Preparation of ethyl [(1S)-5-hydroxy-2,3-dihydro-1H-inden-1-yl]acetate

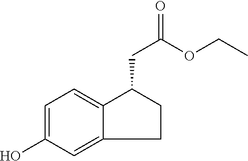 [SMILES: CCOC(=O)C[C@@H]1CCc2cc(O)ccc21]

To a cold solution (ice water bath) of the compound (346 g, 1.48 mol) prepared in Example 134 in 4.2 L CH₂Cl₂, was added AlCl₃ (984.6 g, 7.38 mol) portionwise under Ar such that the reaction temperature was maintained below 10° C. The light brown suspension was stirred 10 minutes, then EtSH (546 mL, 7.38 mol) was added dropwise at such a rate that the reaction temperature was maintained below 5° C. After 2.5 hours of stirring below 10° C., the reaction mixture was slowly poured into 6 L ice water with strong agitation. The organic layer was separated, and the aqueous layer was extracted with CH₂Cl₂ (3×1 L). The combined CH₂Cl₂ layers were washed with water (2×1 L), then dried over Na₂SO₄. The solvent was removed under reduced pressure, giving a brown oil, which was filtered through a pad of silica gel (eluted with 0-10% EtOAc/Hexanes). Fractions were collected and the title compound (314 g, 96%) was obtained as a thick yellow oil after solvent removal and vacuum drying. ¹H NMR (CDCl₃) δ 6.92 (d, 1H), 6.62 (d, 1H), 6.55 (dd, 1H), 4.10 (q, 2H), 3.43 (q, 1H), 2.75 (m, 2H), 2.64 (dd, 1H), 2.31 (dd, 1H), 2.29 (m, 1H), 1.67 (m, 1H), 1.20 (t, 3H). MS (Cl) m/z 221 [M+H]⁺.

### Example 136

### Preparation of ethyl 2-((1S)-5-{2-[5-methyl-2-(4-methylphenyl)(1,3-oxazol-4-yl)]ethoxy}indanyl)acetate

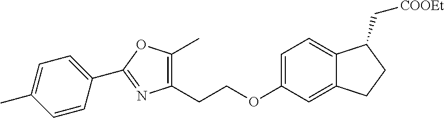 [SMILES: CCOC(=O)C[C@@H]1CCc2cc(OCCc3nc(-c4ccc(C)cc4)oc3C)ccc21]

A suspension of the ethyl [(1S)-5-hydroxy-2,3-dihydro-1H-inden-1-yl]acetate prepared in Example 135 (507.5 mg, 2.30 mmol), and 2-[5-methyl-2-(4-methylphenyl)-1,3-oxazol-4-yl]ethanol prepared in Example 10 (500 mg, 2.30 mmol), TMAD (792.6 mg, 4.60 mmol), and Ph₃P (1.21 g, 4.60 mmol) in 15 mL anhydrous DCM was stirred at rt under Ar for 12 hours. DCM was removed under reduced pressure. Flash chromatograph of the residue over silica gel using 1% CH₃CN/CH₂Cl₂ gave ethyl 2-((1S)-5-{2-[5-methyl-2-(4-methylphenyl)(1,3-oxazol-4-yl)]ethoxy}indanyl)acetate (776.3 mg, 1.85 mmol, 80.5%).

HPLC/MS (M+H)⁺ m/z 420.5.

### Example 137

### Preparation of 2-((1S)-5-{2-[5-methyl-2-(4-methylphenyl)(1,3-oxazol-4-yl)]ethoxy}indanyl)acetic Acid

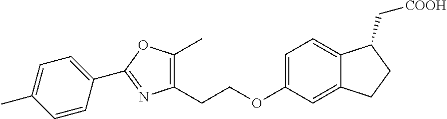 [SMILES: Cc1ccc(-c2nc(CCOc3ccc4c(c3)CC[C@H]4CC(=O)O)c(C)o2)cc1]

Ethyl 2-((1S)-5-{2-[5-methyl-2-(4-methylphenyl)(1,3-oxazol-4-yl)]ethoxy}indanyl)acetate (Example 136, 776.3 mg, 1.85 mmol) in THF (4.0 ml) was added to a mixture of aqueous LiOH (2 M, 3.7 ml, 7.4 mmol), water (2.0 ml), and EtOH (4.0 ml) at rt. The resulting mixture turned cloudy. This mixture was heated at 40° C. (oil-bath temperature). The reaction was completed after 1.5 hours. After cooling to rt, 1 N HCl solution was slowly added to the mixture until pH 4.0. The compound was extracted with EtOAc (3×20 ml). The combined EtOAc layers were dried (Na₂SO₄) and evaporated. Flash chromatography of the residue gave 2-((1S)-5-{2-[5-methyl-2-(4-methylphenyl)(1,3-oxazol-4-yl)]ethoxy}indanyl)acetic acid (616.8 mg, 1.57 mmol, 85%) as a white solid. ¹H NMR (CDCl₃) δ 7.83 (d, 2H), 7.21 (d, 2H), 7.03 (d, 1H), 6.74 (d, 1H), 6.69 (dd, 1H), 4.19 (t, 2H), 3.45 (q, 1H), 2.93 (t, 2H), 2.78 (m, 2H), 2.51 (m, 2H), 2.30 (s, 3H), 2.25 (s, 3H), 1.53 (m, 2H).

By using the methods described above for Examples 129-137 and by substituting the appropriate starting materials, compounds of Formula Ia, listed in Table 4 below, were similarly prepared.

TABLE 4
Preparative Examples of Compounds of Formula (Ia)
(Ia)

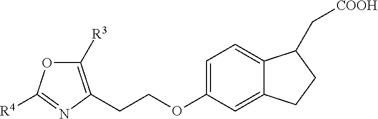

| Ex. | | | LC/MS |
|---|---|---|---|
| No. | R³ | R⁴ | [M + H]⁺ |
| 138 | Me | 4-MeO—Ph | 408.5 |
| 139 | Me | 3-MeO—Ph | 408.5 |
| 140 | Me | 4-Et—Ph | 406.5 |
| 141 | Me | 4-CF3—Ph | 446.5 |
| 142 | Me | 2-naphthyl | 428.5 |
| 143 | Me | 4-(t-Bu)—Ph | 434.6 |
| 144 | Me | 4-(n-Bu)—Ph | 434.6 |
| 145 | Me | 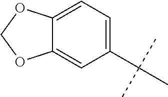 | 422.5 |
| 146 | Me | 3,4-(Me)₂—Ph | 406.5 |
| 147 | Me | 4-Me—Ph | 392.5 |
| 148 | Me | 3-F—Ph | 396.5 |
| 149 | Me | 2-benzothienyl | 434.5 |
| 150 | Me | 4-i-Pr—Ph | 420.6 |
| 151 | Me | cyc-Pentyl | 370.5 |
| 152 | Me | cyc-hexyl | 384.5 |
| 153 | Me | PhCH₂ | 392.5 |
| 154 | Me | 4-F-3-Me—Ph | 410.5 |
| 155 | Me | 3-F-4-Me—Ph | 410.5 |
| 156 | Me | 4-F—Ph | 396.5 |
| 157 | Et | Ph | 392.5 |
| 158 | Me | 3,4-(Cl)₂—Ph | 447.4 |
| 159 | n-Pr | Ph | 406.5 |
| 160 | Me | 4-Ph—Ph | 454.5 |
| 161 | Me | 3-Cl—Ph | 412.4 |
| 162 | Me | 3-Me—Ph | 392.5 |
| 163 | Me | 4-CN—Ph | 403.4 |
| 164 | Me | 3-CN—Ph | 403.4 |
| 165 | Me | 4-Cl—Ph | 412.4 |
| 166 | Me | 3-CF3—Ph | 446.4 |
| 167 | Et | 4-Et—Ph | 420.5 |
| 168 | Et | 4-Me—Ph | 406.5 |
| 169 | Et | 4-MeO—Ph | 422.4 |

### Example 170

### Preparation of methyl 4-bromo-3-oxopentanoate

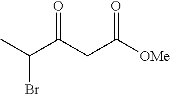

A dry three-neck flask under an Ar atmosphere was charged with a solution of methyl propionylacetate (20 g, 154 mmol) in CHCl₃ (100 mL). Using an addition funnel, bromine (7.9 mL, 24.6 g, 154 mmol) was added dropwise over a period of 2 hours at 0° C. The reaction was then allowed to warm slowly to rt, and the reaction mixture was stirred overnight. A saturated solution of Na₂CO₃ (40 mL) was slowly added, and after stirring the reaction mixture for an additional 15 minutes, the solvents layers were separated and the aqueous layer was extracted with CH₂Cl₂ (50 mL). The combined organic layers were dried (Na₂SO₄), filtered, and concentrated under reduced pressure. The residue was then purified by silica gel flash chromatography (10:1 hexanes/EtOAc) to give the desired bromide as a light yellow oil (25 g, 78%). ¹H NMR (CDCl₃): δ 1.80 (d, 3H), 3.64-3.92 (m, 2H), 3.78 (s, 3H), 4.61 (q, 1H).

### Example 171

### Preparation of methyl (2-amino-5-methyl-1,3-thiazol-4-yl)acetate

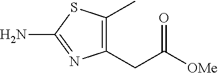

To a solution of bromide of Example 170 (18 g, 86 mmol) in toluene (100 mL) was added thiourea (10.5 g, 138 mmol). The reaction mixture was heated to 100° C. for 1 hour, cooled to rt, and the solvent removed under reduced pressure. The residue was dissolved with CH₂Cl₂ (100 mL), a saturated solution NaHCO₃ (75 mL) added, and the mixture was vigorously stirred for 10 minutes. The organic layer was separated, dried (Na₂SO₄), filtered, and concentrated under reduced pressure. The residue was then recrystallized from CH₂Cl₂/hexanes to provide the product (10 g, 63%) as a white solid. (C₇H₁₀N₂O₂S): LC-MS, RT 0.76 min, M+H 187.0; ¹H NMR (CDCl₃): δ 2.23 (s, 3H), 3.70 (s, 2H), 3.75 (s, 3H), 4.83-4.95 (broad s, 2H).

### Example 172

### Preparation of methyl (2-bromo-5-methyl-1,3-thiazol-4-yl)acetate

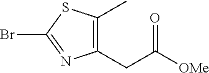

To a solution of CuBr₂ (4.03 g, 18.1 mmol) and t-butyl nitrite (2.82 mL, 23.8 mmol) in MeCN (210 mL) was added the compound of Example 170 (2.95 g, 15.9 mmol) at −20° C. The reaction mixture was slowly warmed to 15° C., at which point the evolution of N₂ was observed. After stirring for an additional 2 hours at 15° C., the reaction mixture was diluted with Et₂O (400 mL) and washed with a 10% solution of HCl (200 mL). The solvent layers were separated, the aqueous re-extracted with Et₂O (2×300 mL), and the combined organic layers dried (MgSO₄), filtered, and concentrated under reduced pressure. The residue was then purified by silica gel flash chromatography (98:2, hexanes/EtOAc) to afford bromide Example 172 (1.6 g, 40%) as a colorless oil that solidifies upon standing. (C₇H₈BrNO₂S): LC-MS, RT 2.56 min., M+H 250.3; ¹H NMR (CDCl₃): δ 2.26 (s, 3H), 3.60 (s, 2H), 3.61 (s, 3H).

### Example 173

### Preparation of 2-(2-bromo-5-methyl-1,3-thiazol-4-yl)ethanol

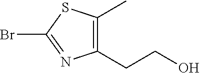

To a solution of ester prepared in Example 172 (3.80 g, 15.2 mmol) in CH₂Cl₂ (100 mL) was added DIBAL-H (33.4 mL, 33.4 mmol of a 1.0 M solution in toluene) at −78° C. After 15 minutes, the solution was warmed to 0° C. and stirred for an additional 90 minutes. An aqueous solution of 2 N HCl (50 mL) was then added dropwise to quench the excess DIBAL-H. The solvent layers were separated and the aqueous layer extracted with CH₂Cl₂ (2×200 mL). The combined organic layers were dried (MgSO₄), filtered, and concentrated under reduced pressure. The residue was purified by silica gel flash chromatography (5:2 hexanes/EtOAc) to yield the product (2.5 g, 74%) as a yellowish oil that solidifies upon standing. (C₆H₈BrNOS) LC-MS, RT 1.38 min., M+H 221.0; ¹H NMR (CDCl₃): δ 2.31 (s, 3H), 2.82 (t, 2H), 2.90-3.00 (broad s, 1H), 3.89 (t, 2H).

### Example 174

### Preparation of ethyl {(1S)-5-[2-(2-bromo-5-methyl-1,3-thiazol-4-yl)ethoxy]-2,3-dihydro-1H-inden-1-yl}acetate

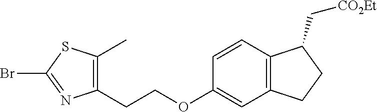 [SMILES: CCOC(=O)C[C@@H]1CCc2cc(OCCc3nc(Br)sc3C)ccc21]

Step 1. To a solution of Example 173 (975 mg, 4.39 mmol) and ethyl [(1S)-5-hydroxy-2,3-dihydro-1H-inden-1-yl]acetate (1.06 g, 4.83 mmol) in THF (20 mL) were added Ph₃P (1.88 g, 7.46 mmol) and ADDP (1.96 g, 7.46 mmol). The mixture was vigorously stirred at rt for 72 hours, the solvent removed under reduced pressure, and the residue purified by silica gel flash chromatography (6:1 hexanes/EtOAc) to yield the product (1.4 g, 76%) as a colorless oil that solidifies upon standing. (C₁₉H₂₂BrNO₃S) LC-MS, RT 3.92 min., M+H 424.5; ¹H NMR (CDCl₃): δ 1.26 (t, 3H), 1.65-1.81 (m, 1H), 2.28-2.45 (m, 2H), 2.37 (s, 3H), 2.69 (dd, 1H), 2.75-2.93 (m, 2H), 3.07 (t, 2H), 3.44-3.56 (m, 1H), 4.15 (t, 2H), 4.18 (q, 2H), 6.67 (dd, 1H), 6.73 (d, 1H), 7.03 (d, 1H).

### Preparation of ethyl ((1S)-5-{2-[2-(4-isopropylphenyl)-5-methyl-1,3-thiazol-4-yl]ethoxy}-2,3-dihydro-1H-inden-1-yl)acetate

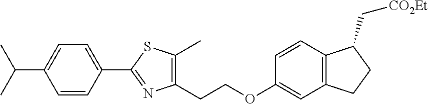 [SMILES: CCOC(=O)C[C@@H]1CCc2cc(OCCc3nc(-c4ccc(C(C)C)cc4)sc3C)ccc21]

Step 2. To a mixture of toluene (15 mL) and 1,4-dioxane (3 mL), were added the compound of step 1 (300 mg, 0.708 mmol), 4-isopropylbenzene boronic acid (464 mg, 2.83 mmol), and PdCl₂(dppf).CH₂Cl₂ (52 mg, 0.071 mmol). A flow of Ar was passed through the mixture for 30 minutes, then a 2 N solution of Na₂CO₃ (3.7 mL, 7.08 mmol) was added and the reaction was heated to 75° C. for 18 hours. The reaction mixture was then cooled to rt, diluted with EtOAc (200 mL), and washed with a saturated solution of NaHCO₃ (50 mL). The organic layer was dried (Na₂SO₄), filtered, and concentrated under reduced pressure. The residue was purified by silica gel flash chromatography (8:1 hexanes/EtOAc), to provide the product (305 mg, 93%) as a colorless oil. (C₂₈H₃₃NO₃S): LC-MS, RT 5.17 min., M+H 464.5; ¹H NMR (CDCl₃): δ 1.17-1.31 (m, 3H), 1.26 (s, 3H), 1.27 (s, 3H), 1.65-1.82 (m, 1H), 2.30-2.43 (m, 2H), 2.46 (s, 3H), 2.72 (dd, 1H), 2.78-3.00 (m, 3H), 3.17 (t, 2H), 3.46-3.57 (m, 1H), 4.17 (q, 2H), 4.27 (t, 2H), 6.71 (d, 1H), 6.78 (s, 1H), 7.04 (d, 1H), 7.55 (AB quartet, 4H).

### Example 175

### Preparation of ((1S)-5-{2-[2-(4-isopropylphenyl)-5-methyl-1,3-thiazol-4-yl]ethoxy}-2,3-dihydro-1H-inden-1-yl)acetic Acid

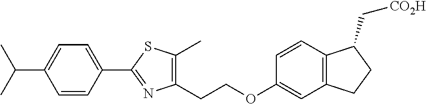 [SMILES: Cc1sc(-c2ccc(C(C)C)cc2)nc1CCOc1ccc2c(c1)CC[C@H]2CC(=O)O]

To a solution of Example 174 (305 mg, 0.657 mmol) in a mixture of THF (8 mL), water (8 mL), and EtOH (4 mL), was added LiOH (63 mg, 2.63 mmol). The reaction mixture was vigorously stirred for 24 hours, diluted with water (20 mL), and washed with

Et₂O (10 mL). The aqueous phase was then acidified to pH ˜1 using 1 N HCl, and then extracted with CH₂Cl₂ (4×50 mL). The combined organic layers were dried (Na₂SO₄), filtered, and concentrated under reduced pressure. The residue was then purified by silica gel flash chromatography (95:5 CH₂Cl₂/MeOH) to afford product (189 mg, 66%) as a white solid. (C₂₆H₂₉NO₃S): LC-MS, RT 3.95 min., M+H 436.4; ¹H NMR (CDCl₃): δ 1.25 (s, 3H), 1.28 (s, 3H), 1.70-1.82 (m, 1H), 2.32-2.43 (m, 2H), 2.45 (s, 3H), 2.74-2.98 (m, 4H), 3.18 (t, 2H), 3.47-3.54 (m, 1H), 4.28 (t, 2H), 6.72 (dd, 1H), 6.78 (s, 1H), 7.08 (d, 1H), 7.51 (AB quartet, 4H).

### Example 176

### Preparation of methyl [5-methyl-2-(4-methylphenyl)-1,3-thiazol-4-yl]acetate

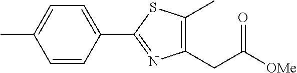 [SMILES: COC(=O)Cc1nc(-c2ccc(C)cc2)sc1C]

To a solution of bromide of Example 170 (1.15 g, 5.52 mmol) in toluene (20 mL) was added 4-methyl thiobenzamide (1.0 g, 6.6 mmol). The reaction mixture was heated to reflux for 15 hours, cooled to rt, diluted with EtOAc (150 mL), and washed with a saturated solution of NaHCO₃ (50 mL), then with a saturated solution of NH₄Cl (50 mL). The organic layer was dried (Na₂SO₄), filtered, and concentrated under reduced pressure. The residue was then purified by silica gel flash chromatography (9:1 hexanes/EtOAc) to afford the product as a pinkish oil that solidified upon standing (1.14 g, 62%). ¹H NMR (CDCl₃): δ 2.38 (s, 3H), 3.45 (s, 3H), 3.74 (s, 3H), 3.80 (s, 2H), 7.49 (AB quartet, 4H); R_{f} (0.4, eluant 9:1 hexanes/EtOAc).

### Example 177

### Preparation of 2-[5-methyl-2-(4-methylphenyl)-1,3-thiazol-4-yl]ethanol

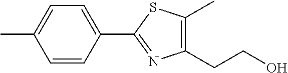 [SMILES: Cc1ccc(-c2nc(CCO)c(C)s2)cc1]

To a solution of the thiazole of Example 176 (1.14 g, 4.37 mmol) in THF (60 mL) at 0° C., was added portion-wise LiAlH₄ (663 mg, 17.5 mmol). After 30 minutes, the reaction mixture was warmed to rt and stirred for an additional 60 minutes. The reaction mixture was then cooled to 0° C., and the excess LiAlH₄ was quenched by dropwise addition of water (5 mL), 1N NaOH (10 mL), and water (5 mL) sequentially. The mixture was then diluted with a saturated solution of Rochelle salt and extracted with EtOAc (4×75 mL). The combined organic phases were dried (Na₂SO₄), filtered, and concentrated under reduced pressure. The residue was purified by silica gel flash chromatography (3:2 hexanes/EtOAc) to afford the product as a white solid (830 mg, 82%). (C₁₃H₁₅NOS): LC-MS, RT 2.50 min., M+H 234.2; ¹H NMR (CDCl₃): δ 2.34 (s, 3H), 2.37 (s, 3H), 2.83 (t, 2H), 3.92-4.01 (broad t, 2H), 4.04-4.15 (broad s, 1H), 7.45 (AB quartet, 4H).

The following compounds below were synthesized using one of the two procedures of Examples 170-177 described above.

### Example 178

### {(1S)-5-[2-(5-Methyl-2-phenyl-1,3-thiazol-4-yl)ethoxy]-2,3-dihydro-1H-inden-1-yl}acetic Acid

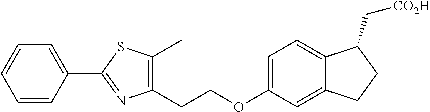 [SMILES: Cc1sc(-c2ccccc2)nc1CCOc1ccc2c(c1)CC[C@H]2CC(=O)O]

(C₂₃H₂₃NO₃S): LC-MS RT 3.56 min., M+H 394.2; ¹H NMR (CDCl₃): δ 1.61-1.78 (m, 1H), 2.19-2.50 (m, 2H), 2.30 (s, 3H), 2.62-2.91 (m, 3H), 3.12 (t, 2H), 3.17-3.26 (m, 1H), 4.12 (t, 2H), 6.70 (d, 1H), 6.79 (s, 1H), 6.98 (d, 1H), 7.21-7.40 (m, 3H), 7.74-7.83 (m, 2H).

### Example 179

((1S)-5-{2-[5-Methyl-2-(4-methylphenyl)-1,3-thiazol-4-yl]ethoxy}-2,3-dihydro-1H-inden-1-yl)acetic Acid

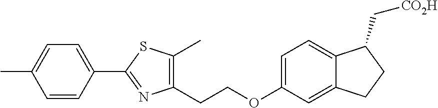 [SMILES: Cc1ccc(-c2nc(CCOc3ccc4c(c3)CC[C@H]4CC(=O)O)c(C)s2)cc1]

(C₂₄H₂₅NO₃S): LC-MS, RT 3.57 min., M+H 408.5; ¹H NMR (CDCl₃): δ 1.61-1.68 (m, 1H), 2.29 (s, 3H), 2.36 (s, 3H), 2.25-2.37[hidden] (m, 2H), 2.63-2.79 (m, 3H), 3.09 (t, 2H), 3.35-3.47 (m, 1H), 4.18 (t, 2H), 6.60 (dd, 1H), 6.68 (s, 1H), 6.97 (d, 1H), 7.42 (AB quartet, 4H), 7.81-8.30 (br, 1H).

### Example 180

### ((1S)-5-{2-[2-(1,3-Benzodioxol-5-yl)-5-methyl-1,3-thiazol-4-yl]ethoxy}-2,3-dihydro-1H-inden-1-yl)acetic acid

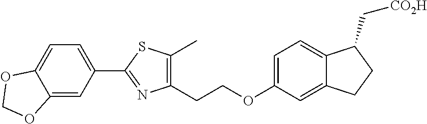 [SMILES: Cc1sc(-c2ccc3c(c2)OCO3)nc1CCOc1ccc2c(c1)CC[C@H]2CC(=O)O]

(C₂₄H₂₃NO₅S): LC-MS, RT 4.04 min., M+H 438.5; ¹H NMR (CDCl₃): δ 1.71-1.83 (m, 1H), 2.36-2.51 (m, 2H), 2.45 (s, 3H), 2.76-2.96 (m, 3H), 3.15 (t, 2H), 3.48-3.58 (m, 1H), 4.29 (t, 2H), 6.00 (s, 2H), 6.72 (dd, 1H), 6.78 (s, 1H), 6.82 (d, 1H), 7.07 (d, 1H), 7.32-7.40 (m, 2H).

### Example 181

### ((1S)-5-{2-[2-(4-Methoxyphenyl)-5-methyl-1,3-thiazol-4-yl]ethoxy}-2,3-dihydro-1H-inden-1-yl)acetic Acid

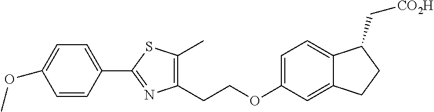 [SMILES: COc1ccc(-c2nc(CCOc3ccc4c(c3)CC[C@H]4CC(=O)O)c(C)s2)cc1]

(C₂₄H₂₅NO₄S): LC-MS, RT 4.01 min., M+H 424.5; ¹H NMR (CDCl₃): δ 1.67-1.82 (m, 1H), 2.43 (s, 3H), 2.34-2.47 (m, 2H), 2.72-2.95 (m, 3H), 3.09 (t, 2H), 3.42-3.57 (m, 1H), 3.84 (s, 3H), 4.13 (t, 2H), 6.72 (d, 1H), 6.79 (s, 1H), 7.12 (d, 1H), 7.37 (AB quartet, 4H).

### Example 182

### [(1S)-5-(2-{5-Methyl-2-[4-(trifluoromethyl)phenyl]-1,3-thiazol-4-yl}ethoxy)-2,3-dihydro-1H-inden-1-yl]acetic Acid

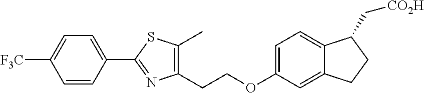 [SMILES: Cc1sc(-c2ccc(C(F)(F)F)cc2)nc1CCOc1ccc2c(c1)CC[C@H]2CC(=O)O]

(C₂₄H₂₂F₃NO₃S): LC-MS, RT 4.47 min., M+H 462.4; ¹H NMR (DMSO_{d6}): δ 1.63-1.81 (m, 1H), 2.28-2.43 (m, 2H), 2.50 (s, 3H), 2.69 (dd, 1H), 2.74-2.95 (m, 2H), 3.19 (t, 2H), 3.31-3.36 (m, 1H), 4.31 (t, 2H), 6.71 (dd, 1H), 6.78 (s, 1H), 7.08 (d, 1H), 7.87 (AB quartet, 4H).

### Example 183

### ((1S)-5-{2-[2-(4-Cyanophenyl)-5-methyl-1,3-thiazol-4-yl]ethoxy}-2,3-dihydro-1H-inden-1-yl)acetic Acid

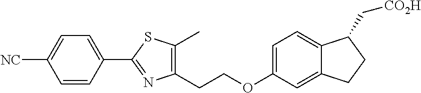 [SMILES: Cc1sc(-c2ccc(C#N)cc2)nc1CCOc1ccc2c(c1)CC[C@H]2CC(=O)O]

(C₂₄H₂₂N₂O₃S): LC-MS, RT 3.43 min., M+H 419.6; ¹H NMR (CDCl₃): δ 1.68-1.85 (m, 1H), 2.31-2.49 (m, 2H), 2.51 (s, 3H), 2.77 (dd, 1H), 2.83-2.94 (m, 2H), 3.18 (t, 2H), 3.43-3.56 (m, 1H), 4.31 (t, 2H), 6.71 (dd, 1H), 6.79 (s, 1H), 7.10 (d, 1H), 7.86 (AB quartet, 4H).

### Example 184

### ((1S)-5-{2-[2-(4-Isopropylphenyl)-5-methyl-1,3-thiazol-4-yl]ethoxy}-2,3-dihydro-1H-inden-1-yl)acetic Acid

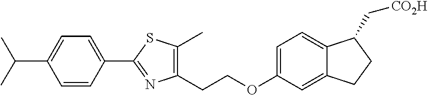 [SMILES: Cc1sc(-c2ccc(C(C)C)cc2)nc1CCOc1ccc2c(c1)CC[C@H]2CC(=O)O]

(C₂₆H₂₉NO₃S): LC-MS, RT 3.95 min., M+H 436.4; ¹H NMR (CDCl₃): δ 1.25 (s, 3H), 1.28 (s, 3H), 1.70-1.82 (m, 1H), 2.32-2.43 (m, 2H), 2.45 (s, 3H), 2.74-2.98 (m, 4H), 3.18 (t, 2H), 3.47-3.54 (m, 1H), 4.28 (t, 2H), 6.72 (dd, 1H), 6.78 (s, 1H), 7.08 (d, 1H), 7.51 (AB quartet, 4H).

### Example 185

### ((1S)-5-{2-[2-(3-Chloro-4-fluorophenyl)-5-methyl-1,3-thiazol-4-yl]ethoxy}-2,3-dihydro-1H-inden-1-yl)acetic Acid

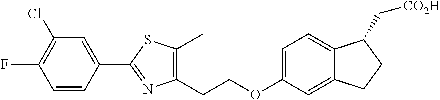 [SMILES: Cc1sc(-c2ccc(F)c(Cl)c2)nc1CCOc1ccc2c(c1)CC[C@H]2CC(=O)O]

(C₂₃H₂₁ClFNO₃S): LC-MS, RT 3.89 min., M+H 446.4; ¹H NMR (CDCl₃): δ 1.68-1.86 (m, 1H), 2.32-2.46 (m, 2H), 2.50 (s, 3H), 2.80 (dd, 1H), 2.84-2.96 (m, 2H), 3.18 (t, 2H), 3.47-3.59 (m, 1H), 4.32 (t, 2H), 6.72 (d, 1H), 6.82 (s, 1H), 7.12 (d, 1H), 7.23 (t, 1H), 7.72-7.82 (m, 1H), 7.97-8.04 (m, 1H).

### Example 186

### ((1S)-5-{2-[2-(3,4-Dichlorophenyl)-5-methyl-1,3-thiazol-4-yl]ethoxy}-2,3-dihydro-1H-inden-1-yl)acetic Acid

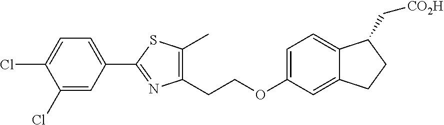 [SMILES: Cc1sc(-c2ccc(Cl)c(Cl)c2)nc1CCOc1ccc2c(c1)CC[C@H]2CC(=O)O]

(C₂₃H₂₁Cl₂NO₃S): LC-MS, RT 4.12 min., M+H 462.0; ¹H NMR (CDCl₃): δ 1.74-1.88 (m, 1H), 2.36-2.48 (m, 2H), 2.50 (s, 3H), 2.73-2.93 (m, 3H), 3.19 (t, 2H), 3.48-3.55 (m, 1H), 4.30 (t, 2H), 6.71 (d, 1H), 6.79 (s, 1H), 7.09 (d, 1H), 7.52 (d, 1H), 7.61 (dd, 1H), 8.02 (d, 1H).

### Example 187

### ((1S)-5-{2-[2-(4-Fluorophenyl)-5-methyl-1,3-thiazol-4-yl]ethoxy}-2,3-dihydro-1H-inden-1-yl)acetic Acid

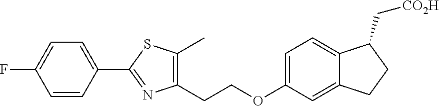 [SMILES: Cc1sc(-c2ccc(F)cc2)nc1CCOc1ccc2c(c1)CC[C@H]2CC(=O)O]

(C₂₃H₂₂FNO₃S): LC-MS, RT 3.58 min., M+H 412.4; ¹H NMR (CDCl₃): δ 1.70-1.77 (m, 1H), 2.37-2.45 (m, 1H), 2.44 (s, 3H), 2.70-2.90 (m, 4H), 3.16 (t, 2H), 3.47-3.52 (m, 1H), 4.27 (t, 2H), 6.70 (d, 1H), 6.76 (s, 1H), 7.00-7.10 (m, 3H), 7.82-7.87 (m, 2H).

### Example 188

((1S)-5-{2-[2-(3,4-Dimethylphenyl)-5-methyl-1,3-thiazol-4-yl]ethoxy}-2,3-dihydro-1H-inden-1-yl)acetic Acid

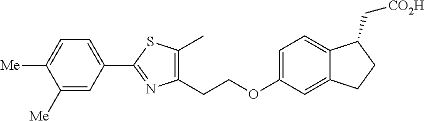 [SMILES: Cc1ccc(-c2nc(CCOc3ccc4c(c3)CC[C@H]4CC(=O)O)c(C)s2)cc1C]

(C₂₅H₂₇NO₃S): LC-MS, RT 4.39 min., M+H 422.3; ¹H NMR (CDCl₃): δ 1.70-1.83 (m, 1H), 2.29 (s, 3H), 2.32 (s, 3H), 2.37-2.50 [hidden] (m, 2H), 2.46 (s, 3H), 2.70-2.90 (m, 3H), 3.32 (t, 2H), 3.45-3.60 (m, 1H), 4.30 (t, 2H), 6.73 (d, 1H), 6.79 (s, 1H), 7.07 (d, 1H), 7.17 (d, 1H), 7.59 (d, 1H), 7.68 (s, 1H).

### Example 189

### ((1S)-5-{2-[2-(4-Acetylphenyl)-5-methyl-1,3-thiazol-4-yl]ethoxy}-2,3-dihydro-1H-inden-1-yl)acetic Acid

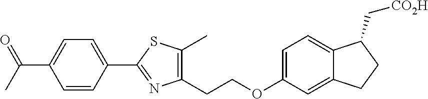 [SMILES: CC(=O)c1ccc(-c2nc(CCOc3ccc4c(c3)CC[C@H]4CC(=O)O)c(C)s2)cc1]

(C₂₅H₂₅NO₄S): LC-MS, RT 4.01 min., M+H 436.3; ¹H NMR (CDCl₃): δ 1.70-1.82 (m, 1H), 2.37-2.49 (m, 2H), 2.50 (s, 3H), 2.63 (s, 3H), 2.70-2.90 (m, 3H), 3.20 (t, 2H), 3.45-3.60 (m, 1H), 4.30 (t, 2H), 6.72 (d, 1H), 6.78 (s, 1H), 7.08 (d, 1H), 7.95-8.03 (m, 4H).

### Example 190

### [(1S)-5-(2-{2-[4-(Dimethylamino)phenyl]-5-methyl-1,3-thiazol-4-yl}ethoxy)-2,3-dihydro-1H-inden-1-yl]acetic Acid

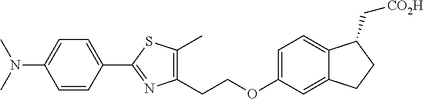 [SMILES: Cc1sc(-c2ccc(N(C)C)cc2)nc1CCOc1ccc2c(c1)CC[C@H]2CC(=O)O]

(C₂₅H₂₈N₂O₃S): LC-MS, RT 2.95 min., M+H 437.2; ¹H NMR (DMSO_{d6}): δ 1.53-1.65 (m, 1H), 2.12-2.24 (m, 2H), 2.36 (s, 3H), 2.63-2.84 (m, 3H), 2.94 (s, 6H), 3.03 (t, 2H), 3.27-3.38 (m, 1H), 4.18 (t, 2H), 6.65 (d, 1H), 6.75 (s, 1H), 7.08 (d, 1H), 7.17 (AB quartet, 4H).

### Example 191

### ((1S)-5-{2-[2-(3-Amino-4-methylphenyl)-5-methyl-1,3-thiazol-4-yl]ethoxy}-2,3-dihydro-1H-inden-1-yl)acetic Acid

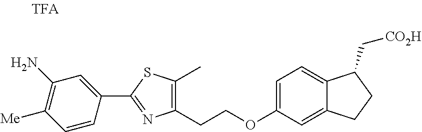 [SMILES: Cc1ccc(-c2nc(CCOc3ccc4c(c3)CC[C@H]4CC(=O)O)c(C)s2)cc1N]

C₂₄H₂₆N₂O₃S.C₂F₃O₂): LC-MS, RT 3.5 min., M+H 423.3; ¹H NMR (CD₃OD): δ 1.67-1.82 (m, 1H), 2.25-2.37 (m, 2H), 2.38 (s, 3H), 2.50 (s, 3H), 2.67-2.90 (m, 3H), 3.20 (t, 2H), 3.41-3.56 (m, 1H), 4.32 (t, 2H), 6.71 (d, 1H), 6.79 (s, 1H), 7.09 (d, 1H), 7.42 (d, 1H), 7.69 (dd, 1H), 7.77 (d, 1H).

### Example 192

### ((1S)-5-{2-[2-(2-Fluorophenyl)-5-methyl-1,3-thiazol-4-yl]ethoxy}-2,3-dihydro-1H-inden-1-yl)acetic Acid

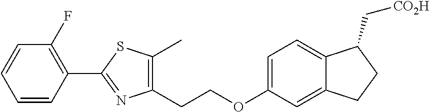 [SMILES: Cc1sc(-c2ccccc2F)nc1CCOc1ccc2c(c1)CC[C@H]2CC(=O)O]

(C₂₃H₂₂FNO₃S): LC-MS, RT 4.25 min., M+H 412.2; ¹H NMR (CDCl₃): δ 1.70-1.82 (m, 1H), 2.37-2.48 (m, 2H), 2.49 (s, 3H), 2.74-2.94 (m, 3H), 3.21 (t, 2H), 3.42-3.60 (m, 1H), 4.31 (t, 2H), 6.72 (d, 1H), 6.79 (s, 1H), 7.06-7.35 (m, 4H), 8.21 (t, 1H).

### Example 193

### ((1S)-5-{2-[2-(4-Chlorophenyl)-5-methyl-1,3-thiazol-4-yl]ethoxy}-2,3-dihydro-1H-inden-1-yl)acetic Acid

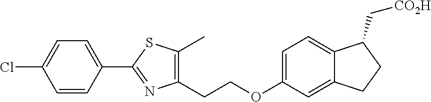 [SMILES: Cc1sc(-c2ccc(Cl)cc2)nc1CCOc1ccc2c(c1)CC[C@H]2CC(=O)O]

(C₂₃H₂₂ClNO₃S): LC-MS, RT 4.44 min., M+H 428.2; ¹H NMR (CDCl₃): δ 1.70-1.81 (m, 1H), 2.35-2.45 (m, 2H), 2.46 (s, 3H), 2.74-2.89 (m, 3H), 3.17 (t, 2H), 3.42-3.60 (m, 1H), 4.28 (t, 2H), 6.71 (d, 1H), 6.77 (s, 1H), 7.07 (d, 1H), 7.36 (d, 2H), 7.79 (d, 2H).

### Example 194

### ((1S)-5-{2-[2-(4-Ethoxyphenyl)-5-methyl-1,3-thiazol-4-yl]ethoxy}-2,3-dihydro-1H-inden-1-yl)acetic Acid

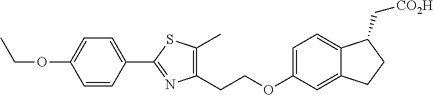 [SMILES: CCOc1ccc(-c2nc(CCOc3ccc4c(c3)CC[C@H]4CC(=O)O)c(C)s2)cc1]

(C₂₅H₂₇NO₄S): LC-MS, RT 3.55 min., M+H 438.5; ¹H NMR (CDCl₃): δ 1.40 (t, 3H), 1.70-1.82 (m, 1H), 2.35-2.47 (m, 2H), 2.45 (s, 3H), 2.74-2.89 (m, 3H), 3.20 (t, 2H), 3.42-3.59 (m, 1H), 4.07 (q, 2H), 4.29 (t, 2H), 6.71 (d, 1H), 6.76 (s, 1H), 6.91 (d, 1H), 7.06 (d, 2H), 7.82 (d, 2H).

### Example 195

### ((1S)-5-{2-[2-(3,4-Dimethoxyphenyl)-5-methyl-1,3-thiazol-4-yl]ethoxy}-2,3-dihydro-1H-inden-1-yl)acetic Acid

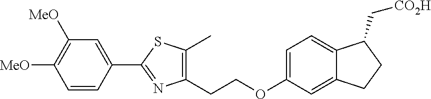 [SMILES: COc1ccc(-c2nc(CCOc3ccc4c(c3)CC[C@H]4CC(=O)O)c(C)s2)cc1OC]

(C₂₅H₂₇NO₅S): LC-MS, RT 3.86 min., M+H 454.2; ¹H NMR (CDCl₃): δ 1.67-1.82 (m, 1H), 2.37-2.48 (m, 2H), 2.49 (s, 3H), 2.71-2.87 (m, 3H), 3.27 (t, 2H), 3.42-3.57 (m, 1H), 3.93 (s, 3H), 3.96 (s, 3H), 4.29 (t, 2H), 6.35-6.64 (broad s, 1H), 6.67 (d, 1H), 6.75 (s, 1H), 6.89 (d, 1H), 7.05 (d, 1H), 7.39 (d, 1H), 7.56 (s, 1H).

### Example 196

### ((1S)-5-{2-[5-Methyl-2-(3-methylphenyl)-1,3-thiazol-4-yl]ethoxy}-2,3-dihydro-1H-inden-1-yl)acetic Acid

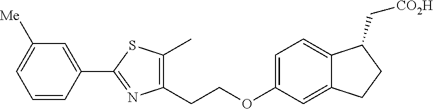 [SMILES: Cc1cccc(-c2nc(CCOc3ccc4c(c3)CC[C@H]4CC(=O)O)c(C)s2)c1]

(C₂₄H₂₅NO₃S): LC-MS, RT 3.71 min., M+H 408.2; ¹H NMR (CDCl₃): δ 1.70-1.82 (m, 1H), 2.38-2.52 (m, 2H), 2.40 (s, 3H), 2.47 (s, 3H), 2.75-2.87 (m, 3H), 3.19 (t, 2H), 3.45-3.60 (m, 1H), 4.29 (t, 2H), 6.72 (d, 1H), 6.78 (s, 1H), 7.07 (d, 1H), 7.19 (d, 1H), 7.30 (t, 1H), 7.64 (d, 1H), 7.75 (s, 1H).

### Example 197

### [(1S)-5-(2-{5-Methyl-2-[3-(trifluoromethyl)phenyl]-1,3-thiazol-4-yl}ethoxy)-2,3-dihydro-1H-inden-1-yl]acetic Acid

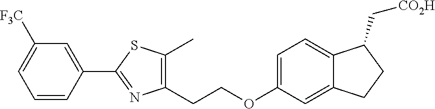 [SMILES: Cc1sc(-c2cccc(C(F)(F)F)c2)nc1CCOc1ccc2c(c1)CC[C@H]2CC(=O)O]

(C₂₄H₂₂F₃NO₃S): LC-MS, RT 3.90 min., M+H 462.1; ¹H NMR (CDCl₃): δ 1.70-1.82 (m, 1H), 2.38-2.48 (m, 2H), 2.49 (s, 3H), 2.75-2.87 (m, 3H), 3.19 (t, 2H), 3.44-3.59 (m, 1H), 4.30 (t, 2H), 6.72 (d, 1H), 6.79 (s, 1H), 7.07 (d, 1H), 7.52 (t, 1H), 7.61 (d, 1H), 8.01 (d, 1H), 8.13 (s, 1H).

### Example 198

### ((1S)-5-{2-[2-(3-Fluorophenyl)-5-methyl-1,3-thiazol-4-yl]ethoxy}-2,3-dihydro-1H-inden-1-yl)acetic Acid

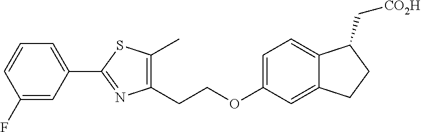 [SMILES: Cc1sc(-c2cccc(F)c2)nc1CCOc1ccc2c(c1)CC[C@H]2CC(=O)O]

(C₂₃H₂₂FNO₃S): LC-MS, RT 3.66 min., M+H 412.1; ¹H NMR (CDCl₃): δ 1.70-1.82 (m, 1H), 2.39-2.47 (m, 2H), 2.48 (s, 3H), 2.76-2.87 (m, 3H), 3.18 (t, 2H), 3.45-3.60 (m, 1H), 4.30 (t, 2H), 6.72 (d, 1H), 6.78 (s, 1H), 7.04-7.09 (m, 2H), 7.36-7.42 (m, 1H), 7.58-7.62 (m, 2H).

### Example 199

### ((1S)-5-{2-[2-(3,5-Dimethylphenyl)-5-methyl-1,3-thiazol-4-yl]ethoxy}-2,3-dihydro-1H-inden-1-yl)acetic Acid

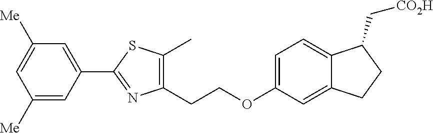 [SMILES: Cc1cc(C)cc(-c2nc(CCOc3ccc4c(c3)CC[C@H]4CC(=O)O)c(C)s2)c1]

(C₂₅H₂₇NO₃S): LC-MS, RT 3.88 min., M+H 422.2; ¹H NMR (CDCl₃): δ 1.72-1.84 (m, 1H), 2.36 (s, 6H), 2.37-2.45 (m, 2H), 2.46 (s, 3H), 2.75-2.87 (m, 3H), 3.19 (t, 2H), 3.45-3.60 (m, 1H), 4.28 (t, 2H), 6.72 (d, 1H), 6.79 (s, 1H), 7.01 (s, 1H), 7.07 (d, 1H), 7.48 (s, 2H).

### Example 200

### [(1S)-5-(2-{5-Methyl-2-[4-(trifluoromethoxy)phenyl]-1,3-thiazol-4-yl}ethoxy)-2,3-dihydro-1H-inden-1-yl]acetic Acid

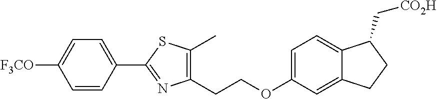 [SMILES: Cc1sc(-c2ccc(OC(F)(F)F)cc2)nc1CCOc1ccc2c(c1)CC[C@H]2CC(=O)O]

(C₂₄H₂₂F₃NO₄S): LC-MS, RT 3.95 min., M+H 478.1; ¹H NMR (CDCl₃): δ 1.72-1.84 (m, 1H), 2.38-2.46 (m, 2H), 2.47 (s, 3H), 2.75-2.87 (m, 3H), 3.18 (t, 2H), 3.45-3.60 (m, 1H), 4.29 (t, 2H), 6.72 (d, 1H), 6.77 (s, 1H), 7.07 (d, 1H), 7.24 (d, 2H), 7.88 (d, 2H).

### Example 201

### ((1S)-5-{2-[2-(3-Methoxyphenyl)-5-methyl-1,3-thiazol-4-yl]ethoxy}-2,3-dihydro-1H-inden-1-yl)acetic Acid

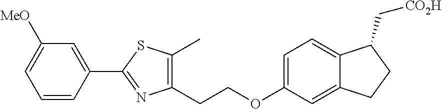 [SMILES: COc1cccc(-c2nc(CCOc3ccc4c(c3)CC[C@H]4CC(=O)O)c(C)s2)c1]

(C₂₄H₂₅NO₄S): LC-MS, RT 3.56 min., M+H 424.2; ¹H NMR (CDCl₃): δ 1.70-1.82 (m, 1H), 2.37-2.52 (m, 2H), 2.49 (s, 3H), 2.75-2.87 (m, 3H), 3.19 (t, 2H), 3.45-3.57 (m, 1H), 3.87 (s, 3H), 4.30 (t, 2H), 6.72 (d, 1H), 6.79 (s, 1H), 6.95 (d, 1H), 7.10 (d, 1H), 7.32 (t, 1H), 7.40-7.45 (m, 2H).

### Example 202

### ((1S)-5-{2-[2-(1,1′-Biphenyl-4-yl)-5-methyl-1,3-thiazol-4-yl]ethoxy}-2,3-dihydro-1H-inden-1-yl)acetic Acid

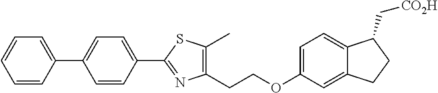 [SMILES: Cc1sc(-c2ccc(-c3ccccc3)cc2)nc1CCOc1ccc2c(c1)CC[C@H]2CC(=O)O]

(C₂₉H₂₇NO₃S): LC-MS, RT 3.96 min., M+H 470.3; ¹H NMR (CDCl₃): δ 1.70-1.81 (m, 1H), 2.38-2.48 (m, 2H), 2.49 (s, 3H), 2.75-2.87 (m, 3H), 3.20 (t, 2H), 3.43-3.59 (m, 1H), 4.31 (t, 2H), 6.72 (d, 1H), 6.79 (s, 1H), 7.08 (d, 1H), 7.36 (t, 1H), 7.45 (t, 2H), 7.61-7.65 (m, 4H), 7.93 (d, 2H).

### Example 203

### Preparation of ethyl {(1S)-5-[2-(4-methyl-2-phenyl-1,3-oxazol-5-yl)ethoxy]-2,3-dihydro-1H-inden-1-yl}acetate

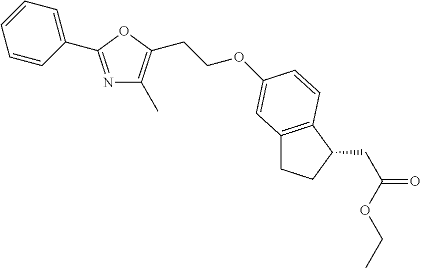 [SMILES: CCOC(=O)C[C@@H]1CCc2cc(OCCc3oc(-c4ccccc4)nc3C)ccc21]

ADDP (0.205 g, 0.81 mmol) was added to a mixture of PPh₃ (0.212 g, 0.81 mmol), ethyl [(1S)-5-hydroxy-2,3-dihydro-1H-inden-1-yl]acetate (0.107 g, 0.49 mmol), and 2-(4-methyl-2-phenyl-1,3-oxazol-5-yl)ethanol (step 4, Example 51, 0.110 g, 0.54 mmol) in THF (5 mL). The reaction was stirred overnight at rt, and additional ADDP (0.136 g, 0.54 mmol) and PPh₃ (0.141 g, 0.54 mmol) were added with CH₂Cl₂ (5 mL). The solution was stirred for 24 hours at rt and filtered. The filtrate was evaporated and the resulting mixture was purified by Biotage using a gradient 0 to 50% EtOAc/hexane. Gave ethyl {(1S)-5-[2-(4-methyl-2-phenyl-1,3-oxazol-5-yl)ethoxy]-2,3-dihydro-1H-inden-1-yl}acetate (0.145 g, 66% yield) as yellowish oil. ES-MS m/z 406.2 ((MH)⁺); HPLC RT (min.) 3.89; ¹H NMR (Acetone-d₆) δ 7.85-7.82 (m, 2H), 7.36-7.30 (m, 3H), 6.94 (d, 1H), 6.65 (s, 1H), 6.60-6.55 (m, 1H), 4.10 (t, 2H), 3.98 (q, 2H), 3.31-3.27 (m, 1H), 3.03 (t, 2H), 3.27-2.51 (m, 3H), 2.24-2.14 (m, 2H), 2.18 (s, 3H), 1.58-1.53 (m, 1H), 1.08 (t, 3H).

### Example 204

### Preparation of {(1S)-5-[2-(4-methyl-2-phenyl-1,3-oxazol-5-yl)ethoxy]-2,3-dihydro-1H-inden-1-yl}acetic Acid

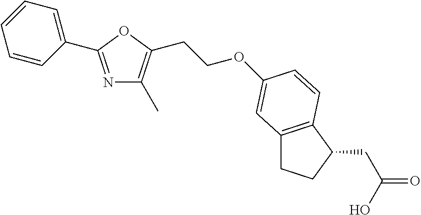 [SMILES: Cc1nc(-c2ccccc2)oc1CCOc1ccc2c(c1)CC[C@H]2CC(=O)O]

Ethyl {(1S)-5-[2-(4-methyl-2-phenyl-1,3-oxazol-5-yl)ethoxy]-2,3-dihydro-1H-inden-1-yl}acetate (0.135 g, 0.33 mmol) was dissolved in EtOH (6 mL) and LiOH (0.024 g, 1.0 mmol) was added. Water (3 mL) was added and THF was added until the cloudy solution became clear. The resulting mixture was stirred overnight at rt. HCl (2 N) was added to adjust the pH to 2, then extracted three times with ethyl acetate. The organic layers were combined, dried, and concentrated to give {(1S)-5-[2-(4-methyl-2-phenyl-1,3-oxazol-5-yl)ethoxy]-2,3-dihydro-1H-inden-1-yl}acetic acid (0.039 g, 30.6% yield) as colorless oil. ES-MS m/z 378.2 ((MH)⁺); HPLC RT (min.) 3.22; ¹H NMR (Acetone-d₆) δ 8.1 (s br 1H) 8.0-7.95 (m, 2H), 7.52-7.43 (m, 3H), 7.15 (d, 1H), 6.81 (s, 1H), 6.73 (d, 1H), 4.27 (t, 2H) 3.47-3.40 (m, 1H), 3.18 (t, 2H), 2.90-2.68 (m, 3H), 2.41-2.29 (m, 2H), 2.18 (s, 3H), 1.77-1.68 (m, 1H).

By using the procedure described above for Examples 51, 203, and 204 and substituting the appropriate starting materials, the following compounds were similarly prepared and characterized.

### Example 205

### Preparation of N-(4-methylbenzoyl)alanine

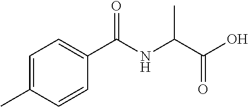

¹H NMR (DMSO-d₆) δ 12.60 (s br, 1H), 8.57 (d, 1H), 7.81 (d, 2H), 7.28 (d, 2H), 4.38 (q, 1H), 2.35 (s, 3H), 1.38 (d, 3H).

### Example 206

### Preparation of N-(3-fluoro-4-methylbenzoyl)alanine

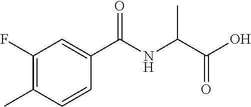 [SMILES: Cc1ccc(C(=O)NC(C)C(=O)O)cc1F]

¹H NMR (DMSO-d₆) δ 12.54 (s br, 1H), 8.67 (d, 1H), 7.65-7.62 (m, 2H), 7.39 (t, 1H), 4.38 (q, 1H), 2.27 (s, 3H), 1.38 (d, 3H).

### Example 207

### Preparation of N-[4-(trifluoromethyl)benzoyl]alanine

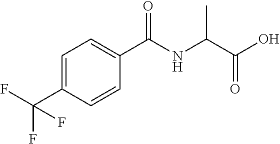 [SMILES: CC(NC(=O)c1ccc(C(F)(F)F)cc1)C(=O)O]

¹H NMR (DMSO-d₆) δ 12.64 (s br, 1H), 8.91 (d, 1H), 8.08 (d, 2H), 7.85 (d, 2H), 4.42 (q, 1H), 1.40 (d, 3H).

### Example 208

### Preparation of ethyl 4-[(4-methylbenzoyl)amino]-3-oxopentanoate

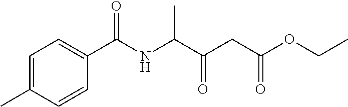 [SMILES: CCOC(=O)CC(=O)C(C)NC(=O)c1ccc(C)cc1]

ES-MS m/z 278.38 ((MH)⁺); HPLC RT (min.) 2.04. ¹H NMR (Acetone-d₆) δ 8.08 (s br, 1H), 7.90 (d, 2H), 7.28 (d, 2H), 4.72-4.67 (m, 1H), 4.13 (q, 2H), 3.66 (s, 2H), 2.40 (s, 3H), 1.41 (d, 3H), 1.12 (t, 3H).

### Example 209

### Preparation of ethyl 4-[(3-fluoro-4-methylbenzoyl)amino]-3-oxopentanoate

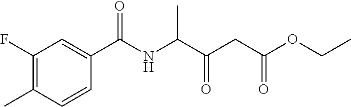 [SMILES: CCOC(=O)CC(=O)C(C)NC(=O)c1ccc(C)c(F)c1]

ES-MS m/z 296.4 ((MH)⁺); HPLC RT (min.) 2.26. ¹H NMR (Acetone-d₆) δ 7.75-7.60 (m, 2H), 7.38 (t, 1H), 4.20 (q, 2H), 3.65 (s, 2H), 2.23 (s, 3H), 1.45 (d, 3H), 1.20 (t, 3H).

### Example 210

### Preparation of ethyl 3-oxo-4-{[4-(trifluoromethyl)benzoyl]amino}pentanoate

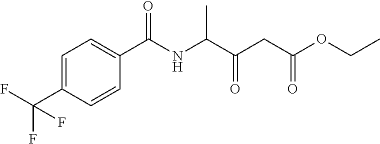 [SMILES: CCOC(=O)CC(=O)C(C)NC(=O)c1ccc(C(F)(F)F)cc1]

ES-MS m/z 332.4 ((MH)⁺); HPLC RT (min.) 2.45. ¹H NMR (Acetone-d₆) δ 8.14 (d, 2H), 7.84 (d, 2H), 4.80-4.74 (m, 2H), 4.20 (q, 2H), 3.70 (s, 2H), 1.48 (d, 3H), 1.21 (t, 3H).

### Example 211

### Preparation of ethyl [4-methyl-2-(4-methylphenyl)-1,3-oxazol-5-yl]acetate

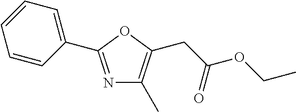 [SMILES: CCOC(=O)Cc1oc(-c2ccccc2)nc1C]

ES-MS m/z 260.2 ((MH)⁺); HPLC RT (min.) 2.96. ¹H NMR (Acetone-d₆) δ 7.86 (d, 2H), 7.30 (d, 2H), 4.15 (q, 2H), 3.81 (s, 2H), 2.37 (s, 3H), 2.14 (s, 3H), 1.24 (t, 3H).

### Example 212

### Preparation of ethyl [2-(3-fluoro-4-methylphenyl)-4-methyl-1,3-oxazol-5-yl]acetate

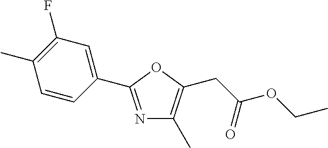 [SMILES: CCOC(=O)Cc1oc(-c2ccc(C)c(F)c2)nc1C]

ES-MS m/z 278.3 ((MH)⁺); HPLC RT (min.) 2.89. ¹H NMR (Acetone-d₆) δ 7.69 (d, 1H), 7.60 (d, 1H), 7.37 (t, 1H), 4.15 (q, 2H), 3.83 (s, 2H), 2.31 (s, 3H), 2.15 (s, 3H), 1.23 (t, 3H).

### Example 213

### Preparation of ethyl {4-methyl-2-[4-(trifluoromethyl)phenyl]-1,3-oxazol-5-yl}acetate

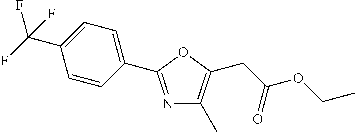 [SMILES: CCOC(=O)Cc1oc(-c2ccc(C(F)(F)F)cc2)nc1C]

ES-MS m/z 314.3 ((MH)⁺); HPLC RT (min.) 3.27. ¹H NMR (Acetone-d₆) δ 8.18 (d, 2H), 7.84 (d, 2H), 4.17 (q, 2H), 3.88 (s, 2H), 2.20 (s, 3H), 1.23 (t, 3H).

### Example 214

### Preparation of 2-[4-methyl-2-(4-methylphenyl)-1,3-oxazol-5-yl]ethanol

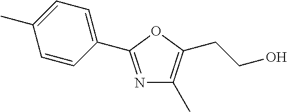 [SMILES: Cc1ccc(-c2nc(C)c(CCO)o2)cc1]

ES-MS m/z 218.2 ((MH)⁺); HPLC RT (min.) 2.35. ¹H NMR (Acetone d₆) δ 7.85 (d, 2H), 7.27 (d, 2H), 3.99 (s br, 1H), 3.83 (t, 2H), 2.90 (t, 2H), 2.37 (s, 3H), 2.12 (s, 3H).

### Example 215

### Preparation of 2-[2-(3-fluoro-4-methylphenyl)-4-methyl-1,3-oxazol-5-yl]ethanol

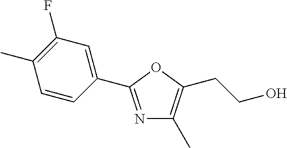 [SMILES: Cc1ccc(-c2nc(C)c(CCO)o2)cc1F]

ES-MS m/z 236.2 ((MH)⁺); HPLC RT (min.) 2.46. ¹H NMR (CDCl₃) δ 7.54 (d, 1H), 7.43 (d, 1H), 7.17 (t, 1H), 3.91 (d, 2H), 3.09 (s br, 1H), 2.88 (t, 2H), 2.29 (s, 3H), 2.13 (s, 3H).

### Example 216

### Preparation of 2-{4-methyl-2-[4-(trifluoromethyl)phenyl]-1,3-oxazol-5-yl}ethanol

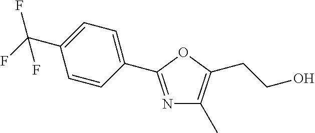 [SMILES: Cc1nc(-c2ccc(C(F)(F)F)cc2)oc1CCO]

ES-MS m/z 272.2 ((MH)⁺); HPLC RT (min.) 2.71. ¹H NMR (CDCl₃) δ 8.03 (2, 2H), 7.66 (d, 2H), 3.95 (t, 2H), 2.96 (t, 2H), 2.21 (s, 3H), 1.97 (s br, 1H).

### Example 217

### Preparation of ethyl [(1S)-5-(2-{4-methyl-2-[4-(trifluoromethyl)phenyl]-1,3-oxazol-5-yl}ethoxy)-2,3-dihydro-1H-inden-1-yl]acetate

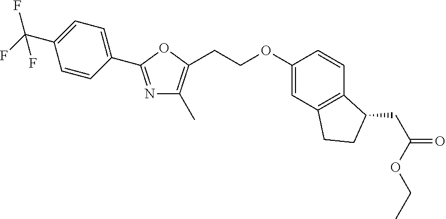 [SMILES: CCOC(=O)C[C@@H]1CCc2cc(OCCc3oc(-c4ccc(C(F)(F)F)cc4)nc3C)ccc21]

ES-MS m/z 474.5 ((MH)⁺); HPLC RT (min.) 4.10. ¹H NMR (Acetone-d₆) δ 8.16 (d, 2H), 7.83 (d, 2H), 7.09 (d, 1H), 6.80 (s, 1H), 6.72 (dd, 1H), 4.28 (t, 2H), 4.12 (q, 2H), 3.46-3.41 (m, 1H), 3.21 (t, 2H), 2.86-2.65 (m, 3H), 2.39-2.26 (m, 2H), 2.20 (s, 3H), 1.75-1.63 (m, 1H), 1.22 (t, 3H).

### Example 218

### Preparation of ethyl ((1S)-5-{2-[4-methyl-2-(4-methylphenyl)-1,3-oxazol-5-yl]ethoxy}-2,3-dihydro-1H-inden-1-yl)acetate

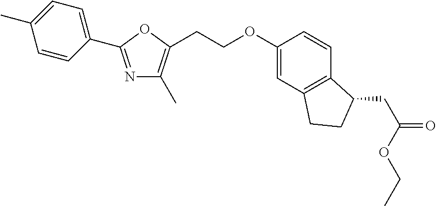 [SMILES: CCOC(=O)C[C@@H]1CCc2cc(OCCc3oc(-c4ccc(C)cc4)nc3C)ccc21]

TCL Rf=0.22 Hexane/EtOAc 4:1

### Example 219

### Preparation of ethyl ((1S)-5-{2-[2-(3-fluoro-4-methylphenyl)-4-methyl-1,3-oxazol-5-yl]ethoxy}-2,3-dihydro-1H-inden-1-yl)acetate

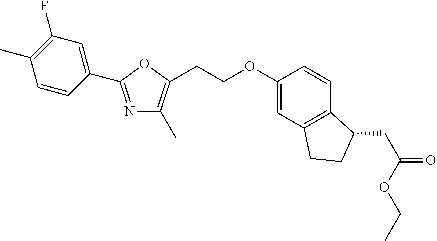 [SMILES: CCOC(=O)C[C@@H]1CCc2cc(OCCc3oc(-c4ccc(C)c(F)c4)nc3C)ccc21]

ES-MS m/z 438.2 ((MH)⁺); HPLC RT (min.) 4.18. ¹H NMR (Acetone-d₆) δ 6.67 (dd, 1H), 7.59 (dd, 1H), 7.37 (t, 1H), 7.08 (d, 1H), 6.80 (s, 1H), 6.72 (dd, 1H), 4.26 (t, 2H), 4.12 (q, 2H), 3.46-3.38 (m, 1H), 3.17 (t, 2H), 2.89-2.65 (m, 3H), 2.39-2.23 (m, 5H), 2.17 (s, 3H), 1.75-1.63 (m, 1H), 1.23 (t, 3H).

### Example 220

### Preparation of ((1S)-5-{2-[4-methyl-2-(4-methylphenyl)-1,3-oxazol-5-yl]ethoxy}-2,3-dihydro-1H-inden-1-yl)acetic Acid

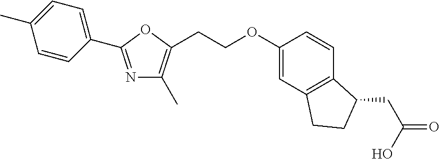 [SMILES: Cc1ccc(-c2nc(C)c(CCOc3ccc4c(c3)CC[C@H]4CC(=O)O)o2)cc1]

ES-MS m/z 392.2 ((MH)⁺); HPLC RT (min.) 3.36. ¹H NMR (Acetone-d₆) δ 7.72 (d, 2H), 7.15 (d, 2H), 6.99 (d, 1H), 6.67 (s, 1H), 6.59 (dd, 1H), 4.12 (t, 2H), 3.33-3.28 (m, 1H), 3.03 (t, 2H), 2.73-2.54 (m, 3H), 2.27-2.21 (m, 5H), 2.02 (s, 3H), 1.64-1.54 (m, 1H).

### Example 221

### Preparation of ((1S)-5-{2-[2-(3-fluoro-4-methylphenyl)-4-methyl-1,3-oxazol-5-yl]ethoxy}-2,3-dihydro-11-inden-1-yl)acetic Acid

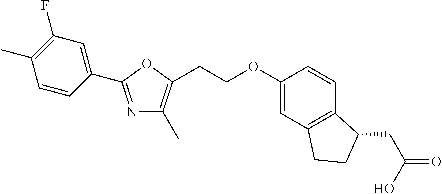 [SMILES: Cc1ccc(-c2nc(C)c(CCOc3ccc4c(c3)CC[C@H]4CC(=O)O)o2)cc1F]

ES-MS m/z 410.2 ((MH)⁺); HPLC RT (min.) 3.49. ¹H NMR (Acetone-d₆) δ 7.68 (dd, 1H), 7.59 (dd, 1H), 7.36 (t, 1H), 7.12 (d, 1H), 6.80 (s, 1H), 6.72 (dd, 1H), 4.26 (t, 2H), 3.47-3.41 (m, 1H, 3.18 (t, 2H), 2.86-2.67 (m, 3H), 2.40-2.28 (m, 5H), 2.17 (s, 3H), 1.18-1.65 (m, 1H).

### Example 222

### Preparation of [(1S)-5-(2-{4-methyl-2-[4-(trifluoromethyl)phenyl]-1,3-oxazol-5-yl}ethoxy)-2,3-dihydro-1H-inden-1-yl]acetic Acid

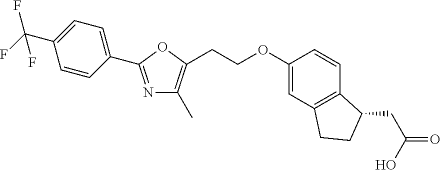 [SMILES: Cc1nc(-c2ccc(C(F)(F)F)cc2)oc1CCOc1ccc2c(c1)CC[C@H]2CC(=O)O]

ES-MS m/z 446.5 ((MH)⁺); HPLC RT (min.) 3.47. ¹H NMR (Acetone-d₆) δ 8.17 (d, 2H), 7.84 (d, 2H), 7.13 (s, 1H), 6.80 (s, 1H), 6.72 (dd, 1H), 4.28 (t, 2H), 3.46-3.41 (m, 1H), 3.21 (t, 2H), 2.86-2.67 (m, 3H), 2.40-2.28 (m, 2H), 2.20 (s, 3H), 1.77-1.67 (m, 1H).

### Example 223

### Preparation of (2S)-2-{(1S)-5-[2-(5-methyl-2-phenyl-1,3-oxazol-4-yl)ethoxy]-2,3-dihydro-1H-inden-1-yl}propanoic Acid and (2R)-2-{(1R)-5-[2-(5-methyl-2-phenyl-1,3-oxazol-4-yl)ethoxy]-2,3-dihydro-1H-inden-1-yl}propanoic acid

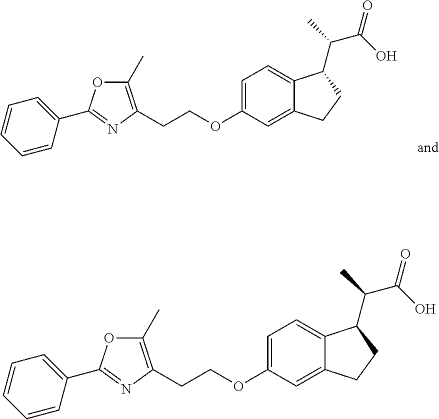 [SMILES: Cc1oc(-c2ccccc2)nc1CCOc1ccc2c(c1)CC[C@H]2[C@H](C)C(=O)O ; Cc1oc(-c2ccccc2)nc1CCOc1ccc2c(c1)CC[C@@H]2[C@@H](C)C(=O)O]

Step 1. Preparation of (2S)-2-[(1S)-5-methoxy-2,3-dihydro-1H-inden-1-yl]propanoic acid and (2R)-2-[(1R)-5-methoxy-2,3-dihydro-1H-inden-1-yl]propanoic acid

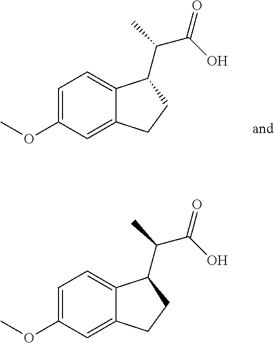 [SMILES: COc1ccc2c(c1)CC[C@H]2[C@H](C)C(=O)O ; COc1ccc2c(c1)CC[C@@H]2[C@@H](C)C(=O)O]

The starting acid (Example 2b) was reacted using a similar procedure as described in Example 4, under 60 psi H₂, and using 4.5 g starting material, 1.04 g catalyst, and 4.5 mL triethylamine in 45 mL ethanol and 5 mL THF. The standard extractive workup gave 3.22 g product. LC/MS retention time 2.41 min., NMR (d6-DMSO): 0.87 (d, 3H, α-methyl), 1.75 (m, 1H), 2.04 (m, 1H), 3.66 (s, 3H, methoxy), 6.65 (m, 1H, aryl), 6.76 (s, 1H, aryl), 7.04 (d, 1H, aryl,) 12.18 (bs, 1H, acid.)

Step 2: Preparation of methyl (2S)-2-[(1S)-5-methoxy-2,3-dihydro-1H-inden-1-yl]propanoate and methyl (2R)-2-[(1R)-5-methoxy-2,3-dihydro-1H-inden-1-yl]propanoate

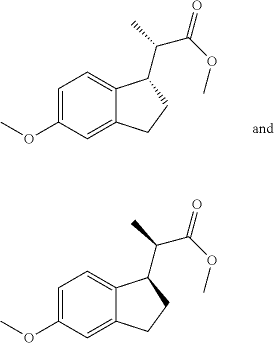 [SMILES: COC(=O)[C@@H](C)[C@@H]1CCc2cc(OC)ccc21 ; COC(=O)[C@H](C)[C@H]1CCc2cc(OC)ccc21]

The compound was prepared by the reaction of 1.5 g starting acid, 0.93 mL iodomethane, and 1.75 g sodium bicarbonate in 10 mL methanol under standard esterification conditions as described in Example 6. Workup gave 1.53 g, 96%. (NMR (CD₂Cl₂): 1.05 (d, 3H, α-methyl), 1.88 (m, 1H), 2.19 (m, 1H), 3.44 (m, 1H), 3.68 (s, 3H, methoxy), 3.77 (s, 3H, ester).

Step 3. Preparation of: methyl (2S)-2-[(1S)-5-hydroxy-2,3-dihydro-1H-inden-1-yl]propanoate and methyl (2R)-2-[(1R)-5-hydroxy-2,3-dihydro-1H-inden-1-yl]propanoate

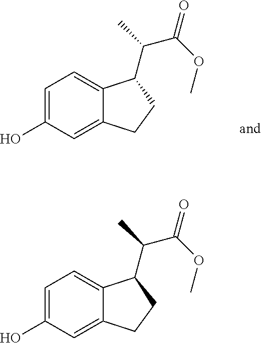 [SMILES: COC(=O)[C@@H](C)[C@@H]1CCc2cc(O)ccc21 ; COC(=O)[C@H](C)[C@H]1CCc2cc(O)ccc21]

Using the demethylation conditions as described in Example 7 (1.53 g starting material, 4.35 g AlCl₃, and 2.4 mL ethanethiol in 20 mL dichloromethane), 1.21 g of product (84%) was obtained. (NMR (CD₂Cl₂): 1.05 (d, 3H, α-methyl), 1.88 (m, 1H), 2.18 (m, 1H), 3.45 (m, 1H), 3.67 (s, 3H, ester), 6.60 (m, 1H, aryl), 6.69 (s, 1H, aryl), 6.93 (d, 1H, aryl.)

Step 4: Preparation of methyl (2S)-2-{(1S)-5-[2-(5-methyl-2-phenyl-1,3-oxazol-4-yl)ethoxy]-2,3-dihydro-1H-inden-1-yl}propanoate and methyl (2R)-2-{(1R)-5-[2-(5-methyl-2-phenyl-1,3-oxazol-4-yl)ethoxy]-2,3-dihydro-1H-inden-1-yl}propanoate

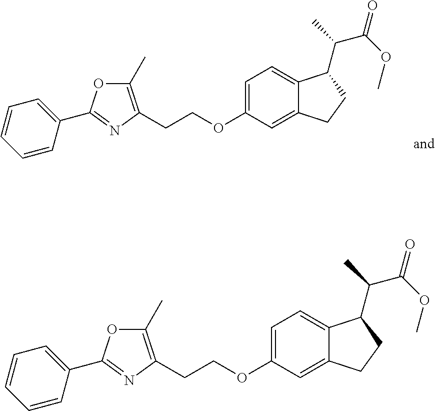 [SMILES: COC(=O)[C@@H](C)[C@@H]1CCc2cc(OCCc3nc(-c4ccccc4)oc3C)ccc21 ; COC(=O)[C@H](C)[C@H]1CCc2cc(OCCc3nc(-c4ccccc4)oc3C)ccc21]

Using the standard Mitsunobu coupling procedure as described in Example 11 (0.100 g starting phenol, 0.110 g oxazolylethanol, 0.143 g triphenylphosphine, and 0.137 g ADDP in 2 mL dichloromethane), 0.107 g (58%) of product was obtained after chromatography in 15% EtOAc/hexane. NMR (CD₂Cl₂): 1.62-1.87 (m, 4H), 2.40 (s, 3H, oxazole methyl), 2.98 (t, 2H, methylene), 3.23 (m, 1H), 3.63 (s, 3H, ester), 6.60 (s, 1H, aryl), 6.64 (m, 1H, aryl), 7.42 (m, 3H, aryl), 8.00 (m, 2H, aryl).

Step 5. (25)-2-{(1S)-5-[2-(5-methyl-2-phenyl-1,3-oxazol-4-yl)ethoxy]-2,3-dihydro-1H-inden-1-yl}propanoic acid and (2R)-2-{(1R)-5-[2-(5-methyl-2-phenyl-1,3-oxazol-4-yl)ethoxy]-2,3-dihydro-1H-inden-1-yl}propanoic acid

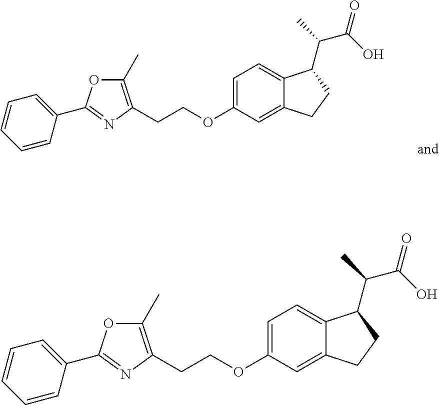 [SMILES: Cc1oc(-c2ccccc2)nc1CCOc1ccc2c(c1)CC[C@H]2[C@H](C)C(=O)O ; Cc1oc(-c2ccccc2)nc1CCOc1ccc2c(c1)CC[C@@H]2[C@@H](C)C(=O)O]

The LiOH hydrolysis conditions were applied to 0.090 g of starting ester, yielding 0.082 g (95%) product. NMR (CD₃OD): 0.4-0.75 (m, 4H), 1.18 (s, 3H), 1.75 (t, 2H, methylene), 2.00 (m, 1H), 2.99 (t, 2H, methylene), 5.39 (s, 1H, aryl), 5.48 (m, 1H, aryl), 5.83 (d, 1H, aryl), 6.27 (m, 3H, aryl), 6.76 (m, 2H, aryl).

Using the methods described above and the appropriate starting materials, additional (2S, 1S) and (2R, 1R) were similarly prepared, either as diastereomeric (i.e., syn, {(2S, 1S)/(2R, 1R)} and or anti {(2R, 1S)/(2S, 1R)}) mixtures, or as individual enantiomers. These compounds are summarized in Table 5.

TABLE 5

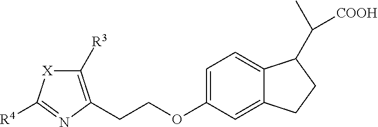

| Ex. | | | | | HPLC RT | LC-MS |
|---|---|---|---|---|---|---|
| No. | R³ | R⁴ | X | Isomer | (min) | [M + H]⁺ |
| 224 | Me | 3,4-(Cl)₂—Ph | O | 2S,1S | 4.10 | 460.0 |
| 225 | Me | 3,4-(Cl)₂—Ph | O | syn | 4.10 | 460.0 |
| | | | | racemate | | |
| 226 | Me | 3,4-(Me)₂—Ph | O | syn | 4.32 | 420.4 |
| | | | | racemate | | |
| 227 | Me | 3,4-(Me)₂—Ph | O | 2S,1S | 4.32 | 420.4 |
| 228 | Me | 3-Me—Ph | O | syn | 4.19 | 406.3 |
| | | | | racemate | | |
| 229 | Me | 4-CF₃—Ph | O | syn | 3.73 | 460.2 |
| | | | | racemate | | |
| 230 | Me | 4-CF₃—Ph | O | 2S,1S | 3.73 | 460.2 |
| 231 | Me | 4-CF₃—Ph | O | 2R,1R | 3.73 | 460.2 |
| 232 | Me | 4-Cl—Ph | O | syn | 3.61 | 426.2 |
| | | | | racemate | | |
| 233 | Me | 4-Et—Ph | O | syn | 3.70 | 420.3 |
| | | | | racemate | | |
| 234 | Me | 4-Et—Ph | O | 2S,1S | 3.70 | 420.3 |
| 235 | Me | 4-Et—Ph | O | 2R,1R | 3.70 | 420.3 |
| 236 | Me | 4-Et—Ph | O | syn/anti | 3.70 | 420.3 |
| | | | | mixture | | |
| 237 | Me | 4-Et—Ph | O | 2R,1S | 3.70 | 420.3 |
| 238 | Me | 4-Et—Ph | O | 2S,1R | 3.70 | 420.3 |
| 239 | Me | 4-MeO—Ph | O | syn | 3.37 | 422.3 |
| | | | | racemate | | |
| 240 | Me | 4-MeO—Ph | O | 2R,1R | 3.37 | 422.3 |
| 241 | Me | 4-MeO—Ph | O | 2S,1S | 3.37 | 422.3 |
| 242 | Me | 4-n-Bu—Ph | O | syn | 4.08 | 448.4 |
| | | | | racemate | | |
| 243 | Me | 4-t-Bu—Ph | O | 2S,1S | 4.59 | 448.4 |
| 244 | Et | 4-t-Bu—Ph | O | syn | 4.59 | 448.4 |
| | | | | racemate | | |
| 245 | Me | 4-MeO—Ph | O | 2S,1S | 3.58 | — |
| 246 | Me | 4-Cl—Ph | S | syn | 3.84 | 442.2 |
| | | | | racemate | | |
| 247 | Me | 4-Me—Ph | S | syn | 4.34 | 422.3 |
| | | | | racemate | | |

### Example 248

### Preparation of ethyl [(1S)-5-(2-{2-[4′-(5-acetyl-2-thienyl)-1,1′-biphenyl-4-yl]-5-methyl-1,3-oxazol-4-yl}ethoxy)-2,3-dihydro-1H-inden-1-yl]acetate

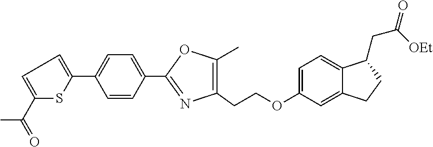 [SMILES: CCOC(=O)C[C@@H]1CCc2cc(OCCc3nc(-c4ccc(-c5ccc(C(C)=O)s5)cc4)oc3C)ccc21]

To a solution containing ethyl ((1S)-5-{2-[2-(4-bromophenyl)-5-methyl-1,3-oxazol-4-yl]ethoxy}-2,3-dihydro-1H-inden-1-yl)acetate (0.100 g, 0.21 mmol) [prepared from 2-[5-methyl-2-(4-bromophenyl)-1,3-oxazol-4-yl]ethanol and ethyl [(1S)-5-hydroxy-2,3-dihydro-1H-inden-1-yl]acetate (Example 135)], 1,1′-bis(diphenylphosphino)-ferrocene]dichloro palladium(II) (16.9 mg, 0.02 mmol), and 5-acetyl-2-thienylboronic acid (0.062 g, 0.41 mmol) in degassed toluene and dioxane (4:1, 2 mL) was added aqueous 2 M sodium carbonate (0.5 mL). The mixture was heated at 85° C. for 16 hours. Solvents were evaporated under vacuum and the residue was dissolved in methanol and acetonitrile and filtered through a C8 reverse phase extraction cartridge. Solvents were evaporated and the residue was dissolved in acetonitrile and purified by HPLC to obtain ethyl [(1S)-5-(2-{2-[4′-(5-acetyl-2-thienyl)-1,1′-biphenyl-4-yl]-5-methyl-1,3-oxazol-4-yl}ethoxy)-2,3-dihydro-1H-inden-1-yl]acetate in 46% yield. (50 mg, 0.09 mmol) MS (electro spray) 530.4 (M+H)⁺, ¹H NMR (CDCl₃) δ 1.24 (t, 3H), 1.71 (m, 1H), 2.37 (m, 5H), 2.57 (s, 3H), 2.68 (m, 1H), 2.83 (m, 2H), 3.03 (m, 2H), 3.48 (m, 1H), 4.17 (m, 4H), 6.67 (m, 2H), 7.02 (d, 1H), 7.39 (d, 1H), 7.67 (d, 1H), 7.73 (d, 2H), 8.01 (d, 2H).

Other compounds, prepared by using analogous starting materials and the method described in Example 248 together with the hydrolysis described in Example 11, are described below in Table 6.

TABLE 6

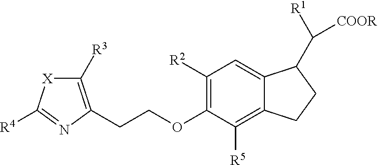

| Ex. | | | | | | | | LC-MS |
|---|---|---|---|---|---|---|---|---|
| No. | R | R¹ | R² | R³ | R⁴ | R⁵ | X | [M + H]⁺ |
| 249 | H | H | H | Me | 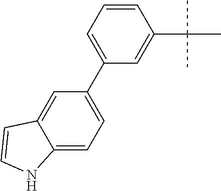 | H | O | 493.3 |
| 250 | H | H | H | Me | 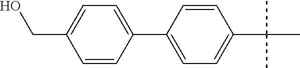 | H | O | 484.2 |
| 251 | H | H | H | Me | 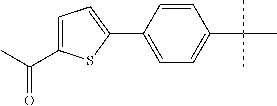 | H | O | 502.2 |

The foregoing is illustrative of the present invention and is not to be construed as limiting thereof. Although a few exemplary embodiments of this invention have been described, those skilled in the art will readily appreciate that many modifications are possible in the exemplary embodiments without materially departing from the novel teachings and advantages of this invention. Accordingly, all such modifications are intended to be included within the scope of this invention as defined in the claims. Therefore, it is to be understood that the foregoing is illustrative of the present invention and is not to be construed as limited to the specific embodiments disclosed, and that modifications to the disclosed embodiments, as well as other embodiments, are intended to be included within the scope of the appended claims. The invention is defined by the following claims, with equivalents of the claims to be included therein.

That which is claimed is:

1. A method of treating or preventing a cognitive disorder comprising administering to a subject in need thereof an effective amount of a compound of Formula I: 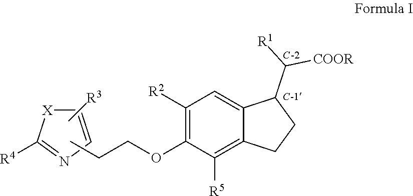
wherein in Formula I
R is H or C₁-C₆ alkyl;
R¹ is H, COOR, C₃-C₈ cycloalkyl, or C₁-C₆ alkyl, C₂-C₆ alkenyl, or C₁-C₆ alkoxy each of which may be unsubstituted or substituted with fluoro, methylenedioxyphenyl, or phenyl which may be unsubstituted or substituted with R⁶;
R² is H, halo, or C₁-C₆ alkyl which may be unsubstituted or substituted with C₁-C₆ alkoxy, oxo, fluoro, or
R² is phenyl, furyl, thienyl, pyrrolyl, oxazolyl, thiazolyl, imidazolyl, pyrazolyl, isoxazolyl, isothiazolyl, triazolyl, oxadiazolyl, thiadiazolyl, tetrazolyl, pyridyl, pyrrolidinyl, piperidinyl, tetrahydropyranyl, tetrahydrothiopyranyl, piperazinyl, or morpholinyl,
each of which may be unsubstituted or substituted with R⁶;
R³ is H, C₁-C₆ alkyl, or phenyl, which may be unsubstituted or substituted with R⁶;
X is O or S;
R⁴ is C₁-C₆ alkyl or C₃-C₈ cycloalkyl, either of which may be unsubstituted or substituted with fluoro, oxo, or C₁-C₆ alkoxy which may be unsubstituted or substituted with C₁-C₆ alkoxy, or phenyl optionally substituted with R⁶,
each of which may be substituted with phenyl, naphthyl, furyl, thienyl, pyrrolyl, tetrahydrofuryl, pyrrolidinyl, pyrrolinyl, tetrahydrothienyl, oxazolyl, thiazolyl, imidazolyl, pyrazolyl, isoxazolyl, isothiazolyl, triazolyl, oxadiazolyl, thiadiazolyl, tetrazolyl, pyridyl, piperidinyl, tetrahydropyranyl, tetrahydrothiopyranyl, pyrimidinyl, pyrazinyl, pyridazinyl, piperazinyl, morpholinyl, benzofuryl, dihydrobenzofuryl, benzothienyl, dihydrobenzothienyl, indolyl, indolinyl, indazolyl, benzoxazolyl, benzothiazolyl, benzimidazolyl, benzisoxazolyl, benzisothiazolyl, benzodioxolyl, quinolyl, isoquinolyl, quinazolinyl, quinoxazolinyl, dihydrobenzopyranyl, dihydrobenzothiopyranyl, or 1,4-benzodioxanyl,
each of which may be unsubstituted or further substituted with R⁶, or
C₁-C₆ alkyl may also be substituted with C₃-C₈ cycloalkyl or with phenoxy which may be unsubstituted or substituted with R⁶ or with phenyl, naphthyl, furyl, thienyl, pyrrolyl, tetrahydrofuryl, pyrrolidinyl, pyrrolinyl, tetrahydrothienyl, oxazolyl, thiazolyl, imidazolyl, pyrazolyl, isoxazolyl, isothiazolyl, triazolyl, oxadiazolyl, thiadiazolyl, tetrazolyl, pyridyl, piperidinyl, tetrahydropyranyl, tetrahydrothiopyranyl, pyrimidinyl, pyrazinyl, pyridazinyl, piperazinyl, morpholinyl, benzofuryl, dihydrobenzofuryl, benzothienyl, dihydrobenzothienyl, indolyl, indolinyl, indazolyl, benzoxazolyl, benxothiazolyl, benzimidazolyl, benzisoxazolyl, benzisothiazolyl, benzodioxolyl, quinolyl, isoquinolyl, quinazolinyl, quinoxazolinyl,
dihydrobenzopyranyl, dihydrobenzothiopyranyl, or 1,4-benzodioxanyl,
each of which may be unsubstituted or substituted with R⁶, or
R⁴ is phenyl, naphthyl, furyl, thienyl, pyrrolyl, tetrahydrofuryl, pyrrolidinyl, pyrrolinyl, tetrahydrothienyl, oxazolyl, thiazolyl, imidazolyl, pyrazolyl, isoxazolyl, isothiazolyl, triazolyl, oxadiazolyl, thiadiazolyl, tetrazolyl, pyridyl, piperidinyl, tetrahydropyranyl, tetrahydrothiopyranyl, pyrimidinyl, pyrazinyl, pyridazinyl, piperazinyl, morpholinyl, benzofuryl, dihydrobenzofuryl, benzothienyl, dihydrobenzothienyl, indolyl, indolinyl, indazolyl, benzoxazolyl, benxothiazolyl, benzimidazolyl, benzisoxazolyl, benzisothiazolyl, benzodioxolyl, quinolyl, isoquinolyl, quinazolinyl, quinoxazolinyl, dihydrobenzopyranyl, dihydrobenzothiopyranyl, or 1,4-benzodioxanyl,
each of which may be unsubstituted or substituted with R⁶, or with phenyl, furyl, thienyl, pyrrolyl, oxazolyl, thiazolyl, imidazolyl, pyrazolyl, isoxazolyl, isothiazolyl, triazolyl, oxadiazolyl, thiadiazolyl, tetrazolyl, pyridyl, pyrrolidinyl, piperidinyl, tetrahydropyranyl, tetrahydrothiopyranyl, piperazinyl, morpholinyl, benzodioxolyl, dihydrobenzofuranyl, indolyl, pyrimidinyl or phenoxy, each of which may be unsubstituted or substituted with R⁶;
R⁵ is H, halo or C₁-C₆ alkyl optionally substituted with oxo; and
R⁶ is halo, CF₃, C₁-C₆ alkyl optionally substituted with oxo or hydroxy, or
C₁-C₆ alkoxy optionally substituted with fluoro;
or a pharmaceutically acceptable salt, ester, prodrug, stereoisomer, diastereomer, enantiomer, racemate or a combination thereof.

2. The method according to claim 1 wherein the compound has the following structure:

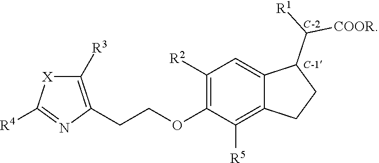

3. The method according to claim 1 wherein:
R is H,
R¹ is H,
R² is H,
R³ is C₁-C₆ alkyl,
X is O, and
R⁴ is a phenyl substituted with R⁶, wherein R⁶ is C₁-C₆ alkoxyl or C₁-C₆ alkyl.

4. The method according to claim 1 wherein the compound has the following structure:

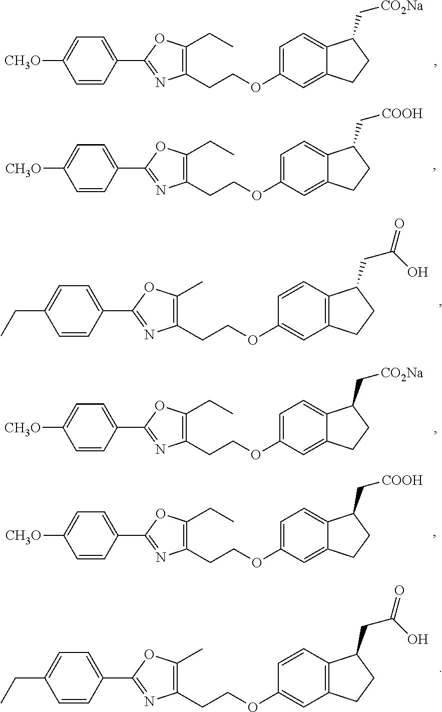

5. The method according to claim 1 wherein the compound is a pharmaceutically acceptable salt thereof, wherein the pharmaceutically acceptable salt is selected from the group consisting of alkali metal salts, alkaline earth metal salts, ammonium salts with organic bases, and basic nitrogen containing groups in the conjugate base that is quaternized with agents selected from the group consisting of alkyl halides and aralkyl.

6. The method according to claim 5 wherein the compound is a meglumine, potassium or sodium salt thereof.

7. The method according to claim 1 wherein said compound is administered intravenously, orally, buccally, transdermally, rectally, nasally, otically, intrathecally or intra-cranially

8. The method according to claim 1 further comprising administration of one or more additional therapeutic agent.

9. The method according to claim 8 wherein said one or more additional therapeutic agent is used to treat or prevent Alzheimer's disease.

10. The method according to claim 9 wherein said one or more additional therapeutic agents is at least one of acetylcholinesterase inhibitor, and a NMDA receptor antagonist.

11. The method according to claim 10 wherein said additional therapeutic agent is selected from the group consisting of tacrine, galantamine, rivastigamine, donepezil and memantine.

12. The method according to claim 9 wherein said one or more additional therapeutic agents reglates beta amyloid plaque disease is selected from the group consisting of an antioxidant, an anti-inflammatory, a gamma secretase inhibitor, a neurotrophic agent, an acetyl cholinesterase inhibitor, HMG-CoA reductase inhibitors (or statin), an A beta peptide, and an anti-A beta peptide.

13. The method according to claim 12 wherein the agent that regulates beta amyloid plaque is selected from the group consisting of:
a) beta-amyloid peptide synthesis inhibitors;
b) amyloid plaque inhibitors that block beta amyloid peptide aggregation through passive immunization;
c) muscarinic receptor modulators;
d) phosphodiesterase-4 inhibitors; and
e) chelating agents.

14. The method according to claim 9 wherein said one or more additional therapeutic agent is selected from the group consisting of bapineuzumab, solanezumab, gammagaard, MABT5102H, AN-1792, ACC-001, affitope AD02, CAD-106, MK-8951, HPP854, RG7129, E2609 and LY2886721.

15. The method according to claim 9 wherein at least one of the additional therapeutic agents regulates tau-mediated neurodegeneration and formation of tau neurofibrillary tangles.

16. The method according to claim 15 wherein the therapeutic agent is selected from the group consisting of tau aggregation inhibitors, tau protease inhibitors and tau kinase inhibitors.

17. The method according to claim 16 wherein the therapeutic agent is selected from the group consisting of rember and epothilone D.

18. The method according to claim 9 wherein the therapeutic agent regulates neurodegeneration.

19. The method according to claim 18 wherein the therapeutic agent is selected from the group consisting of: nicotinic acetylcholine receptor agonists, alpha 7 receptor agonists, ion channel modulators and 5HT receptor modulators.

20. The method according to claim 19 wherein the therapeutic agent is selected from the group consisting of alpha-4 beta-2 receptor agonists.

21. The method according to claim 19 where in the therapeutic agent is selected from the group consisting of Lu AE8054, EVP-6124, A-582941, GTS-21, AZD 3480, MEM3454, ABT-560 and ABT-894.

22. The method according to claim 9 wherein the therapeutic agent regulates inflammation.

23. The method according to claim 22 wherein the therapeutic agent is selected from the group consisting of COX inhibitors and anti-oxidants.

24. The method according to claim 23 wherein the therapeutic agent is selected from the group consisting of naproxen, ibuprofen, diclofenac, indomethacin, nabumetone, piroxicam, celecoxib, and aspirin.

25. The method according to claim 9 wherein the therapeutic agent is selected from the group consisting of agents which inhibit neuronal cell death via inhibition of caspases, Par-4, FAS, Bax, Bad, p53; or are neurotrophic factors; or activate; telomerase, Bcl2, Bcl-X_{L}, Mn-SOD, inhibitor of apoptosis proteins, or NCKAP1.

26. The method according to claim 25 wherein the therapeutic agent is selected from the group consisting of BDNF, NGF, and bFGF.

27. The method according to claim 9 wherein the therapeutic agent is an anti-hypertensive.

28. The method according to claim 27 wherein the therapeutic agent is selected from the group consisting of angiotenisin-converting enzyme inhibitors and angiotensin II receptor blockers.

29. The method according to claim 28 wherein the therapeutic agent is selected from the group consisting of enalapril, ramipril, quinapril, perindopril, lisinopril, benazepril, imidapril, zofenopril, trandolapril, valsartan, telmisartan, losartan, irbesartan, azilsartan, and olmesartan.

30. The method according to claim 9 wherein the therapeutic agent is an antidiabetic agent.

31. The method according to claim 30 wherein the therapeutic agent is selected from the group consisting of insulin, metformin, rosiglitazone, pioglitazone, MSDC-0160, GLP-1 receptor agonists, GLP-1, GLP-1 analogues, DPP-IV inhibitors and sulfonylureas.

32. The method according to claim 9 wherein the therapeutic agent is an RXR nuclear receptor agonist or partial agonist.

33. The method according to claim 32 wherein the therapeutic agent is bexarotene.

34. The method according to claim 9 wherein the therapeutic agent crosses the blood brain barrier.

35. The method according to claim 1 wherein the subject possesses one or more risk factors for developing Alzheimer's disease selected from the group consisting of a family history of the disease, low cerebral glucose metabolism, a genetic predisposition for the disease, elevated serum cholesterol, adult-onset diabetes mellitus, elevated baseline hippocampal volume, elevated cerebrospinal fluid levels of total tau, elevated or lowered cerebrospinal fluid levels of phospho-tau, and lowered cerebrospinal fluid levels of A β(1-42).

36. The method according to claim 1 wherein the compound is:
((1S)-5-{5-ethyl-2-(4-methoxyphenyl)-1,3-oxazol-4-yl]ethoxy}-2,3-dihydro-1H-inden-1-yl)acetic acid, sodium salt.

37. The method according to claim 8 wherein said one or more additional therapeutic agent is used to treat or prevent cognitive impairment.
